SEQUENCE LISTING (53 sequences)

SEQ ID NO: 1 - DNA, 17701 bp, Homo sapiens
atcacttctccctgggctcccaggccctcctgcagcagcccccgcctgggccatgtcttcctcagatgaagagacgctcagtgagcggtcatgtcggagtgagcggtcttgtcggagtgagcgatcttacaggagcgagcggtcggggagcctgtctccctgtcccccaggggacaccttgccctggaacctgccactgcatgagcagaaaaagcggaaaagccaggattcggtgctggaccctgcagagcgtgctgtggtcagagtcgctgatgaacgggaccgggttcagaagaaaacgttcaccaagtgggtcaacaagcacttaatgaaggtccgcaagcacatcaatgatctttatgaagatctgcgggatggccataacctgatctctctgttggaggtcctctcaggcatcaaactgccccgggagaagggcaggatgcgttttcataggctgcagaatgtgcagattgccctggacttcctaaagcagcgacaggtgaaactagtgaatattcgcaatgatgacatcacagatggcaaccccaagttgaccctgggtctgatctggaccattattttgcatttccagatctctgacatctacattagtggagaatcaggggatatgtcagccaaggagaaactactcctgtggacccagaaggtgacagctggttacacaggaatcaaatgcaccaacttttcctcctgctggagtgatgggaagatgttcaatgcactcattcaccgataccgacccgatctagtagacatggagagggtgcaaatccaaagtaaccgagagaatctggaacaggcttttgaagtggcagaaagactgggggtcactcgcctgctggatgcagaagatgtggatgtgccatctccagatgaaaagtctgtaatcacttatgtgtcttcgatttatgatgccttccctaaagttcctgagggtggagaagggatcagtgctacggaagtggactccaggtggcaagaataccaaagccgagtggactccctcattccctggatcaaacagcatacaatactgatgtcagataaaacttttccccaaaaccctgttgaactaaaggcactttataaccaatatatacacttcaaagaaacagaaattctggccaaggagagagaaaaaggaagaattgaggaattatataaattactagaggtgtggattgaatttggccgaattaaactgcctcaaggttatcaccctaatgatgtggaagaagagtggggaaagctcatcatagagatgctggaacgagagaaatcacttcggccggctgtggagaggctggaattgctgctacagattgcaaacaaaatccagaatggtgctttgaactgtgaagaaaaactgacactagctaagaatacactgcaggctgatgctgctcacctggaatcaggacaaccggtacaatgtgagtcagatgtcattatgtacattcaggagtgtgaaggtctcatcaggcagctgcaggtggatctccagatcctgcgggatgagaattactaccagctagaagagctggcttttagggtcatgcgtcttcaggatgagctggtcaccttgcgtctagagtgtacaaacctgtaccggaagggtcatttcacttcacttgaattggttccaccctctactttaaccaccactcatctgaaagcagaacccttaaccaaggcaacccattcttcttctacctcctggttccgaaagcctatgactcgggctgaacttgtggccatcagctcctctgaagatgaaggcaatctccgatttgtgtatgaactactgtcttgggtagaagagatgcagatgaaactggagcgagcagagtggggcaatgacctgcctagtgtggagttgcagctagaaacacagcagcacatccatacgagtgtagaagagctgggctcaagtgtcaaggaggccaggttgtatgagggaaagatgtcccagaatttccataccagctatgctgaaactcttggaaagctggagacacagtattgtaaattgaaggaaacttctagcttccggatgaggcaccttcagagcctgcataaatttgtttccagagctacagctgagttgatctggttgaatgagaaggaggaggaggaactagcatatgactggagtgacaacaattccaatatctcagccaagagaaattacttctctgagttgacaatggaactggaggagaaacaggatgtgtttcgttctctacaagatacagcagaactactttcacttgagaaccacccagccaagcagacagtggaggcttacagtgctgctgtccagtcccagttgcagtggatgaagcagctgtgcctgtgtgttgagcagcatgtgaaagagaatactgcttattttcagttcttcagtgatgcacgagagctggagtcattcttgaggaacctccaagattccattaaacgaaaatattcctgtgaccacaacaccagcttatcccgccttgaagacctgctccaggactccatggatgaaaaggagcagcttatacagtccaagagttccgttgccagtctcgttgggagatcaaaaaccatcgttcagctaaaaccacgcagtccagaccatgtgttaaagaacaccatttctgtcaaggctgtctgtgactacaggcagatcgagattactatttgcaaaaatgatgaatgtgtgctagaagataattctcagcggaccaaatggaaagtgatcagccccacagggaacgaggcaatggtgccgtcagtctgcttcctcatccccccacccaataaggatgccattgagatggccagcagggtcgaacaatcttatcagaaggttatggccctttggcatcagctgcatgttaacaccaaaagccttatctcttggaactatctgcgtaaagaccttgaccttgtacagacctggaacctagaaaagcttcgatcctcagcaccaggggagtgccatcagattatgaagaaccttcaggcccactatgaagactttctgcaggatagtcgtgactctgtgctgttctcagtggctgatcgcttgcgcttggaagaggaggtggaagcttgtaaagcccgcttccagcacctgatgaagtccatggagaatgaggacaaagaggagactgtggccaagatgtacatttcagagttgaagaacatccggctacgcctggaggagtatgaacagagggtggtcaaacgaattcagtctctagccagctctaggactgacagagatgcctggcaggacaatgcattaaggattgcagagcaagagcacacccaggaggatttacagcaattgaggtcagacttggatgcagtttctatgaaatgtgacagctttctccatcagtctccatctagttcaagtgtcccaactctgcgctcagaactgaatctgctggtggagaagatggaccatgtctatggtctctctactgtatatctgaataagttaaagacagttgatgttatagtacgtagcatacaggatgctgaactcttggtcaaaggttatgagattaagctgagtcaagaagaagtagtactggcagatctctcagctctggaggcccattggtcgacattacggcactggcttagtgatgtgaaggacaagaattcagtgttttcagtcctggatgaggaaattgccaaggccaaggtagtggcagagcagatgagtcgtctgacaccagagcgaaatctggatttggagcgctatcaggaaaaaggctcccagctgcaggagcgttggcaccgagtcattgcccagctcgagattcgccaatctgagctagaaagtatccaggaagttctgggagattaccgagcctgccatggaactctcatcaagtggattgaggaaaccactgcccagcaggaaatgatgaagccaggccaggcagaggatagcagagtgctttcggagcagctcagccagcagacggccctatttgcagaaattgagagaaatcagacaaaactggatcaatgtcaaaaattttcccagcagtactctactattgtaaaggactatgaattgcaactgatgacatacaaggcctttgtggaatcgcagcagaaatcccctggcaagcgccgtcgcatgctttcctcttcagatgccatcactcaagagttcatggacttaaggactcgctacacggcattggtgactttaacaactcagcacgtgaaatacatcagtgatgcactccggcgtctggaggaggaggagaaagtggtagaagaggagaaacaagaacatgtggagaaggttaaagaacttttgggctgggtgtctaccctagcgaggaatacacaaggaaaagctacctcatccgagaccaaagaatcaacagacattgaaaaagctattttggaacagcaggttctgtcagaagagctgacaacaaagaaagaacaagtctctgaagctattaaaacatcacagatcttcttggccaagcatggtcataagctctcagaaaaagagaagaaacaaatatctgagcaattgaatgccctaaacaaggcttaccatgacctttgtgatggttctgcaaatcagcttcagcagcttcagagccagttggctcaccagacagaacaaaagaccctgcagaaacaacaaaatacctgtcaccagcaactggaggatctttgcagttgggtaggacaggcagaaagagcactggcaggccaccaaggcagaaccacccagcaggatctctctgctttgcagaagaaccaaagtgacttgaaggatttacaggatgacattcagaatcgtgccacctcatttgccactgttgtcaaggacattgaggggttcatggaagagaatcagaccaagctgagcccacgtgagttgacagctcttcgggaaaagcttcatcaggctaaggagcaatatgaggcgctccaggaagagacacgtgtggcccagaaggaactggaggaagcagtgacctccgccttacagcaggagactgaaaagagtaaagcagcaaaggaactggcagagaacaagaagaagatcgatgctctcctggattgggtaacttcagtaggatcatctggtggacagctgctgaccaaccttccaggaatggagcagctctcgggagctagcttggagaaaggagccttggacaccactgatggttacatgggggtgaatcaagccccagagaaactggacaagcaatgtgagatgatgaaggcccgtcaccaagaattgctgtcccagcagcaaaatttcattctggccacccagtcagctcaggccttcttggatcagcatggccacaatctcacacctgaggagcaacagatgctgcaacagaagctgggagagctaaaggaacaatactctacttccctggcccaatcagaggcagaactgaagcaggtgcagacacttcaggatgagttgcagaaatttctgcaggatcataaagagtttgaaagctggttggaacgatccgagaaagagctggagaacatgcataagggaggcagcagccccgagacccttccctccctgctaaagcggcaaggaagcttctcagaggatgtcatttcccacaaaggagacttgagatttgtgactatctcaggacagaaagtcttggacatggaaaacagttttaaggaaggcaaagaaccatcagaaattggaaacttagtaaaggacaagttgaaggatgcaacagaaagatacactgctctccactcaaagtgtacacgattaggatctcacctgaatatgctgttaggccagtatcatcaattccaaaacagtgctgacagcctgcaggcctggatgcaggcttgtgaggccaacgtggagaagctcctctcagatactgttgcctctgaccctggagttctccaggagcagcttgcaacaacaaagcagttgcaggaggaattggctgagcaccaagtacctgtggaaaaactccaaaaagtagctcgtgacataatggaaattgaaggggagccagccccagaccacaggcatgttcaagaaactacagattccatactcagccacttccaaagcctctcctatagcctggctgagcgatcttctctgctgcagaaagcaattgcccaatctcagagtgtccaggaaagcctggagagcctgttgcagtctattggggaagttgaacaaaacctggaagggaagcaggtgtcatcactctcatcaggagtcatccaggaagccttagccacaaatatgaaattgaagcaggacattgctcggcaaaagagcagcttggaggccacccgtgagatggtgacccgattcatggagacagcagacagtactacagcagcagtgctgcagggcaaactggcagaggtgagccagcggttcgaacagctctgtctacagcagcaagaaaaggagagctccctaaagaagcttctaccccaggcagagatgtttgaacacctctctggtaagctgcagcagttcatggaaaacaaaagtcggatgctggcctctggaaatcagccagatcaagatattacacatttcttccaacagatccaggagctcaatttggaaatggaagaccaacaggagaacctagatactcttgagcacctggtcactgaactgagctcttgtggctttgcgctggacttgtgccagcatcaggacagggtacagaatctaagaaaagacttcacagagctacagaagacagttaaagagagagagaaagatgcatcatcttgccaggaacagttggatgaattccggaagctggtcaggaccttccagaaatggttgaaagaaactgaagggagtattccacctacggaaacttctatgagtgctaaagagttagaaaagcagattgaacacctgaagagtctactagatgactgggcaagtaagggaactctggtggaagaaatcaattgcaaaggtacttctttagaaaatctcatcatggaaatcacagcacctgattcccaaggcaagacaggttccatactgccctctgtaggaagctctgtaggcagtgtaaacggataccacacctgcaaagatctgacggagatccagtgtgacatgtcagatgtaaacttgaagtatgagaaactagggggagtacttcatgaacgccaggaaagccttcaggctatcctcaacagaatggaggaggttcacaaggaggcaaactctgtgctgcagtggctggaatcaaaagaggaagtcctgaaatccatggatgccatgtcatctccaaccaagacagaaacagtgaaagcccaagctgaatctaacaaggccttcctggctgagttggaacagaattctccaaaaattcaaaaagtaaaggaagccctggctggattactggtgacatatcccaactcacaggaagcagaaaattggaagaaaattcaggaagaactcaattcccgatgggaaagggccactgaggttactgtggctcggcaaaggcagctagaggaatctgcaagtcatctggcctgcttccaggctgcagaatcccagctccggccgtggctgatggagaaagaactgatgatgggagtgctggggcccctgtctattgaccccaacatgttgaatgcacaaaagcaacaggtccagtttatgctaaaggaatttgaagcacgcaggcaacagcatgagcaactgaatgaggcagctcagggcatcctaacaggccctggagatgtctctctgtccaccagccaagtacagaaagaactccagagcatcaatcagaaatgggttgagctgactgacaaactcaactcccgttccagccaaattgaccaagctattgttaagagcacccagtaccaggaactgctccaggacttatcagagaaggtgagggcagttggacaacggctgagtgtccagtcagctatcagcacccaaccagaggctgtaaagcagcaattggaagagaccagtgaaattcgatctgacttggagcagttagaccacgaggttaaggaggctcagacactgtgcgatgaactctcagtgctcattggtgagcagtacctcaaggatgaactgaagaagcgtttggagacagttgccctgcctctccaaggtttagaagaccttgcagccgatcgcattaacagactccaggcagctcttgccagcacccagcagttccagcaaatgtttgatgagttgaggacctggttggatgataaacaaagccagcaagcaaaaaactgcccaatttctgcaaaattggagcggctacagtctcagctacaggagaatgaagagtttcagaaaagtcttaatcaacacagtggctcctatgaggtgattgtggctgaaggggaatctctacttctttctgtacctcctggagaagagaaaaggactctacaaaaccagttggttgagctcaaaaaccattgggaagagcttagtaaaaaaactgcagacagacaatccaggctcaaggattgtatgcagaaagctcagaaatatcagtggcatgtggaagaccttgtgccatggatagaagattgtaaagctaagatgtctgagttgcgagtcactctggatccagtgcagctagagtccagtctcctaagatcaaaggctatgctgaatgaggtggagaagcgccgctccctgctggaaatattgaatagtgctgctgacattctgatcaattcttcagaagcagatgaggatggaatccgggatgagaaggctgggatcaaccagaacatggatgctgttacagaagagctgcaggccaaaacagggtcactcgaagaaatgactcagaggctcagggagttccaggaaagctttaagaatattgaaaagaaggttgaaggagccaaacaccaacttgagatctttgatgctctgggttctcaagcctgtagcaacaagaacctggagaagctaagagctcaacaggaagtgctgcaggccctagagcctcaggtagactatctgaggaactttactcagggtctggtagaagatgccccagatggatctgatgcttctcaacttctccaccaagctgaggtcgcccagcaagagttcctcgaagttaagcaaagagtgaacagtggttgtgtgatgatggaaaacaagctggaggggattggccagtttcactgccgggtccgagagatgttctctcaattggcagacctggatgatgagctagatggcatgggtgctattggcagagacactgatagcctccagtcccaaatcgaggatgtccggctattccttaacaaaattcacgtcctcaaattagacatagaggcctctgaagcagagtgtcgacatatgctagaagaagaggggactctggatttgttaggtctcaaaagggagctagaagccctgaacaaacagtgtggcaaactgacagagagggggaaagctcgtcaggaacagctggaactgacactaggccgtgtagaggacttctacaggaaattgaaaggactcaatgacgcgaccacagcagcagaggaggcagaggccctccagtgggtagtggggaccgaagtggaaatcatcaaccaacaattagcagattttaaaatgtttcagaaagaacaagtggatcctcttcagatgaaattgcagcaggtgaatggacttggccagggattaattcagagtgcaggaaaagactgtgatgtacagggtttagaacatgacatggaagagatcaatgctcgatggaatacattgaataaaaaggtcgcacaaagaattgcacagctacaggaagctttgttgcattgtgggaagtttcaagatgccttggagccattgctcagctggttggcagataccgaggagctcatagccaatcagaaacctccatctgctgagtataaagtggtgaaagcacagatccaagaacagaagttgctccagcggctcctagatgatcgaaaggccacagtagacatgcttcaagcagaaggaggcagaatagcccagtcagcagagctggctgatagagagaaaatcactggacagctggagagtcttgaaagtagatggactgaactactcagtaaggcagcagccaggcaaaaacagctggaagacatcctggttctggccaaacagttccatgagacagctgagcctatttctgacttcttatctgtcacagagaaaaagcttgctaactcagaacctgttggcactcagactgccaaaatacagcagcagatcattcggcacaaggctctggaagaagacatagaaaaccatgcaacagatgtgcaccaggcagtcaaaattgggcagtccctctcctccctgacatctcctgcagaacagggtgtgctgtcagaaaagatagactcattgcaggcccgatacagtgaaattcaagaccgctgttgtcggaaggcagccctacttgaccaagctctgtctaatgctaggctgtttggggaggatgaggtggaggtgctcaactggctggctgaggttgaggacaagctcagttcagtgttcgtaaaggatttcaaacaggatgtcctgcacaggcagcatgctgaccacctggctttaaatgaagaaattgttaatagaaagaagaatgtagatcaagctattaaaaatggtcaggctcttctaaaacaaaccacaggtgaggaggtgttacttatccaggaaaaactagatggtataaagactcgttacgcagacatcacagttactagctccaaggccctcagaactttagagcaagcccggcagctggccaccaagttccagtctacttatgaggaactgaccgggtggctgagggaggtggaggaggagctggcaaccagtggaggacagtctcccacaggggaacagataccccagtttcagcagagacagaaggaattaaagaaggaggtcatggagcacaggctggtgttggacacagtgaatgaggtgagccgtgctctcttagagctggtgccctggagagccagagaagggctggataaacttgtgtccgatgctaacgagcagtacaaactagtcagtgacactattggacaaagggtggatgaaattgatgctgctattcagagatcacaacagtatgagcaagctgccgatgcagaactagcttgggttgctgaaacaaaacggaaactgatggctctgggtccaattcgcctggaacaggaccagaccacagctcagcttcaggtacagaaggctttctccattgacattattcgacacaaagattcaatggatgaactcttcagtcaccgtagtgaaatctttggcacatgtggggaggagcaaaaaactgtattacaggaaaagacagagtctctaatacagcaatatgaagccattagcctactcaattcagagcgttatgcccgcctagagcgggcccaggtcttagtaaaccagttttgggaaacttatgaagagctcagcccctggattgaggaaactcgggcactaatagcacagttaccctctccagccattgatcatgagcagctcaggcagcaacaagaggaaatgaggcaattaagggaatctattgctgaacacaaacctcatattgacaaactactaaagataggcccacaactaaaggaattaaaccctgaggaaggggaaatggtggaagaaaaataccagaaagcagaaaacatgtatgcccaaataaaggaggaggtgcgccagcgagccctggctctggatgaagccgtgtcccagtccacacagattacagagtttcatgataaaattgagcctatgttggagacactggagaatctttcctctcgcctgcgtatgccaccactgatccctgctgaagtagacaagatcagagagtgcatcagtgacaataagagtgccaccgtggagctagaaaaactgcagccatcctttgaggccttgaagcgccgtggagaggagcttattggacgatctcagggagcagacaaggatctggctgcaaaagaaatccaggataaattggatcaaatggtattcttctgggaggacatcaaagctcgggctgaagaacgagaaatcaaatttcttgatgtccttgaattagcagagaagttctggtatgacatggcagctctcctgaccaccatcaaagacacccaggatattgtccatgacttggaaagcccaggcattgatccttccatcatcaaacaacaggttgaagctgctgagactattaaggaagagacagatggtctgcatgaagagctggagtttattcggatccttggagcagatttgatttttgcctgtggagaaactgagaagcctgaagtgaggaagagcattgatgagatgaataatgcttgggagaacttaaacaaaacatggaaagagaggctagaaaaacttgaggatgctatgcaagctgctgtgcagtatcaggacactcttcaggctatgtttgactggctagataacactgtgattaaactctgcaccatgccccctgttggcactgacctcaatactgttaaagatcagttaaatgaaatgaaggagttcaaagtagaagtttaccaacagcaaattgagatggagaagcttaatcaccagggtgaactgatgttaaagaaagctactgatgagacggacagagacattatacgagaaccactgacagaactcaaacacctctgggagaacctgggtgagaaaattgcccaccgacagcacaaactagaaggggctctgttggcccttggtcagttccagcatgccttagaggaactaatgagttggctgactcataccgaagagttgttagatgctcagagaccaataagtggagacccaaaagtcattgaagttgagctcgcaaagcaccatgtcctaaaaaatgatgttttggctcatcaagccacagtggaaacagtcaacaaagctggcaatgagcttcttgaatccagtgctggagatgatgccagcagcttaaggagccgtttggaagccatgaaccaatgctgggagtcagtgttacagaaaacagaggagagggagcagcagcttcagtcaactctgcagcaggcccagggcttccacagtgaaattgaagatttcctcttggaacttactagaatggagagccagctttctgcatctaagcccacaggaggacttcctgaaactgctagggaacagcttgatacacatatggaactctattcccagctgaaagccaaggaagagacttataatcaactacttgacaagggcagactcatgcttctaagccgtgacgactctgggtctggctccaagacagaacagagtgtagcacttttggagcagaagtggcatgtggtcagcagtaagatggaagaaagaaagtcaaagctggaagaggccctcaacttggcaacagaattccagaattccctacaagaatttatcaactggctcactctagcagagcagagtttaaacatcgcttctccaccaagcctgattctaaatactgtcctttcccagatagaagagcacaaggtttttgctaatgaagtaaatgctcatcgagaccagatcattgagctggatcaaactgggaatcaattaaagttccttagccaaaagcaggatgttgttctgatcaagaatttgttggtgagcgtgcagtctcgatgggagaaggttgtccagcgatctattgaaagagggcgatcactagatgatgccaggaagcgggcaaaacaattccatgaagcttggaaaaaactgattgactggctagaagatgcagagagtcacctggactcagaactagagatatccaatgacccagacaaaattaaacttcagctttctaagcataaggagtttcagaagactcttggtggcaagcagcctgtgtatgataccacaattagaactggcagagcactgaaagaaaagactttgcttcccgaagatagtcagaaacttgacaatttcctaggagaagtcagagacaaatgggatactgtttgtggcaagtctgtggagcggcagcacaagttggaggaagccctgctcttttcgggtcagttcatggatgctttgcaggcattggttgactggttatacaaggtggagccacagctggctgaggaccagcccgtgcacggggaccttgacctcgtcatgaacctcatggatgcacacaaggttttccagaaggaactgggaaagcgaacaggaaccgttcaggtcctgaagcggtcaggccgagagctgattgagaatagtcgagatgacaccacttgggtaaaaggacagctccaggaactgagcactcgctgggacactgtctgtaaactctctgtttccaaacaaagccggcttgagcaggccttaaaacaagcggaagtgtttcgagacacagtccacatgctgttggagtggctttctgaagcagagcaaacgcttcgctttcggggagcacttcctgatgacacagaggccctgcagtctctcattgacacccataaggaattcatgaagaaagtagaagaaaagcgagtggacgttaactcagcagtagccatgggagaagtcatcctggctgtctgccaccccgattgcatcacaaccatcaaacactggatcaccatcatccgagctcgcttcgaggaggtcctgacatgggctaagcagcaccagcagcgtcttgaaacggccttgtcagaactggtggctaatgctgagctcctggaagaacttctggcatggatccagtgggctgagaccaccctcattcagcgggatcaggagccaatcccgcagaacattgaccgagttaaagcccttatcgctgagcatcagacatttatggaggagatgactcgcaaacagcctgacgtggaccgggtcaccaagacatacaaaaggaaaaacatagagcctactcacgcgcctttcatagagaaatcccgcagcggaggcaggaaatccctaagtcagccaacccctcctcccatgccaatcctttcacagtctgaagcaaaaaacccacggatcaaccagctttctgcccgctggcagcaggtgtggctgttagcactggagcggcaaaggaaactgaatgatgccttggatcggctggaggagttgaaagaatttgccaactttgactttgatgtctggaggaaaaagtatatgcgttggatgaatcacaaaaagtctcgagtgatggatttcttccggcgcattgataaggaccaggatgggaagataacacgtcaggagtttatcgatggcattttagcatccaagttccccaccaccaagttagagatgactgctgtggctgacattttcgaccgagatggggatggttacattgattattatgaatttgtggctgctcttcatcccaacaaggatgcgtatcgaccaacaaccgatgcagataaaatcgaagatgaggttacaagacaagtggctcagtgcaaatgtgcaaaaaggtttcaggtggagcagatcggagagaataaataccggtttggggattctcagcagttgcggctggtccgtattctgcgcagcaccgtgatggttcgcgttggtggaggatggatggccttggatgaatttttagtgaaaaatgatccctgccgagcacgaggtagaactaacattgaacttagagagaaattcatcctaccagagggagcatcccagggaatgacccccttccgctcacggggtcgaaggtccaaaccatcttcccgggcagcttcccctactcgttccagctccagtgctagtcagagtaaccacagctgtacatccatgccatcttctccagccaccccagccagtggaaccaaggttatcccatcatcaggtagcaagttgaaacgaccaacaccaacttttcattctagtcggacatcccttgctggtgataccagcaatagttcttccccggcctccacaggtgccaaaactaatcgggcagaccctaaaaagtctgccagtcgccctgggagtcgggctgggagtcgagccgggagtcgagccagcagccggcgaggaagtgacgcttctgactttgacctcttagagacgcagtctgcttgttccgacacttcagaaagcagcgctgcagggggccaaggcaactccaggagagggctaaacaaaccttccaaaatcccaaccatgtctaagaagaccaccactgcctcccccaggactccaggtcccaagcgataacactgtctaagcacccccaagccactatccactttgaatcctgctccatacattgggtgtatatttattctgaacgggagaagttatattgttaaaagtgtaaaagaataattgtgttatgaagctgccttattttttttctttttgtaagttactattttcatgtgaatatttatgtagataaaatttgcctcctggtaaccctgtaatggatggggcccagaaatgaaatatttgagaaaaacaagtgaaaaggtcaagatacaaatgtgtattaaaaaaaaaaaagcctattaatagggtttctgcgcggtgcagggttgtaaacctgctttatcttttaggattattcctaaatgcatcttctttataaacttgacttgctatctcagcaagataaattatattaaaaaaataagaatcctgcagtgtttaaggaactctttttttgtaaatcacggacacctcaattagcaagaactgaggggagggctttttccattgtttaatgttttgtgatttttagctaaagagagggaacctcatctaagtaacatttgcacatgatacagcaaaaggagttcattgcaatactgtctttggatattgtttcagtactgggtgtttaaaggacaaatagctgctagaattcaggggtaaatgtaagtgttcagaaaacgtcagaacatttggggttttaaactgatttgttgctccctatccagcctagacaccagtaactcttgtgttcaccaggacccagacccttggcaagggataggctcgttggtgacattgtgaatttcagatttgttttatccactttttttgctatttatttaaatggtcgatcaacttcccacaaactgaggaatgaattccacgagcctgttctgaaaatgtggacgtaagacaaacacgtgctcgtcctttaatggagttcaccagcacacttgttaaccagtcctgtttgctttcgtctttttttgtgcgtaataaagtcaactgaccaagtgaccatgaaaaggggctgtctggggctcctgttttttagctgctgttcttcagctccgaccatgttgctgtgtgattatctcaattggttttaattgaggcagaaactgaagctctaccaatgaactgtttagaaacaagacacacttttgtattaaaattgcttgcagtaacaaatattttgtatttcctgattttcttttcaactattaccttatctataaatgttaccctggggtataatcatgttgtaggtacttaaatgcattccgcaaatcaaaatatcttgatggataaattatagagcttaatagatcttgttttatttcaaaaaaaaaaaaa

SEQ ID NO: 2 - protein, 5430 aa, Homo sapiens
MSSSDEETLSERSCRSERSCRSERSYRSERSGSLSPCPPGDTLPWNLPLHEQKKRKSQDSVLDPAERAVVRVADERDRVQKKTFTKWVNKHLMKVRKHINDLYEDLRDGHNLISLLEVLSGIKLPREKGRMRFHRLQNVQIALDFLKQRQVKLVNIRNDDITDGNPKLTLGLIWTIILHFQISDIYISGESGDMSAKEKLLLWTQKVTAGYTGIKCTNFSSCWSDGKMFNALIHRYRPDLVDMERVQIQSNRENLEQAFEVAERLGVTRLLDAEDVDVPSPDEKSVITYVSSIYDAFPKVPEGGEGISATEVDSRWQEYQSRVDSLIPWIKQHTILMSDKTFPQNPVELKALYNQYIHFKETEILAKEREKGRIEELYKLLEVWIEFGRIKLPQGYHPNDVEEEWGKLIIEMLEREKSLRPAVERLELLLQIANKIQNGALNCEEKLTLAKNTLQADAAHLESGQPVQCESDVIMYIQECEGLIRQLQVDLQILRDENYYQLEELAFRVMRLQDELVTLRLECTNLYRKGHFTSLELVPPSTLTTTHLKAEPLTKATHSSSTSWFRKPMTRAELVAISSSEDEGNLRFVYELLSWVEEMQMKLERAEWGNDLPSVELQLETQQHIHTSVEELGSSVKEARLYEGKMSQNFHTSYAETLGKLETQYCKLKETSSFRMRHLQSLHKFVSRATAELIWLNEKEEEELAYDWSDNNSNISAKRNYFSELTMELEEKQDVFRSLQDTAELLSLENHPAKQTVEAYSAAVQSQLQWMKQLCLCVEQHVKENTAYFQFFSDARELESFLRNLQDSIKRKYSCDHNTSLSRLEDLLQDSMDEKEQLIQSKSSVASLVGRSKTIVQLKPRSPDHVLKNTISVKAVCDYRQIEITICKNDECVLEDNSQRTKWKVISPTGNEAMVPSVCFLIPPPNKDAIEMASRVEQSYQKVMALWHQLHVNTKSLISWNYLRKDLDLVQTWNLEKLRSSAPGECHQIMKNLQAHYEDFLQDSRDSVLFSVADRLRLEEEVEACKARFQHLMKSMENEDKEETVAKMYISELKNIRLRLEEYEQRVVKRIQSLASSRTDRDAWQDNALRIAEQEHTQEDLQQLRSDLDAVSMKCDSFLHQSPSSSSVPTLRSELNLLVEKMDHVYGLSTVYLNKLKTVDVIVRSIQDAELLVKGYEIKLSQEEVVLADLSALEAHWSTLRHWLSDVKDKNSVFSVLDEEIAKAKVVAEQMSRLTPERNLDLERYQEKGSQLQERWHRVIAQLEIRQSELESIQEVLGDYRACHGTLIKWIEETTAQQEMMKPGQAEDSRVLSEQLSQQTALFAEIERNQTKLDQCQKFSQQYSTIVKDYELQLMTYKAFVESQQKSPGKRRRMLSSSDAITQEFMDLRTRYTALVTLTTQHVKYISDALRRLEEEEKVVEEEKQEHVEKVKELLGWVSTLARNTQGKATSSETKESTDIEKAILEQQVLSEELTTKKEQVSEAIKTSQIFLAKHGHKLSEKEKKQISEQLNALNKAYHDLCDGSANQLQQLQSQLAHQTEQKTLQKQQNTCHQQLEDLCSWVGQAERALAGHQGRTTQQDLSALQKNQSDLKDLQDDIQNRATSFATVVKDIEGFMEENQTKLSPRELTALREKLHQAKEQYEALQEETRVAQKELEEAVTSALQQETEKSKAAKELAENKKKIDALLDWVTSVGSSGGQLLTNLPGMEQLSGASLEKGALDTTDGYMGVNQAPEKLDKQCEMMKARHQELLSQQQNFILATQSAQAFLDQHGHNLTPEEQQMLQQKLGELKEQYSTSLAQSEAELKQVQTLQDELQKFLQDHKEFESWLERSEKELENMHKGGSSPETLPSLLKRQGSFSEDVISHKGDLRFVTISGQKVLDMENSFKEGKEPSEIGNLVKDKLKDATERYTALHSKCTRLGSHLNMLLGQYHQFQNSADSLQAWMQACEANVEKLLSDTVASDPGVLQEQLATTKQLQEELAEHQVPVEKLQKVARDIMEIEGEPAPDHRHVQETTDSILSHFQSLSYSLAERSSLLQKAIAQSQSVQESLESLLQSIGEVEQNLEGKQVSSLSSGVIQEALATNMKLKQDIARQKSSLEATREMVTRFMETADSTTAAVLQGKLAEVSQRFEQLCLQQQEKESSLKKLLPQAEMFEHLSGKLQQFMENKSRMLASGNQPDQDITHFFQQIQELNLEMEDQQENLDTLEHLVTELSSCGFALDLCQHQDRVQNLRKDFTELQKTVKEREKDASSCQEQLDEFRKLVRTFQKWLKETEGSIPPTETSMSAKELEKQIEHLKSLLDDWASKGTLVEEINCKGTSLENLIMEITAPDSQGKTGSILPSVGSSVGSVNGYHTCKDLTEIQCDMSDVNLKYEKLGGVLHERQESLQAILNRMEEVHKEANSVLQWLESKEEVLKSMDAMSSPTKTETVKAQAESNKAFLAELEQNSPKIQKVKEALAGLLVTYPNSQEAENWKKIQEELNSRWERATEVTVARQRQLEESASHLACFQAAESQLRPWLMEKELMMGVLGPLSIDPNMLNAQKQQVQFMLKEFEARRQQHEQLNEAAQGILTGPGDVSLSTSQVQKELQSINQKWVELTDKLNSRSSQIDQAIVKSTQYQELLQDLSEKVRAVGQRLSVQSAISTQPEAVKQQLEETSEIRSDLEQLDHEVKEAQTLCDELSVLIGEQYLKDELKKRLETVALPLQGLEDLAADRINRLQAALASTQQFQQMFDELRTWLDDKQSQQAKNCPISAKLERLQSQLQENEEFQKSLNQHSGSYEVIVAEGESLLLSVPPGEEKRTLQNQLVELKNHWEELSKKTADRQSRLKDCMQKAQKYQWHVEDLVPWIEDCKAKMSELRVTLDPVQLESSLLRSKAMLNEVEKRRSLLEILNSAADILINSSEADEDGIRDEKAGINQNMDAVTEELQAKTGSLEEMTQRLREFQESFKNIEKKVEGAKHQLEIFDALGSQACSNKNLEKLRAQQEVLQALEPQVDYLRNFTQGLVEDAPDGSDASQLLHQAEVAQQEFLEVKQRVNSGCVMMENKLEGIGQFHCRVREMFSQLADLDDELDGMGAIGRDTDSLQSQIEDVRLFLNKIHVLKLDIEASEAECRHMLEEEGTLDLLGLKRELEALNKQCGKLTERGKARQEQLELTLGRVEDFYRKLKGLNDATTAAEEAEALQWVVGTEVEIINQQLADFKMFQKEQVDPLQMKLQQVNGLGQGLIQSAGKDCDVQGLEHDMEEINARWNTLNKKVAQRIAQLQEALLHCGKFQDALEPLLSWLADTEELIANQKPPSAEYKVVKAQIQEQKLLQRLLDDRKATVDMLQAEGGRIAQSAELADREKITGQLESLESRWTELLSKAAARQKQLEDILVLAKQFHETAEPISDFLSVTEKKLANSEPVGTQTAKIQQQIIRHKALEEDIENHATDVHQAVKIGQSLSSLTSPAEQGVLSEKIDSLQARYSEIQDRCCRKAALLDQALSNARLFGEDEVEVLNWLAEVEDKLSSVFVKDFKQDVLHRQHADHLALNEEIVNRKKNVDQAIKNGQALLKQTTGEEVLLIQEKLDGIKTRYADITVTSSKALRTLEQARQLATKFQSTYEELTGWLREVEEELATSGGQSPTGEQIPQFQQRQKELKKEVMEHRLVLDTVNEVSRALLELVPWRAREGLDKLVSDANEQYKLVSDTIGQRVDEIDAAIQRSQQYEQAADAELAWVAETKRKLMALGPIRLEQDQTTAQLQVQKAFSIDIIRHKDSMDELFSHRSEIFGTCGEEQKTVLQEKTESLIQQYEAISLLNSERYARLERAQVLVNQFWETYEELSPWIEETRALIAQLPSPAIDHEQLRQQQEEMRQLRESIAEHKPHIDKLLKIGPQLKELNPEEGEMVEEKYQKAENMYAQIKEEVRQRALALDEAVSQSTQITEFHDKIEPMLETLENLSSRLRMPPLIPAEVDKIRECISDNKSATVELEKLQPSFEALKRRGEELIGRSQGADKDLAAKEIQDKLDQMVFFWEDIKARAEEREIKFLDVLELAEKFWYDMAALLTTIKDTQDIVHDLESPGIDPSIIKQQVEAAETIKEETDGLHEELEFIRILGADLIFACGETEKPEVRKSIDEMNNAWENLNKTWKERLEKLEDAMQAAVQYQDTLQAMFDWLDNTVIKLCTMPPVGTDLNTVKDQLNEMKEFKVEVYQQQIEMEKLNHQGELMLKKATDETDRDIIREPLTELKHLWENLGEKIAHRQHKLEGALLALGQFQHALEELMSWLTHTEELLDAQRPISGDPKVIEVELAKHHVLKNDVLAHQATVETVNKAGNELLESSAGDDASSLRSRLEAMNQCWESVLQKTEEREQQLQSTLQQAQGFHSEIEDFLLELTRMESQLSASKPTGGLPETAREQLDTHMELYSQLKAKEETYNQLLDKGRLMLLSRDDSGSGSKTEQSVALLEQKWHVVSSKMEERKSKLEEALNLATEFQNSLQEFINWLTLAEQSLNIASPPSLILNTVLSQIEEHKVFANEVNAHRDQIIELDQTGNQLKFLSQKQDVVLIKNLLVSVQSRWEKVVQRSIERGRSLDDARKRAKQFHEAWKKLIDWLEDAESHLDSELEISNDPDKIKLQLSKHKEFQKTLGGKQPVYDTTIRTGRALKEKTLLPEDSQKLDNFLGEVRDKWDTVCGKSVERQHKLEEALLFSGQFMDALQALVDWLYKVEPQLAEDQPVHGDLDLVMNLMDAHKVFQKELGKRTGTVQVLKRSGRELIENSRDDTTWVKGQLQELSTRWDTVCKLSVSKQSRLEQALKQAEVFRDTVHMLLEWLSEAEQTLRFRGALPDDTEALQSLIDTHKEFMKKVEEKRVDVNSAVAMGEVILAVCHPDCITTIKHWITIIRARFEEVLTWAKQHQQRLETALSELVANAELLEELLAWIQWAETTLIQRDQEPIPQNIDRVKALIAEHQTFMEEMTRKQPDVDRVTKTYKRKNIEPTHAPFIEKSRSGGRKSLSQPTPPPMPILSQSEAKNPRINQLSARWQQVWLLALERQRKLNDALDRLEELKEFANFDFDVWRKKYMRWMNHKKSRVMDFFRRIDKDQDGKITRQEFIDGILASKFPTTKLEMTAVADIFDRDGDGYIDYYEFVAALHPNKDAYRPTTDADKIEDEVTRQVAQCKCAKRFQVEQIGENKYRFGDSQQLRLVRILRSTVMVRVGGGWMALDEFLVKNDPCRARGRTNIELREKFILPEGASQGMTPFRSRGRRSKPSSRAASPTRSSSSASQSNHSCTSMPSSPATPASGTKVIPSSGSKLKRPTPTFHSSRTSLAGDTSNSSSPASTGAKTNRADPKKSASRPGSRAGSRAGSRASSRRGSDASDFDLLETQSACSDTSESSAAGGQGNSRRGLNKPSKIPTMSKKTTTASPRTPGPKR

SEQ ID NO: 3 - DNA, 4153 bp, Homo sapiens
aaacctccacgaaaataaggctcaccttgcgtaaccacgtagtccttcgccgcattggggcaaaataatcccttcatttttgtgaaggtaccgtggaaaatatttcatttttcttctcaccggagcaattgtaaatgctatgcggtaagaggagttacctgtggaaaggtggttaagagattaggtaaagaaaaggaaaggacaccaaaataaagtgctgcggaagaatttttgtccagctgtgagacgacgagtgcgtgaagtgaaggcgattgagaggggctgagggaattgtcctctgtggaagggactttcttttggccctaggccccttcctgcccctgtcgtcagcagagtctctacaaggaagataacggactgtaaaattctataaagcaaagctacacatcacttgacaccatacaccatcttggttacataatgaagagagatggaaaacttgatgactagctccaccctaccgcccctttttgcagatgaagacggttccaaggagagtaatgatctggctaccactgggttaaatcacccagaggttccatacagtagtggcgccacatcatccaccaacaatccagaatttgtggaggatctctctcaaggtcagttgcttcagagtgagtcttcaaatgcagcagaaggcaatgaacagaggcatgaagatgagcaacgaagtaaacgaggaggttggtccaaaggaagaaagaggaagaaacctcttcgagacagcaatgcacccaaatccccccttacaggatatgttcggttcatgaatgagcgtcgagaacaacttcgagcaaagagaccagaagtcccatttccagaaatcacaaggatgttaggcaatgaatggagtaaactgcctcctgaggaaaaacagcgctaccttgatgaagcagacagagataaggagcgttacatgaaggaactggaacagtatcagaaaacagaggcctacaaggtcttcagtaggaaaacccaggaccgtcagaaaggcaaatctcataggcaagatgcagcccggcaggccactcatgatcatgagaaagaaacagaggtaaaggaacggtctgtttttgacatccctatatttacagaggaattcttgaaccatagcaaagctcgggaagcagagctccgccagcttcgcaaatccaacatggagtttgaggagaggaatgcagccctgcaaaagcacgtggagagcatgcgcacagcagtggagaagctggaggtggatgtgatccaggagcggagccgcaacacagtcttacagcagcacctggagaccctgcggcaggtgctgaccagcagctttgccagcatgcccttgcctggaagtggagagacacctacagtggacaccattgactcatatatgaacagactgcacagtattattttagctaatccccaagacaatgaaaacttcatagctacagttcgagaagttgtgaacagactcgatcgttagggaatggtcttagaactccaagatgttccataagtgtttttacttgtgaggaatgagaagccatccatggaaatttgaactgagtgggggcagagaaagagtgcagatccctttgcttgtgaaagaattatcagtgagtgaaaggccatcaccccaggaagccaaatgagggagcagcaacatgtatatgagcttcctatggaattgtccttatgtgaagctttgaaggtgtacagccactctcccgggtcttcaggttcctaccatttccatttctgttaaagtggatctgcatatcttcagcttactaggtgacccggatgctgacatctgctgctgcagaaaggaagacttttcattgtaatttcgcttagacccttttatcagtggagctccagttttcttacctagctgtcacttttttaaatgcctctgggggttatttttgctttccttggcccccaccaatttatacatctccattttctgacctctggactaactggttgctcagcaaggttctgaaggagagtttcttgcattggacaggcccagtcttctcccatcattgccctgctgtgactccaaagaaaggagcttcttgctgacagtgccctgtggagcaaggctgtgtttcctaccccacacggtgctcagtgggtgccagccctcagtgtggctttgtgattgctgccctaaaggagaatgctctttccttcctcactggtactgcctgctgttttctaagcattgctcctgcacagacatggagtcccagccccagcaaggctcttctgttcccatctgttgacaatgtcttgtggagcatttttgctgaggaaaaggtcacttgtaaacagaggagaaagggaaagagtacaaagccctaagtttattgtaagtgaaaactgagggaattcctgtcttctttaggagtaatgattcatagatctagataggtggaaatatcattcaaaatagtcacttgagctcacaaaaaaagcaaggaagaattctcatgtcctttgtcttccttctgtagccattaactgctgaatccatgtgaggaagacaggcttcccttccttccccctccttagtgattttttctttaacagcataagtaaagaggactttctggttcatttttgtttgttttgttttgttttgttttgtttacagatgaggtcttcctgtgttgcccaggctggagtgcggtggctattcacagatgctatcatagcacactacagcctacaactcttgggctcaagcatcacgcctagcagtttctggttcctttaacagcaaaaggaaagagaggttctgattcttacctcagggttttttggttgttcattgtttttgtttttgtttttgttttgacactgcagagcacaaggctaaaggttacagctgagatctttggaaccaaaggcagagcaagcagagcccgttgtctgggccccacaccactgcaggcaggtggatagaagtgcggcccctctcatagtatgcccataagtcagggcatagggcagaactacctgtcatgttgctacaccatcctgtcttctcagcatctccttgcctgttttctttattagtccaaaggaaaacaacagcaacaaaatctgtttttaaaatgtcttatatgaacatatatcaaatatccatgcgctgaaacccacataccatcacttggcaattttttagaataagaccccattattatctattgctataaacctagccagttctcttgctcttctgtattttcctatttccctgccatcatctgctatttctgccacttctcttagactccttgtctgcaaagcccaagctagaactcactgtctatggcagaaggacatccagagcccattctggagttttgttttttccttctgccagatgctttgtgtcctgtcttccttcctcctcatatttctgtttctcatttgtgttcagttttgtgcagcattgctagcactgcttttgtgaccagaaaaggccataacatggtccaggatcatcattcttctgactctagatgggacacttgacagtgacttgaaacatttgcatattcaggaatgcatgagatttcaagagagcctacagtatgaaatcattttcacaaaataagcagcttgcttctgaaatgctgtctttcccagtagctactcacctgcctctggtggctgggattcagatgccacaaaactgtcagtatctatagaccaggtctgtgccacctcctctctcctctgtgctcagtgaggaggcagtaaatgaagttacaggctagcacaatacctaactcatgtttcccagtacacctgtagatattactgtacttttatgttctcaagaaataagttgttgcctattcagtgttacagatttctttgtttctttttaattaaaatacaagaagcagctgaggaaagggagacaaggtattttatttctgactgattttagaaaaaacttgtgtacatgtgtttggaactgttgaaatgccaagttttctgtataagtgtttttgtaattaaactttcagattttctttgttttttaagaagttgatgtgcttgtttgacatttgtctcattaaaacttttctacgttgaaaaaaaaaaaaaaaaaaaa

SEQ ID NO: 4 - protein, 347 aa, Homo sapiens
MENLMTSSTLPPLFADEDGSKESNDLATTGLNHPEVPYSSGATSSTNNPEFVEDLSQGQLLQSESSNAAEGNEQRHEDEQRSKRGGWSKGRKRKKPLRDSNAPKSPLTGYVRFMNERREQLRAKRPEVPFPEITRMLGNEWSKLPPEEKQRYLDEADRDKERYMKELEQYQKTEAYKVFSRKTQDRQKGKSHRQDAARQATHDHEKETEVKERSVFDIPIFTEEFLNHSKAREAELRQLRKSNMEFEERNAALQKHVESMRTAVEKLEVDVIQERSRNTVLQQHLETLRQVLTSSFASMPLPGSGETPTVDTIDSYMNRLHSIILANPQDNENFIATVREVVNRLDR

SEQ ID NO: 5 - DNA, 2125 bp, Homo sapiens
aatccgtggtctggtacaggtttcagggcaaagcggccatgcgttccgggggccgcgggcgaccccgcctgcggctgggggaacgtggcctcatggagccactcttgccgccgaagcgccgcctgctaccgcgggttcggctcttgcctctgttgctggcgctggccgtgggctcggcgttctacaccatttggagcggctggcaccgcaggactgaggagctgccgctgggccgggagctgcgggtcccattgatcggaagcctccccgaagcccggctgcggagggtggtgggacaactggatccacagcgtctctggagcacttatctgcgccccctgctggttgtgcgaaccccgggcagcccgggaaatctccaagtcagaaagttcctggaggccacgctgcggtccctgacagcaggttggcacgtggagctggatcccttcacagcctcaacacccctggggccagtggactttggcaatgtggtggccacactggacccaagggctgcccgtcacctcacccttgcctgccattatgactcgaagctcttcccacccggatcgaccccctttgtaggggccacggattcggctgtgccctgtgccctgctgctggagctggcccaagcacttgacctggagctgagcagggccaaaaaacaggcagccccggtgaccctgcaactgctcttcttggatggtgaagaggcgctgaaggagtggggacccaaggactccctttacggttcccggcacctggcccagctcatggagtctatacctcacagccccggccccaccaggatccaggctattgagctctttatgcttcttgatctcctgggagcccccaatcccaccttctacagccacttccctcgcacggtccgctggttccatcggctgaggagcattgagaagcgtctgcaccgtttgaacctgctgcagtctcatccccaggaagtgatgtacttccaacccggggagccctttggctctgtggaagacgaccacatccccttcctccgcagaggggtacccgtgctccatctcatctccacgcccttccctgctgtctggcacacccctgcggacaccgaggtcaatctccacccacccacggtacacaacttgtgccgcattctcgctgtgttcctggctgaatacctggggctctagcgtgcttggccaatgactgtggagaggactgtgagagagaaggtcccagcgggggccagtgaagctcaggcaggatctgcctagggtgtgctggtttgtccttttcatacctttgtctcctaattgtgctacaattggaagaccttctttcttttgattgtctcaagctgccacccttcaaggacagggaagagaccactgtgggatgacagccagaggaataagaacttgctccctccccagaggtaaacacttggtccaaaggtttgcagggaccaaatactgttctttttttttttgagacggagtctcactgtgttgcccaggctggagtgcagtggtgcgatctcggctcactgcaaactccgcctcctgggttcacgccattctcctgcctcagcctcccaagtagctgggactacaggtgcccgccaccacgctggctaattttttgtatttttagtagagacggggtttcaccgtgttagccaggatggtctcgatctcctgaccttgtaatccgccagcctcggcctcccaaagtgctgggattacaggtgtgagccaccgcacctggcaaaatgctgttctttaagtcagcgaactgaaaaagtaaaagactgctgggtgtggtggctcacacctgtaatcccaacactttgagaggctgagggggaaggatcacccgaggtcaggagtttgagaccagcctggtcaacatggcgaaaccctgtctctactaaaaatacaataattagctgggcatggtggtgggcgcctgtaatctcagctgcttgggaggctgaggcaggagaatcacttgtacgcaggaggcggaggttgcagtgagccaagatcacaccactgcactccagcctgggcaacagagcgagactccatctcaataaataaataaataaataaatattttttaaaaa

SEQ ID NO: 6 - protein, 382 aa, Homo sapiens
MRSGGRGRPRLRLGERGLMEPLLPPKRRLLPRVRLLPLLLALAVGSAFYTIWSGWHRRTEELPLGRELRVPLIGSLPEARLRRVVGQLDPQRLWSTYLRPLLVVRTPGSPGNLQVRKFLEATLRSLTAGWHVELDPFTASTPLGPVDFGNVVATLDPRAARHLTLACHYDSKLFPPGSTPFVGATDSAVPCALLLELAQALDLELSRAKKQAAPVTLQLLFLDGEEALKEWGPKDSLYGSRHLAQLMESIPHSPGPTRIQAIELFMLLDLLGAPNPTFYSHFPRTVRWFHRLRSIEKRLHRLNLLQSHPQEVMYFQPGEPFGSVEDDHIPFLRRGVPVLHLISTPFPAVWHTPADTEVNLHPPTVHNLCRILAVFLAEYLGL

SEQ ID NO: 7 - DNA, 5476 bp, Homo sapiens
aagctcttgagctcctctacctcttagaaagcacaattgaatcagatatcatatgaaagacatacacacttcatgtaatgctacctgcaagtctccctagaaaagcagtttttgtaggtgaaaacaatgaagccaggtaatattgcaaggaggctgtaattttagcagacctaccaacaacactgatgtaggaagctcattattttaatttctggagccttttaattttttctttagaaagtgtataaataattgcagtgctgctttgcttccaaaactgggcagtgagttcaacaacaacgacaacaacagccgcagctcatcctggccgtcatggagtttcttgaaagaacgtatcttgtgaatgataaagctgccaagatgtatgctttcacactagaaagtgtggaactccaacagaaaccggtgaataaagatcagtgtcccagagagagaccagaggagctggagtcaggaggcatgtaccactgccacagtggctccaagcccacagaaaagggggcgaatgagtacgcctatgccaagtggaaactctgttctgcttcagcaatatgcttcattttcatgattgcagaggtcgtgggtgggcacattgctgggagtcttgctgttgtcacagatgctgcccacctcttaattgacctgaccagtttcctgctcagtctcttctccctgtggttgtcatcgaagcctccctctaagcggctgacatttggatggcaccgagcagagatccttggtgccctgctctccatcctgtgcatctgggtggtgactggcgtgctagtgtacctggcatgtgagcgcctgctgtatcctgattaccagatccaggcgactgtgatgatcatcgtttccagctgcgcagtggcggccaacattgtactaactgtggttttgcaccagagatgccttggccacaatcacaaggaagtacaagccaatgccagcgtcagagctgcttttgtgcatgcccttggagatctatttcagagtatcagtgtgctaattagtgcacttattatctactttaagccagagtataaaatagccgacccaatctgcacattcatcttttccatcctggtcttggccagcaccatcactatcttaaaggacttctccatcttactcatggaaggtgtgccaaagagcctgaattacagtggtgtgaaagagcttattttagcagtcgacggggtgctgtctgtgcacagcctgcacatctggtctctaacaatgaatcaagtaattctctcagctcatgttgctacagcagccagccgggacagccaagtggttcggagagaaattgctaaagcccttagcaaaagctttacgatgcactcactcaccattcagatggaatctccagttgaccaggaccccgactgccttttctgtgaagacccctgtgactagctcagtcacaccgtcagtttcccaaatttgacaggccaccttcaaacatgctgctatgcagtttctgcatcatagaaaataaggaaccaaaggaagaaattcatgtcatggtgcaatgcacattttatctatttatttagttccattcaccatgaaggaagaggcactgagatccatcaatcaattggattatatactgatcagtagctgtgttcaattgcaggaatgtgtatatagattattcctgagtggagccgaagtaacagctgtttgtaactatcggcaataccaaattcatctcccttccaataatgcatcttgagaacacataggtaaatttgaactcaggaaagtcttactagaaatcagtggaagggacaaatagtcacaaaattttaccaaaacattagaaacaaaaaataaggagagccaagtcaggaataaaagtgactctgtatgctaacgccacattagaacttggttctctcaccaagctgtaatgtgatttttttttctactctgaattggaaatatgtatgaatatacagagaagtgcttacaactaatttttatttacttgtcacattttggcaataaatccctcttatttctaaattctaacttgtttatttcaaaactttatataatcactgttcaaaaggaaatattttcacctaccagagtgcttaaacactggcaccagccaaagaatgtggttgtagagacccagaagtcttcaagaacagccgacaaaaacattcgagttgaccccaccaagttgttgccacagataatttagatatttacctgcaagaaggaataaagcagatgcaaccaattcattcagtccacgagcatgatgtgagcactgctttgtgctagacattgggcttagcattgaaactataaagaggaatcagacgcagcaagtgcttctgtgttctggtagcaactcaacactatctgtggagagtaaactgaagatgtgcaggccaacattctggaaatcctatgtcaatgggtttggtttggaacctggacttctgcatttttaaaagttacccagagatgcttctaaagatgagccatagtctagaagattgtcaaccacaggagttcattgagtgggacagctagacacatacattggcagctacaatagtatcatgaattgcaatgatgtagtggggtataaaaggaaagcgatggatattgccggatgggcatggccagtgatgtttcacgtcattgaggtgacagctctgctggactttgaattacatatggaggctctccaggaagacgaagaagagaaggacattctaggcaaaaagaagactaggcacaaggcacacttatgtttgtctgttagcttttagttgaaaaagcaaaatacatgatgcaaagaaacctctccacgctgtgatttttaaaactacatactttttgcaactttatggttatgagtattgtagagaacaggagataggtcttagatgatttttatgttgttgtcagactctagcaaggtactagaaacctagcaggcattaataattgttgaggcaatgactctgaggctatatctgggccttgtcattatttatcatttatatttgtatttttttctgaaatttgagggccaagaaaacattgactttgactgaggaggtcacatctgtgccatctctgcaaatcaatcagcaccactgaaataactacttagcattctgctgagctttccctgctcagtagagacaaatatactcatcccccacctcagtgagcttgtttaggcaaccaggattagagctgctcaggttcccaacgtctcctgccacatcgggttctcaaaatggaaagaatggtttatgccaaatcacttttcctgtctgaaggaccactgaatggttttgtttttccatattttgcataggacgccctaaagactaggtgacttggcaaacacacaagtgttagtataattctttgcttctgcttctttttgaaaatcatgtttagatttgattttaagtcagaaattcactgaatgtcaggtaatcattatggagggagatttgtgtgtcaaccaaagtaattgtcccatggccccagggtatttctgttgtttccctgaaattctgcttttttagtcagctagattgaaaactctgaacagtagatgtttatatggcaaaatgcaagacaatctacaagggagattttaaggattttgagatgaaaaaacagatgctactcaggggctttatgaaccatccatcaattctgaagttctgactctcccattaccctttccctggtgtggtcagaactccaggtcactggaagttagtggaatcatgtagttgaattctttacttcaagacattgtattctctccagctatcaaaacattaatgatcttttatgtcttttttttgttattgttatactttaagttctggggtacatgtgcggaacatgtaggtttgttacataggtatacatgtgccatggtggtttgctgcactcatcaacctgtcatctacattcttttatgtctgtctttcaaagcaacactctgttcttctgagtagtgaaatcaggtcaactttaccaccagcctccatttttaatatgcttcaccatcatccagcacctacttaagatttatctagggctctgtggtgatgttaggacccataaaagaaatttatgccttccatatgtttggttacagatgggaaatgggaatgttgaaggacatgaaagaaaggatgtttacacattaagcatcagttctgaagctagattgtctgagtttgaatcttagctcttccctttattagctctgtgacctcgagctagttacttaaatgctctgatcctctatttcctgatcagtgaaacctccctattcaaatgtgtgagagtttaataaattaggacacttaaaaatgttggagcagtgcatagcatgtagtgttcagtacatgttaaatgttgttttttattatgtacaaacatgagtgggcacagaattttaaatcatctcaacttttgagaaattttgagttatcaacaccgttcccacaagacagtggcaaaattattggtgagaattaaacagctgtttctcagaggaagcaatggaggcttgctgggataaaggcatttactgagaggctgttacctagtgagagtgatgaattaattaaaatagtcgaatccctttctgactgtctctgaaagcttccgcttttatctttgaagagcagaattgtcactccaaggacatttattaataaaaagaacaactgtccagtgcaatgaaggcaaagtcataggtctcccaagtcttaccccattcctgtgaaatatcaagttcttggcttttctctgtcatgtagcctcaactttctctgaccgggtgcatttctttctctggtttctaaattgccagtggcaaatttggatcacttacttaatatctgttaaattttgtgacccaacaaagtcttttagcactgtggtgtcaaaaagaaaaacacctcccaggcatatacattttatagattcctggagaatgttgctctccagctccatccccacccaatgaaatatgatccagagagtcttgcaaagagacaagcctcattttccacaattagctctaaagtgcctccaggaaatgattttctcagctcatctctctgtattccctgttttggatcacagggcaatctgtttaaatgactaattacagaaatcattaaaggcaccaagcaaatgtcatctctgaatacacacatcccaagctttacaaatcctgcctggcttgacagtgatgaggccacttaacagtccagcgcaggcggatgttaaaaaaaataaaaaggtgaccatctgcggtttagttttttaactttctgatttcacacttaacgtctgtcattctgttactgggcacctgtttaaattctattttaaaatgttaatgtgtgttgtttaaaataaaatcaagaaagagaga

SEQ ID NO: 8 - protein, 369 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 9 - DNA, 2300 bp, Homo sapiens
agagcggagcggtgggccgggggctggaggacaggtttgtgcgctggacgcaagcaccaggcgcagcctcgctcgccgagacccggccagaacgtgttacgagtcagtttttagtgaaaaaacattgagctaggagccaagacccatctcttcactattttggtattgtgcaagtcatcttacctctctggatctcagttgtctcatctgtaaaaaggagataaaaattatttacctgcctgaacatgaggtggaggaccatcctgctacagtattgctttctcttgattacatgtttacttactgctcttgaagctgtgcctattgacatagacaagacaaaagtacaaaatattcaccctgtggaaagtgcgaagatagaaccaccagatactggactttattatgatgaatatctcaagcaagtgattgatgtgctggaaacagataaacacttcagagaaaagctccagaaagcagacatagaggaaataaagagtgggaggctaagcaaagaactggatttagtaagtcaccatgtgaggacaaaacttgatgaactgaaaaggcaagaagtaggaaggttaagaatgttaattaaagctaagttggattcccttcaagatataggcatggaccaccaagctcttctaaaacaatttgatcacctaaaccacctgaatcctgacaagtttgaatccacagatttagatatgctaatcaaagcggcaacaagtgatctggaacactatgacaagactcgtcatgaagaatttaaaaaatatgaaatgatgaaggaacatgaaaggagagaatatttaaaaacattgaatgaagaaaagagaaaagaagaagagtctaaatttgaagaaatgaagaaaaagcatgaaaatcaccctaaagttaatcacccaggaagcaaagatcaactaaaagaggtatgggaagagactgatggattggatcctaatgactttgaccccaagacatttttcaaattacatgatgtcaatagtgatggattcctggatgaacaagaattagaagccctatttactaaagagttggagaaagtatatgaccctaaaaatgaagaggatgatatggtagaaatggaagaagaaaggcttagaatgagggaacatgtaatgaatgaggttgatactaacaaagacagattggtgactctggaggagtttttgaaagccacagaaaaaaaagaattcttggagccagatagctgggagacattagatcagcaacagttcttcacagaggaagaactaaaagaatatgaaaatattattgctttacaagaaaatgaacttaagaagaaggcagatgagcttcagaaacaaaaagaagagctacaacgtcagcatgatcaactggaggctcagaagctggaatatcatcaggtcatacagcagatggaacaaaaaaaattacaacaaggaattcctccatcagggccagctggagaattgaagtttgagccacacatttaaagtctgaagtccaccagaacttggaagaaagctgttaactcaacatctatttcatctttttagctcccttcctttttctctgctcaataaatattttaaaagcatatttgaaataaagggagatactttttaaatgaaaacactttttttgggacacagatattaaaggattgaagtttatcagaaccaggaagaaaacaaactcactgtctgctctctgctctcacattcacacggctcttttatttatttttttgttctcctttaatgatttaattaagtggctttatgccataatttagtgaaactattaggaactatttaagtgagaaaactctgcctcttgcttttaaattagattgctctcacttactcgtaaacataggtattcttttatgggtgcttatcattccttctttcaataaatgtctgtttgatattaacaattctggaaaggccacagtatttccctgtgtttcctggtaacgtttttctagttttggcaacctcaactgctagaaattcttcacctgaatcacttttgctaccacttcaggtcatttttcattcttttttattttgctctatactttatcatttaagattaggttatgttacatataacagaaaaaacaaagataacagtggtttaaactaaataggagtttttcttcttacataagtctagaagtaggtggtgtccaggttcctatctttctgctctgctatcctcagtgcatgatttttatcctcagattacctcactctcactgttcaagtttgctcctggag

SEQ ID NO: 10 - protein, 420 aa, Homo sapiens
MRWRTILLQYCFLLITCLLTALEAVPIDIDKTKVQNIHPVESAKIEPPDTGLYYDEYLKQVIDVLETDKHFREKLQKADIEEIKSGRLSKELDLVSHHVRTKLDELKRQEVGRLRMLIKAKLDSLQDIGMDHQALLKQFDHLNHLNPDKFESTDLDMLIKAATSDLEHYDKTRHEEFKKYEMMKEHERREYLKTLNEEKRKEEESKFEEMKKKHENHPKVNHPGSKDQLKEVWEETDGLDPNDFDPKTFFKLHDVNSDGFLDEQELEALFTKELEKVYDPKNEEDDMVEMEEERLRMREHVMNEVDTNKDRLVTLEEFLKATEKKEFLEPDSWETLDQQQFFTEEELKEYENIIALQENELKKKADELQKQKEELQRQHDQLEAQKLEYHQVIQQMEQKKLQQGIPPSGPAGELKFEPHI

SEQ ID NO: 11 - DNA, 9661 bp, Homo sapiens
ggatgaagagtaacgccattaccgccggagccgccgagagccttagccgacggaaactggacactggaccggcagcgccatgagactcctcccccgcttgctgctgcttctcttactcgtgttccctgccactgtcttgttccgaggcggccccagaggcttgttagcagtggcacaagatcttacagaggatgaagaaacagtagaagattccataattgaggatgaagatgatgaagccgaggtagaagaagatgaacccacagatttggtagaagataaagaggaagaagatgtgtctggtgaacctgaagcttcaccgagtgcagatacaactatactgtttgtaaaaggagaagattttccagcaaataacattgtgaagttcctggtaggctttaccaacaagggtacagaagattttattgttgaatccttagatgcctcattccgttatcctcaggactaccagttttatatccagaatttcacagctcttcctctgaacactgtagtgccaccccagagacaggcaacttttgagtactctttcattcctgcagagcccatgggcggacgaccatttggtttggtcatcaatctgaactacaaagatttgaacggcaatgtattccaagatgcagtcttcaatcaaacagttacagttattgaaagagaggatgggttagatggagaaacaatctttatgtatatgttccttgctggtcttgggcttctggttattgttggccttcatcaactcctagaatctagaaagcgtaagagacccatacagaaagtagaaatgggtacatcaagtcagaatgatgttgacatgagttggattcctcaggaaacattgaatcaaatcaataaagcttcaccaagaaggttgcccaggaaacgggcacagaagagatcagtgggatctgatgagtaaatgttcctttgtgcaacaattcggtctttacttaacctgccctaatatttttcggcctgatgggaattagtgcagagaagccatgtcaccatagaaggcaactcctacttgtgtgtggactgagcaatcagagtctgtggcgataatattgctgaaaatgcactgcattcatttttctaaagtaacaaatttggtttttttttaaaccattaaaatctatgtgtgtgcgtgtgtatgtatgtgagcagttggtcttaccagaatcattgttgaactacctgaaacaagtctttagaatactaaatataatgctgttgtctcttcctttttgacattttctgattttttcccccaaaactcagttaatatttacccactatgattattgatgtcctgccttgaacagttttaaagaaaacaatttttggaatagctcaaatttcaattgatggcacaaatcagcattttgttgttgttactgtattacaattagtattctaaaggcagaagcagaagtagctgctttttagcaatagaattgtttcagtattttgctgctgtttaatgcgcatcttcagaaaacttcccagtggcttcaaggaatttggggatctctctggcaacaaattgtgaaacatgaaatttctgctgactttaatatatgaaacctaatcctacccccttttttaacaaaaagaaactagtacatttgtgaaaattgtgttgtgttgtccattgttgctctagttctgacccagaggtagctctggagtgattttagacctactcactcagttgtgtgtaggtttttttgttttgttttgagagagaatttttctctccttaatagaagcatcctttttaaagagaagttgccttggtccacacactaagcagaaaaccaagttatcaggacagagatatttcccagttactcctaatcaatgaagaaagtgagttggatatttttaaagcagttaactaattttttcttacctaatcttttgggagttttgcttgttgatataacctttttagttaacctgaaagattccaaaaattgttcttaagtgcttgagactggaaccaaaattaaattgtacttcataaaatcctcttatagagttactcttgccctagattgtaaattaagtttggcattattgtcagactggatggagggtgaagtaaaatagtatgaacaattaagaggctctccccctcttgtctttaagccatattctcctacatgtattttataagaaaatgttaagtcaaattttagtggctctttaattcctgacctcttcattctccttttcagtataacctcccctatgctcatgcccacacagacaaaaaaacaaaacgaaatacacacagaaaaaagtctttccaaactgtttaagtatttaaacatctgagccaaagcagatagaagttattgtataattgttaatcactttgcaaataggggctatcaaattacctatattggcattgctggattataaactctatatctgtaatataaagtgtttgagtttttaattgggctgttatgatcagtagttgattttgagaaagctctatgagctctaagtaactgcatggttttttgtttaatgtaatataggagacccttcacattcccaaggaatatattccaaaacatttttgtgaatatctaagtttgtgaaactactagggcatgatacagtaaggtgtaattacagaatttacgaaatgtaaatggcctctacagagttttatggaatacctggtactaacgtaggcagctgcaaaaccacactgagttacagctgtcagccctcctcattcctaaataacttgccttacatatcagccctcccacttctgaagttcaaattagtgcctcggaaatgtagaatttattatttgtcatttttttttttttagcatagattgagaacagttgaactcttaaatcctcagatgccaggggtctgctctagcatcagtaagtatttagcagaaactaactccgtaatgaatggaattcaattccacacatggtttgttcaagcacacttaataagtagcctattttttaaatgtctttttaaaatgtaaatatttggatgaagtttttctttgttttgatatattcatttgctacaccaactatgttttcagaattcatcttttgaacaacttggtttcagaatatgtaaaatgactttaaggatcttgtgtatcaaacctatccccggatgtgtgagaataatgtgttcataaagcatggatctcgctttggttgtatagcttcctcatttacttcatggtcttacatagctggtgactttcagggctaatctgccctctaaagcattgtcccaggagaggaaaaggaaatgggacctcagaagtagaagcctcagggaaggagtaaagtagaaatcagaagaaaagaagcttcacttgatagtaataaggtttttaacttcaagtaccttcagaaaatgtgattttgataagaggaaagggcaaatttagaccttaaaaaatatggaagaactactgccttaaaagtgcatttgtggcacatcagcctagaactgtatcatggctgtgctggggagaagtaaatggtggtaatgtaacattgccacctttacttaatgatgtgttattttcgaggtacagtagatcaatatagtaataggcgagcctcatatatagcattcatcttgtacaatgatatccatacccttgatatgaaggaaaattgacttggtttgtgcatttgaatactgaaataattttttaaaactcagtgacacataccatctcttgccaagactagaccctgtattttagttcctaaattagatgtttaaatttaaaaacattttcagatgtacttaagtacttccatagtacttttttttttttttttttttttgccccctgagacggagtctctgtgtcacccaggctggagtgcagtggtgcgatctcggctcactgcaacttctgcctcttgggttcaagcagttctccctgtctcagcctcctgagtagcttggattacaggcgcccgtcaccgcacctgcctaatttttgcatttttagtagagacggggtttcgtcatttggccaggctggtcttgaactcctgaccacaggtgatacgcctgccttggcctcccaaagtgtgctgggattacaggcgtgagccactgtgcccggcccactgttcactttttgaatggcatcattttatagctgtagaactaaaatcaatgtttgccccaattttcttaagtaaaactctactttgagctcttacctccaacttagtaaaaagcagcttcacacacaaacaagattcttactggtggaatgttaggtttcgttgttaagttaatctgtctataagctcatccttagaggatatttgaggaggaagaacaccttgcagctgacttgcaaacatctaaataatttatttcgggtgcttatgaatgttactaatggattttgtatgaatttttatcccttttcatttatacaaaaacctgggcttttatgttaattatatcatctgaggttctaaggttttttttttagattttgaaatttagggataatagctcttaggtttgggtaccactttgctgcagtttaagaaagggggaagggaactcatttattaaacatcaatcacgtgctgtgttctgtttgttttctagtcatcatatcacacacctttacgacagctcactgaaggaaggtgatactgttcccattttgtagatggaatagacaaaacctgaatttaagtagcttgctcaaggttccatattgaatatggaaagttcaaatcatctcagtaatgaatataccatatatacttgctgtattgtatctatgataattcagttacccacaatacccttttaaatttctgttaatgacatacctttaaatgtctccttgatgaacagaatcatggtctttaaaaacattttcatgggttgattgcattttcaagctctaaaggattgaaagataaatcttcacgttaaaggtaagagtgaagtatctgctcttgggttacagaaccagatagtactagaactaagattacagggtaaagctgcttttatcttttttctttttctttttctttttttttttgacatggggtctcactgtattgcccaggctggaatgcagtggcatgatctcagctcacggcagcctctgcctcttgggctcaagcgattctcctgcttcagctttccaagtatttgggaccacaggcgcacaccacaggcctggctaatgtttttgttttgtttttggtagagacggggtttcaccatgttgccaggctggtctcgaactcctgagctcaagtgattcacccaccttggcctcacaaagtgtcaggcttacaggcgtgagccactgcgcccggctcacagggtaaggcttctgtctggtgtgttgtattacggattttgcttaataggcacagtgaggcattaaaaagaaaattcagtatgcctgtagaaaggataatccttgtttaaagtctccaaattgcagtcaaagatgttttgactgtgcctttttttgttcccctgctgtcccttatgtagacttctgtcagtacccatggcagcctgtcatcttgttgacatctccttctggactgtgagctctgtatctggcttgtttttcatccccagcttctagttcacaattaggtagaaccctattactctttgaagaaggaacaagaaaatgtgggccagttttcatttgccattcttccatgtgagttagtatggttcgtaagtattcctggtgatacgctagtattggcaattctgtgaggttgaacaaaggggtggtatggtgtgctagcgtgggaattaggagacctctgggtcttgacagtgccctggccactaagcaaaggcagttcatccttggagtctcaatgtgcttttttgttaattgagatatgcttgaagtatcagccctaaatagtctgattctgtgacctacaaacccttacttaattcagtgttactataaatgattcttcccttaaacctactttttacttagcaaaagagaaaaaaaaaaaaaggaaacgctcatgtcaggctgcctgggtttgaaccccagatcccctcttagttggaggcaagttgtatgatgcttcagcttttggttcctgcctcctaaggttgttaggagttactgtgtgtagctgcttagaacattgcctgggtctcagtaagccgcttaagtgactgctctcattttcgctgtaaagcaccatactgtaataacatcccatgaagcatggggcggggaagagtatatggtaccttatggactttgatgtggtggggtagtaggtaaattctgaatatgtaagctacatagtattctttttgtaactaaaggataaaagttttaagatgggcatgtaatatggctagcactggattttaatgatgagccagagtaataaggctggcaaggggagtttttgttttgtaataaatctcttacacctgcacatccagttctttttaaaaacacgtttgaagaggctccatatttctcagtgaagctgttggggttcatgttatttttgaaaatcatcttgcaatttatttcagcatcagactgaaccacccaaagaaaattagtctatttctgaacagttttttcagaaaccatattggtctgatcacccaacatttatagaaataggcgcttaaggcctagaggcatttcagaaaggagaatgagaaatactgtggtcaagagtagtgttcagatggagaacatcaagcatctgctctgcccttccacacacatcctcatttcatcttcacaactgccctaggtaaccttgttatctgtaatgaaacagcctcaaggagcacagaggcactcactcctcacgtttggtctgttgccatttccggcttggttggaatagggtaggagccttttggcagggagcacattctcagtaatgcagagtgcactcacctgggttctagcttcaacacttaggatttgcttgatattttgtattctctgaggcactgcctgtatttgtttctgctatcacagtccagagtcaagcttcatttataaagactgggcagggcatggtggctcactcctgtaattccagcactttgggaggctgaggtgggtggatcactttaggtcaggagttcaagaccagcctggccaacatggtgaaaccccatctctactaaaaaatacaaaaaggccgggcacggtggctcacacctgtaatcccaacactttgggaggccaaggtgggcagaacacctgaggtcaggagttcaagaccatcctggcgaacatagtgaaacctcgcctctactaaaaatacaaaaattagccaggtgtggtggtgcatgcctgtaatcccagctacttgggaggctgaggcaggagaattgcttaaacctgggaggtggagattgtggtgagctgagatcgtgccactgcactccagcctgggagatagaacgagactccatctcaaaaaagaacaaaaacaattagccgagcgaggtggtgcacgcctgtaatcccagctactcatgaggctgaggcaggagaatcacttgaacccaggaggaggaggttgtagtgagtcaaggttgcaccactgcactccagcctgggtgacagagtgagactgtctcaaaaaataagtacataaataataagtaaaagctactaacaattaaaaaataaataaataaagacaagactgtctggaaaatggctctcctaaaaggaccagttgccatcatccacagtggaagattcaaagcagttggtccttggtacgtatgagaagcggatttcattcccttgaattctacagagcagtttattagagtgaatgcattttaaggccttgcatttgatatgtcatccagttcataatcaagttgcctttttctggctaaaacataatgattatgtatttttctcatttggtcctacaagctgctggccctttgtccctccactgtgggaatcagatctagaggaggctgagcctgcagacacagcagtggccaaaaggtcactctaagtgttttgtcttgactccttacttgaagtccacccagctagcacacatctggtttatactgaagccccctgcctagaaatactcatttcaggaaccaccagtaagcatctgtgaccacacaggctttttgactgatggcttcccggatctggtttcaagggataaccccgtctgtgtgcatctatggtcttctctctacagcgaggactttgcagtgctgcttgtggtccacacaaggggctcagagctgagtctgaactgcttcatggtcaccagctcctgtcccttccagtcttgagaggcttttttctccagatggaacctttccttcccgccgttttctcggtctctggctgtttttctcttgtgcccgtctaattggacacctcctggcttccatctctgtggttctcctgcctcacttcctgttctgttgtttttccgttttgtcaaaatatctcctatgttcttggcttccttttcgtcgccaggttttcagctttcctttagctcttcttctaatatggcttctgcccacaaaagcctgctctgtcaggatctcatggttctccacttgccagaaccttcttcagcctcagttcctcggcctcaacttgtacgtttaacccattgaccaccaccccccaaattcaccttcatttctttgaccctgctcctcactccttttctgttgaggaatctgttgactaactccaggctcactcaggctcaccgtcctgctctctgcaccagcctttccagagcgtgccagttctcatggcttcatctgttaactgttgatcacttcagtcctgatttttagacctaaatggtttccttaacgccattctaactgcctgtgactcattttcacttacagtgtttattgtaacgccaaaccaacaaatcacaggtgcttgcttctctccataaatctccccagtctaactttttgtcattcaacatgactcgtttatccaacctgaaatcgcatatagccccaagtatggtgttttgtacacaggtatttaataagtgacttccagttttggctctgctatgaataaaaagagatttcagttctcttcactttgaaatctaacaactcagagaacattgaagaaattggaatttagttgggatgaaatacttgtggtttaaaatatttctgttcatattttctaatttgttgccggaggtcttgggttttctatttgagtgcttgcaaactcaatgtgatttctgtcagcatatcttaggtttgtttgttatgaaacttatgcagtgtgaggttctatctgaaaatgttatttagctatcttctgggactatttaatgaaagtggggtcatgaatccttaaaattcttgtgcagctttgagaaacatttctgttatttgggtatcagtttgtaagtgtggtaaagccaagatggaaacgagcactttgctttcttggttgttgttactggtctaacctcctgcttgaactagtctgctgtcctgtcaaatgcatctttttatttacatgtcccttaaattaaagctgatcatgaaagta

SEQ ID NO: 12 - protein, 286 aa, Homo sapiens
MRLLPRLLLLLLLVFPATVLFRGGPRGLLAVAQDLTEDEETVEDSIIEDEDDEAEVEEDEPTDLVEDKEEEDVSGEPEASPSADTTILFVKGEDFPANNIVKFLVGFTNKGTEDFIVESLDASFRYPQDYQFYIQNFTALPLNTVVPPQRQATFEYSFIPAEPMGGRPFGLVINLNYKDLNGNVFQDAVFNQTVTVIEREDGLDGETIFMYMFLAGLGLLVIVGLHQLLESRKRKRPIQKVEMGTSSQNDVDMSWIPQETLNQINKASPRRLPRKRAQKRSVGSDE

SEQ ID NO: 13 - DNA, 2140 bp, Homo sapiens
gggaggaacgcgccgctaggcgggagagcgcggccatggcggggccgggcgtccccggtgcccccgcagcgcgctggaaacgccacatcgtgcggcagctgcggcttcgggaccgtacgcaaaaggcgcttttcctggagctggtgccggcctataaccatctcttagagaaggctgagctgctggacaagttctcaaagaagctgcagccggagccaaacagtgtcactcccaccacccaccagggcccctgggaggagtcagagcttgactcagaccaagtcccatcactggtcgcactgagggtgaagtggcaggaggaggaggaggggctccggctggtctgtggtgagatggcctaccaggtggtggagaagggcgcggccctgggcacgctggagtcggagctgcagcagaggcaaagcaggctggcagccctggaggcccgcgtggcgcagctgcgagaggcgcgggcgcagcaggcccagcaggtggaggagtggcgggcgcagaatgcggtgcagcgggcagcctacgaggcgctgcgcgcgcacgtcgggctccgggaggcggcactgcgcaggctccaggaagaggcgcgcgacctgctggagaggctcgtgcagcgcaaggcgcgcgccgcggccgagcgcaacctgcgcaacgagcgccgggagcgggccaagcaggcgcgggtgtcccaggagctgaagaaggctgccaagcggaccgtgagcatcagcgagggcccggacaccctaggcgatgggatgagggagagaagggagactctggctctggcccctgagccagagcccctggagaaggaagcttgtgagaagtggaagaggcccttcaggtctgcctcagccacctccctgacgctgtcccactgtgtggatgtggtgaaggggcttctggattttaagaagaggagaggtcactcaattgggggagcccctgagcagcgataccagatcatccctgtgtgtgtggctgcccgacttcctacccgggctcaggatgtgctggatgcccacctctctgaggtcaatgctgttcgttttggccccaacagcagcctcctggccactggaggggctgaccgcctgatccacctctggaatgttgtgggaagtcgcctggaggccaaccagaccctggagggagctggtggcagcatcaccagtgtggactttgacccctcgggctaccaggttttagcagcaacttacaaccaggctgcccagctctggaaggtgggggaggcacagtccaaggagacactgtctggacacaaggataaggtgacagctgccaaattcaagctaacgaggcaccaggcagtgactgggagccgcgaccggacagtgaaggagtgggacctcggccgtgcctattgctccaggaccatcaatgtcctttcctactgtaatgacgtggtgtgtggggaccatatcatcattagtggccacaatgaccagaagatccggttctgggacagcagggggccccactgcacccaggtcatccctgtgcagggccgggtcacctccctgagcctcagccacgaccaactgcacctgctcagctgttcccgagacaacacactcaaggtcatcgacctgcgtgtcagcaacatccgccaggtgttcagggccgatggcttcaagtgtggttctgactggaccaaagctgtgttcagcccggacagaagctatgcactggcaggctcctgtgatggggccctttacatctgggatgtggacaccgggaaactggagagcagactacagggaccccattgcgctgccgtcaacgccgtggcctggtgctactccgggagccacatggtgagcgtggaccagggcaggaaggttgtgctctggcagtagggccacgacctgcctgcctgggctggagctcttgcccgaagcctgaagcttccttcggcgccatgcaggggttggggttgggactggagctggccttgggatttaatggggaagaaggcctggcaggacctggcctgtttgtttaaaaatgaagtatgggttgggggattacgctagtttttctttgtatttttatctctatctcctcactttttctcccaaagtagaaaaaaatgatatctgaa

SEQ ID NO: 14 - protein, 619 aa, Homo sapiens
MAGPGVPGAPAARWKRHIVRQLRLRDRTQKALFLELVPAYNHLLEKAELLDKFSKKLQPEPNSVTPTTHQGPWEESELDSDQVPSLVALRVKWQEEEEGLRLVCGEMAYQVVEKGAALGTLESELQQRQSRLAALEARVAQLREARAQQAQQVEEWRAQNAVQRAAYEALRAHVGLREAALRRLQEEARDLLERLVQRKARAAAERNLRNERRERAKQARVSQELKKAAKRTVSISEGPDTLGDGMRERRETLALAPEPEPLEKEACEKWKRPFRSASATSLTLSHCVDVVKGLLDFKKRRGHSIGGAPEQRYQIIPVCVAARLPTRAQDVLDAHLSEVNAVRFGPNSSLLATGGADRLIHLWNVVGSRLEANQTLEGAGGSITSVDFDPSGYQVLAATYNQAAQLWKVGEAQSKETLSGHKDKVTAAKFKLTRHQAVTGSRDRTVKEWDLGRAYCSRTINVLSYCNDVVCGDHIIISGHNDQKIRFWDSRGPHCTQVIPVQGRVTSLSLSHDQLHLLSCSRDNTLKVIDLRVSNIRQVFRADGFKCGSDWTKAVFSPDRSYALAGSCDGALYIWDVDTGKLESRLQGPHCAAVNAVAWCYSGSHMVSVDQGRKVVLWQ

SEQ ID NO: 15 - DNA, 1960 bp, Homo sapiens
gagacgctaagcggcgccgggcgggagaagagcggagcagtggtcggagatgtggcgaccggtgcagctctgtcatttccactctgctctgctgcacagcaggcagaagccctggccgtcccctgctgtgttcttcaggagaaacgtcaggggccttcctccaaggttctccagccccacacccctgtggaggaaggtgctctccaccgcggtagtgggggcgcccctgctcctcggagcccgctatgtcatggcagaggcacgggagaagaggaggatgcggctcgtggtggatggcatggggcgctttggcaggtctctgaaggtcggcctgcagatctccctggactactggtggtgcaccaatgttgtccttcgaggggtggaagagaacagcccaggctacttggaggtgatgtctgcgtgtcaccagcgggcggctgatgccctggtggcaggggccatcagcaacgggggcctctacgtgaagctgggccaggggctgtgctccttcaaccacctgcttccccccgagtatacccggaccctgcgcgtgctagaggacagggccctcaagcggggcttccaggaggtggatgagttgttccttgaggacttccaggccctcccccacgagctcttccaggagtttgactaccagccaattgctgccgccagcctggcacaggtgcacagagccaagctgcacgatggcaccagcgtggctgtgaaggtgcagtacatcgacctgcgggaccgctttgatggggacatccacaccctggagctcctgctgcggctcgttgaggtcatgcaccccagctttggcttcagctgggtcctccaggacctgaaggggaccctggcccaggagctggacttcgagaatgagggccgcaacgcagagcgctgtgcgcgggagctggcgcacttcccctacgtcgtggtgccccgcgtgcactgggacaagtccagcaagcgcgtgctcactgccgacttctgcgccggctgcaaggtcaacgatgtggaggccatcaggagccaggggctggcagtgcatgacatagcagaaaagctcatcaaggcctttgctgagcagatattttacaccggcttcatccactcggacccacatcctggcaacgttctggtgcggaaaggcccggacgggaaagcggagctggtgctgctggaccacgggctctaccagttcctggaggagaaggaccgcgcagccctctgccagctgtggcgggccatcatcctgcgggacgacgccgccatgagggcgcacgcagccgcactgggggtgcaagactacctcctgttcgccgagatgctcatgcagcgccccgtgcgcctggggcagctgtggggctcgcacctactgagccgcgaagaggcggcctacatggtggacatggcccgcgagcgcttcgaggccgtcatggcggtgctcagggagctgccgcggcccatgctgctggtgctgcgcaacatcaacaccgtgcgcgctatcaacgtggccctcggcgcccccgtggaccgctacttccttatggctaaaagggctgtccggggctggagccgcctggcgggcgccacgtatcggggtgtctacggcaccagcctcctgcgccacgccaaggtcgtctgggagatgctcaagtttgaagtggcgctcaggctggagaccttggccatgcggctgaccgccctcctggctcgtgctctggtccacctgagcctcgtgcccccagcggaggagctctaccagtacctggagacctagggtgcagccgcccagggccggcggggcccttttcaccttgggctgacggaggtggcggggctagaggtgtagacaccccgagccccgtgggcactcgcactggggggctgtgacagcagctgggccaggaggccgtgtaatgaccacacactcctctcaagcaaaaaa

SEQ ID NO: 16 - protein, 580 aa, Homo sapiens
MWRPVQLCHFHSALLHSRQKPWPSPAVFFRRNVRGLPPRFSSPTPLWRKVLSTAVVGAPLLLGARYVMAEAREKRRMRLVVDGMGRFGRSLKVGLQISLDYWWCTNVVLRGVEENSPGYLEVMSACHQRAADALVAGAISNGGLYVKLGQGLCSFNHLLPPEYTRTLRVLEDRALKRGFQEVDELFLEDFQALPHELFQEFDYQPIAAASLAQVHRAKLHDGTSVAVKVQYIDLRDRFDGDIHTLELLLRLVEVMHPSFGFSWVLQDLKGTLAQELDFENEGRNAERCARELAHFPYVVVPRVHWDKSSKRVLTADFCAGCKVNDVEAIRSQGLAVHDIAEKLIKAFAEQIFYTGFIHSDPHPGNVLVRKGPDGKAELVLLDHGLYQFLEEKDRAALCQLWRAIILRDDAAMRAHAAALGVQDYLLFAEMLMQRPVRLGQLWGSHLLSREEAAYMVDMARERFEAVMAVLRELPRPMLLVLRNINTVRAINVALGAPVDRYFLMAKRAVRGWSRLAGATYRGVYGTSLLRHAKVVWEMLKFEVALRLETLAMRLTALLARALVHLSLVPPAEELYQYLET

SEQ ID NO: 17 - DNA, 6098 bp, Homo sapiens
atgtccggctccaaaagcgtgagccccccgggctacgcggcgcagaagactgcggcgccggcgccccggggaggccccgaacaccgctctgcgtggggcgaggccgattcccgcgcgaatggctacccccatgcccccgggggctctgcccgcggctccaccaagaaacccgggggggcggtgaccccgcagcagcagcagcgcctggccagccgctggcgcagcgacgacgacgacgatcctccgctgagcggtgacgaccccctggccgggggcttcggcttcagcttccgctccaagtccgcctggcaggagcgcggcggcgacgactgcggtcgcggcagccgccggcagcggcggggcgcggccagcgggggcagcacccgggcgccccctgcgggcggcggcggcggctcggcggcggcggctgcctcggcgggcgggacggaggtgcgccctcgctcggtggaggtgggtctggaggagcggcggggcaaggggcgcgcggccgacgagctggaggccggcgccgtcgagggcggcgaggggtccggggatggcggcagctcggcggactcgggctcgggcgcggggcccggcgcggtgctgtccctgggcgcctgctgcctggcgttgctgcagatattccgctccaagaagttcccgtcggacaaactggagcggctgtaccagcgctacttcttccgcctgaaccagagcagcctcaccatgctcatggccgtgctggtgctcgtgtgcctggtcatgttggccttccacgcggcgcggcccccgctccagctgccctacctggccgtgctggcggccgccgtcggcgtgatcctcatcatggctgtgctttgcaaccgcgccgccttccaccaggaccacatgggcctggcctgctatgcgctcatcgccgtggtgctggccgtccaggtggtgggcctgctgctgccgcagccacgcagcgcctctgagggcatctggtggaccgtgttcttcatctacaccatctacacgctgctgcccgtgcgcatgcgggccgcagtgctcagcggggtgctcctgtccgccctccacctggccatcgccctgcgcaccaacgcccaggaccagttcctgctgaagcagcttgtctccaatgttctcattttctcctgcaccaacatcgtgggtgtctgcacccactatccggctgaggtctcccagagacaggctttccaggagacccgagagtgcatccaggcgcggctccactcgcagcgggagaaccagcagcaggaacggctcctgctgtctgtccttccccgtcatgttgccatggagatgaaagcagacatcaacgccaagcaggaggatatgatgttccataagatttacatccagaaacatgacaacgtgagcatcctgtttgctgacatcgagggcttcaccagcctggcgtcccagtgcactgcacaggaactggtcatgaccctcaacgagctcttcgcccgctttgacaagctggccgcagagaatcactgtttacgtattaagatccttggggattgttattactgcgtctcggggctgcctgaagcaagggctgaccacgcccactgctgtgtggagatgggcatggacatgatcgaggccatctcgttggtccgggaggtgacaggggtgaacgtgaacatgcgtgtgggaattcacagcgggcgagtacactgcggtgtccttggtctcaggaagtggcagttcgacgtctggtctaacgatgtcacgctagccaaccacatggaggctggcggcaaggcaggacgcatccacatcaccaaggctacactcaactacctgaatggggactacgaggtggagccaggctgtgggggcgagcgcaacgcctacctcaaggagcacagtatcgagaccttcctcatcctgcgctgcacccagaagcggaaagaagagaaggccatgatcgccaagatgaaccgccagagaaccaactccatcgggcacaacccaccacactggggggctgagcgccccttctacaaccacctgggtggcaaccaggtgtccaaggagatgaagcggatgggctttgaagaccccaaggacaagaacgcccaggagagtgcgaaccctgaggatgaagtggatgagtttctgggccgtgccattgacgccaggagcattgataggcttcggtctgagcacgtccgcaagttcctcctgaccttcagggagcctgacttagagaagaagtactccaagcaggtagacgaccgatttggtgcctatgtggcgtgtgcctcgctcgtcttcctcttcatctgctttgtccagatcaccatcgtgccccactccatattcatgctcagcttctacctgacctgttccctgctgctgaccttggtggtgtttgtgtctgtgatctactcctgcgtaaagctcttcccctccccactgcagaccctctccaggaagatcgtgcggtccaagatgaacagcaccctggttggggtgttcaccatcaccctggtgttcctggcggcttttgtcaacatgttcacgtgcaactccagggacctgctgggctgcttggcacaggagcacaacatcagcgcgagccaggtcaacgcgtgtcacgtggcggagtcggccgtcaactacagcctgggcgatgagcagggcttctgtggcagcccctggcccaactgcaacttccccgagtacttcacctacagcgtgctgctcagcctgctggcctgctccgtgttcctgcagatcagctgcatcgggaagctggtgctcatgctggccatcgagctcatctacgtgctcatcgtggaggtgccaggtgtcacgctcttcgacaacgccgacctgctggtcaccgccaacgccatagacttcttcaacaacgggacctcccagtgccctgagcatgcaaccaaggtggcattgaaggtggtgacgcccatcatcatctcagtctttgtgctggccctgtacctgcacgcccagcaggtggagtccactgcccgcctcgacttcctctggaaactgcaggccacagaggagaaagaggagatggaggagctgcaggcctacaaccggcggctgctgcacaacatcctgcccaaggacgtggccgctcacttcctggcccgcgagcggcgcaatgatgagctctactatcagtcctgtgagtgtgtggcggtcatgttcgcctccatcgccaacttctccgagttctacgttgagctggaggccaacaacgagggtgtcgagtgcctgcggctactcaatgagatcatcgctgactttgatgagatcatcagcgaggatcggttccggcagctggagaagatcaagaccatcggcagcacctacatggctgcctccggcctcaacgactctacctacgacaaggtgggcaagacccacatcaaggcactggccgactttgccatgaagctgatggaccagatgaagtacatcaatgagcactccttcaacaacttccagatgaagatcgggctcaacatcggccccgtggtggccggggtgataggggcacgaaagcctcagtacgacatctggggcaataccgtgaacgtggccagccgcatggacagcaccggtgtacccgaccgcatccaggtcaccacagacatgtaccaggtgctggctgccaacacgtaccagctggagtgccggggcgtggtcaaggtcaagggcaaaggcgagatgatgacctacttcctcaatggagggcccccgctcagttagcagctgttggccaatggtgccaggcagcctggcctccagaggcatggaagcagcttctctgtgtgccgggggtggcggggaagccatgctccagcccgcagggctgcgctgctgagattttccacttggactccagagcagcttctgcctttgctggtgggcagcggcctctgtcccaggccccggggtgccagcgtcctgcgagcacccagctgaccaaagatgtttccctctgtagaagactctgctagactgggtctgaagcttgagttttctaacaggtgctgctgcacaggtggaaaggagccgtgggaatgtgtgtgtggcacggcccagacaagggcagggctgaggggcctccgactcagctgggggtagacgggctcgaatgtggcctgggagagcctagggggccccaggggtctgcttttctatgtgagcctttaaacttcagacaggccaccaccctgcacctgcaggggctttggcacaggagtgctggctttggagggactgtggccttcatcgtggtcctctgcccacacctccacgcacacagacagtgccctaggagggaaacagaactaattacgagggggaggcaagaggacgccaagcaaggagtggtgattctgagaaaaatatttattaaataaaacaaaacaagttctccgtgcccttctttagactatgctagttgtatgcgtgtaagagacacacaagcaaacgaggacgccactcggggggagggcggggatccccacttgtcttttttgtattttttattttgtattattgaaagccttggagatctcacagatagatatgccaaattctatattttgtaaattctctatattagaaaacagctgtgcacagcagggcgggtgtgctcatttgtactgtgtgtatgtcggtgtatgtactggtgtatatgtgtgtgtgttcatgctgtggaactggtctcacacaggatgtgtttccctcatttcagatttggcagttttgggttttccaaggtaccaccagagcagtgggtgtgtgcttttggggtaccatatgctcagattaagtaggaggatgcatggacacactgccccatcttttctgacacacgcacacgtatgtacacacatgcacacaccctccttcccctaagcaaaacgcagatggaataagaaaacaaaaagctgctttcccatcccaggccgagctggaaccaagggaagcaatctcatcctgcgacaggcagtgtggtgccctccacaccctgagatttcagacgtttgcggcttacagaggcagcgcccacagattccagagtgcttacagaaggccaggtgctttgcaggctgggacgaggaagccaagcctccctggcctactcagttggccaaggtgcaggtggctcttcctggagatgttcactcagactgggggatgcaatgtgcagccttcaggtttgcggaaagggagtggccttgacctccaccggcaaaccaggcagaggaatgggtagagcccagctttagagtccacagggaaagctagcaggaattttgttttagtgggagggggcagttaaacataccaagaaaaaaatactatttttataacctatgaggaagacatttggaaaatgatactctagcacagaattcagtggaatccttagggcccatgcccaaatctttccattgcttctcaggttagaatgatcttcacctccaacatgagcttggaggtgatgaggcagtggctctgtgccagctgccacaatgtgactttgatgtccacctgtaccacctctcactgggctctagcaccaccctcccctccccgcacaccaactgaacacagctctgagaagcaaagtgtgtggacccaaaactgccaagcctgagtctgtcccgtgcttctgctgctccatcctttgagttctgcattgccatcctgacgtcggccacaggaggcctgctttcttccagctgttgttctcaagttccctgcccctccacatcgcccgccagtggtgttgggttttcgttctgcttccaacctgagtaaagtgtgtgtgctgagttcatcccatgttctcccatggtcatggcttcccggccccatggggacccctctcccatcccagcagtgactggtgacagtgtgcaggtgcagtgctagctcttcgttccctctaaagggtgtgcactctttttattcctactcttgcaaaaacagatacgattatgatttcccatggaaattgaaaagtctatttaaataatttaactattaaacactttcactggtaa

SEQ ID NO: 18 - protein, 1261 aa, Homo sapiens
MSGSKSVSPPGYAAQKTAAPAPRGGPEHRSAWGEADSRANGYPHAPGGSARGSTKKPGGAVTPQQQQRLASRWRSDDDDDPPLSGDDPLAGGFGFSFRSKSAWQERGGDDCGRGSRRQRRGAASGGSTRAPPAGGGGGSAAAAASAGGTEVRPRSVEVGLEERRGKGRAADELEAGAVEGGEGSGDGGSSADSGSGAGPGAVLSLGACCLALLQIFRSKKFPSDKLERLYQRYFFRLNQSSLTMLMAVLVLVCLVMLAFHAARPPLQLPYLAVLAAAVGVILIMAVLCNRAAFHQDHMGLACYALIAVVLAVQVVGLLLPQPRSASEGIWWTVFFIYTIYTLLPVRMRAAVLSGVLLSALHLAIALRTNAQDQFLLKQLVSNVLIFSCTNIVGVCTHYPAEVSQRQAFQETRECIQARLHSQRENQQQERLLLSVLPRHVAMEMKADINAKQEDMMFHKIYIQKHDNVSILFADIEGFTSLASQCTAQELVMTLNELFARFDKLAAENHCLRIKILGDCYYCVSGLPEARADHAHCCVEMGMDMIEAISLVREVTGVNVNMRVGIHSGRVHCGVLGLRKWQFDVWSNDVTLANHMEAGGKAGRIHITKATLNYLNGDYEVEPGCGGERNAYLKEHSIETFLILRCTQKRKEEKAMIAKMNRQRTNSIGHNPPHWGAERPFYNHLGGNQVSKEMKRMGFEDPKDKNAQESANPEDEVDEFLGRAIDARSIDRLRSEHVRKFLLTFREPDLEKKYSKQVDDRFGAYVACASLVFLFICFVQITIVPHSIFMLSFYLTCSLLLTLVVFVSVIYSCVKLFPSPLQTLSRKIVRSKMNSTLVGVFTITLVFLAAFVNMFTCNSRDLLGCLAQEHNISASQVNACHVAESAVNYSLGDEQGFCGSPWPNCNFPEYFTYSVLLSLLACSVFLQISCIGKLVLMLAIELIYVLIVEVPGVTLFDNADLLVTANAIDFFNNGTSQCPEHATKVALKVVTPIIISVFVLALYLHAQQVESTARLDFLWKLQATEEKEEMEELQAYNRRLLHNILPKDVAAHFLARERRNDELYYQSCECVAVMFASIANFSEFYVELEANNEGVECLRLLNEIIADFDEIISEDRFRQLEKIKTIGSTYMAASGLNDSTYDKVGKTHIKALADFAMKLMDQMKYINEHSFNNFQMKIGLNIGPVVAGVIGARKPQYDIWGNTVNVASRMDSTGVPDRIQVTTDMYQVLAANTYQLECRGVVKVKGKGEMMTYFLNGGPPLS

SEQ ID NO: 19 - DNA, 4480 bp, Homo sapiens
gagttcgctgctgtcccggttcctctcgagtcggctccaactgccagcccgggttggcgccatgtacgccgtgtacaaacaggcgcatccgcccaccggtctggagttctccatgtactgcaacttcttcaacaacagcgagcgcaacctggtagtggccgggacctcgcagctctacgtgtaccgcctcaaccgcgacgccgaggctctgaccaagaatgacaggagcacagaggggaaggcccaccgggagaagctcgagcttgctgcctccttctccttctttggcaacgtcatgtccatggccagcgtgcagctggcaggagccaagcgggatgccctgctcctaagcttcaaggatgccaagctgtctgtggtggagtacgacccgggcacccatgacctgaagaccctgtcactgcactactttgaggagcctgagcttcgggacgggtttgtgcagaatgtacacacgccgcgagtgcgggtggaccccgacgggcgctgtgcagccatgcttgtctacggcacgcggctggtggtcctgcccttccgcagggagagcctggctgaggagcacgaggggctcgtgggtgaggggcagaggtccagcttcctgcccagctacatcatcgacgtgcgggccctagacgagaagctgctcaacatcatcgacctgcagttcctgcatggctactacgagcctaccctcctcatcctgtttgagcccaaccagacctggcctgggcgcgtggccgtgcggcaggacacgtgctccattgtggccatctcactgaacatcacgcagaaggtgcaccccgtcatctggtccctcaccagcctgccctttgactgcacccaggctctggctgtgcccaagcccataggtggggtggtggtgtttgccgtcaactcgctgttgtacctgaaccagagcgtccccccgtatggcgtggctctcaacagcctcaccacaggaaccacggctttcccgcttcgcacccaggagggtgtgcggatcaccctggactgcgcccaggccaccttcatctcctacgacaagatggtcatctccctcaagggcggcgagatctacgtgctgaccctcatcaccgacggcatgcgcagtgtccgagcgttccactttgacaaggcggccgccagcgtcctcaccaccagcatggtcaccatggagcccgggtacctgttcctgggttctcgcctgggcaattccctcctcctcaagtacacggagaagctgcaggagcccccggccagtgctgtccgtgaggctgccgacaaggaagagcctccctcaaagaagaagcgagtggatgcgacggccggctggtcagctgcgggtaagtcggtgccgcaggatgaggtggacgagattgaagtgtacggcagcgaggcccagtcgggaacacagctggccacctactcctttgaggtgtgtgacagcatcctgaacattggaccctgtgccaatgccgccgtgggcgagcctgccttcctctctgaagagtttcagaacagccccgagccggacctggagattgtggtttgctccggccacgggaagaacggggctttgtcggtgctgcagaagagcatccggccccaggtggtgacaacctttgagcttcccggctgctatgacatgtggacagtcatcgccccggtgcgtaaggaggaggaggacaatcccaagggggagggcacagagcaggaacccagcaccacccctgaagcagacgacgacggccgcagacacggattcctgattctgagccgggaagactccaccatgatcctgcagacggggcaggagatcatggagctggacaccagtggcttcgccactcagggccccacggtctttgctgggaacatcggggacaaccgctacattgtccaagtgtcaccactgggcatccgcctgctggaaggagtgaatcagctgcacttcatccccgtggacctgggcgcccccatcgtgcagtgcgccgtggccgacccctatgtggtcatcatgagtgccgagggccacgtcaccatgttcctgctgaagagtgactcctacggtggccgccaccaccgcctggcgctgcacaagcccccgctgcaccatcagtccaaggtgattacgctgtgcctgtaccgagacctcagcggcatgttcaccactgagagccgcctgggtggggcccgtgacgagctcgggggccgcagtggcccggaggccgagggcctgggctcagagactagccccacagtggatgacgaggaggagatgctgtatggggattcgggctccctcttcagccccagcaaggaggaggcccgaagaagcagccagccccctgctgaccgggaccctgcacccttccgggcagagcctacccactggtgcctgctggtgcgggagaatggcaccatggagatctaccagcttcccgactggcggctggtgttcctggtgaagaacttccctgtggggcagcgggtccttgtggacagctcctttggacagcccactacacagggcgaggcccgcagggaggaggccacgcgccagggggagctgcccctcgtcaaggaggtgctgctggtggcgctgggcagccgccagagcaggccctacctgctggtgcatgtggaccaagagctgcttatctacgaggccttcccccacgactctcagctcggccagggcaatctcaaagtccgctttaagaaggtccctcacaacatcaacttccgtgagaagaagccaaagccatccaagaagaaagcagaaggtggcggcgcagaggagggggctggggcccggggccgcgtggcgcgtttccgctacttcgaggatatttatggctactcaggggtcttcatctgcggcccctcccctcactggctcttggtgaccggccgaggggctctgcggctacaccccatggccatcgacggcccggtcgactctttcgctccattccacaatgtcaactgtccccgcggcttcctgtacttcaacagacagggcgagctgaggatcagtgtcctgcctgcctacctgtcctatgatgccccatggcctgtcaggaagatcccgctgcgctgcacggcccactatgtggcttaccacgtggagtctaaggtgtatgctgtggccaccagcaccaacacgccgtgtgcccgcatcccacgcatgactggcgaggagaaggagtttgagaccatcgagagagatgagcggtacatccacccccagcaggaggccttctccatccagctcatctccccggtcagctgggaggctattcccaatgccaggatcgagctgcaggagtgggagcatgtgacctgcatgaagacagtgtctctgcgcagtgaggagaccgtgtcgggcctcaaaggctacgtggccgccgggacctgcctcatgcagggggaggaggtcacgtgccgagggcggatcttgatcatggatgtgattgaggtggtgcccgagcctggccagcccttgaccaagaacaagttcaaagtcctttacgagaaggagcagaaggggcccgtgaccgccctgtgccactgcaatggccacctggtgtcggccatcggccagaagattttcctgtggagcctgcgggccagcgagctgacgggcatggccttcatcgacacgcagctctacatacaccagatgatcagcgtcaagaacttcatcctggcagccgacgtcatgaagagcatttcgctgctgcgctaccaggaggaaagcaagacgctgagcctggtgtcgcgggatgccaagcccctggaggtgtacagcgtggacttcatggtggacaatgcccagctgggttttctggtgtctgaccgcgaccgcaacctcatggtgtacatgtacctgcccgaagccaaggagagtttcgggggcatgcgcctgctgcgtcgggcagacttccacgtgggtgcccacgtgaacacgttctggaggaccccgtgccggggggccactgaagggctcagcaaaaagtcggtcgtgtgggagaataagcacatcacgtggtttgccaccctggacggcggcatcgggctgctgctgcccatgcaggagaagacctaccggcggctgctgatgctgcagaacgcgctgaccaccatgctgccacaccacgccggcctcaacccccgcgccttccggatgctgcacgtggaccgccgcaccctccagaatgccgtgcgcaacgtgctggatggggagctgctcaaccgctacctgtacctgagcaccatggagcgcagcgagctagccaagaagatcggcaccacaccagacataatcctggacgacttgctggagacggaccgcgtcaccgcccacttctagccccgtggatgccgtcaccaccagcacacggaactacctcccaccccctttttgtacaaaacacaaggaaaaacattttttgcttga

SEQ ID NO: 20 - protein, 1443 aa, Homo sapiens
MYAVYKQAHPPTGLEFSMYCNFFNNSERNLVVAGTSQLYVYRLNRDAEALTKNDRSTEGKAHREKLELAASFSFFGNVMSMASVQLAGAKRDALLLSFKDAKLSVVEYDPGTHDLKTLSLHYFEEPELRDGFVQNVHTPRVRVDPDGRCAAMLVYGTRLVVLPFRRESLAEEHEGLVGEGQRSSFLPSYIIDVRALDEKLLNIIDLQFLHGYYEPTLLILFEPNQTWPGRVAVRQDTCSIVAISLNITQKVHPVIWSLTSLPFDCTQALAVPKPIGGVVVFAVNSLLYLNQSVPPYGVALNSLTTGTTAFPLRTQEGVRITLDCAQATFISYDKMVISLKGGEIYVLTLITDGMRSVRAFHFDKAAASVLTTSMVTMEPGYLFLGSRLGNSLLLKYTEKLQEPPASAVREAADKEEPPSKKKRVDATAGWSAAGKSVPQDEVDEIEVYGSEAQSGTQLATYSFEVCDSILNIGPCANAAVGEPAFLSEEFQNSPEPDLEIVVCSGHGKNGALSVLQKSIRPQVVTTFELPGCYDMWTVIAPVRKEEEDNPKGEGTEQEPSTTPEADDDGRRHGFLILSREDSTMILQTGQEIMELDTSGFATQGPTVFAGNIGDNRYIVQVSPLGIRLLEGVNQLHFIPVDLGAPIVQCAVADPYVVIMSAEGHVTMFLLKSDSYGGRHHRLALHKPPLHHQSKVITLCLYRDLSGMFTTESRLGGARDELGGRSGPEAEGLGSETSPTVDDEEEMLYGDSGSLFSPSKEEARRSSQPPADRDPAPFRAEPTHWCLLVRENGTMEIYQLPDWRLVFLVKNFPVGQRVLVDSSFGQPTTQGEARREEATRQGELPLVKEVLLVALGSRQSRPYLLVHVDQELLIYEAFPHDSQLGQGNLKVRFKKVPHNINFREKKPKPSKKKAEGGGAEEGAGARGRVARFRYFEDIYGYSGVFICGPSPHWLLVTGRGALRLHPMAIDGPVDSFAPFHNVNCPRGFLYFNRQGELRISVLPAYLSYDAPWPVRKIPLRCTAHYVAYHVESKVYAVATSTNTPCARIPRMTGEEKEFETIERDERYIHPQQEAFSIQLISPVSWEAIPNARIELQEWEHVTCMKTVSLRSEETVSGLKGYVAAGTCLMQGEEVTCRGRILIMDVIEVVPEPGQPLTKNKFKVLYEKEQKGPVTALCHCNGHLVSAIGQKIFLWSLRASELTGMAFIDTQLYIHQMISVKNFILAADVMKSISLLRYQEESKTLSLVSRDAKPLEVYSVDFMVDNAQLGFLVSDRDRNLMVYMYLPEAKESFGGMRLLRRADFHVGAHVNTFWRTPCRGATEGLSKKSVVWENKHITWFATLDGGIGLLLPMQEKTYRRLLMLQNALTTMLPHHAGLNPRAFRMLHVDRRTLQNAVRNVLDGELLNRYLYLSTMERSELAKKIGTTPDIILDDLLETDRVTAHF

SEQ ID NO: 21 - DNA, 2086 bp, Homo sapiens
ggagacgcaagatggcggctgtggtgctggcggcgacgcggttgctgcggggctcgggttcttggggctgttcgcggctgaggtttggacctcctgcgtacagacggtttagtagtggtggtgcctatcccaacatccccctctcttctcccttacctggagtacccaagcctgtttttgctacagttgatggacaggaaaagtttgaaaccaaagtaaccacattggataatgggcttcgcgtggcatctcagaataagtttggacagttttgtacagtaggaattcttatcaattcaggatcgagatatgaagcgaaataccttagtggaattgctcactttttggaaaaattggcattttcgtctactgctcgatttgacagcaaagatgaaattctgcttacgttggaaaagcatgggggtatctgtgactgccagacatcaagagacaccaccatgtatgctgtgtctgctgatagcaaaggcttggacacggtggttgccttactggctgatgtggttctgcagccccggctaacagatgaagaagtcgagatgacgcggatggcggtccagtttgagctggaggacctgaacctgcggcctgacccagagccacttctcaccgagatgattcatgaagcggcttacagggagaacacagttggcctccaccgtttctgccccacagaaaacgtagcaaagatcaaccgagaggtgctgcattcctacctgaggaactactacactcccgaccgcatggtgctggccggcgtgggcgtggagcacgagcatctggtggactgtgcccggaagtacctcctgggggtccagccggcctgggggagcgcagaggccgtggatattgacagatctgtggcccagtacactggggggattgccaagctagaaagagacatgtccaatgtcagcctgggcccgacccccatccccgagctcacgcacatcatggttggactggagagctgctccttcctggaggaggacttcatcccctttgcagtgttgaacatgatgatgggcggaggtggctccttctcggctggtgggcccggcaagggcatgttctccaggctctacctcaacgtgctcaacaggcaccactggatgtataacgcgacctcctaccaccacagctacgaggacactggcctcctttgcatccatgccagcgccgacccaagacaggttcgagaaatggtagaaatcatcacaaaggagtttattttaatgggcggaaccgtggacacggtggagctggaacgagccaagacgcagctgacatcaatgctcatgatgaacctggaatccaggcctgtgatcttcgaggatgtggggaggcaggtgctggccactcgctccagaaagctgccgcacgagctgtgcacgctcatccgcaacgtgaagccggaagatgtgaagagagtcgcttctaagatgctccgagggaagccggcagtggccgccctgggtgacctgactgacctgcccacgtatgagcacatccagaccgccctgtcgagtaaggacgggcgcctgcccaggacgtaccggctcttccggtagaaccgctccccggcctgacagacccagggagctgcagctggagcccgttcccgtgcgtgttagtttggacacgaatttagtctaaaaagctgtctggttgtataaacggtgcaaacaatgtcgccacagcacccacgcggtttgcattcttttggaactcaatgtgccgatcagtggagtcagtatcgagcctgaccaccgcaagccaggaagcaggtgaagtgcccagcgctggagtgcagcgtgccacgaggagggcggtcggtgcttccctcctcgggctgtgggcacatggggccccgcaggttccttggaggagccctgagctgggaggcagcaaaggctgacctatcaaagcctcccggaggccaccgtgctgggtaccaggactcacctctgacaagcaggagaaggtaagggcccggtcagctccaaggagcgcgctccacgcgcgtgcacacagcttccctggtaataaagagctggcatctttctta

SEQ ID NO: 22 - protein, 525 aa, Homo sapiens
MAAVVLAATRLLRGSGSWGCSRLRFGPPAYRRFSSGGAYPNIPLSSPLPGVPKPVFATVDGQEKFETKVTTLDNGLRVASQNKFGQFCTVGILINSGSRYEAKYLSGIAHFLEKLAFSSTARFDSKDEILLTLEKHGGICDCQTSRDTTMYAVSADSKGLDTVVALLADVVLQPRLTDEEVEMTRMAVQFELEDLNLRPDPEPLLTEMIHEAAYRENTVGLHRFCPTENVAKINREVLHSYLRNYYTPDRMVLAGVGVEHEHLVDCARKYLLGVQPAWGSAEAVDIDRSVAQYTGGIAKLERDMSNVSLGPTPIPELTHIMVGLESCSFLEEDFIPFAVLNMMMGGGGSFSAGGPGKGMFSRLYLNVLNRHHWMYNATSYHHSYEDTGLLCIHASADPRQVREMVEIITKEFILMGGTVDTVELERAKTQLTSMLMMNLESRPVIFEDVGRQVLATRSRKLPHELCTLIRNVKPEDVKRVASKMLRGKPAVAALGDLTDLPTYEHIQTALSSKDGRLPRTYRLFR

SEQ ID NO: 23 - DNA, 5359 bp, Homo sapiens
gttctgaatgatgactgacgcgggtttgggtgatacccctcacagcccctgtcattccggagtcataaggcacccgcgcgtctagccccagcgccagggcacgcgagcggcgctggagggaggaaagcttccgcctgcgggccggacaaaagtcccgcctgcccacggctttttgcccgccgctcgtgaccgagacgcctcgccgcggccagctcgctgctctcgctggcggatggtgtgtggccgccgcaggacgcccgccgtgcccgggccatgaagtagcggctgctggcggcgccgctgcccaaccgccagccccagccccgcgctgcgctgcccggtcctctcccggcggggtcgtatcggcgtggacatggctggccgcgtccctagcctgctagttctccttgtttttccaagcagctgtttggctttccgaagcccactttctgtctttaagaggtttaaagaaactaccagaccattttccaatgaatgtcttggtaccaccagacccgtagttcctattgattcatcagattttgcattggatattcgcatgcctggggttacacctaaacagtccgatacatacttctgcatgtctatgcgaataccagtggatgaggaagccttcgtgattgacttcaagcctcgagccagcatggatactgtccatcacatgttactttttggatgcaatatgccttcatccactggaagttactggttttgtgatgaaggaacctgtacagataaagccaatattctgtatgcctgggcgagaaatgctccccctacccggctccccaaaggtgttggattcagagttggaggagagactggaagtaaatactttgtactacaggtacactatggggatattagtgcttttagagataataacaaggactgttctggtgtgtccttacacctcacacgtctgccacagcctttaattgctggcatgtaccttatgatgtctgttgacactgttatcccagcaggagaaaaagtggtgaattctgacatttcatgccattataaaaattatccaatgcatgtctttgcctatagagttcacactcaccatttaggtaaggtagtaagtggatacagagtaagaaatggacagtggacactgattggacggcagagccctcagctgccacaggctttctaccctgtggggcatccagttgatgtaagttttggtgacctactggctgcaagatgtgtattcactggtgaaggaaggacagaagccacacacattggtggcacgtctagtgatgaaatgtgcaacttatacattatgtattacatggaagccaagcatgcagtttctttcatgacctgtacccagaatgtagctccagatatgttcagaaccataccaccagaggccaacattccaattcccgtgaagtctgatatggttatgatgcatgaacatcataaagaaacagaatataaagataagattcctttactacagcagccaaaacgagaagaagaagaagtgttagaccagggtgatttctattcactactttccaagctgctaggagaaagggaagatgttgttcatgtgcacaaatataatcctacagaaaaggcagaatcagagtcagacctggtagctgagattgcaaatgtagtccaaaaaaaggatcttggtcgatctgatgccagagagggtgcagaacatgagaggggtaatgctattcttgtcagagacagaattcacaaattccacagactagtatctaccttgaggccaccagagagcagagttttctcattacagcagcccccacctggtgaaggcacctgggaaccagaacacacaggagatttccacatggaagaggcactggattggcctggagtatacttgttaccaggccaggtttctggggtggctctagaccctaagaataacctggtgattttccacagaggtgaccatgtctgggatggaaactcgtttgacagcaagtttgtttaccagcaaataggactcggaccaattgaagaagacactattcttgtcatagatccaaataatgctgcagtactccagtccagtggaaaaaatctgttttacttgccacatggcttgagtatagataaagatgggaattattgggtcacagacgtggctctccatcaggtgttcaaactggatccaaacaataaagaaggccctgtattaatcctgggaaggagcatgcaaccaggcagtgaccagaatcacttctgtcaacccactgatgtggctgtggatccaggcactggagccatttatgtatcagatggttactgcaacagcaggattgtgcagttttcaccaagtggaaagttcatcacacagtggggagaagagtcttcagggagcagtcctctgccaggccagttcactgttcctcacagcttggctcttgtgcctcttttgggccaattatgtgtggcagaccgggaaaatggtcggatccagtgttttaaaactgacaccaaagaatttgtgagagagattaagcattcatcatttggaagaaatgtatttgcaatttcatatataccaggcttgctctttgcagtgaatgggaagcctcattttggggaccaagaacctgtacaaggatttgtgatgaacttttccaatggggaaattatagacatcttcaagccagtgcgcaagcactttgatatgcctcatgatattgttgcatctgaagatgggactgtgtacattggagatgctcataccaacaccgtgtggaagttcaccttgactgagaaattggaacatcgatcagttaaaaaggctggcattgaggtccaggaaatcaaagaagccgaggcagttgttgaaaccaaaatggagaacaaacccacctcctcagaattgcagaagatgcaagagaaacagaaactgatcaaagagccaggctcgggagtgcctgttgttctcattacaacccttctggttattccggtggttgtcctgctggccattgccatatttattcggtggaaaaaatcaagggcctttggagcagattctgaacacaaactcgagacgagttcaggaagagtactgggaagatttagaggaaagggaagtggaggcttaaaccttggtaatttctttgcaagccgtaagggctacagtcgaaaagggtttgaccggcttagcactgagggcagtgaccaagagaaagaggatgatggaagtgaatcagaagaggagtattcagcacctctgcctgcgctcgcaccttcctcctcctgaaaaccaagctttgatttagattgagtaagatttacccagaatgtcagattcctttccctttagcacgtttaaagttctgtgtatttaattgtaaactgtactagtctgtgtgggactgtacacactttatttacttcgttttggttaagttggcttctgtttctagttgaggagtttcctaaaagttcataacagtgccattgtctttatatgaacatagactagagaaaccgtcctctttttccatcataattctaatctaacaatggaagatttgcccatttacacttttgagactttttggtggatgtaaataaccccattctttgcttgaacacagtattttcccaatagcactttcattgccagtgtctttctttggtgcctttcctgttcagcattcttagcctgtggcagtaaagagaaactttgtgctacatgacgacaaagctgctaaatctcctatttttttaaaatcactaacattatattgcaatgaaggaaataaaaaagtctctatttaaattcttttttaaattttcttcagttggtgtgtttttgggatgtcttatttttagatggttacactgttagaacactattttcagaatctgaatgtaatttgtgtaataaagtgttttcagagcattagctgtcagtgtattttccagtttttgcgtatttgcagattttacatacaacttttataataattacacaaacccacaaatattagtgaaacttactcgatgtcttcaactaaaagaaatgtgtgtattgtacaaaatttagaagatactttagccaatataaattaaaaaccagcctgagtttacataaatttgtaaagtcaggctcttctaaaatccaaagagggtttttgcctatatatcaatcagaggataaatactttaataaaaggtaatcacagctaagtggatacctgtgtctcaaattacatatgcaaatgatccatcagtagggatcactaataatagttttccttttaaaaaataatttcagggcaggtacagtggctcaagcctatacttccaggactttgggaggccaagcagaaggattgtgggaggccaagcagaaggattgtttgagcccagaaattcaaggctgcagtgagctatgatcaatccactgtactccagcctaggctacagagtgagatcctttctctaaaataaaatacaatttccatgtatccataggaatatattcatcttttacagtgattgcagattagttttaaaacgtctttttcgtaattcgtcaatgcaagttagtaagaaccagataattttccattttaaaatgataggaatctaaaactctttttcaaaaatgccaacctgtttttccggcatcatagtagttggaataatacagatataattgactaatcataaagtacatgagagtacagaagggaaatttggaaacggtaagtctgctagggcattacaatcctgtcctagcactccacctttattctgccaacctgggttaattaaagatggtagcctggaaagtaatgaatgacattgacttcaggcaatctttccactgattattcttggctgaacttcatttatcgagtcagagaatgcactgcctgagaaatgtcccagaggagtgaatcctaggcctggccacagtagaatcgcctggagaatttaagaaaaaaattgttgggcactcactctttccagtgatctttatttagttggcttacaatggaatacaggtatggttcatgtttttaaatttgtccaggtgtttctattgtgcagtcacagttgaaaaccattgtcttcgagaatagctattctatcttgccagttacaataagtggatagctatattttcacataaattatagtttacagatgtttggagggggaagacaggatctgaggtgttttgatatgactgttagcaccaaaatctgaatgccttaattgttgaatgtgttaaattggataattaaaatgggcataaatgacttattaaaaaagcaaagggaaaaaaaaaaaaaaaaaaaaaaaa

SEQ ID NO: 24 - protein, 974 aa, Homo sapiens
MAGRVPSLLVLLVFPSSCLAFRSPLSVFKRFKETTRPFSNECLGTTRPVVPIDSSDFALDIRMPGVTPKQSDTYFCMSMRIPVDEEAFVIDFKPRASMDTVHHMLLFGCNMPSSTGSYWFCDEGTCTDKANILYAWARNAPPTRLPKGVGFRVGGETGSKYFVLQVHYGDISAFRDNNKDCSGVSLHLTRLPQPLIAGMYLMMSVDTVIPAGEKVVNSDISCHYKNYPMHVFAYRVHTHHLGKVVSGYRVRNGQWTLIGRQSPQLPQAFYPVGHPVDVSFGDLLAARCVFTGEGRTEATHIGGTSSDEMCNLYIMYYMEAKHAVSFMTCTQNVAPDMFRTIPPEANIPIPVKSDMVMMHEHHKETEYKDKIPLLQQPKREEEEVLDQGDFYSLLSKLLGEREDVVHVHKYNPTEKAESESDLVAEIANVVQKKDLGRSDAREGAEHERGNAILVRDRIHKFHRLVSTLRPPESRVFSLQQPPPGEGTWEPEHTGDFHMEEALDWPGVYLLPGQVSGVALDPKNNLVIFHRGDHVWDGNSFDSKFVYQQIGLGPIEEDTILVIDPNNAAVLQSSGKNLFYLPHGLSIDKDGNYWVTDVALHQVFKLDPNNKEGPVLILGRSMQPGSDQNHFCQPTDVAVDPGTGAIYVSDGYCNSRIVQFSPSGKFITQWGEESSGSSPLPGQFTVPHSLALVPLLGQLCVADRENGRIQCFKTDTKEFVREIKHSSFGRNVFAISYIPGLLFAVNGKPHFGDQEPVQGFVMNFSNGEIIDIFKPVRKHFDMPHDIVASEDGTVYIGDAHTNTVWKFTLTEKLEHRSVKKAGIEVQEIKEAEAVVETKMENKPTSSELQKMQEKQKLIKEPGSGVPVVLITTLLVIPVVVLLAIAIFIRWKKSRAFGADSEHKLETSSGRVLGRFRGKGSGGLNLGNFFASRKGYSRKGFDRLSTEGSDQEKEDDGSESEEEYSAPLPALAPSSS

SEQ ID NO: 25 - DNA, 1630 bp, Homo sapiens
gaagcaccggtgagtgggcaggggctccctccccatcaatagggccgacccaagtcttcctcccccttcccccatgccgggccccacgatagtgtgaatgtcaggggcttcaggtttccctaaatataggtccctgccagaggatccgtggcgggaaaagggcaggggtcattagagaagatcggggacacatgtggggcgggcaggagctgccttataaccagcccgggaacccctagctcactcgctgctgaccaggctctgccggctccttcggcctcgccgcaggaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtacaagaaggacggagctgacttcgccaagtggcgttgtgtgctgaagattggggaacacaccccctcagccctcgccatcatggaaaatgccaatgttctggcccgttatgccagtatctgccagcagaatggcattgtgcccatcgtggagcctgagatcctccctgatggggaccatgacttgaagcgctgccagtatgtgaccgagaaggtgctggctgctgtctacaaggctctgagtgaccaccacatctacctggaaggcaccttgctgaagcccaacatggtcaccccaggccatgcttgcactcagaagttttctcatgaggagattgccatggcgaccgtcacagcgctgcgccgcacagtgccccccgctgtcactgggatcaccttcctgtctggaggccagagtgaggaggaggcgtccatcaacctcaatgccattaacaagtgccccctgctgaagccctgggccctgaccttctcctacggccgagccctgcaggcctctgccctgaaggcctggggcgggaagaaggagaacctgaaggctgcgcaggaggagtatgtcaagcgagccctggccaacagccttgcctgtcaaggaaagtacactccgagcggtcaggctggggctgctgccagcgagtccctcttcgtctctaaccacgcctattaagcggaggtgttcccaggctgcccccaacactccaggccctgccccctcccactcttgaagaggaggccgcctcctcggggctccaggctggcttgcccgcgctctttcttccctcgtgacagtggtgtgtggtgtcgtctgtgaatgctaagtccatcaccctttccggcacactgccaaataaacagctatttaagggggagtcggcaaaaaaaaaaaaaaaaaa

SEQ ID NO: 26 - protein, 364 aa, Homo sapiens
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 27 - DNA, 4592 bp, Homo sapiens
agaatgcactgctgacacgtgtgcgcgcgcgcggctccactgccgggcgaccgcgggaaaattccaaaaaaactcaaaaagccaatacgaggcaaagccaaattttcaagccacagatcccgggcggtggcttcctttccgccactgcccaaactgctgaagcagctcccgcgaggaccacccgatttaatgtgtgccgaccatttccttcagtgctggacaggctgctgtgaagggacatcaccttttcgctttttccaagatggctcaagattcagtagatctttcttgtgattatcagttttggatgcagaagctttctgtatgggatcaggcttccactttggaaacccagcaagacacctgtcttcacgtggctcagttccaggagttcctaaggaagatgtatgaagccttgaaagagatggattctaatacagtcattgaaagattccccacaattggtcaactgttggcaaaagcttgttggaatccttttattttagcatatgatgaaagccaaaaaattctaatatggtgcttatgttgtctaattaacaaagaaccacagaattctggacaatcaaaacttaactcctggatacagggtgtattatctcatatactttcagcactcagatttgataaagaagttgctcttttcactcaaggtcttgggtatgcacctatagattactatcctggtttgcttaaaaatatggttttatcattagcgtctgaactcagagagaatcatcttaatggatttaacactcaaaggcgaatggctcccgagcgagtggcgtccctgtcacgagtttgtgtcccacttattaccctgacagatgttgaccccctggtggaggctctcctcatctgtcatggacgtgaacctcaggaaatcctccagccagagttctttgaggctgtaaacgaggccattttgctgaagaagatttctctccccatgtcagctgtagtctgcctctggcttcggcaccttcccagccttgaaaaagcaatgctgcatctttttgaaaagctaatctccagtgagagaaattgtctgagaaggatcgaatgctttataaaagattcatcgctgcctcaagcagcctgccaccctgccatattccgggttgttgatgagatgttcaggtgtgcactcctggaaaccgatggggccctggaaatcatagccactattcaggtgtttacgcagtgctttgtagaagctctggagaaagcaagcaagcagctgcggtttgcactcaagacctactttccttacacttctccatctcttgccatggtgctgctgcaagaccctcaagatatccctcggggacactggctccagacactgaagcatatttctgaactgctcagagaagcagttgaagaccagactcatgggtcctgcggaggtccctttgagagctggttcctgttcattcacttcggaggatgggctgagatggtggcagagcaattactgatgtcggcagccgaaccccccacggccctgctgtggctcttggccttctactacggcccccgtgatgggaggcagcagagagcacagactatggtccaggtgaaggccgtgctgggccacctcctggcaatgtccagaagcagcagcctctcagcccaggacctgcagacggtagcaggacagggcacagacacagacctcagagctcctgcacaacagctgatcaggcaccttctcctcaacttcctgctctgggctcctggaggccacacgatcgcctgggatgtcatcaccctgatggctcacactgctgagataactcacgagatcattggctttcttgaccagaccttgtacagatggaatcgtcttggcattgaaagccctagatcagaaaaactggcccgagagctccttaaagagctgcgaactcaagtctagaaggcacgcaggccgtgtgggtgcccggcgtgagggatcaggctcgccagggccacaggacaggtgatgacctgtggccacgcatttgtggagtaagtgccctcgctgggctgtgagaatgagctgtacacatcttgggacaatctgctagtatctattttacaaaatgcagagccaggtccctcagcccagactcagtcagacatgttcactaatgactcaagtgagccttcggtactcctggtgcccgcccggccagaccgtcagcttgataattactaaagcaaaggcctgggtgggagaacaggtttctagtttttacccaagtcaagctgcacatctattatttaaaaattcaaagtcttagaaccaagaatttggtcatgaaccattaaagaatttagagagaacttagctctttttagactctttttaggagtcagggatctgggataaagccacactgtcttgctgtatggagaaattcttcaaggggagtcagggtccctcaggcttcccttgtgtctccctggacctgcctgacaggccacaggagcagacagcacacccaagcccgggcctccggcacactctttccactctgtatttgctaaatgatgctaactgctaccaaaaggcccttgggacatcagaggagccggcaggcgaaggtagaggatgtgttccagaaacattagaaggcaggattaattcagttagttagttctcttgttaaatggaaatgggaattggaaattcctgataaagaattggcctggctgggtgcagtggctcacacctgtgatcccagcactttgggaggccaaggcagggggattacttcagcccaggagttccagactgcctggctaacatggcaataccctatctctactaaaaatacaaaaattatcggggtgcaatggcatgcatctgtaatcccagctattcaagaggctgaggcatgaggatctcttgaacccgggaggtgggagttgtagtgagccgagatcatgacactgcactccagcctgggcaacagagcgagaccatctcttaaaaaaaggcattgttagtgtaatctcaaggttaacatttatttcatgtcagtacagggtgctttttcctttcagggacattctggaattgtattggttgtacattcttttgtgtctattctgtttgtcaagtgagtcaagacttgcttttgtccattttgatttgtgtgtattagtctgagtcttggctccgttttgaggtatgagcaaagttttgctggattagaagttaacctttagggaaattccttattttggtatgtggcaatgctaatagatccactgaagatctggaaaattccaggaacttttcacctgagcctttcttctgagaaatgctgcagtcagaagggtgtgctggtaaagtattttggtggcagctgccatcatggtcattgccttcatataacatgcttcgtgctcatggtcattgccttcatataacatgcttcgtgccatcatgatccttgccttcatataacaaacatgcttcgtcagaggtgttggggttgaaaaaggagctgcatgcttcactggagttgagggcctctctcctgttctgactttaagccagaacttgtggctgggccatggaagctgtgactcctctgtggacatggtggcagcagggaacccctagagagaggggccactgggaccaggcctcctgttgtggagggactcctgggacagtcctccaccctgtcctgtggtcctgtgtacagggttggcctcttcctcctcccctgccaggcctctgcccatgccccttccttccttctcctgggactggtgaagctaggcatctggaagacttcttcctagcctggaagccctgacctcggcccatctgcagaatctcccagttccttcacagctgccgagtcctctcacgggtgcggtggaggcggccttgccggtggtgctttctgggcagccaggggttcctgggtgggaggactgtccctctggggacgtggcactgaagtgcctgctggcttcatgtggccctttgccctttcccagcctgagagatgctcaaaggtggggagctgggggagccacccctcggccattccctccacctccaagacaggtggcggccgggcaggcactcttaagcccacctccccctcttgttgccttcgatttcggcaaagcctgggcaggtgccaccgggaaggaatggcatccgagatgctgggcggggacgcggcgtggccgagggggccttgacggcgttggcggggcctgggcacaggggcagccgcagggaggcagggatggcaaggcgtgaagccaccctggaaggaactggaccaaggtcttcagaggtgcgacagggtctggaatctgaccttactctagcaggagtttttgtagactctccctgatagtttagtttttgataaagcatgctggtaaaaccactaccctcagagagagccaaaaatacagaagaggcggagagcgcccctccaaccaggctgttattcccctggactccgtgacatctgtggaattttttagctctttaaaatctgtaatttgttgtctattttttcattctaaataaaacttcagtttgcacctaa

SEQ ID NO: 28 - protein, 558 aa, Homo sapiens
MAQDSVDLSCDYQFWMQKLSVWDQASTLETQQDTCLHVAQFQEFLRKMYEALKEMDSNTVIERFPTIGQLLAKACWNPFILAYDESQKILIWCLCCLINKEPQNSGQSKLNSWIQGVLSHILSALRFDKEVALFTQGLGYAPIDYYPGLLKNMVLSLASELRENHLNGFNTQRRMAPERVASLSRVCVPLITLTDVDPLVEALLICHGREPQEILQPEFFEAVNEAILLKKISLPMSAVVCLWLRHLPSLEKAMLHLFEKLISSERNCLRRIECFIKDSSLPQAACHPAIFRVVDEMFRCALLETDGALEIIATIQVFTQCFVEALEKASKQLRFALKTYFPYTSPSLAMVLLQDPQDIPRGHWLQTLKHISELLREAVEDQTHGSCGGPFESWFLFIHFGGWAEMVAEQLLMSAAEPPTALLWLLAFYYGPRDGRQQRAQTMVQVKAVLGHLLAMSRSSSLSAQDLQTVAGQGTDTDLRAPAQQLIRHLLLNFLLWAPGGHTIAWDVITLMAHTAEITHEIIGFLDQTLYRWNRLGIESPRSEKLARELLKELRTQV

SEQ ID NO: 29 - DNA, 3072 bp, Homo sapiens
aacggctcgcggagcggctacgcggagtgacatcgccggtgtttgcgggtggttgttgctctcggggccgtgtggagtaggtctggacctggactcacggctgcttggagcgtccgccatgaggagaagtgaggtgctggcggaggagtccatagtatgtctgcagaaagccctaaatcaccttcgggaaatatgggagctaattgggattccagaggaccagcggttacaaagaactgaggtggtaaagaagcatatcaaggaactcctggatatgatgattgctgaagaggaaagcctgaaggaaagactcatcaaaagcatatccgtctgtcagaaagagctgaacactctgtgcagcgagttacatgttgagccatttcaggaagaaggagagacgaccatcttgcaactagaaaaagatttgcgcacccaagtggaattgatgcgaaaacagaaaaaggagagaaaacaggaactgaagctacttcaagagcaagatcaagaactgtgcgaaattctttgtatgccccactatgatattgacagtgcctcagtgcccagcttagaagagctgaaccagttcaggcaacatgtgacaactttgagggaaacaaaggcttctaggcgtgaggagtttgtcagtataaagagacagatcatactgtgtatggaagcattagaccacaccccagacacaagctttgaaagagatgtggtgtgtgaagacgaagatgccttttgtttgtctttggagaatattgcaacactacaaaagttgctacggcagctggaaatgcagaaatcacaaaatgaagcagtgtgtgaggggctgcgtactcaaatccgagagctctgggacaggttgcaaatacctgaagaagaaagagaagctgtggccaccattatgtctgggtcaaaggccaaggtccggaaagcgctgcaattagaagtggatcggttggaagaactgaaaatgcaaaacatgaagaaagtgattgaggcaattcgagtggagctggttcagtactgggaccagtgcttttatagccaggagcagagacaagcttttgcccctttctgtgctgaggactacacagaaagtctgctccagctccacgatgctgagattgtgcggttaaaaaactactatgaagttcacaaggaactctttgaaggtgtccagaagtgggaagaaacctggaggcttttcttagagtttgagagaaaagcttcagatccaaatcgatttacaaaccgaggaggaaatcttctaaaagaagaaaaacaacgagccaagctccagaaaatgctgcccaagctggaagaagagttgaaggcacgaattgaattgtgggaacaggaacattcaaaggcatttatggtgaatgggcagaaattcatggagtatgtggcagaacaatgggagatgcatcgattggagaaagagagagccaagcaggaaagacaactgaagaacaaaaaacagacagagacagagatgctgtatggcagcgctcctcgaacacctagcaagcggcgaggactggctcccaatacaccgggcaaagcacgtaagctgaacactaccaccatgtccaatgctacggccaatagtagcattcggcctatctttggagggacagtctaccactcccccgtgtctcgacttcctccttctggcagcaagccagtcgctgcttccacctgttcagggaagaaaacaccccgtactggcaggcatggagccaacaaggagaacctggagctcaacggcagcatcctgagtggtgggtaccctggctcggcccccctccagcgcaacttcagcattaattctgttgccagcacctattctgagtttgcgaaggatccgtccctctctgacagttccactgttgggcttcagcgagaactttcaaaggcttccaaatctgatgctacttctggaatcctcaattcaaccaacatccagtcctgagaagccctgatcagtcaaccagctgtggcttcctgtgcctagactggacctaattatatgggggtgactttagtttttcttcagcttaggcgtgcttgaaaccttggccaggttccatgaccatgggcctaacttaaagatgtgaatgagtgttacagttgaaagcccatcataggtttagtggtcctaggagacttggttttgacttatatacatgaaaagtttatggcaagaagtgcaaattttagcatatggggcctgacttctctaccacataattctacttgctgaagcatgatcaaagcttgttttatttcaccactgtaggaaaatgattgactatgcccatccctgggggtaattttggcatgtatacctgtaactagtaattaacatcttttttgtttaggcatgttcaattaatgctgtagctatcatagctttgctcttacctgaagccttgtccccaccacacaggacagccttcctcctgaagagaatgtctttgtgtgtccgaagttgagatggcctgccctactgccaaagaggtgacaggaaggctgggagcagctttgttaaattgtgttcagttctgttacacagtgcattgccctttgttgggggtatgcatgtatgaacacacatgcttgtcggaacgctttctcggcgtttgtcccttggctctcatctcccccattcctgtgcctactttgcctgagttcttctacccccgcagttgccagccacattgggagtctgtttgttccaatgggttgagctgtctttgtcgtggagatctggaactttgcacatgtcactactggggaggtgttcctgctctagcttccacgatgaggcgccctctttacctatcctctcaatcactactcttcttgaagcactattatttattcttccgctgtctgcctgcagcagtactactgtcaacatagtgtaaatggttctcaaaagcttaccagtgtggacttggtgttagccacgctgtttactcatacagtacgtgtcctgtttttaaaatatacaattattcttaaaaataaattaaaatctgtatacttacatttcaaaaa

SEQ ID NO: 30 - protein, 620 aa, Homo sapiens
MRRSEVLAEESIVCLQKALNHLREIWELIGIPEDQRLQRTEVVKKHIKELLDMMIAEEESLKERLIKSISVCQKELNTLCSELHVEPFQEEGETTILQLEKDLRTQVELMRKQKKERKQELKLLQEQDQELCEILCMPHYDIDSASVPSLEELNQFRQHVTTLRETKASRREEFVSIKRQIILCMEALDHTPDTSFERDVVCEDEDAFCLSLENIATLQKLLRQLEMQKSQNEAVCEGLRTQIRELWDRLQIPEEEREAVATIMSGSKAKVRKALQLEVDRLEELKMQNMKKVIEAIRVELVQYWDQCFYSQEQRQAFAPFCAEDYTESLLQLHDAEIVRLKNYYEVHKELFEGVQKWEETWRLFLEFERKASDPNRFTNRGGNLLKEEKQRAKLQKMLPKLEEELKARIELWEQEHSKAFMVNGQKFMEYVAEQWEMHRLEKERAKQERQLKNKKQTETEMLYGSAPRTPSKRRGLAPNTPGKARKLNTTTMSNATANSSIRPIFGGTVYHSPVSRLPPSGSKPVAASTCSGKKTPRTGRHGANKENLELNGSILSGGYPGSAPLQRNFSINSVASTYSEFAKDPSLSDSSTVGLQRELSKASKSDATSGILNSTNIQS

SEQ ID NO: 31 - DNA, 4519 bp, Homo sapiens
gcactccaccacctctggccgctgggaagacgtcatcgcttccgccctacttcctcctcctgttggcggcggtgagaatccaatatggagtaaatcggtggagtcgagagatggggctcggacggggcgccctgcaccgcctcccaggcgcacctcccccggccgccgaccgctccccggaaccgtgattggcccggccccccgggggcgaccccggactgagcccccccacccgcggccggcgctctcctcctcctcgcagcagtccctgccgctccgaggcgcgcttgtttttctcctgctctcccccaccgccgtccccttcccaaatctcgccccctctgcccgctcccgagcccccggagcctccgcccgcctctccccacggcgctgcggcgttcactccccgcgcagcctgcctttgcaccccttcccccaaaccctatcccgcgccctgcttccccttctgctgcggcgccctcttcatctctagccgccccccctccccaaatcaggcgatctccggagatgtgaagaaggggggcgagcggacaggaagatgaagggagcaaagctgcccgccgcgggacaggcgtctaggtgaacaagaaaatgaccgaagaaacacacccagacgatgacagctatattgtgcgtgtcaaggctgtggttatgaccagagatgactccagcgggggatggttcccacaggaaggaggcgggatcagtcgcgtcggggtctgtaaggtcatgcaccccgaaggcaatggacgaagcggctttctcatccatggtgaacgacagaaagacaaactggtggtattggaatgctatgtaagaaaggacttggtctacaccaaagccaatccaacgtttcatcactggaaggtcgataataggaagtttggacttactttccaaagccctgctgatgcccgagcctttgacaggggagtaaggaaagcaatcgaagaccttatagaaggttcaacaacgtcatcttccaccatccataatgaagctgagcttggcgatgatgacgtttttacaacagctacagacagttcttctaattcctctcagaagagagagcaacctactcggacaatctcctctcccacatcctgtgagcaccggaggatttataccctgggccacctccacgactcataccccacagaccactatcacctcgatcagccgatgccaaggccctaccgccaggtgagcttcccggacgacgacgaggagatcgtgcgcatcaacccccgggagaagatctggatgacggggtacgaggattaccggcacgcacccgtcaggggcaagtacccggacccctcggaggacgcggactcctcctacgtgcgcttcgccaagggcgaggtccccaagcatgactacaactacccctacgtggactcctcagactttggcctaggcgaggaccccaaaggccgcgggggcagcgtgatcaagacgcagccctcccggggcaagtcgcggcggcggaaggaggacggagagcgctcgcggtgcgtgtactgcagggacatgttcaaccacgaggagaaccgccggggccactgccaggacgcgcccgactccgtgagaacttgcatccgccgggtgagctgcatgtggtgcgcggacagcatgctctatcactgtatgtcggaccccgagggagactatacagacccttgctcgtgcgatactagcgacgagaagttttgcctccggtggatggctcttattgccttgtctttcctggccccctgtatgtgctgttacctgccccttcgggcctgctaccactgcggagtgatgtgcaggtgctgtggcgggaagcacaaagcggccgcgtgactcagtttccctcccttctccctccatccgcagccacaggggaactcgtctcttacatactctcatcttctcccccgctcccttccactccaaggagcgaggagggcaagcggcctcccagctccctggtacctcgaggcaccattccagccagggacgctgccgggtagactctccactccccctgccgcccacactgcagcagccacatccatacacacacgctcgcacagtgttctgaggaaggaaccttcgccacagactcctgtactattaacaatctgtaaccaagctaactgtctcatccatgtgttgatttcctgtttcctcctcccccgcctcttccagttcaaaggagtctgcaattggaactgctgattttcggtgggttttgtagttgatttttccaagagcgtcgaagactctctttctcttggttcaccttgcctgtcgctagcaagcatctggttcagcggaaatgggatgtgagaatgatgaaacccgacagaagtatctcagcctgcagtcagttattatgtataggaggtgagctagttaacaaacttgtaccacaaaacaatatcgctttaacttttctaaagccaaatttcccatgtaagctgcagtttctatctttagcccatcatcattttctgcccccccaaaatctgttgaaatgattcactgatgcaaaacattcacccgtaatagactgagaattgagccttaacttcagattaacttgtgagaatcaggaaaattccttcaactgcattgcatcctcttggaccagggctagaatggggatttcaggtttctatgagcctccccattacccctaaagtagaacattttttaaattgtgttgccaccacttctaaaaatttagcaatattagaataggatactagtgaagtaagaaattttgcttgttgttttgcaaaccaaatagtttcctcaaacacaaatcagtgttcccacgaacaagttccagttgagaaacactaaggttatggtgaataaaaccatagggagcttcttccccccaacccctggctattttatattaatggggagaggggatttttaaatgtcataaatttgaagagtggtgggttgcattttcttcatgggtttatgttttcgtttcattttggactcaatttcacatcaccaaattcctcatttatacttggggaaaaaacaaggccatatgtaaaaacccttccaatgcctaagtgtctttctcctgcaactccaaacccagactcgccactttgggtgcacaggtggttaggtcagccaactggttctgcctgtcgccttgccacggaggaggcttctaattagctggaaaagagtatttttctaatacgttgcaaggattagccaaatcttcttattgaagaaagaagaaaagtgaagagtggttacctatacctagcatagtacaatcagaacctcgtggagaccaccggggacaggcttgcggacgccggctgttcttcccgccacgatctttctgtggtagcggccagcagaagacatggcctgctccccactccctttccccactccttctttattgcacacaggacaccagtcttcaaggaaagggacttttttccagtctgccaatcatattgggaaagtgctagctgtgctcaccttcatggggctgtttccagctcgtccacaagctcatcgattttctttagtagattaccggtgtaaatacccagtgtgcttatgagtcagttagtagacgtcttcattcattggagtaactggtttaggctttccagtttggaaaaggagcagagagctgtccatctggattgatggaagaaaagagaacctcatccatgcctggagaacatctagaaaaccttcagccagcctccagtgctgtcgagagaccaccttcccccgacccggaggcacttccttggggtctttctctagggtctcttctctacaaagcacaacactaatgttcgtttccttagacctcagttcaagtgcccctatttatttcaataagaacgcacatatcccagctgttttttgtttgtcacctctatttagttgttacctgtttctctcttctttcaccccttgtccttttccacccttttaagagttacgctagcagatcttactccacgtatactttttggtttgtgaaggcatcggttaagggcacaaagacagccatggggacatttatgtaaatacgtctctaattgccacactgcagctgaacagtgtgtagtattttcccagtcagctttgccatactgacgtcaatcatttgagagaaattattcagattttatttttgtatctgtggtaacaaaacattaaccaaaagattttctgtccagaagcctccccgaccccccaagctatttgctcacattaacaaattaaagtgcctgaagcataattcattctttacctgtatactaaaaaccctgttgtattgatttttttataataagcctttttacctctgtgtaaaaaatatatatacaagtgtatgatgtacattttagttcttaacttttttttatggtttctaatatgtatgaccaatgtagccattgctttaaaatgtaccgtgtaaatataaacacatcctatcaga

SEQ ID NO: 32 - protein, 418 aa, Homo sapiens
MTEETHPDDDSYIVRVKAVVMTRDDSSGGWFPQEGGGISRVGVCKVMHPEGNGRSGFLIHGERQKDKLVVLECYVRKDLVYTKANPTFHHWKVDNRKFGLTFQSPADARAFDRGVRKAIEDLIEGSTTSSSTIHNEAELGDDDVFTTATDSSSNSSQKREQPTRTISSPTSCEHRRIYTLGHLHDSYPTDHYHLDQPMPRPYRQVSFPDDDEEIVRINPREKIWMTGYEDYRHAPVRGKYPDPSEDADSSYVRFAKGEVPKHDYNYPYVDSSDFGLGEDPKGRGGSVIKTQPSRGKSRRRKEDGERSRCVYCRDMFNHEENRRGHCQDAPDSVRTCIRRVSCMWCADSMLYHCMSDPEGDYTDPCSCDTSDEKFCLRWMALIALSFLAPCMCCYLPLRACYHCGVMCRCCGGKHKAAA

SEQ ID NO: 33 - DNA, 6682 bp, Homo sapiens
atcagtctccgcacgcggttccgcaggtggcagcgatggcccagtcctgaactccccgccatggccggcgcccccggcccgctgcgccttgcgctgctgctgctcgggatggtgggcagggccggcccccgcccccagggtgccactgtgtccctctgggagacggtgcagaaatggcgagaataccgacgccagtgccagcgctccctgactgaggatccacctcctgccacagacttgttctgcaaccggaccttcgatgaatacgcctgctggccagatggggagccaggctcgttcgtgaatgtcagctgcccctggtacctgccctgggccagcagtgtgccgcagggccacgtgtaccggttctgcacagctgaaggcctctggctgcagaaggacaactccagcctgccctggagggacttgtcggagtgcgaggagtccaagcgaggggaaagaagctccccggaggagcagctcctgttcctctacatcatctacacggtgggctacgcactctccttctctgctctggttatcgcctctgcgatcctcctcggcttcagacacctgcactgcaccaggaactacatccacctgaacctgtttgcatccttcatcctgcgagcattgtccgtcttcatcaaggacgcagccctgaagtggatgtatagcacagccgcccagcagcaccagtgggatgggctcctctcctaccaggactctctgagctgccgcctggtgtttctgctcatgcagtactgtgtggcggccaattactactggctcttggtggagggcgtgtacctgtacacactgctggccttctcggtcttatctgagcaatggatcttcaggctctacgtgagcataggctggggtgttcccctgctgtttgttgtcccctggggcattgtcaagtacctctatgaggacgagggctgctggaccaggaactccaacatgaactactggctcattatccggctgcccattctctttgccattggggtgaacttcctcatctttgttcgggtcatctgcatcgtggtatccaaactgaaggccaatctcatgtgcaagacagacatcaaatgcagacttgccaagtccacgctgacactcatccccctgctggggactcatgaggtcatctttgcctttgtgatggacgagcacgcccgggggaccctgcgcttcatcaagctgtttacagagctctccttcacctccttccaggggctgatggtggccatattatactgctttgtcaacaatgaggtccagctggaatttcggaagagctgggagcgctggcggcttgagcacttgcacatccagagggacagcagcatgaagcccctcaagtgtcccaccagcagcctgagcagtggagccacggcgggcagcagcatgtacacagccacttgccaggcctcctgcagctgagactccagcgcctgccctccctggggtccttgctgcaggccgggtggccaatccaggtgggagagacactcccagggacaagggaaggaagggacacacacacacacacacacacacacacacacacacacatacatcctgctttccctccccaaacccatcagacaggtaaatgggcagtgcctcctgggaccatggacacattttctcctaggagaagcagcctcctaatttgatcacagtggcgagaggagaggaaaaacgatcgctgtgaaaatgaggaggattgcttcttgtgaaaccacaggcccttggggttcccccagacagagccgcaaatcaaccccagactcaaactcaaggtcaacggcttattagtgaaactggggcttgcaagaggaggtggttctgaaagtggctcttctaacctcagccaaacacagagcgggagtgacgggagcctcctctgcttgcatcacttggggtcaccaccctcccctgtcttctctcaaagggaagctgtttgtgtgtctgggttgcttatttccctcatcttgccccctcatctcactgcccagtttctttttgaggggctttgtttgggccactgccagcagctgtttctggaaatggctgtaggtggtgttgagaaagaatgagcattgagacggtgctcgcttctcctccaggtatttgagttgttttggtgcctgcctctgccatgcccagagaatcagggcaggcttgccaccggggaacccagccctggggtatgagctgccaagtctattttaaagacgctcaagaatcctctggggttcatctagggacacgttaggaatgtccagactgtgggtgtagattacctgccacttccaggagcccagagggccaagagagacattgcctccacctctccttggaaatactttatctgtgaccacacgctgtctcttgagaatttggatacactctctagctttaggggaccatgaagagactctcttagggaaaccaatagtccccatcagcaccatggaggcaggctccccctgcctttgaaattcccccacttgggagcttgtatatacttcactcacttttctttattgctgtgaatagtctgtgtgcacaatgggcaattctgacttctcccatctagtggaaatgagcgaaatcatggttgtagtgatgttgtttgggagagtgcagtagtaattgatttgacccactcacacttggagctaattaaggtttgccctgcctgcagcctcccccacaaataatgaacagcagaaagactggacggggaaacctatcaatcctgcccccagccatggtgaggaagccccaagccatggtgacacacagcagcactgcagatagccagacacatggctatcctagagaggctggcaaggagttcgtggctgcaaaagaagtttctggagcaagagagagctcgctcttgggagtcaggacctccggggagagcagagggttccgacggattcctttatgagtcagtctctctctcccttttaaatggtgggaaccctccccaaaacctttccccagacacattctcctgtgcccctcagagaggcatgtgatgtgcaaggaaaataataggatataaaacacatcaagtagaaaatttcttatacttcagcttcagtgaagtgttgtctatgttaataggcaagttgaacctcgggctaaagaaaggaattgtgtggatggctgccttgactgagacaatatgggcaggggggcgttcctgcttccccagaacaagggcaggcttcccaaaggcaccccttatttgctgtctcttcgtaagcgtgggcaccagtgggttgatgtgggaaacagtgctggtgcaagtgtttaagttttggataaactgaggattttgagaatcattattacattacatagcaacaaagaaacagattgatataatctgtgtgaaataaattgctgaggagagggattgtagggaagaagttgcttagttaggcacctgggaagctcaaatcatttagtttaaactgtaagtagagttgtcttcccaacgagaactggtgattcctgatttcagaaggctcatcagacccctacacccaagtcttttaacacggaggaagtttttccttcatgaatacatacaagataccgaactgaagaaatcttttctttgctccaacccccgctcccccggccccccgaagctattaatagtcattcaaatggcactgcactctttccaagctttcctaagctaaatatggctccaacaggtctgggaactattgaatgcaatgtactctgaaattccagcagctgtttacatgcctgatgcctaatgccatacgcctggaacttgctgagaactctcggctgcctctgtcctggggtactattgctgaccagcagggtgtaggcgctgcttttgtttgagctttgtagccacaaggtctgatgtctgttgtgaacagcagctgacttcagcactctggccagcttcctcaaacttgttagtggcttgcattttgtcaatttttctttgccatttcagtctttcaagcagctgaatcgcagaactgaaacaaaacacttatattgcaaagttccttccctttaaaatgagcttctggattgcagagatgattttttatggtcttagcgtgcctcatgctgtagaccacatcgttggagctctgggagagctctgagcagagaaagaccttggtgatcccccctggcccgatctataggattgaggaatgaggcttgtagaggtggcttgcccaaggtcacaacactagagggtggttgagctgggccaggacccagacaagcccttttctctcagtctagtgaggcctctgggaaggataggtgaaaactataaatagctgttcaagatactctgagtaaaagtcagtggtgttatctggaatttctactgaagaggaaatacagcagcaaaggtagactcatcagatggtgaaagtgtcatctcctttgtaatttatactggtgagaggaggggaaggatgtgctttcctatttttacactggtacttcattcatccttgtcttaaacttaagaaatgaaatgcatcagggcacacatatacatagaaacagcacataagccattttggatggcaattccagcactcttttaatggattggcttaatttttttttctgatgcctctctatgtgtttgaaaagttgtattgatagtcacacagagtgtactaaaataacctaaaaggtcttgaacctcccctttacctctttagaacctctgaatttggagacagctagcaaacggcaactgtagcctagcaaagatggcacagcaccacaaagggcaaatggagtcaactcccttccatgctctgaaggtcttggccactctcatcagggttggctgtgtactatccagcagctcacagtcaggagacctcccctcctcagtccctcccatttctttcttccgcaaggaaagaacattgcatgtgggcgtgtgtgtgtgtatgtgtgtgcatgtgtgtgtgcatgtttcttctgtgcatagcagcataaagttaggagatgatgactccagaatcatcacacccgctgtgcaggtcataggaccgcacacccgaggctgggagtctaagtggtagatgcttcctctccatgcgcagacagggctcccattatttgctaatgcctgcgacgtgctcagggaaggcaaaatctattccaaaggtttattttccctctgttgaattgattctaatctacagtgcttaccctgcttttggctcctacgaggcaaactataaaaattctcaaataaaatacagaggcttactctgcaaagacacctgtgtgaatgacgtgctaaacattcagcagaaaccaaattaggatcaactcttaacattttttccctcttttgtggctggaatgagttcccctgggaaagaaaatcgcccaagtgagaaacttatgccaaccacattcccatgcccctacagttacctgctggcccagatcagctctctgggtctgcagagggccgagacaggaagcattattaagcagtataatttcaaacactttacttagtgccacagtgtgtctgcagtatctataagtacttccgctctgtcaaggaatacagactggcaggaagcagataagcataattgttttccaataactccccaagtctcttggaatatatatgtatctgaatctctcagaggagagagttttctctcttctttggccattttagcctattttccaggaccttgtctagcagtggctccatttttccccgatgcatccaacttccctccctgcactcctcctccccgacagctgcctcaggaataggtgaccctgctggccatactgtgaatgctgttctttcagctgtgaccaatttgggggtgcagctgagagtgaggtgctgtcagggctgaaaaggacgcacctcattctacatttggatgtgatgaggagcagggcagccaccagcttggatcctaacttacattgggtggtgcaacctttctgacggggcctgccaaactatcagtaattagccagcctagacaatgtagcatgactaattattgctgtgtcaaagcaattcaggacaagaaaaacatatctaaatgagaatgtttagatgtggtgagaaaagagctctcaccacaatttgaaacatataaaaagttgagttctttgcaggggagccaacgggggcattgagggaagggcaaaattcaccccagacagccagtgcttgatcttgccaaacagtttacactttacctccaaacctgcagttctaagctactccactaaaataatcatcccatcctcaaagcagaccaactccagcatcccatcggttctggagggatttgatgcaaacctttaaatgcaccaacagagaggtaagccaagctctcaggagggacaaagatagatttctatcttcagtgctcttgggggatgtttgctcctcatgagtgtttaaacaagatcatttccaggtgatttcttaacatataaattcctgcaaaataggaccagcttctataggtagcgaatgtgtaggtcatataattccttgtacagatgtgaatggatgcagactctttcctaatttgccttgtaagccaaataaaagtctgtctctactgtc

SEQ ID NO: 34 - protein, 463 aa, Homo sapiens
MAGAPGPLRLALLLLGMVGRAGPRPQGATVSLWETVQKWREYRRQCQRSLTEDPPPATDLFCNRTFDEYACWPDGEPGSFVNVSCPWYLPWASSVPQGHVYRFCTAEGLWLQKDNSSLPWRDLSECEESKRGERSSPEEQLLFLYIIYTVGYALSFSALVIASAILLGFRHLHCTRNYIHLNLFASFILRALSVFIKDAALKWMYSTAAQQHQWDGLLSYQDSLSCRLVFLLMQYCVAANYYWLLVEGVYLYTLLAFSVLSEQWIFRLYVSIGWGVPLLFVVPWGIVKYLYEDEGCWTRNSNMNYWLIIRLPILFAIGVNFLIFVRVICIVVSKLKANLMCKTDIKCRLAKSTLTLIPLLGTHEVIFAFVMDEHARGTLRFIKLFTELSFTSFQGLMVAILYCFVNNEVQLEFRKSWERWRLEHLHIQRDSSMKPLKCPTSSLSSGATAGSSMYTATCQASCS

SEQ ID NO: 35 - DNA, 8853 bp, Homo sapiens
agtggcaggagcgccgcgcaccgccagccgcagggggcgtgggatgggggcggccggggaggggggcgcccacactgactagagccaaccgcgcacttcaaaagggtgtcggtgccgcgctcccctcccgcggcccgggaacttcaaagcgggccgtgctgccccggctgcctcgctctgctctggggcctcgcagccccggcgcggccgcctggtggcgatgacccgggcgctctgctcagcgctccgccaggctctcctgctgctcgcagcggccgccgagctctcgccaggactgaagtgtgtatgtcttttgtgtgattcttcaaacttcacctgccaaacagaaggagcatgttgggcatcagtcatgctaaccaatggaaaagagcaggtgatcaaatcctgtgtctcccttccagaactgaatgctcaagtcttctgtcatagttccaacaatgttaccaaaaccgaatgctgcttcacagatttttgcaacaacataacactgcaccttccaacagcatcaccaaatgccccaaaacttggacccatggagctggccatcattattactgtgcctgtttgcctcctgtccatagctgcgatgctgacagtatgggcatgccagggtcgacagtgctcctacaggaagaaaaagagaccaaatgtggaggaaccactctctgagtgcaatctggtaaatgctggaaaaactctgaaagatctgatttatgatgtgaccgcctctggatctggctctggtctacctctgttggttcaaaggacaattgcaaggacgattgtgcttcaggaaatagtaggaaaaggtagatttggtgaggtgtggcatggaagatggtgtggggaagatgtggctgtgaaaatattctcctccagagatgaaagatcttggtttcgtgaggcagaaatttaccagacggtcatgctgcgacatgaaaacatccttggtttcattgctgctgacaacaaagataatggaacttggactcaactttggctggtatctgaatatcatgaacagggctccttatatgactatttgaatagaaatatagtgaccgtggctggaatgatcaagctggcgctctcaattgctagtggtctggcacaccttcatatggagattgttggtacacaaggtaaacctgctattgctcatcgagacataaaatcaaagaatatcttagtgaaaaagtgtgaaacttgtgccatagcggacttagggttggctgtgaagcatgattcaatactgaacactatcgacatacctcagaatcctaaagtgggaaccaagaggtatatggctcctgaaatgcttgatgatacaatgaatgtgaatatctttgagtccttcaaacgagctgacatctattctgttggtctggtttactgggaaatagcccggaggtgttcagtcggaggaattgttgaggagtaccaattgccttattatgacatggtgccttcagatccctcgatagaggaaatgagaaaggttgtttgtgaccagaagtttcgaccaagtatcccaaaccagtggcaaagttgtgaagcactccgagtcatggggagaataatgcgtgagtgttggtatgccaacggagcggcccgcctaactgctcttcgtattaagaagactatatctcaactttgtgtcaaagaagactgcaaagcctaatgatgataattatgttaaaaagaaatctctcatagctttcttttccattttcccctttatgtgaatgtttttgccattttttttttgttctacctcaaagataagacagtacagtatttaagtgcccataaggcagcatgaaaagataactctaaagttaagcatgggcaggagttgacttcatccaatctctatgttatgtttaattttattttgaaagcaacacctcaactcatctttttatttaataaggaagaaatatattacaaaagtataaaataagctctataaaaatgttatagtcattaagtttttattttacttgaaccaagagcacatgaatgaacaggaaaagatgtaaaaacatttttttctgagatgaaaacatattaattaaacatgcaaattagagcatgctatctttaggtgatgcaatctatgtttccccctttttaagttagcaggactttttaaaaataaatattgctctaaactttaatatatcgaacgtgagagtggagctgcttagtggaagatgtaagtgaggtgggtgtcccatgtgcttggtctccccttctgctgttctcctgttcttcataatccactactgcagcagtccctgaaccactaaacttgttcctttcatttacaaaagagatacctgacatcctgagacactgagaaatgtcctgaagtcacacagctaatggcagaactggcactaggtccaaatcttgtgataatgaacaccgtaaggttagctagcttcctactttcccttgaatagtgcttttctccctatgtaatatcttttattatgatatttgtggtttagaaggcatattgagttattttgcagaatcataatggacccgcacaaaatctcagaaccatatctgttgacattttttctcatagaaatatcatggttaccccatttgttaatgagcattaatgttttctgaacacttccaaagattaatcaaacataaatattcattgtctgaaaatgtctttaagatacaattcagaggtccctatttcctttgtacatacacacttagaaagaaaagacagaaaaggaagaggaaggaaggaaatattttgagaatatattgagaagaattaagaaaactcttcaatgaagtgttaacaaccaaaccctacagacggtatcagaaacagcaaatagatattcctctaccctttcacagtgagtgagtgagtacagaagaatgctcatgatagttttgccttcattctactttctgtggacacagagtaatgaatatttaatgggacattaaatatgcccttcaaatctataattttactttggtaaacgagatttaacatgatgtcttttatgctcctaaaacatcttttttcaaactccattccttagaacattcttctactgagatgatccaagaccaaaagtgttctttggtacttgcttataaagtgatagtacatgttagcatataatgtattttgaagagtgaagtaaatgctattgataacagtaaaaaaaaaaaaaaaaactaataacagtaaagaaatgctacttgatttttttttaaactggattgctcagattacctgatcgtggtggaacccttttattaagaggaggggaaactttttactatcccatatttaactgttctataaagcaaagcacagcttgggtaatagcgttctgaaggatatacttctgtattttctcatagagtacaatttagtgattatgcttcatttcactatggaaatatgttactgaatctatcttcattttactgagttgaaataaggaaggcaaaaaactgacagctatggagtttgcgtgtacttccatactcgttaatgctctcatccacttattaaataatcatagagcacccatatcttgcttgccacaatatcgggtacaagagggaatacaaagatagataggtcctgccctcagggattttaaagtctaatttgggaatgggaatagggatgtgagtgtgtgggggaagagattaaattgacagataaaatacgaagtgggatgtcttgagttctgcatgacagtgggtttctaggataggtctgaaaattgctttcatttgcaacacatttagaaagtagctttatttggatattacagacaatctaaatatatcatcagtttttaaaagtgcctatgtgaagtgatttttaaaaagagcctatgtgaaggggtaatcttgcttgttcttgttactaatttctcatagattgtttttctgcatatataagaacgaaattatttatttactatggttgtacgtgcctcaaataaacaagaatgatatttcctgttttatttacttatgttgggtaaatatgcttattgaatttttaagagaggattttttaccatctccatttttcttgtcattatgttttgtagcttatttgagggtgtctaaatataatttcatattttattggttcaactttcactctgaagaaatccgtatgttagtacattttgaggtatttttcttgttcttgtgttgtttaactatgactcctaactgagtagtcttatatttcaattacaaaatacattttttaagaaagggaatagagcagcaaaaatgataaggaaaatgttaaaagttgtaatatttcctttactcttaacaggattatatatagaacatgctcacttacaaaaataggatgatgaagtttagagcataaggcaggcttcttgtatatacttatgctgtcaaatgttatattgtttttaatggagtcccattgtgtaatatttatttcttttacattttgttataagcaaaaaaaaaaaaaattctccttaggttatgttcagagtatcagtgttctttactccttacagatattttggctttggggtataatacaagacttgggaaaacactattatgaattttcagtactgtataaagtggtgatgggatttaaatgcagcatcactttctgaaaataagagaaacattatttgttgtcagtatttcagcatgaacttgttgccttgtaaattttgcctttaagtttgtaattggtacagattctgttgtatgctttcttctatgtctaaaatatttggcatgtcacatctagaattcttaatttatgttctgacttgagagttaagtgaaacatgactgtcgtgcactattttaggcatagcacttgcttttcatctttatactttcaattaactttgcattttaaatttccatgattgtatgaaaatagtaacctggttgcagtatctgaaaaaattaaaattagcttttatttaaaaatgaaaatctacaatagattcattaggttagaagttccagaataatttattattttattacacctactattgtagaattactaataaaacaaaacaaaactactcctattttcctggaattttgccaccatgtgacttattggggcagagaaaactcagggttgtctttgagtctgcacaaaagcaccagggaacctgcttagcaaatcgtctgaaaacagggagctgatgtttgccattatacaaagtttgagtaaacaacttaaaattgcttgttagggcatagtctttgattgaaataagtatgagaatgtatttggctaaataaatgtatttaaaatatacaaattttatttcccactggaaaattaagaaagtagcagtaccaaatgaataaaagctggcagttgatgtcttcaataatcattcctttaaaataaattcacaaacacatcattacaagctacttagaaatgtttagtattcgtatcttaaaatggcaccattgtggttttcaaagatattttaaacatcttttgtgaaacatactatttctgtttgacaatgctttgttctagctctgtgtgctgatacctacatggagtttttctgctattttagtgaaaacagtaattcttttatcttgagagctgcttctaaataacaaaaaagttaattggaatgtaagtttttaaaaaatgttaatattaaatagaatttttataatatgggcatttttcaaaacattttaatgaaaatatatagatatttgactttctttttttcatctagccttgctttatatttcattatttttctcactttttttcttaatattccctcactatctttcaacattatcattagttcctaattttaaaaataactcttatttaacttaaccgtctggaatttagccttctaatgaaagagaatcccttagtactctcagagattatataaagttattccaattttgtgtagatgacgaaaccaaggatcagagattaaatgactactagttactagcagaactcttatgaaagcctggtggtgacacccagcactgttccctgccatatccccaacttttactgattaaaaattaattcgcatgcatctaaacctaccttgaatgcacactaacgtaatgtgccattcaatgatgatgataaaatcccacttttctttgggtttctactaaaataatttactcactcagagtgaggtcaatgagaaaaactaaactaggctaagaaagagttgtagaatggttgttgagctaagtaggcaaccactggggtgctgataaaattaatggataaaatttaggaactgtgagagtatagaatttcttagtgcaagtaatgattagagaagcttcctgacatcccccaccctttctgtaaggactgttctttctctttgtaccttggaatgggggtgaaaggtgatttgatagctgaatttgggactatgtccagtgggatattatctaacttttctctctttctcttttttttcccctcagtttctcattagtttgtctttggcattcatcttcttttagtgcattaaaaaatgtttggcagatctcatcaatcccaagtcactctataattcctgtatttctttagttgtcgtttaacctgtccaaacttctacacaatgaacttcttaacaagattttaagttctctgtataagaggtttcacctcataacttctccagtaatccttcatttggcactatagagtatttgttaatggcagagatgatttttcttttaaaacctaaaaagactagctgttatttgtattctagcttttagctaacatataaagaatgtctacttttgctttaatgctaaattccgcttgagaaatagtaactgggaaagacaatttgaaatatatgccccataatgtgattgttaaatttatttctgctgttccataccattgctttgttttgcttttgacaaactaagccattatgctattagtttggaatataatactacagcaaaattaggtaacaatccctattttaaaattcccctaacaataatagaactgccagcatacttttctctttcagttgtagatgaatacattcgagagaatatgagctgtatttcatcctagattttaatattttcagatgtgactgtatttcctgatcattggtccaagttgtcctaaaagaaatttttctctccagacctaacagttttaactgcaagagtttactgtgggttatgttaatctgaattttaatagggccactaagaatctgagtgccttaggagattacccttatacccactgccatcacatccagtcaggcctgttgtgctctatataaatcttcccagctgaggggcaggtgcgggctaaaatccaactgcaattggctcccagacataattttatattttacagagaagcatcttattggcttatatgtgtttaaagaatggtctggcttatacatcttcagaaaatgagaattaaaaagtcaaaataattcttgacatctacagattgaacaaagaacttagaagaaataatactttatcttttcatcctggcattcctgagagaagagaaattgattgtttatcatgttggtttaatttttcaacccagacaatctgcagcaaggcacatggaccccaattttgatatcgtccatacagttttcattctatgcatggagctaattactgactttgcctgtaaagagaggattgtgtgcctaaattttgtctaacaaatgcaagcgtagaatgacatttactaatatttctatttcttccataggctaaataatagtaactaagtatttttaaggacacagccctttttttctctttatacaaaatgagagtatctgagccaaaatattaaattctagttcttttccgcaatgactagtgtcaagctcatgtactcttctgattctagactggagaagattattcaaacttgatctgtgtttcaggtttttaaatgtcctaaaaacagaaaattagattcagatctcaaaaaaggaattttggattgactttcaaagtactaatactaattatacttttcttttggtagcgtgactcttcttatacctaagaacatattacaaatgtcaaaaccattgcattttgacattgcaaaacatgccttgaactcttgaactactgtgaaaagaatcaccgttgtaaagactttttgtaagctagctgatactcttaagtatgtaaaaagattgtctttcagccgacaggcccaaaggaatgtatataaggaaggaatatgaaaaaataaattaggttttaaaataggaattgggcaataaactgtatcaaaaatatgtagatggattttagtagttgtaatttaaatgtggaaggtgaagagaatttcaaactccaaagagaaatgaatgatattcagatgtttcattaatttctagtctgtgaaaatatgcattttatagtaatatgtatagacttattttatttagaaataatagtgttttagaatttattaaaaactcagtgatagcctttataccaaaatgtttaactttaccaacagcaagtcataaaagtatttattttaaagctttttaatattatcgtgtaactttcatctgtcttcagatgtaaataattatctgcctaaatgttatatttttatgtatgcattttctgaaaatgtattgttttgtaaagtgggaaagataataaatcaagcacttcttgcacttgtttctgtgaagcatatagaactctattttaaataaaggaagatgtgtcgta

SEQ ID NO: 36 - protein, 493 aa, Homo sapiens
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 37 - DNA, 4359 bp, Homo sapiens
gggggccccgccgccccagcagcccaggacagccccctctccccgcccccagccccctcccccggcgcggccatggatgtgaccagcagctcgggcggcggcggcgacccccggcagatcgaggagaccaagccgctgctggggggcgacgtgtcggcccccgaaggcacgaagatgggcgccgtgccctgccgccgggctcttctgctttgcaacgggatgaggtacaaactgctgcaggagggcgacattcaggtctgtgtcatccggcacccgcggacctttctcagcaagatcctcacctcgaaattcctgaggcgctgggagccgcaccacctaacgctggccgacaacagcctggcgtccgccacgccaactgggtacatggaaaactcagtctcctacagcgcaattgaagacgttcagctgctgtcctgggagaatgccccgaagtactgtttacagctcacgattcctgggggaactgtcttactgcaggctgccaatagctacctgcgagaccagtggttccattctctgcaatggaagaaaaagatttacaaatataagaaagtgctgagtaacccaagccgctgggaagttgtcttgaaagagatccggaccctggtggacatggccctgacatcccccctgcaggatgactccatcaaccaggccccactggaaatcgtctcgaaactgctctcagagaacacaaacttgaccacccaggagcatgaaaacatcattgtggcaatcgctcctttgctggaaaacaaccacccaccaccagatctctgtgaattcttttgcaagcactgcagagagcggccccggtccatggtggtcatcgaggtgttcacccccgtggtgcagcgaatcctcaagcataacatggactttgggaagtgcccgcgactgaggctgtttactcaggagtacatccttgccttgaacgagctcaacgcggggatggaagtggtgaagaagttcattcagagcatgcacggccccacagggcactgcccccacccccgggtcctgcccaacctggtggccgtgtgcctggctgccatctactcctgctatgaagagttcatcaacagccgcgacaattccccaagcctgaaggaaatccggaacggctgccagcagccgtgcgaccggaagcccactttacctctgcgccttctgcaccccagcccggacctggtgtctcaggaagccacgctgtctgaggcccggctcaagtcggtggtcgtggcctccagtgagatccacgtggaggtggaacgcaccagcactgccaagccggcgctgacggccagcgcaggcaacgacagcgagcccaacctcatcgactgcctcatggtcagccccgcctgcagcaccatgagcatcgagctgggcccccaggccgaccgcacgctcggctgctacgtggaaatcctcaagctgctgtcagactatgatgactggagaccgtctctggccagtttgcttcaacccattccattccccaaagaagctctcgcacatgagaagttcaccaaggaactgaagtacgtgattcagaggttcgccgaagaccccaggcaagaggtccactcatgcctgctgagcgtgcgggccggcaaagatggctggttccagctctacagccccggaggggtggcctgcgacgatgacggggagctgttcgccagcatggtgcacatcctcatgggctcctgttacaagaccaaaaaattcctgctctccctggcagaaaacaagctgggtccctgcatgctcctggcactgagggggaaccagaccatggtggagatcctgtgcttgatgctggaatacaacatcatcgacaacaacgacacccaactgcagatcatctcaaccctggagagcacagacgtggggaagcgcatgtacgagcagctgtgtgaccggcagcgggagctgaaggagctgcaaaggaaaggcgggcccaccaggctaacactgccctccaagtccacagacgctgacttggctcgtttgctgagctccggctccttcggaaacctggagaacctcagtttggccttcaccaatgtaaccagtgcctgcgccgagcacctcatcaaactgccttcgctcaagcagctgaacctgtggtccactcagtttggagacgctggccttcggctcctgtcggaacacctcaccatgctccaggtgctgaacctgtgcgagaccccggtcacagacgctggcctgctggccctgagctccatgaagagtctctgcagtttaaacatgaacagcaccaagctctcagctgacacctacgaagatctgaaggccaagcttcccaatttgaaggaagtggacgtccgctacaccgaagcctggtgaagctcccagctcaaggcaggaagacgtttgcaaccgcgacaaaataactcttgactaacagccgcagagcagccggtcctggggtcccaccctggtgccctggctgtgagatagatggggagtctttctgggggcggaggggggagggggtggggagggggcccacaagcacgcccagcccccgccgaattcttttagcttcgtaattggaacctttgacctgatctaaagtggactttgtagcaacaagaggagcatcagcgggtcggggaggggtttgggggtgggctggggggtgggggaccctttgtggattttctttgcctttgtgtttgatgccgtcgtgtgggaaaagtcaactccgatgccaccattgcgggccggacgaaggatgctttcttcctagaggctccgagctgagctgcgaactcgccccccgcccttgggacaagaagacccagtcacatcactgcacccgtcctgtgtcctcaccattgctatgcaaagtgattcttgttgtacataagatttaaataatgcacctatttaagacatgttgacaaattgcgggtctgggacccgcctcttatttatgaagtctttgaccgtcccccccgcccgaccccaccgccctcccgcccccacctggcgtgtagtactgtataaaccagtcagctgtcgggttagtggtagtattattgttatttttttaaaggaaacaaacagacaacaaaaagaagaaaaaaaaaaagaacctccttggaaaaattaattgctttttcgtaatggattctctatgctaatgctctctcgtctgtctgtctgtctgcccactcccccacccaccactgtgcgtttctgatttccaaatgtctccaactccctcacgaggtggggctcaggctggaggaggagggattaagatccccttgctccactaaggcccaagctctttctctcggcaccttttagacttgaatgggaggctgctaacccgccctctccagtccaccccggtaaaagagctgttccccacccccagggagctcctgtccctgtcagcctttgctgtcccctgtccccaacggagactctgtcacccctgggctccccctgccatcgtgtgcttcacgtggccccatgcatgcccgcctctctgcatggtctcttgggaaaagagagatgtgtcgcctccgccagtccgactgccctccccaccccacccccgccaccccccacatgtgaccactgcaacgaagacactccttctgtccccacctgctccgaagacaaaccaacctccgtttcttttataaacagtcggctttttcttaataagccctcactgtacagaacagcccgttgatggtttatttggggtccccctctccccccagcccttttttctgttggtttagcacaaatacttccctcctccggcacctccaaacctaccccacagtcagtgtacttgttttatatatatttaatcttattcaatggaaaccatgcttttgtcgttttatactttgctaggtagactttattacccccccactatgccctcatttttttaaaaaaggaaaaaaaaaagaaactgggttccagtcttaattcattttccgtgccaggttttatttcgtgtgtgtgtgagtgtgttctgttttgtgttttgttttttgttgttgtttttagttgtttggttttcttttctttcccccctccggtcccatacttcacagcactctggtgcgggaagaagcagaagcaaaaaaaataaaaataaaaaaataaataaaaataaaaaaaataaaaaaggaaaaaaaaaaaagaagaaacaagacatgccacctttcccctcgcactgttgcttttcctgatggttaatactactgtcacgtagctgtgtacaaagagatgtgaaatactttcaggcaaaaataaactgtaagtgactcatcaaaa

SEQ ID NO: 38 - protein, 773 aa, Homo sapiens
MDVTSSSGGGGDPRQIEETKPLLGGDVSAPEGTKMGAVPCRRALLLCNGMRYKLLQEGDIQVCVIRHPRTFLSKILTSKFLRRWEPHHLTLADNSLASATPTGYMENSVSYSAIEDVQLLSWENAPKYCLQLTIPGGTVLLQAANSYLRDQWFHSLQWKKKIYKYKKVLSNPSRWEVVLKEIRTLVDMALTSPLQDDSINQAPLEIVSKLLSENTNLTTQEHENIIVAIAPLLENNHPPPDLCEFFCKHCRERPRSMVVIEVFTPVVQRILKHNMDFGKCPRLRLFTQEYILALNELNAGMEVVKKFIQSMHGPTGHCPHPRVLPNLVAVCLAAIYSCYEEFINSRDNSPSLKEIRNGCQQPCDRKPTLPLRLLHPSPDLVSQEATLSEARLKSVVVASSEIHVEVERTSTAKPALTASAGNDSEPNLIDCLMVSPACSTMSIELGPQADRTLGCYVEILKLLSDYDDWRPSLASLLQPIPFPKEALAHEKFTKELKYVIQRFAEDPRQEVHSCLLSVRAGKDGWFQLYSPGGVACDDDGELFASMVHILMGSCYKTKKFLLSLAENKLGPCMLLALRGNQTMVEILCLMLEYNIIDNNDTQLQIISTLESTDVGKRMYEQLCDRQRELKELQRKGGPTRLTLPSKSTDADLARLLSSGSFGNLENLSLAFTNVTSACAEHLIKLPSLKQLNLWSTQFGDAGLRLLSEHLTMLQVLNLCETPVTDAGLLALSSMKSLCSLNMNSTKLSADTYEDLKAKLPNLKEVDVRYTEAW

SEQ ID NO: 39 - DNA, 6324 bp, Homo sapiens
gtcgtccgttcccaagctggtttgaaactaggggtcgggctcggccgtcgtcgttgtttgtcgccgcatccccgcttccgggttaggccgttcctgcccgccccctcctctcctcccttcggacccatagatctcaggctcggctccccgcccgccgcagcccactgttgacccggcccgtactgcggccccgtggccaccatgtccctgcacggcaaacggaaggagatctacaagtatgaagcgccctggacagtctacgcgatgaactggagtgtgcggcccgataagcgctttcgcttggcgctgggcagcttcgtggaggagtacaacaacaaggttcagcttgttggtttagatgaggagagttcagagtttatttgcagaaacacctttgaccacccataccccaccacaaagctcatgtggatccctgacacaaaaggcgtctatccagacctactggcaacaagcggtgactatctccgtgtgtggagggttggtgaaacagagaccaggctggagtgtttgctaaacaataataagaactctgatttctgtgctcccctgacctcctttgactggaatgaggtggatccttatcttttaggtacctcaagcattgatacgacatgcaccatctgggggctggagacagggcaggtgttagggcgagtgaatctcgtgtctggccacgtgaagacccagctgatcgcccatgacaaagaggtctatgatattgcatttagccgggccgggggtggcagggacatgtttgcctctgtgggtgctgatggctcggtgcggatgtttgacctccgccatctagaacacagcaccatcatttacgaagacccacagcatcacccactgcttcgcctctgctggaacaagcaggaccctaactacctggccaccatggccatggatggaatggaggtggtgattctagatgtccgggttccctgcacacctgtcgccaggttaaacaaccatcgagcatgtgtcaatggcattgcttgggccccacattcatcctgccacatctgcactgcagcggatgaccaccaggctctcatctgggacatccagcaaatgccccgagccattgaggaccctatcctggcctacacagctgaaggagagatcaacaatgtgcagtgggcatcaactcagcccgactggatcgccatctgctacaacaactgcctggagatactcagagtgtagtgttggtggcgctgtgcccacgaggcaggggcttttgtatttcctgcctctgccccacccccaaagtaagaagaaacatgtttccagtggccagtatgtctttcattgctttgcacccactgttaccagaagctgctctaggagttcctggccagtcaccccatcgccctctgtggcagactcagtgctgtgtggcgcctcctcagcccagggctgagttttaagattttctctcctttcctcttctcctttggttcctcaattaaaaaatgtgtgtatatttgtttgtcaggcgttgtgttgaggagcagttcacgcactggctgtgtctattcctctgcccaggtgtctctgtttgctgcccaaggcagcagttcatgtctcgtccatgtccatgttcgtgttagcacttacgtgggaacaaataccaatttgtcttttctcctagtatcagtgtgtttaacaaattttaactttgtatatttgttatctatcaggctaatttttttatgaaaagaattttactctcctgcttcatttctttgtcttatagtcctccctctttgcaccttcttctcttccctcagtgcctggagctggtactgggcccctggccccatgagcagtttgccttcttgagtcactgcctgtgtagtacatacctgaccgggagtccaaaccaccttggtgctctgaagtccactgactcatcacacctttcttagcctggctcctctcaagggcattctgggcttgtaaacagacataggaagcctctgtttaccctgaagcaccactgtccagcccattggttcccactggcagcatggtagagctgagagaaacaggctctcagggtacctgacttgaggggaatcgtttcatgaagctgaacttcaagcatatttccagtacattctttcagagtctgtttttccatccaaatataagccccaggccattccacttagtgtcttttcaatgataggcaagaatgatatctgagttgaacttcggtgcttctgttgtttgagtttactgtgcctggtggtatattgggcattctttggattgagtgttctgaggtgagagagtcttcccgaggcatcctgtctgtgcttccaaccctgaacaagaccttacatgagagatggactgatggactgcggcaatcctgggctgtcaagtggatagatagttaaaaagcattatactgtgggtaatgaaaagggaggaaaaaaaaagaaggaaaaggaattatagacccccagggtcagccagttaagagctctacccacacctgtcaacccctctctcccccagtttaggttctgagcagtattggacttgtagcctgcagttgtcttttgacttgcaggccgcaggtgtctttctgttatgtgaatgagttccatggaggggcatatgtgtgattccaccgttagatgagcccttggggcaggcagtttgggatgtgctcttgggggaaagttggctgtttccttgcgctctgctcctacccgaaggtttttaagtccctctgaattgctcatctgagattagtagagtagcaggcctgaaggatgatggttttgtcctctttggttctcacctgcttgagaagtaaaacagtaactttgttcttctgggcccttaagcttttttggttaagtcttccttttcagaagtagatgtcattatatgccaaaagtctagctctttgctttaccatacagggacctgtcccaaagaaaaaggctctttttttagccagcatatttccccttctacccttttactttgttgttctgattttaggactctggctggccatgtgcttgtggttgcctctcctgcatttgccactggatttgcactgcatcgtttggagatacaaagcgagcagttcttggtcagaaccctcctctgcttttcattgtgtttgataatggttactgggtccttctctcaagggtagcaaggccaagctgatggctgcttgtttaggaggccatcagttccttcctgtggagaagggtctgaaatggaagtcagtggtagaaggggctggtctgctgggcagggcttacatccactgagttctaagattcctttcctgatctgcacctacgcctggtctgtatggtggaatttgtcagctggaactcagaaacaacaacttgaaaaaaaaataataattagaacatatttgcataagatagctatttactctggaaaccaacaacttttgagatttcccttgccctgtggacgcccagctcctgtcatccttccttaggtcctgcagtacagtcttcccctgaatgccaccggggacccagggggactccacccccctaagcaagcacacacatactcacagttgatgagttgctggtctttgagtcccagctctcttaccctccctttactccaccagcccgacgacccatgactgaggaggggatttctacagtctcaggatttagaaagtctgtaagccatccatgctccagaaagcaccgatctgttgtagttgcaaaaacaactctgtaatttgttgaggttctcaaactgacagccagcgagactgggtgggaggccctggatctgttctccctgactgcgggaggagcagccactaggactttagcaggaagcccacatggaggctccgccaggctgtggcccagctggtgatggcccttttgctcctggcagcctgaggcacagctgcctgtattgtcctcatctgttctgactgaaggatggaggtgctgaataaattaggcctcaggcctctaccaccagagagctggagaatgggtccacgtcattcaaggacctgaattttttatgctcaggagcattggaatcctcttcttccagggaggaattagcctgcaaggttaggacttgaagagggaaggtatttaataactgggcgaggatgggtgtggtggctcacacctgtaatcccagcattttgggaggctgaggtggccagatcccaaggtcagaagatcgagaccatcctggctaacatggtgaaaccccatctctactaaaaatacaaaaaaaaattagccgggggtggtggcgggtacctgtagtcctagctacttgggaggctgaggcaggagaatggcgtgaacctgggaggtggagcttgcagtgagccaagatcgtgccactgcactccagcctgggcgacagagcaagactccgtctcaaaaaataaaaaaaaaaaaaaaataggtgaaaattccttataaatccaggattggctctgagagaactggctaagattcaggaagaaacaaaaaattcagaatcctacaaggttttgatgacaattagggccaaaattttaggaggagatgtaggatgcaggagaaaattaaagtgttttctttatatcagaggaggaaatagtagaggtcagtgaaggtctggggtagggaaacattcagactgtccattgcatggctgtggagtgagactgcccttagcctgggtcagccttcctgggccataaattgggcatccgtgatgctaggtaactgtgggaacaaaatgacagcttagagcagccatgggtgatgtttggtggtaaaaaacctacaggcgtttggggtcccatgattgttccagaccatgactcttcctggttgtgggtttgttacagagcaggagaagcagaggttatgacagttatgcagactttccccctcctttttctcttttctcttccccttgcttttccactgtttcttcctgctgccacctgggccttgaattcctgggctgtgaagacatgtagcagctgcagggtttaccacacgtgggagggcagcccagtactgtccctctgccttccccactttgagaatatggcagcccctttcattcctggcttggggtaggggagaccattgaagtagaagcctcaaagcagacttttccctttactgtgtgtactccaggacgaagaaggaagatcatgcttgatacttagattggttttcccagggaagagggcggagcagagcaaagtcactgtgaaccctgggccaggccctggctgggccagctcctgagagcgtctcgtgttgcagacccttgcccacttcacccacctgcaccttctccccctctcacagtgtcactgctgctaatggtcaaagtcaaatgtgtggccacatgggatgggccaggtcctctcaggctactttctggatgtcatttttaaaatatggaaacatgcaggtgccttcccaaagaggcttggactggtatatccaacgagaaacaaataagctaaagaaagtttaaactcaagaagaaagatgttgacagtctatgtaacagctggaaagtttataggcacccacctttgggacaacccagtgattatgaacatgtgatatctactatttaaaagaaatgttctcaccttgggttgattgtggtataccatgtgttatgaaaattgttgagctgaagctttgaatcgatttagttgagtctgactcacttgctttggttcctgtgtattttactacccctcttgtcagtgaccttccttccccaccccacccagagtgaatttgtagcatgattgtataaacctctatgtagaaaatggagatttcttgctctgaaatgttaagctctaactgatccatttctgtgtcctttagcctagtatgtctgaacttccattcttgttatatatttaaactttccctctatattataggttttgtggcatccacggtcaggtgtagaggaagctgccccttgcagaactgtactgtaatatttttcttttataaatattttcacaggactgattgtacacagggcttgtaataaaattttaacactgtgctgtgaaacaactatggggaatctccattgaaggctacttcatgggcacctgaaagtggagtgttatagctatgactttctatttcttgtttcctaagtaaattaaacctaattttcaccctttcattctgtttcagcctcctgtataagaagtaccgtattttctgcccatcatactttgtaataaaacttgaacatgta

SEQ ID NO: 40 - protein, 342 aa, Homo sapiens
MSLHGKRKEIYKYEAPWTVYAMNWSVRPDKRFRLALGSFVEEYNNKVQLVGLDEESSEFICRNTFDHPYPTTKLMWIPDTKGVYPDLLATSGDYLRVWRVGETETRLECLLNNNKNSDFCAPLTSFDWNEVDPYLLGTSSIDTTCTIWGLETGQVLGRVNLVSGHVKTQLIAHDKEVYDIAFSRAGGGRDMFASVGADGSVRMFDLRHLEHSTIIYEDPQHHPLLRLCWNKQDPNYLATMAMDGMEVVILDVRVPCTPVARLNNHRACVNGIAWAPHSSCHICTAADDHQALIWDIQQMPRAIEDPILAYTAEGEINNVQWASTQPDWIAICYNNCLEILRV

SEQ ID NO: 41 - DNA, 1787 bp, Homo sapiens
gaggagtggagatggcggcggcggcggctcaggggggcgggggcggggagccccgtagaaccgagggggtcggcccgggggtcccgggggaggtggagatggtgaaggggcagccgttcgacgtgggcccgcgctacacgcagttgcagtacatcggcgagggcgcgtacggcatggtcagctcggcctatgaccacgtgcgcaagactcgcgtggccatcaagaagatcagccccttcgaacatcagacctactgccagcgcacgctccgggagatccagatcctgctgcgcttccgccatgagaatgtcatcggcatccgagacattctgcgggcgtccaccctggaagccatgagagatgtctacattgtgcaggacctgatggagactgacctgtacaagttgctgaaaagccagcagctgagcaatgaccatatctgctacttcctctaccagatcctgcggggcctcaagtacatccactccgccaacgtgctccaccgagatctaaagccctccaacctgctcatcaacaccacctgcgaccttaagatttgtgatttcggcctggcccggattgccgatcctgagcatgaccacaccggcttcctgacggagtatgtggctacgcgctggtaccgggccccagagatcatgctgaactccaagggctataccaagtccatcgacatctggtctgtgggctgcattctggctgagatgctctctaaccggcccatcttccctggcaagcactacctggatcagctcaaccacattctgggcatcctgggctccccatcccaggaggacctgaattgtatcatcaacatgaaggcccgaaactacctacagtctctgccctccaagaccaaggtggcttgggccaagcttttccccaagtcagactccaaagcccttgacctgctggaccggatgttaacctttaaccccaataaacggatcacagtggaggaagcgctggctcacccctacctggagcagtactatgacccgacggatgagccagtggccgaggagcccttcaccttcgccatggagctggatgacctacctaaggagcggctgaaggagctcatcttccaggagacagcacgcttccagcccggagtgctggaggccccctagcccagacagacatctctgcaccctggggcctggacctgcctcctgcctgcccctctcccgccagactgttagaaaatggacactgtgcccagcccggaccttggcagcccaggccggggtggagcatgggcctggccacctctctcctttgctgaggcctccagcttcaggcaggccaaggccttctcctccccacccgccctccccacggggcctcgggacctcaggtggccccagttcaatctcccgctgctgctgctgcgcccttaccttccccagcgtcccagtctctggcagttctggaatggaagggttctggctgccccaacctgctgaagggcagaggtggagggtggggggcgctgagtagggactcagggccatgcctgcccccctcatctcattcaaaccccaccctagtttccctgaaggaacattccttagtctcaagggctagcatccctgaggagccaggccgggccgaatcccctccctgtcaaagctgtcacttcgcgtgccctcgctgcttctgtgtgtggtgagcagaagtggagctggggggcgtggagagcccggcgcccctgccacctccctgacccgtctaatatataaatatagagatgtgtctatggctga

SEQ ID NO: 42 - protein, 379 aa, Homo sapiens
MAAAAAQGGGGGEPRRTEGVGPGVPGEVEMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPGVLEAP

SEQ ID NO: 43 - DNA, 5733 bp, Homo sapiens
agacggacactgtgccggggcgagctgacaggagttcacggctgcgatagaacatggagatgtcatgggcgcgacagagcctggcggggataccagcagcgtgtgatgagttccacccgttcatcgaggcactgctgcctcacgtccgcgctttctcctacacctggttcaacctgcaggcgcggaagcgcaagtacttcaagaagcatgaaaagcggatgtcgaaggacgaggagcgggcggtgaaggacgagctgctgggcgagaagcccgagatcaagcagaagtgggcatcccggctgctggccaagctgcgcaaggacatccggcccgagttccgcgaggacttcgtgctgaccatcacgggcaagaagcccccctgctgcgtgctctccaaccccgaccagaagggcaagatccggcggattgactgcctgcgccaggctgacaaggtgtggcggctggacctggtcatggtgattttgtttaaggggatccccctggaaagtactgatggggagcggctctacaagtcgcctcagtgctcgaaccccggcctgtgcgtccagccacatcacattggagtcacaatcaaagaactggatctttatctggcttactttgtccacactccggaatccggacaatcagatagttcaaaccagcaaggagatgcggacatcaaaccactgcccaacgggcacttaagtttccaggactgttttgtgacttccggggtctggaatgtgacggagctggtgagagtatcacagactcctgttgcaacagcatcagggcccaacttctccctggcggacctggagagtcccagctactacaacatcaaccaggtgaccctggggcggcggtccatcacctcccctccttccaccagcaccaccaagcgccccaagtccatcgatgacagtgagatggagagccctgttgatgacgtgttctatcccgggacaggccgttccccagcagctggcagcagccagtccagcgggtggcccaacgatgtggatgcaggcccggcttctctaaagaagtcaggaaagctggacttctgcagtgccctctcctctcagggcagctccccgcgcatggctttcacccaccacccgctgcctgtgcttgctggagtcagaccagggagcccccgggccacagcatcagccctgcacttcccctccacgtccatcatccagcagtcgagcccgtatttcacgcacccgaccatccgctaccaccaccaccacgggcaggactcactgaaggagtttgtgcagtttgtgtgctcggatggctcgggccaggccaccggacagcccaacggtagcggccagggcaaagtcccggggtcatttttgctaccgccgccgcctccagtggccagacctgtgccccttcctatgcctgattccaaatccaccagcactgccccagacggcgccgccttgactcctccatcaccttcattcgcaacgacaggcgcctcctctgccaaccggtttgtcagcatcggaccccgggacggcaactttctgaacatcccacagcagtctcagtcctggttcctctgataagatcgacaaaagaaacaacaaaatgagaagaagaggttcctcgaaaggggggagaagaaattttgagaatggaaaaatcccccagcccagcccagccccaccgaaaagcaaaaattacacgtcgtcagccactcagcccttctctcctccagcccggggacccccgcgggccccagaagcagcccagttctcagagagcccttggaaggggtctcggtggagctgtgcaccagcagccaagcagaaagaaacacgcgacatggactctgtcaagtagaggacagaaagcaagaaaggatgcagaactgccttcctccccctgaccccgccccggccttctggggaaggaacaaagtccccaaacaaagcaaccagcacaattctgaaggggcctggcctccaccctcaccccttcctaggggaaccccaccctccacacagccggagctgccctagggagcctggagggccagcttgtaaagatgatggggtttagatccctcaggctctcccctccagactccgcccttccctccctccctccctccctccctctctgccaaggctccagcttcttcccccagctgctcccgaccaggagggggagagcagcctccacttaccccaccccacccttgggctaaaagcccccaggcgggcagggggtgacccctggagctagttgcgtgtcccagaatggagggtgttctgacaccccaccctgagccgcaagagcagtcctggggccctggacccctctgtacagtccgtaggaaaaagtcggaatgctctcgacggcctcgtcccagcctgggacaggccccctttcccctctctctgcaggccaggagggcctccttcctgccacgagggaggggagtcgggccccaggtcgcccccgcccccagccctgcatgcaggtgccctcgctccgccccatcagttcctgcccctgcccctcatgcagactgccctgctggggccgggccggagggtggagcagaaaggggaccccggagccgagcgaggaggaccaggcagccgccgctgccgcgctaagccaccacctgcgcttaggtaggcgtcctgctcgccgactttcagttccttgggagggtgttgggtgtcgtccttttcaaaagtgttttggagctttctgtgccccccgactttcccccgcctccccgccccccacgtggccacttttctctggattttagctgtaatgtctttactctttatttaggggtggggcattcattgtttgggtcttttgctgttggaatgggaactcctcctccatttgagcaacttgggaacaatttggtaacacaccacaggaagtagctctcccccccagccccctcctccctcaagggagggttggggggcctgtccagagggtcttcagaagcccccctgggagggaggggaggatgagcacgcccagctcccctccagggtgtgacttggcccctctggcttgtctttctgtgccttactcctcctcctgcgtctcccgttcctggccccttcttgagtccttgtgcctctctctttctctctctttcttaattgtatgaaaacacaaagcacaggtcaggatcctctgagagaaaatcaacattgcaccacgtaggggtgggctatgggctgtatttattgtgaatctagtttgtgaggctgtggccccgagctggcggagggagggaagaggagggagtgacgggaggggaggaggtcagcgacctggggccgtagcggcaggcgaacggtgcctgctacccagctggaagccacaaggtggctggctccaggggcggcttttgttggaagttgagtgaagccctccccctgtcctcagcgtgcagccctagaggaccccagggctgaggggcagtggatcctgcgggagtctcccggggcgtggggagtaaggccccgggggtggggggccgggtgggccgggcgtgacgcgcggtcaaagtgcaatgatttttcagttcggttggctaaacagggtcagagctgagagcgaagcagaaggggctccctgtccggcccacgtgccctttccctcgacgacagtcgagggctcgggctctgtgggactgtgggagctagggtctgcggggcgcctgcccgggcgaggtcggaagctgcaggccagctgggcccgggccggagcgtgcccggcggggctgcccgggcgggcagggggtgggggctgctcctttcccaagtggtgttgtgaggggcaatgagggcaacaggagatgtggggacgtgttaggagagaaaaaaaaaaaaacaaaaatatatatgggggaaattaacttttttttttcattgaaccaagtgcaatgcatcagagagttttcctatctttgtatgttaagagattaagaaaaaaaaattctatttttgttgtaatgtcctcgcggctctggggacgctaaaagaaccgggcctgccccgccctgcgcggggataacgaaagctgagtgtttttcccttttttttgttcgtttttagtttttttttttttaagtcgttttcctgcgttgacgaggatgatctggggtttttatttgtttcgtcgttcgttctgtttcggtgggagggctgaaggaaacgttcacattttagagtttaaaaaaaacacctcgacatttaaaaaatcaaccaacacaagatcaaaaaggaaaaggacgagagaaaaattatttttaagataattaaacataaaaccctggtgcttcttacattataaagtacgttttaaagaacccacaaactattatacataagtttatgaatcaattaaatatcctgcacttgttaggaatacgcatatcccttctttgttgagtttaacggaacgggacagcggcgtgcccccggcggctggactgctccggccgcgggtctccccgggcgcccctccctggggcccagcacccctcctcgccccatccccgtccgggtacgggggcgcggcaggggtccccggcccctcccccgcagaggtcaatgccaacgaacaaacgtcccctccctccctccctctccgccccgagcgcccttctttgagccagacgccaacttgaccctcaccagcattatcaggagcgcgctcagcaagttggtagtttcctcccccctttcccggcgcccctcccgcccccattcaacatctctcatcctatccccgaccccctccggggaacaccgggaaggctcgacgctccaggacaggaccagccacgctgacaggtcgatttgcccaggcccgcgcccgcacgcacgcacgcacacggccccgcacacagccccgccccaccccgcaaccagccctgtcgactgccttatacacccgcccccgcgctggccggccgacctagtgccttgttctcacccccgtgctggcggagcggacgccgcgctctgggtcccagaggggccgggtggctcagacgacccaccactcccccaccctgaccgtgctgaacagacccccccacacgagagaaaataaaggagcaataaagtcacgagaactttcgtcccccaatcgagagcccgaggggcaccccagccccgcctctgctcccccccaccccacccaccctcggggcgcccccctccccccgcaagccagcctgggccagccccgcttcggcccctcccgggagatccgtgcgcccgaccagcaccagcatcgcggaccgcaaaggccgcccgtcccgtcaaacaagtttcttcttaggctaagaaacgcagtatatacgagtatctctatatatagtactaatggatttggtgtgcttcccccttagcgtccccctccctctgctcctcctccttcagcctggtctccccctcttctctgccctccacccccgtctctgcactgagatacataagaaacaagggtagtttactgtctgttttgttttctgggttttcagtgtcctagcggaatgcaagtaggcagccagcccgtctgttccctctccgccccgccccgccccgcccccgtcactgcgcttctgttataccatctttgcctgactctctccggcttctccattgaatggctaatgtgtatgtgaaataaagaaataaagaaaaacaaacgcga

SEQ ID NO: 44 - protein, 510 aa, Homo sapiens
MEMSWARQSLAGIPAACDEFHPFIEALLPHVRAFSYTWFNLQARKRKYFKKHEKRMSKDEERAVKDELLGEKPEIKQKWASRLLAKLRKDIRPEFREDFVLTITGKKPPCCVLSNPDQKGKIRRIDCLRQADKVWRLDLVMVILFKGIPLESTDGERLYKSPQCSNPGLCVQPHHIGVTIKELDLYLAYFVHTPESGQSDSSNQQGDADIKPLPNGHLSFQDCFVTSGVWNVTELVRVSQTPVATASGPNFSLADLESPSYYNINQVTLGRRSITSPPSTSTTKRPKSIDDSEMESPVDDVFYPGTGRSPAAGSSQSSGWPNDVDAGPASLKKSGKLDFCSALSSQGSSPRMAFTHHPLPVLAGVRPGSPRATASALHFPSTSIIQQSSPYFTHPTIRYHHHHGQDSLKEFVQFVCSDGSGQATGQPNGSGQGKVPGSFLLPPPPPVARPVPLPMPDSKSTSTAPDGAALTPPSPSFATTGASSANRFVSIGPRDGNFLNIPQQSQSWFL

SEQ ID NO: 45 - DNA, 4342 bp, Homo sapiens
agtcttaaccgggtgtgcggggagcgcagtccgggtgcgtaggggccgctcggcgggggccgcgcgggcaagatggtgtgcgctcgggcggccctcggtcccggcgcgctctgggccgcggcctggggcgtcctgctgctcacagcccctgcgggggcgcagcgtggccggaagaaggtcgtgcacgtgctggagggtgagtcgggctcggtagtggtacagacagcgcctgggcaggtggtaagccaccgtggtggcaccatcgtcttgccctgccgctaccactatgaggcagccgcccacggtcacgacggcgtccggctcaagtggacaaaggtggtggacccgctggccttcaccgacgtcttcgtggcactaggcccccagcaccgggcattcggcagctaccgtgggcgggctgagctgcagggcgacgggcctggggatgcctccctggtcctccgcaacgtcacgctgcaagactacgggcgctatgagtgcgaagtcaccaatgagctggaagatgacgctggcatggtcaagctggacctggaaggcgtggtctttccctaccacccccgtggaggccgatacaagctgaccttcgcggaggcgcagcgcgcgtgcgccgagcaggacggcatcctggcatctgcagaacagctgcacgcggcctggcgcgacggcctggactggtgcaacgcgggctggttgcgcgacggctcagtgcaataccccgtgaaccggccccgggagccctgcggcggcctgggggggaccgggagtgcagggggcggcggtgatgccaacgggggcctgcgcaactacgggtatcgccataacgccgaggaacgctacgacgccttctgcttcacgtccaacctgccggggcgcgtgttcttcctgaagccgctgcgacctgtacccttctccggagctgcgcgcgcgtgtgctgcgcgtggcgcggccgtggccaaggtggggcagctgttcgccgcgtggaagctgcagctgctagaccgctgcaccgcgggttggctggccgatggcagtgcgcgctaccccatcgtgaacccgcgagcgcgctgcggaggccgcaggcctggtgtgcgcagcctcggcttcccggacgccacccgacggctcttcggcgtctactgctaccgcgctccaggagcaccggacccggcacctggcggctggggctggggctgggcgggcggcggcggctgggcagggggcgcgcgcgatcctgctgcctggacccctctgcacgtctaggctgggagtaggcggacagccagggcgcttgaccactggtctagagccctgtggtcccctggagcctggccacgcccttgaagccctggacactggccacattccctgtggtcccttacaaactaactgtgcccctggggtccctgaagactggctagtcctggcagaacagtactttggagttccctggagcctggccagccctcacctcttctggatagaggattcccccaactccccaactttctccatgagggtcacgccccctgaggacctcaggaggccagcagaacccgcaggctcctgaagactggccacgcctcctgagaccacttggaaacagaccaactgcccccgtggtcgcctggtggctggacccccgggattgactagagaccggccgtacaccttctgcatctcactggagactgaacactagtcccttgcggtcacgtgggacactgggcgcctcctcctccccctcctcctcacctggagagactacaggaacttcagggtcactccccgtggtcacatggaggttgtgggccgaggcgcttattttcccttatggtgacctgagtcctggagactcccattctccccctctccctgagagtcccctgcagtttctgggtaacagggcacacccctctagtttcatgggcgagcacccccatctgccacctcagactgacacacagccagctggctcacttactgggggccacgtcccacccctcagatatttctttgaagggagagcaaacccaccctgtcctctgacgtccctttcccaactgtcaccaaacagaccatcttcccaggcctggggaccggtaagatccatgtcactagttatgcagagcagttgccttgggtcccactgtcaccaaggcaaccagtcctgctgctacctgtcacctagagtcacacaccccttccctcatcaggcacacccatgaagacagtgcctccctcctccagctgtaaccatggataccacacatttctcatctcattggcccccaccccagagacctccacctcaacttctggctgtccctaccctgactcaccgccatggagatcaccctccccgaagctgtcgccagggtgacccaacatccagttctccggctctcaccatggaaacaaactgtccctgtccccaggcccactccagttccagaccaccctccatgctccacccccaggcggtttggaccccaccactgttgccatggtgaccaaactctggagtccgaggtaacagaacacctgtccccctaggcttttccttgtggacaacggggccctgttcaccaagctgttgccatagagactgtcaacgttgtcctcatgacaaccagacttccagttctcaggaacttctcattgtgggccagaagtcctgggtgcctcctactagggctaccctactgcaccccatcaggggcctgatggctgccccttccccagacagggctggacttctggagctgctaagccaccctccgtttgcacgttaactctatgccggatagcagctgtgcacgagacaatcttgcaacacccgggcatgtttgtcgtcgtcctacaaatgaggaaaccgagcctatggcgtgccctggtctgttgagatatgcaagcactgagctcctcttttgtcctctgagaccccatctccattctcacccagttcctctctccttccctgacccccacccacatttccctccttagagatccaggagggatggaatgttctttaaaattcaacacccaccaggctctaagcggcgatctgtgctaagaggtcaggacccagccgaagtcctcggcgttgacaggcagctggggggacatgatccatggacaaggccatcccggccgtgggagaccccagtcccgaagtcttgcctgcaggagtactggggtccccctggggccctctttactgtcacgtcatctctaggaaacctatctctgagttttgggaccaggtcggtttgggtttgaattctgcctcttcttgctcactgtgtgaccaagtgacaaactccttctgaacctgtgttctcccactgtaccagggctgttctgtggtccccgtgagtgccaagcatacagtaggggctcaataaatccttgtttcttttgatgaatgagaaaatgaggcagccagtgggtaattcctgtataaatgcactttggtagataagatgttacaagcttggggggcttggggttttttttgttttgtttttttgagatggagtcctgccctgtcgcccaggctggagtgcagtcgtgcaatcttggctcactgcaacctctgcctcccgggttcaagtgattctcctgcctcagcctcccgagtagctgggattacaggtgcctgccaccacacccggctaatttttgtatttttagtagagacagggtttcaccatgttggccaggctggtcttgaactcctgacctcaggtgatctacccacctcggcctcccaaagtgttggggttacaggtgtgagccactgcgcctggccgggcttgatttttattctctttggaagtggggtccttcattcctcccccacctccccaatctcttgctcctctttcttcccccatcctgtgcccactgtctccctttagccacccaccatgggtctgctccttgtcaactctgtcctgactgggtcattgacaagatccagggcatgaaattcgggaaactggaaggggggttcctgtaccaggaagggagagacattacaaactttccctgacatgaatatgtgggctgagggcaggggctgaggaagcactggaagtttctaggatacaacaaagcatgaagagaaaaaatagtgtaaggtcctgaccgtgcacagtggatcatgcctataatcccagcactttaagagaccaaggtgggaggattgcttgagccagaaggtcgaggctgcagtgagccatgattgtaccactgcactccagcttgggcgacggagtgagaccctgtctcaaaaaataaaaataagtaaatctcc

SEQ ID NO: 46 - protein, 402 aa, Homo sapiens
MVCARAALGPGALWAAAWGVLLLTAPAGAQRGRKKVVHVLEGESGSVVVQTAPGQVVSHRGGTIVLPCRYHYEAAAHGHDGVRLKWTKVVDPLAFTDVFVALGPQHRAFGSYRGRAELQGDGPGDASLVLRNVTLQDYGRYECEVTNELEDDAGMVKLDLEGVVFPYHPRGGRYKLTFAEAQRACAEQDGILASAEQLHAAWRDGLDWCNAGWLRDGSVQYPVNRPREPCGGLGGTGSAGGGGDANGGLRNYGYRHNAEERYDAFCFTSNLPGRVFFLKPLRPVPFSGAARACAARGAAVAKVGQLFAAWKLQLLDRCTAGWLADGSARYPIVNPRARCGGRRPGVRSLGFPDATRRLFGVYCYRAPGAPDPAPGGWGWGWAGGGGWAGGARDPAAWTPLHV

SEQ ID NO: 47 - DNA, 1670 bp, Homo sapiens
aatggggacctggaacctgggcttactagagtgccgcgcgtagggctccaggtcgctggcttctgcgcttccttcctctccaaagttgagtatctcctatctgtgtcctcgtacatactgccgcctgaggtgccatggcccccaagccgggggccgagtggagcacagccctgtcccatctggtgctgggagtggtgtctctgcacgcagccgtgagcacagccgaggcaagtcgaggggctgctgctggcttcctgctccaggtcttggctgccaccaccacgctggccccagggctgagcacacacgaagactgccttgctggagcctgggtggccaccgtcatcggccttccccttctggccttcgatttccactggtgtactaatggtcacttgatgtgcgctggctgttttatccacctactagcagatgcccggctgaaggaggagcaggccacgtgccccaattgtcgttgtgagatcagtaagagcctctgctgccggaacctggccgtggagaaagccgtgagcgagctgccttcagagtgtggcttctgcctgcgccagtttccccgctccctcctggagaggcaccagaaagaggaatgccaggacagggtaacccagtgcaagtacaaacgcatcggctgcccatggcacggccccttccatgagctgacggtgcacgaggctgcgtgcgcccacccgaccaagacaggcagtgagctgatggagatcctggatgggatggaccagagccaccgcaaggagatgcagctgtacaacagcatcttcagcctgctcagcttcgagaagattggctacacagaggtccagttccggccgtaccgcacagacgacttcatcacgcgcctgtactatgagacgcccaggttcacagtgctgaaccagacgtgggtcctgaaggctcgagtcaacgactcggagcgtaaccccaacctgtcctgcaagcgtacgctctccttccagctcctcctcaagagcaaggtcacggcaccgctggagtgctccttcctgctgctcaagggcccctacgacgacgtgaggatcagccccgtcatctaccactttgtcttcaccaacgagagcaacgagacggactacgtgccactgcccatcattgactccgtggagtgcaacaagctgctggctgccaagaacatcaacctgcggctcttcctgttccagatacagaagtagggcggggcctcaggatgtccgaggagcccacgggcggcatcccagcaccgctgccctgtccacctggctggcagctgcttcacaggactatctgatcactttagcaaaggaggagaacaaacgaagccaacacagggcaagtctgcatgcgtgcgcgacggggcccccgcctccggctcacccccccgacccctgcctcccctccttccgagggccgccagaggctgggctgacccgaagaggagacggtgcaccaggcgccccgaggctcagagacggtggcagcaaggaggccgagaggcacagcgaccctgccccagcccttctgtgcagtcaggcggcggtgctgctccatccctgcgggttccggcggggcgcgggggccttgctgacatcagacgggatatccgaatatctgatagcaattaaaaggcagccttgtttcgt

SEQ ID NO: 48 - protein, 362 aa, Homo sapiens
MAPKPGAEWSTALSHLVLGVVSLHAAVSTAEASRGAAAGFLLQVLAATTTLAPGLSTHEDCLAGAWVATVIGLPLLAFDFHWCTNGHLMCAGCFIHLLADARLKEEQATCPNCRCEISKSLCCRNLAVEKAVSELPSECGFCLRQFPRSLLERHQKEECQDRVTQCKYKRIGCPWHGPFHELTVHEAACAHPTKTGSELMEILDGMDQSHRKEMQLYNSIFSLLSFEKIGYTEVQFRPYRTDDFITRLYYETPRFTVLNQTWVLKARVNDSERNPNLSCKRTLSFQLLLKSKVTAPLECSFLLLKGPYDDVRISPVIYHFVFTNESNETDYVPLPIIDSVECNKLLAAKNINLRLFLFQIQK

SEQ ID NO: 49 - DNA, 3145 bp, Homo sapiens
gagactgaaaggaaccataatttgtgacatcagttgttttctttgataagcagctatttatgattctggaagattaaggcagataggaaaccccatctgagattttaataaatccctcaaacaataaaccacatcatggacatacagctggaccctgccagagatgacctgcctctcatggccaacaccagccacatacttgtgaagcactatgtactggatttggatgtggattttgaaagtcaagtcattgaggggaccatagtgcttttcctcgaggatggaaacagattcaagaaacagaatagctctattgaggaagcctgccaatcagaatcaaacaaagcctgcaaatttgggatgcctgaaccctgccatattcccgtgacaaatgcaaggaccttctcatctgaaatggaatataatgattttgcaatctgtagtaaaggtgaaaaagatacttctgataaagatggtaaccatgacaaccaggaacatgcttctgggatttctagctcaaagtactgctgtgacacagggaatcatgggagtgaggattttttgctagtgttggactgctgtgatttatctgtgttaaaagtcgaggaggtggatgttgctgctgtgccaggtctggaaaaatttacaaggtctcctgagctcacggttgtttctgaggagttcaggaatcagattgtacgtgaacttgtgactttgcctgcaaatcgttggagggagcagttagactattacgctcgctgcagccaggctcctggctgtggggaactcctctttgacactgacacttggagcttgcagataaggaagacaggggctcagacagctactgactttcctcatgctatcaggatatggtacaaaactaaacctgaagggcgatcggttacatggacctcagaccagagtggcaggccatgtgtttatactgtgggatctcccataaacaacagggccctttttccatgccaggagccacccgttgccatgtcaacatggcaggctacagttcgagcagctgcatcttttgttgttttaatgagtggggaaaattctgccaaaccaacgcagctttgggaagagtgctcaagctggtattactatgtaactatgccaatgccagcctccaccttcacaattgcagtgggatgctggacagaaatgaagatggagacatggtcatcaaatgatttggcaacagagagacccttctcaccttctgaggccaacttcaggcatgttggtgtttgcagtcacatggaatacccctgccgcttccagaatgcttctgccaccacccaggagatcattcctcatcgggtctttgcccctgtgtgcctcacgggtgcctgccaagagacccttctgcggctgatccctccttgcctctcagcagcacattctgttctgggagcacacccgttctctcggctggatgttctcatcgtccctgccaactttccaagtctggggatggccagcccacacatcatgttcctctctcagagcatcttgacaggagggaaccatctctgtgggacccgcctctgccatgaaattgcccatgcctggtttggcctagccatcggggcccgagactggacggaggagtggctgagtgaaggcttcgccactcacttggaggatgtgttttgggccacagcacagcagctggccccctatgaggcccgggagcagcaggagctgagggcttgtctgcgctggcgtcgcctccaggacgagatgcaatgctcccccgaggagatgcaggtgttaagacccagtaaagacaaaactggccacacaagtgactcgggagcatctgttatcaagcatggacttaatccggagaagatcttcatgcaggtgcattatttaaagggctacttccttcttcggtttcttgccaaaagacttggagatgaaacctatttttcatttttaagaaaatttgtgcacacatttcatggacagctgattctttcccaggatttccttcaaatgctactggagaacattccagaagaaaaaaggcttgagctgtctgttgaaaacatctaccaagactggcttgagagttccggaataccaaagccgctgcagagggagcgtcgcgccggggcggagtgcgggcttgcgcggcaagtgcgcgccgaggtcacgaaatggattggagtgaaccggagaccccgaaaacggaagcgcagggagaaggaagaggtgtttgaaaagcttcttccagaccagctggtcttgcttctggagcatctcttggagcagaagactctgagcccccgaactctgcaaagcctccagaggacataccacctccaggatcaggatgcagaggttcgccatcggtggtgtgaactcattgttaagcacaagttcacgaaagcctacaaaagtgtggagaggttccttcaggaggatcaggccatgggtgtgtacctctacggggagctgatggtgagtgaggacgccagacagcagcagctcgcccgtaggtgcttcgagcggaccaaggagcagatggataggtcctcagcccaggtggtggccgaaatgttattttaacgaggaaagaccacagcaagattctttcattcgtctcctcctagcctgggggaccaggctcgaactgaccctggacatcaaaggagggattatgtggctgctaaagccatcggcccacagccctgttcacatcttggtgcttctctttcccagaggctggtcccagccaggcacacacaaaaggcagattctcgtaaacgcagcctccctccctggaggctgcctcctgccctggatctggagtggagctgctctgagattttgagttcttctgcagagatgattaaatatatccaagagacattggaaaacctgctgaacattttacattggtctgctcagcacatggctggatgcggatatttctataattccagaaagtcacacagctcctctgtatgagaccagtgggcgccatttaaaagaacaggatgagaatctaagatatattattaataaatgtaatggattttttttttgtatacgtgtttgcttctaaatttcatactgtttaaaaataataaaggccaggtgcggtggcaaaaaaaaa

SEQ ID NO: 50 - protein, 819 aa, Homo sapiens
MDIQLDPARDDLPLMANTSHILVKHYVLDLDVDFESQVIEGTIVLFLEDGNRFKKQNSSIEEACQSESNKACKFGMPEPCHIPVTNARTFSSEMEYNDFAICSKGEKDTSDKDGNHDNQEHASGISSSKYCCDTGNHGSEDFLLVLDCCDLSVLKVEEVDVAAVPGLEKFTRSPELTVVSEEFRNQIVRELVTLPANRWREQLDYYARCSQAPGCGELLFDTDTWSLQIRKTGAQTATDFPHAIRIWYKTKPEGRSVTWTSDQSGRPCVYTVGSPINNRALFPCQEPPVAMSTWQATVRAAASFVVLMSGENSAKPTQLWEECSSWYYYVTMPMPASTFTIAVGCWTEMKMETWSSNDLATERPFSPSEANFRHVGVCSHMEYPCRFQNASATTQEIIPHRVFAPVCLTGACQETLLRLIPPCLSAAHSVLGAHPFSRLDVLIVPANFPSLGMASPHIMFLSQSILTGGNHLCGTRLCHEIAHAWFGLAIGARDWTEEWLSEGFATHLEDVFWATAQQLAPYEAREQQELRACLRWRRLQDEMQCSPEEMQVLRPSKDKTGHTSDSGASVIKHGLNPEKIFMQVHYLKGYFLLRFLAKRLGDETYFSFLRKFVHTFHGQLILSQDFLQMLLENIPEEKRLELSVENIYQDWLESSGIPKPLQRERRAGAECGLARQVRAEVTKWIGVNRRPRKRKRREKEEVFEKLLPDQLVLLLEHLLEQKTLSPRTLQSLQRTYHLQDQDAEVRHRWCELIVKHKFTKAYKSVERFLQEDQAMGVYLYGELMVSEDARQQQLARRCFERTKEQMDRSSAQVVAEMLF

SEQ ID NO: 51 - DNA, 54 bp, Artificial
  synthetic
agcagtggtatcaacgcagagtactttttttttttttttttttttttttttttt

SEQ ID NO: 52 - DNA, 28 bp, Artificial
  synthetic
aagcagtggtatcaacgcagagtacggg

SEQ ID NO: 53 - DNA, 23 bp, Artificial
  synthetic
aagcagtggtatcaacgcagagt

(19) United States
(12) Patent Application Publication
(10) Pub. No.: US 2022/0404336 A1
(43) Pub. Date: Dec. 22, 2022

(54) COMPOSITIONS, METHODS AND USES FOR FREE FATTY ACID SCREENING OF CELLS AT SCALE

(71) Applicants: THE BROAD INSTITUTE, INC., Cambridge, MA (US); THE BRIGHAM & WOMEN'S HOSPITAL, INC., Boston, MA (US); PRESIDENT AND FELLOWS OF HARVARD COLLEGE, Cambridge, MA (US)

(72) Inventors: Anna Greka, Boston, MA (US); Nicolas Wieder, Cambridge, MA (US); Juliana Coraor, Cambridge, MA (US)

(73) Assignees: THE BROAD INSTITUTE, INC., Cambridge, MA (US); THE BRIGHAM & WOMEN'S HOSPITAL, INC., Boston, MA (US); PRESIDENT AND FELLOWS OF HARVARD COLLEGE, Cambridge, MA (US)

(21) Appl. No.: 17/641,683

(22) PCT Filed: Sep. 9, 2020
(86) PCT No.: PCT/US2020/049905

§ 371 Date: Mar. 9, 2022

Related U.S. Application Data
(60) Provisional application No. 62/898,514, filed on Sep. 10, 2019.

(51) Int. Cl. G01N 33/50; C07K 14/765; C07K 1/107; C12Q 1/6883

(52) U.S. Cl. CPC G01N 33/5023; C07K 14/765; C07K 1/1077; C12Q 1/6883; C12Q 2600/158; G01N 2800/7085

(57) ABSTRACT

The present disclosure relates to compositions and methods for the preparation and use of free fatty acids as crystals and in solution, optionally in array format, for assay of lipotoxicity and related effects (in certain cases, indicators of diseases or disorders, such as type II diabetes) in contacted cells. Identification and therapeutic targeting of high-value gene targets discovered via joint assessment of lipotoxicity/transcriptome data and genetic association study data are also provided.

### CROSS REFERENCE TO RELATED APPLICATION

This application claims the benefit of U.S. Provisional Application No. 62/898,514, filed Sep. 10, 2019, entitled “Compositions, Methods, and Uses for Free Fatty Acid Screening of Cells at Scale.” The entire contents of the aforementioned application are incorporated herein by reference.

### FIELD OF THE INVENTION

The current disclosure relates to preparation of free fatty acids (FFAs) and use of FFAs to screen for lipotoxic and other effects upon cells, tissue and/or organisms, as well as to identification of high value gene targets for drug development and treatment of lipotoxicity-associated diseases and/or disorders.

### BACKGROUND OF THE INVENTION

Genomic studies have to date identified a number of disease-associated risk variants, yet integrating genetic risk factors with environmental risk has thus far posed a significant challenge. The obesogenic environment, characterized by an overconsumption of dietary lipids, results in the accumulation of toxic free fatty acids (FFAs) in many tissues (7). Certain FFAs therefore pose a clear environmental risk to cells, tissues and/or organisms. However, FFAs are poorly soluble in aqueous solutions. As a result, a need exists for compositions and methods that might allow for assessment of the effects of a large panel of FFAs upon contacted cells, at scale, and for improved methods of integrating both environmental data and genetic association study data.

### BRIEF SUMMARY OF THE INVENTION

The current disclosure relates, at least in part, to the identification herein of compositions and methods for the preparation and use of free fatty acids (FFAs) as crystals and in solution, optionally in an array format, for assay of lipotoxicity and related effects (e.g., indicators of lipotoxicity-related diseases or disorders such as type II diabetes (T2D)) in contacted cells, tissues and/or organisms. Performance of FFA screening of mammalian cells at scale (as enabled by the FFA arrays of the instant disclosure, in certain embodiments produced via use of a high-throughput evaporator upon arrayed FFAs) has succeeded in identifying herein both a new class of lipotoxic FFAs and—when transcriptome analysis of FFA effect was combined with gene lists derived from a genome-wide association study (GWAS) of T2D genetic associations—new high value target genes for therapeutic modulation of T2D. Therapeutic modulation of these high value T2D target genes is therefore also expressly contemplated herein.

A scalable cell-based platform for the study of lipotoxicity has been developed and described herein as a model for the obesogenic environment. An unbiased transcriptomic signature of lipotoxicity in pancreatic beta cells was derived and annotated with several functional assays including cell viability, insulin secretion and ER Ca²⁺ levels. It was thereby shown that the integration of transcriptomic signatures of lipotoxicity with T2D genomic risk profiles has the potential to nominate genes of interest at the intersection of genetic and environmental risk as promising therapeutic targets.

In one aspect, the instant disclosure provides a method for producing a bovine serum albumin (BSA)-conjugated free fatty acid (FFA) crystal, the method involving: a) providing a FFA dissolved in a solvent; b) transferring the FFA to a well of a plate, where the plate well includes a BSA solution, thereby forming a FFA-BSA solution; c) incubating the FFA-BSA solution for a duration of time and under conditions suitable to conjugate the FFA to the BSA; and d) drying the FFA-BSA solution to form a FFA-BSA crystal, thereby producing a BSA-conjugated free fatty acid (FFA) crystal.

In one embodiment, the solvent is DMSO or ethanol.

In another embodiment, the BSA solution includes ddH₂O.

In certain embodiments, the FFA-BSA solution has a FFA:BSA concentration ratio of approximately 6.67:1. Optionally, the FFA concentration in the FFA-BSA solution is approximately 500 μM.

In some embodiments, the FFA-BSA solution is incubated for 12-48 hours, optionally about 24 hours. Optionally, the FFA-BSA solution is incubated at about 37° C.

In one embodiment, drying of the FFA-BSA solution in step (d) is performed using a high-throughput evaporator.

In another embodiment, the FFA-BSA crystal formed in step (d) is free of the solvent.

In embodiments, drying of the FFA-BSA solution is performed under vacuum.

Optionally, drying is performed for a duration of approximately 6-24 hours, optionally approximately 12 hours. In a related embodiment, drying is performed at about 37° C. In some embodiments, the drying step further involves centrifugation. In a related embodiment, centrifugation is performed at about 400 g.

In certain embodiments, the method further involves resuspending the FFA-BSA crystal in cell culture media, thereby creating a resuspended FFA-BSA solution. Optionally, the cell culture media is pancreatic beta cell culture media, endothelial cell culture media, hepatocyte cell culture media, macrophage cell culture media, skeletal muscle cell culture media, or adipocyte cell culture media. In a related embodiment, the pancreatic beta cell culture media is MIN6 cell culture media.

In some embodiments, the method further involves filtering the resuspended FFA-BSA solution through a filter. Optionally, the filter has an approximately 0.45 μm pore size. In a related embodiment, the filter is a spin filter. In certain embodiments, the resuspended FFA-BSA solution is filtered into a well of an array plate, optionally a microwell of a 384 well microarray plate.

In some embodiments, a method of the instant disclosure is repeated to produce an array of BSA-conjugated FFA crystals and/or resuspended FFA-BSA solutions.

In embodiments, the array of BSA-conjugated FFA crystals is produced by drying with a high-throughput evaporator in step (d).

In certain embodiments, preparation of the array of BSA-conjugated FFA crystals and/or resuspended FFA-BSA solutions is performed in parallel.

In some embodiments, the method is repeated with different FFAs to produce an array of BSA-conjugated FFA crystals and/or resuspended FFA-BSA solutions. In related embodiments, preparation of the array of BSA-conjugated FFA crystals and/or resuspended FFA-BSA solutions is performed in parallel.

In another embodiment, the method further involves contacting the resuspended FFA-BSA solution(s) with a cell or array of cells. Optionally, the cell or array of cells is a pancreatic beta cell or array of cells (optionally a MIN6 cell or array of cells), an endothelial cell or array of cells, a hepatocyte cell or array of cells, a macrophage cell or array of cells, a skeletal muscle cell or array of cells, or an adipocyte cell or array of cells.

In an additional embodiment, an FFA of the FFA-BSA solution is delivered into the cell. In embodiments, the FFA is incorporated into (identified in) cellular lipids of the cell. In related embodiments, each of the FFAs of the array of FFAs is successfully delivered to a distinct cell/array element/well of cells. In embodiments, each of the FFAs is incorporated into cellular lipids of each of the targeted wells of cells.

An additional aspect of the instant disclosure provides a composition that includes an array of FFA-BSA crystals or FFA-BSA solutions, where each element of the array includes a single FFA and the array includes two or more distinct FFAs.

In one embodiment, the array includes five or more, ten or more, fifteen or more, twenty or more, thirty or more, forty or more, fifty or more, or all FFAs of Table 1.

In certain embodiments, the array is assembled in wells of a plate. Optionally, the array is present in wells of a 96 well plate, or in microwells of a 384 well (or higher well count) microplate.

In embodiments, each element of the array further includes cells or tissues in culture. Optionally, the cells or tissues in culture are pancreatic beta cells or pancreatic tissue, endothelial cells or endothelial tissue, hepatocyte cells or liver tissue, macrophage cells, skeletal muscle cells or skeletal muscle tissue, or adipocyte cells or fat tissue. Optionally, the pancreatic beta cells are MIN6 cells. In embodiments, each cell or tissue in culture includes a distinct FFA that has been incorporated into cellular lipids of the cell or tissue.

In one embodiment, the array is prepared by a method as disclosed herein.

Another aspect of the instant disclosure provides a method for identifying a lipotoxic FFA, the method involving a) providing a composition that includes an array as described herein; b) contacting the composition with cells or tissues in culture; and c) assessing levels of cell death and/or biomarkers of apoptosis and/or lipotoxicity in the cells or tissues in culture contacted with the composition, as compared to an appropriate control, thereby identifying a lipotoxic FFA.

A further aspect of the instant disclosure provides a method for identifying a lipotoxic FFA disease or disorder-associated gene, the method involving a) providing a composition including an array as described herein; b) contacting the composition with cells or tissues in culture; c) measuring the transcriptome of the cells or tissues in culture and identifying transcripts that are differentially expressed between lipotoxic FFAs and non-lipotoxic FFAs; d) producing a rank ordered list of genes that encode for the transcripts identified as most differentially expressed between lipotoxic FFAs and non-lipotoxic FFAs; e) comparing the rank ordered list of genes of step (d) with a rank ordered list of genes identified as most genetically associated with the lipotoxic FFA disease or disorder; and f) identifying a gene that both (i) encodes for a transcript identified as highly differentially expressed between lipotoxic FFAs and non-lipotoxic FFAs and (ii) is highly genetically associated with the lipotoxic FFA disease, thereby identifying a lipotoxic FFA disease or disorder-associated gene.

In one embodiment, the lipotoxic FFA disease or disorder is type 2 diabetes (T2D), obesity, cardiovascular diseases (CVD), non-alcoholic fatty liver disease (NAFLD), obesity-mediated inflammation/metaflammation or insulin resistance.

In certain embodiments, the cells or tissues in culture are pancreatic beta cells or pancreatic tissue, endothelial cells or endothelial tissue, hepatocyte cells or liver tissue, macrophage cells, skeletal muscle cells or skeletal muscle tissue, or adipocyte cells or fat tissue. Optionally, the cells or tissues in culture are MIN6 cells.

In some embodiments the lipotoxic FFAs are

| | |
|---|---|
| CC\C═C/C\C═C/C\C═C/CCCCCCCCCCCC(O)═O | 13(Z),16(Z),19(Z)-Docosatrienoic acid |
| CCCCCCCC\C═C/CCCCCCCCCC(O)═O | 11(Z)-Eicosenoic acid |
| CCCCCCCC\C═C/CCCCCCCCCCC(O)═O | 12(Z) Heneicosenoic acid |
| CCCCCCCC\C═C/CCCCCCCCCCCC(O)═O | 13(Z)-Docosenoic acid |
| CCCCCCCC\C═C/CCCCCCCCCCCCC(O)═O | 14(Z)-Tricosenoic acid |
| CCCCCCCC\C═C/CCCCCCCCCCCCCC(O)═O | 15(Z)-Tetracosenoic acid |
| CCCCCCCC\C═C\CCCCCCCCC(O)═O | 10(E)-Nonadecenoic acid |
| CCCCCCCC\C═C\CCCCCCCCCC(O)═O | 11(E)-Eicosenoic acid |
| CCCCCCCC\C═C\CCCCCCCCCCCCC(O)═O | 14(E)-Tricosenoic acid |
| CCCCCCCCCCC\C═C/CCCCCC(O)═O | 7(Z)-Nonadecenoic acid |
| CCCCCCCCCCC\C═C\CCCCC(O)═O | 6(E)-Octadecenoic acid |
| CCCCCCCCCCC\C═C\CCCCCC(O)═O | 7(E)-Nonadecenoic acid |
| CCCCCCCCCCCC(O)═O | Dodecanoic acid |
| CCCCCCCCCCCCCC\C═C/CCCC(O)═O | 5(Z)-Eicosenoic acid |
| CCCCCCCCCCCCCCC(O)═O | Pentadecanoic acid |
| CCCCCCCCCCCCCCCC(O)═O | Hexadecanoic acid |
| CCCCCCCCCCCCCCCCC(O)═O | Heptadecanoic acid |
| CCCCCCCCCCCCCCCCCC(O)═O | Octadecanoic acid |
| CCCCCCCCCCCCCCCCCCC(O)═O | Nonadecanoic acid |
| CCCCCCCCCCCCCCCCCCCC(O)═O | Eicosanoic acid |

Optionally, the non-lipotoxic FFAs

| | |
|---|---|
| CCCCCCCC\C═C\CCCCCCCCCCCC(O)═O | 13(E)-Docosenoic acid |
| CCCCCCCCCC(O)═O | Decanoic acid |
| CCCCCCCCCCC(O)═O | Undecanoic acid |
| CCCCCCCCCCCCC(O)═O | Tridecanoic acid |
| CCCCCCCCCCCCCC(O)═O | Tetradecanoic acid |
| CCCCCCCCCCCCCCCCCCCCC(O)═O | Heneicosanoic acid |
| C═CCCCCCCCCC(O)═O | 10-Undecenoic acid |
| C═CCCCCCCCCCC(O)═O | 11-Dodecenoic acid |
| C═CCCCCCCCCCCC(O)═O | 12-Tridecenoic acid |
| CCCC\C═C\CCCCCCCCC(O)═O | 10(E)-Pentadecenoic acid |
| CCCCCC\C═C/CCCCCCCC(O)═O | 9(Z)-Hexadecenoic acid |
| CCCCCC\C═C/CCCCCCCCCC(O)═O | 11(Z)-Octadecenoic acid |
| CCCCCCCC\C═C/C\C═C/C\C═C/CCCC(O)═O | 5(Z),8(Z),11(Z)-Eicosatrienoic Acid |
| CCCCCCCC\C═C/CCCCCCCC(O)═O | 9(Z)-Octadecenoic acid |
| CCCCCCCCCCC\C═C/C\C═C/CCCC(O)═O | 5(Z),8(Z)-Eicosadienoic acid |
| CCCCCCCCCCC\C═C/CCCCC(O)═O | 6(Z)-Octadecenoic acid |
| CCCCCCCCCCC\C═C/CCCCCCC(O)═O | 8(Z)-Eicosenoic acid |
| CCCC\C═C/CCCCCCCC(O)═O | 9(Z)-Tetradecenoic acid |
| CCCC\C═C/CCCCCCCCC(O)═O | 10(Z)-Pentadecenoic acid |
| CCCC\C═C\CCCCCCCC(O)═O | 9(E)-Tetradecenoic acid |
| CCCCC\C═C/C\C═C/CCCCCCCCC(O)═O | 10(Z),13(Z)-Nonadecadienoic acid |
| CCCCC\C═C/C\C═C/CCCCCCCCCC(O)═O | 11(Z),14(Z)-Eicosadienoic acid |
| CCCCC\C═C\C\C═C\CCCCCCCC(O)═O | 9(E),12(E)-Octadecadienoic acid |
| CCCCCC\C═C/CCCCCCCCC(O)═O | 10(Z)-Heptadecenoic acid |
| CCCCCC\C═C\CCCCCCCC(O)═O | 9(E)-Hexadecenoic acid |
| CCCCCC\C═C\CCCCCCCCC(O)═O | 10(E)-Heptadecenoic acid |
| CCCCCC\C═C\CCCCCCCCCC(O)═O | 11(E)-Octadecenoic acid |
| CCCCCCCC\C═C/CCCCCCCCC(O)═O | 10(Z)-Nonadecenoic acid |
| CCCCCCCC\C═C\CCCCCCCC(O)═O | 9(E)-Octadecenoic acid |
| CC\C═C/C\C═C/C\C═C/C\C═C/C\C═C/C\C═C/CCC(O)═O | 4(Z),7(Z),10(Z),13(Z),16(Z),19(Z)-Docosahexaenoic |
| | acid |
| CC\C═C/C\C═C/C\C═C/C\C═C/C\C═C/CCCC(O)═O | 5(Z),8(Z),11(Z),14(Z),17(Z)-Eicosapentaenoic acid |
| CC\C═C/C\C═C/C\C═C/C\C═C/C\C═C/CCCCCC(O)═O | 7(Z),10(Z),13(Z),16(Z),19(Z)-Docosapentaenoic acid |
| CC\C═C/C\C═C/C\C═C/C\C═C/CCCCC(O)═O | 6(Z),9(Z),12(Z),15(Z)-Octadecatetraenoic acid |
| CC\C═C/C\C═C/C\C═C/CCCCCCCC(O)═O | 9(Z),12(Z),15(Z)-Octadecatrienoic Acid |
| CC\C═C/C\C═C/C\C═C/CCCCCCCCCC(O)═O | 11(Z),14(Z),17(Z)-Eicosatrienoic Acid |
| CCCCC\C═C/C\C═C/C\C═C/C\C═C/CCCC(O)═O | 5(Z),8(Z),11(Z),14(Z)-Eicosatetraenoic Acid |
| CCCCC\C═C/C\C═C/C\C═C/C\C═C/CCCCCC(O)═O | 7(Z),10(Z),13(Z),16(Z)-Ocosatetraenoic Acid |
| CCCCC\C═C/C\C═C/C\C═C/CCCCC(O)═O | 6(Z),9(Z),12(Z)-Octadecatrienoic Acid |
| CCCCC\C═C/C\C═C/C\C═C/CCCCCCC(O)═O | 8(Z),11(Z),14(Z)-Eicosatrienoic Acid |
| CCCCC\C═C/C\C═C/CCCCCCCC(O)═O | 9(Z),12(Z)-Octadecadienoic acid |
| CCCCCC\C═C\C═C/CCCCCCCC(O)═O | 9(Z),11(E)-octadecadienoic acid |

In one embodiment, comparing step (e) involves comparing a rank ordered list of 500 genes that encode for transcripts identified as the most differentially expressed between lipotoxic FFAs and non-lipotoxic FFAs with a rank ordered list of genes identified as most genetically associated with the lipotoxic FFA disease or disorder.

In another embodiment, comparing step (e) involves comparing a rank ordered list of genes that encode for transcripts identified as the most differentially expressed between lipotoxic FFAs and non-lipotoxic FFAs with a rank ordered list of genes identified as in the top 5% or top 10% of genes most genetically associated with the lipotoxic FFA disease or disorder.

A further aspect of the instant disclosure provides a method for treating or preventing a lipotoxic FFA disease or disorder in a subject having or at risk of developing the lipotoxic FFA disease or disorder, the method involving administering to the subject an agent capable of modulating expression of one or more of the following genes: MACF1, HMG20A, QPCTL, NUCB2, SSR1, ATG16L2, ADCK5, ADCY5, CPSF1, PMPCA, ALDOA, FANCC, PRC1, SPRED2, ACVR1C, CMIP, DCAF7, MAPK3, NFIX, HAPLN4, CYHR1 and C9orf3, thereby treating or preventing the lipotoxic FFA disease or disorder in the subject.

In one embodiment, the agent is a nucleic acid that specifically targets the gene.

In another embodiment, the agent is a small molecule or a non-nucleic acid macromolecule.

In an additional embodiment, the agent is PQ912, 2-pyridine-3-yl-methylene-indan-1,3-dione (PRT4165), BC1753, PD98059, arphamenine A, and TDZD-8.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

The term “administration” refers to introducing a substance into a subject. In general, any route of administration may be utilized including, for example, parenteral (e.g., intravenous), oral, topical, subcutaneous, peritoneal, intraarterial, inhalation, vaginal, rectal, nasal, introduction into the cerebrospinal fluid, or instillation into body compartments. In some embodiments, administration is oral. Additionally or alternatively, in some embodiments, administration is parenteral. In some embodiments, administration is intravenous.

By “agent” is meant any small compound (e.g., small molecule), antibody, nucleic acid molecule, or polypeptide, or fragments thereof or cellular therapeutics such as allogeneic transplantation and/or CART-cell therapy.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

As used herein, the term “free fatty acid” or “FFA” refers to free fatty acids and their common salts. a carboxylic acid with a long aliphatic chain, either unsaturated or saturated. Most naturally occurring fatty acids have an unbranched chain of an even number of carbon atoms from 4 to 28. Fatty acids are usually not found in organisms, but instead exist as three main classes of esters: triglycerides, phospholipids, and cholesterol esters. Fatty acids are either derived from the hydrolysis of fats or synthesized from two carbon units (acetyl- or malonyl-CoA) in the liver, mammary gland, and adipose tissue. When circulating in the plasma fatty acids are non-esterified, or free from the ester. Free fatty acids in vivo are bound to transport proteins, albumin, for example. Exemplary free fatty acids include those recited in Table 1, among others known in the art.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

The term “lipotoxic FFA disease or disorder” as used herein refers to any disease or disorder associated with a deleterious and/or lipotoxic effect of one or more FFAs in a subject. Exemplary lipotoxic FFA diseases or disorders include type 2 diabetes (T2D), obesity, cardiovascular diseases (CVD), non-alcoholic fatty liver disease (NAFLD), obesity-mediated inflammation/metaflammation and insulin resistance, among others.

“Beta-cell”, “β-cell” or “pancreatic beta-cell”, as used herein, refers to the insulin producing cell type located in the islets of Langerhans in the pancreas.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease or condition in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

Unless specifically stated or obvious from context, as used herein, the term “or” is understood to be inclusive. Unless specifically stated or obvious from context, as used herein, the terms “a”, “an”, and “the” are understood to be singular or plural.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “therapeutically effective amount” of an agent described herein is an amount sufficient to provide a therapeutic benefit in the treatment of a condition or to delay or minimize one or more symptoms associated with the condition. A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1C show that unsupervised clustering of 61 free fatty acids (FFAs) revealed distinct biological effects among 5 newly-derived and structurally diverse FFA clusters. FIG. 1A shows a hierarchically clustered heatmap (rows and columns) of the signal to noise ratio (SNR) of the top 500 most commonly differentially expressed genes across the transcriptomic dataset (n=6 replicates). Clusters were extracted with the Dynamic Tree Cut function and assigned different colors (dendrogram color bar). FIG. 1B shows a circular depiction of hierarchical clustering by structure SMILES (simplified molecular-input line-entry system) for each FFA in the library. FIG. 1C shows the structural characterization of FFAs, wherein each dot represents a single FFA, summarized by cluster (x-axis). Clusters were arranged according to their calculated adjacency (see FIG. 6D).

FIGS. 2A to 2E show that functional characterization of FFA clusters elucidated a novel lipotoxicity signature. FIG. 2A shows a summary of the 25 most differentially enriched gene sets identified via global hallmark Gene Set Enrichment Analysis (GSEA). Gene rankings are based on log₂ fold changes resulting from cluster-centric differential expression analysis. FIGS. 2B to 2D show scatter plots of cell viability assay (FIG. 2B), ER Ca²⁺ levels (FIG. 2C) and glucose stimulated insulin secretion (GSIS) assay (FIG. 2D). Closed dots represent FFAs that showed significant deviations (p<0.05, Bonferroni) from controls in both replicates, open dots represent non-significant FFAs in at least one replicate. Colors indicate cluster affiliation (according to FIG. 1A above). FIG. 2E shows a summary of all functional assays. The top color bar represents transcriptomically defined FFA clusters arranged by their calculated adjacency (based on FIG. 6D below). The heatmap shows log₂ fold changes of respective functional readouts. X-axis labels show simplified FFA structure (SMILES). The box highlights the 20 FFAs in cluster 2 (C2), which exhibited a unique signature of lipotoxicity validated across all three functional assays. Highlighted FFAs were chosen as cluster representatives for further downstream validation studies. PA, palmitic acid; EA, erucic acid; PSA, petroselenic acid; OA, oleic acid; AA, arachidonic acid; GLA: gamma-linoleic acid.

FIGS. 3A to 3E show that cell biological assays independently validated the newly-derived lipotoxicity cluster. FIG. 3A shows a Western blot of selected FFAs (PA/EA, lipotoxicity cluster) inducing ATF4 and CHOP, confirming activation of the UPR. CPT1A was a positive control for successful FFA delivery. BSA, negative control. FIG. 3B, at left, shows median trajectories (n=5) of representative FFAs based on cytosolic fluorometric Ca²⁺ imaging (Fluo-4) after stimulation with thapsigargin (SERCA inhibitor, 10 μM, black triangle indicates addition; total recording time 10 min; f=1 Hz) (see Example 1 and FIG. 7D below), while at right is shown quantification of the peak amplitude as a readout for ER Ca²⁺ levels, relative to negative control (BSA). Data are mean±SD. Student's t-test (two-sided) *p<0.05, ****p<0.0001, corrected for multiple testing (Benjamini-Hochberg, whole FFA library). Bar color represents cluster identity (see FIG. 2E above). FIG. 3C shows fluorescence imaging of cells treated with representative FFAs for 48 h. Apoptotic cells (positive y axis) were measured by caspase activity, while dead cells (negative y axis) were measured by propidium iodide positive nuclei (n=12). Reduction in cell viability was defined as fraction of caspase positive and/or propidium iodide positive cells. Data are mean±SD. Student's t-test (two-sided) ****p<0.0001, corrected for multiple testing (Bonferroni). Bar color represents cluster identity (see FIG. 2E above). PA and EA reduced cell viability, whereas AA and GLA (cluster 5) trended towards decreased viability, in line with the high throughput screen of FIG. 2B above. FIG. 3D shows fluorescence imaging of nuclear translocation of RELA (green) upon treatment with EA (18 hours). BSA, negative control. Nuclei were stained with Hoechst 33342 (red) and phalloidin served as cytoplasmic marker (grey). Complete translocation of RELA from the cytosol to the nucleus (white arrows) was only detected in EA-treated cells, highlighted in the merged image. FIG. 3E shows quantification of RELA translocation events, as percentage of total number of cells (y axis), in all representative FFAs (t=18 hours, n=6). Data are mean±SD. Student's t-test (two-sided) ****p<0.0001, corrected for multiple testing (Bonferroni). Bar color represents cluster identity (see FIG. 2E above). PA, palmitic acid; EA, erucic acid; PSA, petroselenic acid; OA, oleic acid; AA, arachidonic acid; GLA: gamma-linoleic acid).

FIGS. 4A to 4E show that integration of lipotoxicity signature gene set with T2D GWAS data highlighted genes of interest including putative drug targets. FIG. 4A shows MAGMA gene set analysis results. A schizophrenia GWAS dataset served as negative control (41). Gene sets were defined as top genes (1%, 5%, or 10%) ranked by p-values emerging from lipotoxicity cluster-centric differential expression analysis. Gene set analysis (GSA) (40) shows significant enrichment (FDR<0.05) for the top 5% (highlighted in blue) and 10% lipotoxicity gene sets. FIG. 4B shows a scatter plot of genes based on T2D MAGMA rank (x axis) and lipotoxicity rank (y axis). Horizontal cutoff defined the top 5% lipotoxicity genes (blue), vertical cutoff defined the top 500 T2D genes emerging from the MAGMA analysis. Bottom left quadrant of the figure defined genes of interest (red) driving enrichment of the lipotoxicity signature. FIG. 4C shows a dot plot highlighting top 25 genes of interest, with expression patterns across all FFA clusters represented. Dot sizes represent the percentage of FFAs/cluster that induce significant differential expression (p<0.05, Benjamini-Hochberg), while color represents the strength and direction of transcriptional changes (log₂ fold change). Highlighted genes are of particular interest. FIG. 4D, left, shows violin plots of variance stabilized gene counts across all clusters arranged by calculated proximity (see FIG. 6C). FIG. 4D, right, shows a scatter plot of gene expression (y axis) and log₂ fold change of respective functional readout (x axis). Closed dots represent FFAs significantly different (p<0.05, Bonferroni) from controls in both replicates of functional readout, open dots represent non-significant FFAs in at least one replicate. Linear regression showed significant correlation (p<0.05, Bonferroni) with the corresponding functional readout, as indicated, for each of three selected genes (SLC30A8, PAM, ACVR1C). FIG. 4E shows an outline of analysis pipeline for the integration of transcriptomic lipotoxicity signatures with MAGMA ranked T2D GWAS data.

FIGS. 5A to 5E depict a FFA library preparation protocol of the instant disclosure. FIG. 5A shows a graphic of a protocol overview, displaying the major steps of the procedure: DMSO dissolved lipids were coupled to BSA overnight and evaporated in a full vacuum (37° C., spinning at 400 g). Solvent-free FFA preparations were then resuspended in cell culture medium. FIG. 5B shows the shift in albumin melting temperature Tm (y axis) observed; after library preparation, structurally representative FFAs (structure SMILES, x-axis) consistently increased Tm confirming successful conjugation. Colors represent transcriptomically defined cluster identities (see FIG. 1B above). FIG. 5C shows CPT1A expression for selected FFAs. Consistently significant differential expression (p_{adj}<0.05) confirmed successful delivery of FFAs. FIG. 5D shows a schematic overview of sample preparation employed for the lipidomics screen. FIG. 5E shows the correlation of structural features (number of C atoms, number of double bonds) of externally applied FFAs (x-axis) and structural features of triglycerides (TAGs, y-axis) detected in the lipidomic screen. The upper two panels (red dots) summarize absolute increases in summed triglyceride intensities per FFA, while the lower two panels (blue dots) summarize absolute decreases in summed triglyceride intensities per FFA, thereby serving as a negative control. This lipidomic analysis indicated that cells exposed to externally applied FFAs incorporated them into their triglyceride fraction.

FIGS. 6A to 6D demonstrate transcriptomic characterization of the FFA library. FIG. 6A presents a schematic showing the RNAseq protocol overview. FIG. 6B shows the hierarchically clustered heatmap based on z-scores of the top 500 most commonly differentially expressed genes across the entire dataset showing individual replicates (n=6). Clusters were extracted with the Dynamic Tree Cut function (55). FIG. 6C shows a corner plot of the first four Principal Components of all replicates, which captured 75% of the total variance in the dataset. Colors represent cluster identities. FIG. 6D shows cluster correlation map similarity between different transcriptomically defined FFA clusters. The first principal component of all FFAs in each cluster among the top 500 most commonly differentially expressed genes served as cluster representatives (meta-samples).

FIGS. 7A to 7D show functional characterization screens. FIG. 7A shows summary heatmap results for cell viability as a functional characterization screen, based on log₂ fold changes. The color bar at left denotes transcriptomically defined FFA clusters (FIG. 1B). Each column represents a full library screen at the indicated time point. FIG. 7B shows summary heatmap results for endoplasmic reticulum calcium (ER Ca²⁺) levels as a functional characterization screen, based on log₂ fold changes. The color bar at left denotes transcriptomically defined FFA clusters (FIG. 1B). Each column represents a full library screen at the indicated time point. FIG. 7C shows summary heatmap results for glucose stimulated insulin secretion as a functional characterization screen, based on log₂ fold changes. The color bar at left denotes transcriptomically defined FFA clusters (FIG. 1B). Each column represents a full library screen at the indicated time point. FIG. 7D at left shows a schematic overview of the Ca²⁺ imaging protocol that was employed herein to quantify fluorometrically dynamic changes in cytosolic Ca²⁺ concentrations in microplates using the Molecular Devices FLIPR Tetra® High-Throughput Cellular Screening System. FIG. 7D on the right shows representative example trajectories (n=5) of thapsigargin (10 μM) stimulated (black arrow) Ca²⁺ signals, as fold changes over baseline (y-axis). The inset shows the measurement protocol.

FIG. 8 shows that, similar to the C2 lipotoxicity cluster of free fatty acids observed to inhibit mouse MIN6 pancreatic cell viability (see FIGS. 2B and 7A above), among a selection of C2 cluster free fatty acids tested for toxicity against human pancreatic islet cells, all C2 cluster free fatty acids also greatly decreased human pancreatic cell viability. Notably, 13Z-docosenoic acid, 14Z-tricosenoic acid, and 15Z-tetracosenoic acid decreased human pancreatic cell viability by more than 50% relative to the C3 and tested C4 free fatty acids.

### DETAILED DESCRIPTION OF THE INVENTION

The present disclosure relates, at least in part, to the discovery of compositions and methods that provide for improved screening of independent FFAs and their effects upon contacted cells in culture. Compositions and methods for the preparation and use of free fatty acids (FFAs) as crystals and in solution, optionally in an array format, for assay of lipotoxicity and related effects (e.g., indicators of lipotoxicity-related diseases or disorders such as type II diabetes (T2D)) in contacted cells, are therefore provided. Performance of FFA screening of mammalian cells at scale (as enabled by the FFA arrays of the instant disclosure) succeeded herein at identifying a new class of lipotoxic FFAs. When transcriptome analysis of lipotoxic FFA effect was combined with gene lists derived from a genome-wide association study (GWAS) of T2D genetic associations, new high value target genes for therapeutic modulation of T2D were also discovered. Modulation of these high value T2D target genes is therefore also provided for by the instant disclosure.

The compositions and methods of the instant disclosure allow for systematic investigation of the structurally heterogenous group of lipophilic compounds (free fatty acids, FFAs). In prior art use of FFAs, single FFAs were dissolved manually, mostly containing remnants of solvents typically used to dissolve FFAs (DMSO, ethanol, etc.). Resulting insights for individual FFAs were then extrapolated to other FFAs based on structural similarities. Disadvantages of this prior art approach have included the fact that such approaches are inherently low throughput, remaining solvents in the preparations impede such assessments, and such approaches have not offered the ability to systematically study the effects of all FFAs. The instant disclosure therefore resolves these issues of the prior art by (1) providing a procedure that enables the generation of solvent-free FFA solutions in high content screening-ready microplates without remaining solvents, and (2) providing methods for high throughput comprehensive and systematic study of all FFAs, thereby removing the need for assumptions and extrapolations between/to other FFAs based on manual experiments performed only with a few FFAs.

In certain aspects, a key advance of the instant disclosure is the use of a high-throughput evaporator, which has allowed for gentle drying down of previously conjugated BSA-FFA solutions in a microplate format. The remaining BSA crystals thereby produced are essentially solvent-free and readily soluble in aqueous solutions.

Comprehensive investigation of a large group of FFAs has, as disclosed herein, enabled improved assessment of lipotoxicity—a key feature of many metabolic diseases such as diabetes, cardio vascular diseases and kidney disease—under standardized in vitro conditions. The integration of results across different affected cell types is expected to enable enhanced understanding of conserved mechanisms of lipotoxicity. Further, the combination of transcriptomic insights from the assays of the instant disclosure with large scale GWAS studies is expected to yield orthogonally validated genes of interest that possess large potential therapeutic relevance. In short, the compositions and methods of the instant disclosure are expected to help prioritize therapeutic targets from genomic studies of metabolic diseases.

The dataset of the instant disclosure has thus provided a critical first step towards deeper understanding of lipid biology, and specifically the structural landscape of FFAs and their biological sequelae. It was found herein, for example, that the mono-unsaturated and saturated FFAs of cluster 2 robustly induced lipotoxicity, which indicated a need for a paradigm shift: previously defined structural features alone (saturated vs unsaturated) were identified herein not to correlate with biological effect (e.g. lipotoxicity). While the instant analysis has not focused upon cell-protective FFAs, additional work in this area using the platform provided by the instant disclosure should reveal additional insights into lipid biology. Importantly, the instant disclosure has identified 20 FFAs in the lipotoxicity cluster (C2) that are likely responsible for the well characterized association between high triglycerides measured in patients' blood and risk for T2D (and other metabolic diseases). It is likely that understanding genetic risk profiles for T2D in the context of lipotoxic risk factors, such as coupling individual T2D polygenic risk scores (48) with measuring the abundance of lipotoxic FFAs in patients' blood, will allow for identification of the patients highest at risk for developing T2D. Such identification is also expected to lead to more comprehensive personalized risk profiles, ultimately helping to provide the right preventative or therapeutic strategy to the right patient at the right time.

Complex diseases are caused by an interplay of environmental and genetic risk factors (1). Genomic studies continuously identify novel disease-associated risk variants (2-4), yet prior to the instant disclosure, little has been known about how to integrate these with environmental risk. Obesity is a major risk factor for several metabolic diseases (5, 6). The obesogenic environment, characterized by an overconsumption of dietary lipids, results in the accumulation of toxic free fatty acids (FFAs) in many tissues (7). The resulting lipotoxicity induces cellular stress states linked to diseases such as type 2 diabetes (T2D) (8). The instant disclosure describes a systematic investigation of FFAs in a pancreatic beta cell line and the identification of a previously unrecognized lipotoxicity group, which includes 20 saturated and mono-unsaturated FFAs, breaking a long-held paradigm about FFA structure-function relationships. Importantly, this scalable, cell-based platform derived and functionally validated a transcriptomic signature associated with lipotoxic FFAs. This signature has been integrated herein with a T2D genome-wide association study (GWAS) dataset, and thus 25 genes were nominated at the intersection of the lipotoxic environment and genetic risk for T2D. Among them, GLP1R, a known T2D drug target (9, 10), and T2D GWAS/whole exome sequencing (WES) genes PAM and SLC30A8 (11, 12), served as independent validation for the instant approach. This scalable platform provides an enhanced process for nominating genes at the intersection of genetic and environmental risk in not only T2D/obesity but also other lipotoxicity-driven diseases, including cardiovascular diseases (CVD) and non-alcoholic fatty liver disease (NAFLD), among others, and offers to illuminate therapeutic targets for these additional disease states (as have been identified and disclosed herein for T2D).

The association of FFAs with risk for T2D was postulated more than five decades ago (13). Recent large scale epidemiological studies, including the Framingham Heart Study, provided support for this hypothesis by showing that structural features of triglycerides (such as degree of saturation and carbon chain length), can predict the development and progression of T2D (14). Since FFAs are the major building blocks of triglycerides (14), a fundamental question was how FFAs exert cellular effects that ultimately lead to T2D. Most cell biological studies have largely focused on two FFAs: the saturated FFA palmitic acid (PA) and the mono-unsaturated FFA oleic acid (OA). This led to the widely-held notion that saturated FFAs were toxic to pancreatic beta cells, liver cells and others, whereas unsaturated FFAs were not (and were even protective) (15-17). However, it was not clear that conclusions drawn from a few FFAs could be extrapolated to infer biological effects across the entire spectrum of FFAs. Therefore, it was necessary to ask—as has now been successfully addressed herein—how to comprehensively define the cellular effects mediated by structurally diverse FFAs (largely obtained by humans through the diet), as well as how to specifically identify FFAs mechanistically linked to lipotoxicity and T2D.

### Cell Culture/Tissue Models of Lipotoxic FFA Diseases or Disorders

Mammalian cell culture can be performed my methods known in the art. In certain embodiments of the disclosure, cells and tissue models employed and used to model lipotoxic free fatty acid diseases or disorders include, without limitation, pancreatic beta cells or pancreatic tissue as a type 2 diabetes (T2D) model, endothelial cells or endothelial tissue as a cardiovascular diseases (CVD) model, hepatocyte cells or liver tissue as a non-alcoholic fatty liver disease (NAFLD) model, macrophage cells as an obesity-mediated inflammation/metaflammation model, skeletal muscle cells or skeletal muscle tissue as an insulin resistance model, and adipocyte cells or fat tissue as an obesity model.

### Cell Culture Media

Cell culture can be performed in art-recognized media. For example, pancreatic beta cells can be grown in DMEM (as in the instant disclosure), in RPMI media (e.g., as described in U.S. Pat. No. 10,302,663), or in other media, optionally including 50-60 μM β-mercaptoethanol. Endothelial cells can be grown in, for example, DMEM or Vascular Basal Cell Media (ATCC). Hepatocytes can be grown in, for example, arginine-free Willows E Standard Media or DMEM (e.g., as described in U.S. Patent Application No. 2008/01314014). Macrophages can be grown in, for example, IMDM or DMEM. Skeletal muscle cells and adipose tissue can be grown in, for example, DMEM (e.g., as described in U.S. Pat. No. 8,883,498 and U.S. Patent Application No. 2015/0191696) or Mesenchymal Stem Cell Basal Media for adipose, umbilical, and bone marrow derived MSCs (ATCC).

### Free Fatty Acids (FFAs)

Exemplary free fatty acids (FFAs) include, but are not limited to, the following:

Saturated Fatty Acids
Butyric acid (Butanoic acid) CH3(CH2)2COOH
Valeric acid (Pentanoic acid) CH3(CH2)3COOH
Caproic acid (Hexanoic acid) CH3(CH2)4COOH
Enanthic acid (Heptanoic acid) CH3(CH2)5COOH
Caprylic acid (Octanoic acid) CH3(CH2)6COOH
Pelargonic acid (Nonanoic acid) CH3(CH2)7COOH
Capric acid (Decanoic acid) CH3(CH2)8COOH
Undecylic acid (Undecanoic acid) CH3(CH2)9COOH
Lauric acid (Dodecanoic acid) CH3(CH2)10COOH
Tridecylic acid (Tridecanoic acid) CH3(CH2)11COOH
Myristic acid (Tetradecanoic acid) CH3(CH2)12COOH
Pentadecylic acid (Pentadecanoic acid) CH3(CH2)13COOH
Palmitic acid (Hexadecanoic acid) CH3(CH2)14COOH
Margaric acid (Heptadecanoic acid) CH3(CH2)15COOH
Stearic acid (Octadecanoic acid) CH3(CH2)16COOH
Nonadecylic acid (Nonadecanoic acid) CH3(CH2)17COOH
Arachidic acid (Eicosanoic acid) CH3(CH2)18COOH
Heneicosylic acid (Heneicosanoic acid) CH3(CH2)19COOH
Behenic acid (Docosanoic acid) CH3(CH2)20COOH
Tricosylic acid (Tricosanoic acid) CH3(CH2)21COOH
Lignoceric acid (Tetracosanoic acid) CH3(CH2)22COOH
Pentacosylic acid (Pentacosanoic acid) CH3(CH2)23COOH
Cerotic acid (Hexacosanoic acid) CH3(CH2)24COOH
Carboceric acid (Heptacosanoic acid) CH3(CH2)25COOH
Montanic acid (Octacosanoic acid) CH3(CH2)26COOH
Nonacosylic acid (Nonacosanoic acid) CH3(CH2)27COOH
Melissic acid (Triacontanoic acid) CH3(CH2)28COOH
Hentriacontylic acid (Hentriacontanoic acid) CH3(CH2)29COOH
Lacceroic acid (Dotriacontanoic acid) CH3(CH2)30COOH
Psyllic acid (Tritriacontanoic acid) CH3(CH2)31COOH
Geddic acid (Tetratriacontanoic acid) CH3(CH2)32COOH
Ceroplastic acid (Pentatriacontanoic acid) CH3(CH2)33COOH
Hexatriacontylic acid (Hexatriacontanoic acid) CH3(CH2)34COOH
Heptatriacontylic acid (Heptatriacontanoic acid) CH3(CH2)35COOH
Octatriacontylic acid (Octatriacontanoic acid) CH3(CH2)36COOH
Nonatriacontylic acid (Nonatriacontanoic acid) CH3(CH2)37COOH
Tetracontylic acid (Tetracontanoic acid) CH3(CH2)38COOH

Unsaturated Fatty Acids
Crotonic acid ((E)-but-2-enoic acid) C3H5CO2H
Myristoleic acid ((Z)-tetradec-9-enoic acid) C13H25CO2H
Palmitoleic acid ((Z)-hexadec-9-enoic acid) C15H29CO2H
Sapienic acid ((Z)-6-Hexadecenoic acid) C15H29CO2H
Oleic acid ((Z)-octadec-9-enoic acid) C17H33CO2H
Elaidic acid ((E)-octadec-9-enoic acid) C17H33CO2H
Vaccenic acid ((Z)-octadec-11-enoic acid) C17H33CO2H
Gadoleic acid ((Z)-icos-9-enoic acid) C19H37CO2H
Eicosenoic acid ((Z)-icos-11-enoic acid) C19H37CO2H
Erucic acid ((Z)-docos-13-enoic acid) C21H41CO2H
Nervonic acid ((Z)-tetracos-15-enoic acid) C23H45CO2H
Linoleic acid ((9Z, 12Z)-octadeca-9,12-dienoic acid) C17H31CO2H
Eicosadienoic acid ((11Z, 14Z)-icosa-11,14-dienoic acid) C19H35CO2H
Docosadienoic acid ((13Z, 16Z)-docosa-13,16-dienoic acid) C21H39CO2H
α-linolenic acid ((9Z, 12Z, 15 Z)-octadeca-9,12,15-trienoic acid) C17H29CO2H
F-linolenic acid ((6Z, 9Z, 12Z)-octadeca-6,9,12-trienoic acid) C17H29CO2H
Pinolenic acid ((5Z, 9Z, 12 Z)-octadeca-5,9,12-trienoic acid) C17H29CO2H
α-eleostearic acid ((9E, 11E, 13 Z)-octadeca-9,11,13-trienoic acid) C17H29CO2H
β-eleostearic acid ((9E, 11E, 13E)-octadeca-9,11,13-trienoic acid) C17H29CO2H
Dihomo-γ-linolenic acid ((8Z, 11Z, 14 Z)-icosa-8,11,14-trienoic acid) C19H33CO2H
Eicosatrienoic acid ((11Z, 14Z, 17 Z)-icosa-11,14,17-trienoic acid) C19H33CO2H
Stearidonic acid ((6Z, 9Z, 12 Z, 15Z)-octadeca-6,9,12,15-tetraenoic acid) C17H27CO2H
Arachidonic acid ((5Z, 8Z, 11 Z, 14Z)-icosa-5,8,11,14-tetraenoic acid) C19H31CO2H
Eicosatetraenoic acid ((8Z, 11Z, 14 Z, 17Z)-icosa-8,11,14,17-tetraenoic acid) C19H31CO2H
Adrenic acid ((7Z, 10Z, 13 Z, 16Z)-docosa-7,10,13,16-tetraenoic acid) C21H35CO2H
Bosseopentaenoic acid ((5Z, 8Z, 10 E, 12E, 14Z)-eicosa-5,8,10,12,14-pentaenoic acid) C17H25 CO2H
Eicosapentaenoic acid ((5Z, 8Z, 11 Z, 14Z, 17Z)-icosa-5,8,11,14,17-pentaenoic acid) C19H29CO2H
Ozubondo acid ((4Z, 7Z, 10Z, 13Z, 16Z)-docosa-4,7,10,13,16-pentaenoic acid) C21H33CO2H
Sardine acid ((7Z, 10Z, 13 Z, 16Z, 19Z)-docosa-7,10,13,16,19-pentaenoic acid) C21H33CO2H
Tetracosanolpentaenoic acid (9Z, 12Z, 15 Z, 18Z, 21Z)-tetracosa-9,12,15,18,21-pentaenoic acid) C23H37CO2H
Docosahexaenoic acid ((4Z, 7Z, 10Z, 13Z, 16Z, 19Z)-docosa-4,7,10,13,16,19-hexaenoic acid) C21H31CO2H
Herring acid ((6Z, 9Z, 12 Z, 15Z, 18Z, 21Z)-tetracosa-6,9,12,15,18,21-hexaenoic acid) C23H35CO2H

### FFA Preparation for In Vitro Studies

Fatty acids are poorly soluble, and thus usually studied when complexed to albumins such as bovine serum albumin (BSA). The conjugation of fatty acids to albumin requires attention to preparation of the solutions, effective free fatty acid concentrations, use of different fatty acid species, and appropriate controls to ensure cellular fatty acid uptake.

Albumin-FA interactions maximize the amount of FA transported and cleared from circulation in vivo. In the presence of albumin, FA uptake by cells is more efficient, an effect that is not mediated by receptors. In in vitro systems, albumin-promoted FA delivery is a function of FA unbound concentration in the medium at the physiological concentration of albumin (˜200 μM). At lower concentrations (<150 μM), delivery will depend on albumin concentration.

BSA has six to seven high affinity binding sites for FAs and is thus an efficient carrier serving to substantially increase the solubility of FAs in aqueous solutions. During lipid/albumin conjugation, FA:BSA ratios should be considered since they determine FA availability. In healthy humans, serum FA:BSA ratios range from 1:1 to 3:1. These ratios can be higher than 5:1 in disease states. Accordingly, the use of high FA:BSA ratios in experiments enhances the biological effects of lipids.

BSA free of endogenous FAs should be used in order to form the desired FFA-BSA conjugate. However, BSA free of FAs makes the selection of proper controls challenging. Cell culture sera typically consist of 2% albumin, to which FAs obviously also bind, thus necessitating a reduction in the serum concentration of the media to avoid a change in the FA:BSA ratio. FA-free BSA also serves as a sink for cellular lipids; the addition of FA-free BSA in the media traps and absorbs free FAs, thereby altering secretory function. In order to avoid these effects, an alternative solution is to dissolve the FA of interest and complex the FA directly to the BSA before adding to the FBS containing cell culture media. The advantage of this method is that it minimizes the undesirable effects of FA-free BSA while maintaining the serum concentration at 10% during culture conditions.

### FFA Solvents

FFAs are poorly soluble when non-esterified and not bound to proteins in aqueous solutions. Accordingly, organic solvents are generally used for dissolving unbound FFAs in solution, with DMSO commonly used. Additional exemplary FFA solvents include ethanol, N-hexane and methylene chloride (DCM), among others known in the art.

### BSA Solvents

BSA is soluble in aqueous solutions. Purified water is an exemplary BSA solvent, with double-distilled water (ddH₂O) employed herein in view of ddH₂O being free of any contaminating salts that could alter crystal complex formation. It is contemplated herein that the instant methods of the disclosure could be performed with another protein substituted for BSA; however, BSA is noted to be a natural “carrier” for FFAs.

### FFA Crystal Resuspension

FFA crystals of the instant disclosure can be resuspended in a range of solvents, but are primarily exemplified herein as dissolved directly in cell culture media (e.g., in embodiments where the dissolved FFA crystal solutions are then used to contact cells in culture).

### Arrays (Array Formats) Plates/Microplates

In certain aspects, FFAs are assembled and/or provided in an array format. In embodiments, individual elements/containers/wells of the array hold solutions of individual FFAs, with elements differing by individual FFA and element/well location across the array. FFAs can be arrayed, for example, in plate formats known in the art, including, e.g., 96 well, 192 well, 384 well, 1536 well, 3456 well, 6144 well, etc. plates. Cell screening using such arrayed FFAs can also be performed in such array plates, with per-well assay volumes typically ranging from about 200 μl to about 0.5 μl, depending upon individual well volume.

Measuring Lipotoxicity, Cell Death and/or Biomarkers of Apoptosis

In certain aspects, lipotoxicity, cell death and/or biomarkers of apoptosis are assessed. Such assessment can be performed upon cells in culture, upon tissues and/or upon organisms contacted with the FFAs, using methods described herein and as known in the art. Notably, the methods and assays disclosed herein to characterize lipotoxicity have been specifically developed herein, for practice in certain aspects in a microplate format, which has robustly enabled high content screening.

### Rank Ordering of Genome-Wide Association Study (GWAS) Genes

Certain aspects of the instant disclosure feature integrative assessment of lipotoxicity/cell death/apoptosis data and/or gene lists with gene lists derived from genome-wide association studies (GWAS). Rank ordered lists of genes identified as potentially genetically associated with a disease state or phenotype assessed in a GWAS can be obtained by any art-recognized method, including the method(s) specifically exemplified herein.

### Type II Diabetes Lipotoxic Target Genes

The following genes were identified herein as highly responsive to lipotoxic free fatty acids and also within the top 5% of genetic associations with Type II diabetes (T2D) drawn from a recent genome-wide association study (GWAS): MACF1, HMG20A, QPCTL, SLC30A8, NUCB2, SSR1, ATG16L2, ADCK5, ADCY5, CPSF1, PMPCA, PAM, ALDOA, FANCC, PRC1, SPRED2, GLP1R, ACVR1C, CMIP, DCAF7, MAPK3, NFIX, HAPLN4, CYHR1 and C9orf3. Primary transcript cDNA and amino acid sequences for the human forms of each of these genes are:

MACF1 cDNA; NM_012090
(SEQ ID NO: 1)
ATCACTTCTCCCTGGGCTCCCAGGCCCTCCTGCAGCAGCCCCCGCCTGGGCCATGT
CTTCCTCAGATGAAGAGACGCTCAGTGAGCGGTCATGTCGGAGTGAGCGGTCTTGT
CGGAGTGAGCGATCTTACAGGAGCGAGCGGTCGGGGAGCCTGTCTCCCTGTCCCC
CAGGGGACACCTTGCCCTGGAACCTGCCACTGCATGAGCAGAAAAAGCGGAAAAG
CCAGGATTCGGTGCTGGACCCTGCAGAGCGTGCTGTGGTCAGAGTCGCTGATGAA
CGGGACCGGGTTCAGAAGAAAACGTTCACCAAGTGGGTCAACAAGCACTTAATGA
AGGTCCGCAAGCACATCAATGATCTTTATGAAGATCTGCGGGATGGCCATAACCTG
ATCTCTCTGTTGGAGGTCCTCTCAGGCATCAAACTGCCCCGGGAGAAGGGCAGGAT
GCGTTTTCATAGGCTGCAGAATGTGCAGATTGCCCTGGACTTCCTAAAGCAGCGAC
AGGTGAAACTAGTGAATATTCGCAATGATGACATCACAGATGGCAACCCCAAGTT
GACCCTGGGTCTGATCTGGACCATTATTTTGCATTTCCAGATCTCTGACATCTACAT
TAGTGGAGAATCAGGGGATATGTCAGCCAAGGAGAAACTACTCCTGTGGACCCAG
AAGGTGACAGCTGGTTACACAGGAATCAAATGCACCAACTTTTCCTCCTGCTGGAG
TGATGGGAAGATGTTCAATGCACTCATTCACCGATACCGACCCGATCTAGTAGACA
TGGAGAGGGTGCAAATCCAAAGTAACCGAGAGAATCTGGAACAGGCTTTTGAAGT
GGCAGAAAGACTGGGGGTCACTCGCCTGCTGGATGCAGAAGATGTGGATGTGCCA
TCTCCAGATGAAAAGTCTGTAATCACTTATGTGTCTTCGATTTATGATGCCTTCCCT
AAAGTTCCTGAGGGTGGAGAAGGGATCAGTGCTACGGAAGTGGACTCCAGGTGGC
AAGAATACCAAAGCCGAGTGGACTCCCTCATTCCCTGGATCAAACAGCATACAAT
ACTGATGTCAGATAAAACTTTTCCCCAAAACCCTGTTGAACTAAAGGCACTTTATA
ACCAATATATACACTTCAAAGAAACAGAAATTCTGGCCAAGGAGAGAGAAAAAG
GAAGAATTGAGGAATTATATAAATTACTAGAGGTGTGGATTGAATTTGGCCGAATT
AAACTGCCTCAAGGTTATCACCCTAATGATGTGGAAGAAGAGTGGGGAAAGCTCA
TCATAGAGATGCTGGAACGAGAGAAATCACTTCGGCCGGCTGTGGAGAGGCTGGA
ATTGCTGCTACAGATTGCAAACAAAATCCAGAATGGTGCTTTGAACTGTGAAGAA
AAACTGACACTAGCTAAGAATACACTGCAGGCTGATGCTGCTCACCTGGAATCAG
GACAACCGGTACAATGTGAGTCAGATGTCATTATGTACATTCAGGAGTGTGAAGG
TCTCATCAGGCAGCTGCAGGTGGATCTCCAGATCCTGCGGGATGAGAATTACTACC
AGCTAGAAGAGCTGGCTTTTAGGGTCATGCGTCTTCAGGATGAGCTGGTCACCTTG
CGTCTAGAGTGTACAAACCTGTACCGGAAGGGTCATTTCACTTCACTTGAATTGGT
TCCACCCTCTACTTTAACCACCACTCATCTGAAAGCAGAACCCTTAACCAAGGCAA
CCCATTCTTCTTCTACCTCCTGGTTCCGAAAGCCTATGACTCGGGCTGAACTTGTGG
CCATCAGCTCCTCTGAAGATGAAGGCAATCTCCGATTTGTGTATGAACTACTGTCT
TGGGTAGAAGAGATGCAGATGAAACTGGAGCGAGCAGAGTGGGGCAATGACCTG
CCTAGTGTGGAGTTGCAGCTAGAAACACAGCAGCACATCCATACGAGTGTAGAAG
AGCTGGGCTCAAGTGTCAAGGAGGCCAGGTTGTATGAGGGAAAGATGTCCCAGAA
TTTCCATACCAGCTATGCTGAAACTCTTGGAAAGCTGGAGACACAGTATTGTAAAT
TGAAGGAAACTTCTAGCTTCCGGATGAGGCACCTTCAGAGCCTGCATAAATTTGTT
TCCAGAGCTACAGCTGAGTTGATCTGGTTGAATGAGAAGGAGGAGGAGGAACTAG
CATATGACTGGAGTGACAACAATTCCAATATCTCAGCCAAGAGAAATTACTTCTCT
GAGTTGACAATGGAACTGGAGGAGAAACAGGATGTGTTTCGTTCTCTACAAGATA
CAGCAGAACTACTTTCACTTGAGAACCACCCAGCCAAGCAGACAGTGGAGGCTTA
CAGTGCTGCTGTCCAGTCCCAGTTGCAGTGGATGAAGCAGCTGTGCCTGTGTGTTG
AGCAGCATGTGAAAGAGAATACTGCTTATTTTCAGTTCTTCAGTGATGCACGAGAG
CTGGAGTCATTCTTGAGGAACCTCCAAGATTCCATTAAACGAAAATATTCCTGTGA
CCACAACACCAGCTTATCCCGCCTTGAAGACCTGCTCCAGGACTCCATGGATGAAA
AGGAGCAGCTTATACAGTCCAAGAGTTCCGTTGCCAGTCTCGTTGGGAGATCAAA
AACCATCGTTCAGCTAAAACCACGCAGTCCAGACCATGTGTTAAAGAACACCATTT
CTGTCAAGGCTGTCTGTGACTACAGGCAGATCGAGATTACTATTTGCAAAAATGAT
GAATGTGTGCTAGAAGATAATTCTCAGCGGACCAAATGGAAAGTGATCAGCCCCA
CAGGGAACGAGGCAATGGTGCCGTCAGTCTGCTTCCTCATCCCCCCACCCAATAAG
GATGCCATTGAGATGGCCAGCAGGGTCGAACAATCTTATCAGAAGGTTATGGCCC
TTTGGCATCAGCTGCATGTTAACACCAAAAGCCTTATCTCTTGGAACTATCTGCGT
AAAGACCTTGACCTTGTACAGACCTGGAACCTAGAAAAGCTTCGATCCTCAGCACC
AGGGGAGTGCCATCAGATTATGAAGAACCTTCAGGCCCACTATGAAGACTTTCTGC
AGGATAGTCGTGACTCTGTGCTGTTCTCAGTGGCTGATCGCTTGCGCTTGGAAGAG
GAGGTGGAAGCTTGTAAAGCCCGCTTCCAGCACCTGATGAAGTCCATGGAGAATG
AGGACAAAGAGGAGACTGTGGCCAAGATGTACATTTCAGAGTTGAAGAACATCCG
GCTACGCCTGGAGGAGTATGAACAGAGGGTGGTCAAACGAATTCAGTCTCTAGCC
AGCTCTAGGACTGACAGAGATGCCTGGCAGGACAATGCATTAAGGATTGCAGAGC
AAGAGCACACCCAGGAGGATTTACAGCAATTGAGGTCAGACTTGGATGCAGTTTC
TATGAAATGTGACAGCTTTCTCCATCAGTCTCCATCTAGTTCAAGTGTCCCAACTCT
GCGCTCAGAACTGAATCTGCTGGTGGAGAAGATGGACCATGTCTATGGTCTCTCTA
CTGTATATCTGAATAAGTTAAAGACAGTTGATGTTATAGTACGTAGCATACAGGAT
GCTGAACTCTTGGTCAAAGGTTATGAGATTAAGCTGAGTCAAGAAGAAGTAGTAC
TGGCAGATCTCTCAGCTCTGGAGGCCCATTGGTCGACATTACGGCACTGGCTTAGT
GATGTGAAGGACAAGAATTCAGTGTTTTCAGTCCTGGATGAGGAAATTGCCAAGG
CCAAGGTAGTGGCAGAGCAGATGAGTCGTCTGACACCAGAGCGAAATCTGGATTT
GGAGCGCTATCAGGAAAAAGGCTCCCAGCTGCAGGAGCGTTGGCACCGAGTCATT
GCCCAGCTCGAGATTCGCCAATCTGAGCTAGAAAGTATCCAGGAAGTTCTGGGAG
ATTACCGAGCCTGCCATGGAACTCTCATCAAGTGGATTGAGGAAACCACTGCCCA
GCAGGAAATGATGAAGCCAGGCCAGGCAGAGGATAGCAGAGTGCTTTCGGAGCA
GCTCAGCCAGCAGACGGCCCTATTTGCAGAAATTGAGAGAAATCAGACAAAACTG
GATCAATGTCAAAAATTTTCCCAGCAGTACTCTACTATTGTAAAGGACTATGAATT
GCAACTGATGACATACAAGGCCTTTGTGGAATCGCAGCAGAAATCCCCTGGCAAG
CGCCGTCGCATGCTTTCCTCTTCAGATGCCATCACTCAAGAGTTCATGGACTTAAG
GACTCGCTACACGGCATTGGTGACTTTAACAACTCAGCACGTGAAATACATCAGTG
ATGCACTCCGGCGTCTGGAGGAGGAGGAGAAAGTGGTAGAAGAGGAGAAACAAG
AACATGTGGAGAAGGTTAAAGAACTTTTGGGCTGGGTGTCTACCCTAGCGAGGAA
TACACAAGGAAAAGCTACCTCATCCGAGACCAAAGAATCAACAGACATTGAAAAA
GCTATTTTGGAACAGCAGGTTCTGTCAGAAGAGCTGACAACAAAGAAAGAACAAG
TCTCTGAAGCTATTAAAACATCACAGATCTTCTTGGCCAAGCATGGTCATAAGCTC
TCAGAAAAAGAGAAGAAACAAATATCTGAGCAATTGAATGCCCTAAACAAGGCTT
ACCATGACCTTTGTGATGGTTCTGCAAATCAGCTTCAGCAGCTTCAGAGCCAGTTG
GCTCACCAGACAGAACAAAAGACCCTGCAGAAACAACAAAATACCTGTCACCAGC
AACTGGAGGATCTTTGCAGTTGGGTAGGACAGGCAGAAAGAGCACTGGCAGGCCA
CCAAGGCAGAACCACCCAGCAGGATCTCTCTGCTTTGCAGAAGAACCAAAGTGAC
TTGAAGGATTTACAGGATGACATTCAGAATCGTGCCACCTCATTTGCCACTGTTGT
CAAGGACATTGAGGGGTTCATGGAAGAGAATCAGACCAAGCTGAGCCCACGTGAG
TTGACAGCTCTTCGGGAAAAGCTTCATCAGGCTAAGGAGCAATATGAGGCGCTCC
AGGAAGAGACACGTGTGGCCCAGAAGGAACTGGAGGAAGCAGTGACCTCCGCCTT
ACAGCAGGAGACTGAAAAGAGTAAAGCAGCAAAGGAACTGGCAGAGAACAAGAA
GAAGATCGATGCTCTCCTGGATTGGGTAACTTCAGTAGGATCATCTGGTGGACAGC
TGCTGACCAACCTTCCAGGAATGGAGCAGCTCTCGGGAGCTAGCTTGGAGAAAGG
AGCCTTGGACACCACTGATGGTTACATGGGGGTGAATCAAGCCCCAGAGAAACTG
GACAAGCAATGTGAGATGATGAAGGCCCGTCACCAAGAATTGCTGTCCCAGCAGC
AAAATTTCATTCTGGCCACCCAGTCAGCTCAGGCCTTCTTGGATCAGCATGGCCAC
AATCTCACACCTGAGGAGCAACAGATGCTGCAACAGAAGCTGGGAGAGCTAAAGG
AACAATACTCTACTTCCCTGGCCCAATCAGAGGCAGAACTGAAGCAGGTGCAGAC
ACTTCAGGATGAGTTGCAGAAATTTCTGCAGGATCATAAAGAGTTTGAAAGCTGGT
TGGAACGATCCGAGAAAGAGCTGGAGAACATGCATAAGGGAGGCAGCAGCCCCG
AGACCCTTCCCTCCCTGCTAAAGCGGCAAGGAAGCTTCTCAGAGGATGTCATTTCC
CACAAAGGAGACTTGAGATTTGTGACTATCTCAGGACAGAAAGTCTTGGACATGG
AAAACAGTTTTAAGGAAGGCAAAGAACCATCAGAAATTGGAAACTTAGTAAAGGA
CAAGTTGAAGGATGCAACAGAAAGATACACTGCTCTCCACTCAAAGTGTACACGA
TTAGGATCTCACCTGAATATGCTGTTAGGCCAGTATCATCAATTCCAAAACAGTGC
TGACAGCCTGCAGGCCTGGATGCAGGCTTGTGAGGCCAACGTGGAGAAGCTCCTC
TCAGATACTGTTGCCTCTGACCCTGGAGTTCTCCAGGAGCAGCTTGCAACAACAAA
GCAGTTGCAGGAGGAATTGGCTGAGCACCAAGTACCTGTGGAAAAACTCCAAAAA
GTAGCTCGTGACATAATGGAAATTGAAGGGGAGCCAGCCCCAGACCACAGGCATG
TTCAAGAAACTACAGATTCCATACTCAGCCACTTCCAAAGCCTCTCCTATAGCCTG
GCTGAGCGATCTTCTCTGCTGCAGAAAGCAATTGCCCAATCTCAGAGTGTCCAGGA
AAGCCTGGAGAGCCTGTTGCAGTCTATTGGGGAAGTTGAACAAAACCTGGAAGGG
AAGCAGGTGTCATCACTCTCATCAGGAGTCATCCAGGAAGCCTTAGCCACAAATAT
GAAATTGAAGCAGGACATTGCTCGGCAAAAGAGCAGCTTGGAGGCCACCCGTGAG
ATGGTGACCCGATTCATGGAGACAGCAGACAGTACTACAGCAGCAGTGCTGCAGG
GCAAACTGGCAGAGGTGAGCCAGCGGTTCGAACAGCTCTGTCTACAGCAGCAAGA
AAAGGAGAGCTCCCTAAAGAAGCTTCTACCCCAGGCAGAGATGTTTGAACACCTC
TCTGGTAAGCTGCAGCAGTTCATGGAAAACAAAAGTCGGATGCTGGCCTCTGGAA
ATCAGCCAGATCAAGATATTACACATTTCTTCCAACAGATCCAGGAGCTCAATTTG
GAAATGGAAGACCAACAGGAGAACCTAGATACTCTTGAGCACCTGGTCACTGAAC
TGAGCTCTTGTGGCTTTGCGCTGGACTTGTGCCAGCATCAGGACAGGGTACAGAAT
CTAAGAAAAGACTTCACAGAGCTACAGAAGACAGTTAAAGAGAGAGAGAAAGAT
GCATCATCTTGCCAGGAACAGTTGGATGAATTCCGGAAGCTGGTCAGGACCTTCCA
GAAATGGTTGAAAGAAACTGAAGGGAGTATTCCACCTACGGAAACTTCTATGAGT
GCTAAAGAGTTAGAAAAGCAGATTGAACACCTGAAGAGTCTACTAGATGACTGGG
CAAGTAAGGGAACTCTGGTGGAAGAAATCAATTGCAAAGGTACTTCTTTAGAAAA
TCTCATCATGGAAATCACAGCACCTGATTCCCAAGGCAAGACAGGTTCCATACTGC
CCTCTGTAGGAAGCTCTGTAGGCAGTGTAAACGGATACCACACCTGCAAAGATCT
GACGGAGATCCAGTGTGACATGTCAGATGTAAACTTGAAGTATGAGAAACTAGGG
GGAGTACTTCATGAACGCCAGGAAAGCCTTCAGGCTATCCTCAACAGAATGGAGG
AGGTTCACAAGGAGGCAAACTCTGTGCTGCAGTGGCTGGAATCAAAAGAGGAAGT
CCTGAAATCCATGGATGCCATGTCATCTCCAACCAAGACAGAAACAGTGAAAGCC
CAAGCTGAATCTAACAAGGCCTTCCTGGCTGAGTTGGAACAGAATTCTCCAAAAAT
TCAAAAAGTAAAGGAAGCCCTGGCTGGATTACTGGTGACATATCCCAACTCACAG
GAAGCAGAAAATTGGAAGAAAATTCAGGAAGAACTCAATTCCCGATGGGAAAGG
GCCACTGAGGTTACTGTGGCTCGGCAAAGGCAGCTAGAGGAATCTGCAAGTCATC
TGGCCTGCTTCCAGGCTGCAGAATCCCAGCTCCGGCCGTGGCTGATGGAGAAAGA
ACTGATGATGGGAGTGCTGGGGCCCCTGTCTATTGACCCCAACATGTTGAATGCAC
AAAAGCAACAGGTCCAGTTTATGCTAAAGGAATTTGAAGCACGCAGGCAACAGCA
TGAGCAACTGAATGAGGCAGCTCAGGGCATCCTAACAGGCCCTGGAGATGTCTCT
CTGTCCACCAGCCAAGTACAGAAAGAACTCCAGAGCATCAATCAGAAATGGGTTG
AGCTGACTGACAAACTCAACTCCCGTTCCAGCCAAATTGACCAAGCTATTGTTAAG
AGCACCCAGTACCAGGAACTGCTCCAGGACTTATCAGAGAAGGTGAGGGCAGTTG
GACAACGGCTGAGTGTCCAGTCAGCTATCAGCACCCAACCAGAGGCTGTAAAGCA
GCAATTGGAAGAGACCAGTGAAATTCGATCTGACTTGGAGCAGTTAGACCACGAG
GTTAAGGAGGCTCAGACACTGTGCGATGAACTCTCAGTGCTCATTGGTGAGCAGTA
CCTCAAGGATGAACTGAAGAAGCGTTTGGAGACAGTTGCCCTGCCTCTCCAAGGTT
TAGAAGACCTTGCAGCCGATCGCATTAACAGACTCCAGGCAGCTCTTGCCAGCACC
CAGCAGTTCCAGCAAATGTTTGATGAGTTGAGGACCTGGTTGGATGATAAACAAA
GCCAGCAAGCAAAAAACTGCCCAATTTCTGCAAAATTGGAGCGGCTACAGTCTCA
GCTACAGGAGAATGAAGAGTTTCAGAAAAGTCTTAATCAACACAGTGGCTCCTAT
GAGGTGATTGTGGCTGAAGGGGAATCTCTACTTCTTTCTGTACCTCCTGGAGAAGA
GAAAAGGACTCTACAAAACCAGTTGGTTGAGCTCAAAAACCATTGGGAAGAGCTT
AGTAAAAAAACTGCAGACAGACAATCCAGGCTCAAGGATTGTATGCAGAAAGCTC
AGAAATATCAGTGGCATGTGGAAGACCTTGTGCCATGGATAGAAGATTGTAAAGC
TAAGATGTCTGAGTTGCGAGTCACTCTGGATCCAGTGCAGCTAGAGTCCAGTCTCC
TAAGATCAAAGGCTATGCTGAATGAGGTGGAGAAGCGCCGCTCCCTGCTGGAAAT
ATTGAATAGTGCTGCTGACATTCTGATCAATTCTTCAGAAGCAGATGAGGATGGAA
TCCGGGATGAGAAGGCTGGGATCAACCAGAACATGGATGCTGTTACAGAAGAGCT
GCAGGCCAAAACAGGGTCACTCGAAGAAATGACTCAGAGGCTCAGGGAGTTCCAG
GAAAGCTTTAAGAATATTGAAAAGAAGGTTGAAGGAGCCAAACACCAACTTGAGA
TCTTTGATGCTCTGGGTTCTCAAGCCTGTAGCAACAAGAACCTGGAGAAGCTAAGA
GCTCAACAGGAAGTGCTGCAGGCCCTAGAGCCTCAGGTAGACTATCTGAGGAACT
TTACTCAGGGTCTGGTAGAAGATGCCCCAGATGGATCTGATGCTTCTCAACTTCTC
CACCAAGCTGAGGTCGCCCAGCAAGAGTTCCTCGAAGTTAAGCAAAGAGTGAACA
GTGGTTGTGTGATGATGGAAAACAAGCTGGAGGGGATTGGCCAGTTTCACTGCCG
GGTCCGAGAGATGTTCTCTCAATTGGCAGACCTGGATGATGAGCTAGATGGCATG
GGTGCTATTGGCAGAGACACTGATAGCCTCCAGTCCCAAATCGAGGATGTCCGGCT
ATTCCTTAACAAAATTCACGTCCTCAAATTAGACATAGAGGCCTCTGAAGCAGAGT
GTCGACATATGCTAGAAGAAGAGGGGACTCTGGATTTGTTAGGTCTCAAAAGGGA
GCTAGAAGCCCTGAACAAACAGTGTGGCAAACTGACAGAGAGGGGGAAAGCTCG
TCAGGAACAGCTGGAACTGACACTAGGCCGTGTAGAGGACTTCTACAGGAAATTG
AAAGGACTCAATGACGCGACCACAGCAGCAGAGGAGGCAGAGGCCCTCCAGTGG
GTAGTGGGGACCGAAGTGGAAATCATCAACCAACAATTAGCAGATTTTAAAATGT
TTCAGAAAGAACAAGTGGATCCTCTTCAGATGAAATTGCAGCAGGTGAATGGACT
TGGCCAGGGATTAATTCAGAGTGCAGGAAAAGACTGTGATGTACAGGGTTTAGAA
CATGACATGGAAGAGATCAATGCTCGATGGAATACATTGAATAAAAAGGTCGCAC
AAAGAATTGCACAGCTACAGGAAGCTTTGTTGCATTGTGGGAAGTTTCAAGATGCC
TTGGAGCCATTGCTCAGCTGGTTGGCAGATACCGAGGAGCTCATAGCCAATCAGA
AACCTCCATCTGCTGAGTATAAAGTGGTGAAAGCACAGATCCAAGAACAGAAGTT
GCTCCAGCGGCTCCTAGATGATCGAAAGGCCACAGTAGACATGCTTCAAGCAGAA
GGAGGCAGAATAGCCCAGTCAGCAGAGCTGGCTGATAGAGAGAAAATCACTGGA
CAGCTGGAGAGTCTTGAAAGTAGATGGACTGAACTACTCAGTAAGGCAGCAGCCA
GGCAAAAACAGCTGGAAGACATCCTGGTTCTGGCCAAACAGTTCCATGAGACAGC
TGAGCCTATTTCTGACTTCTTATCTGTCACAGAGAAAAAGCTTGCTAACTCAGAAC
CTGTTGGCACTCAGACTGCCAAAATACAGCAGCAGATCATTCGGCACAAGGCTCT
GGAAGAAGACATAGAAAACCATGCAACAGATGTGCACCAGGCAGTCAAAATTGG
GCAGTCCCTCTCCTCCCTGACATCTCCTGCAGAACAGGGTGTGCTGTCAGAAAAGA
TAGACTCATTGCAGGCCCGATACAGTGAAATTCAAGACCGCTGTTGTCGGAAGGC
AGCCCTACTTGACCAAGCTCTGTCTAATGCTAGGCTGTTTGGGGAGGATGAGGTGG
AGGTGCTCAACTGGCTGGCTGAGGTTGAGGACAAGCTCAGTTCAGTGTTCGTAAA
GGATTTCAAACAGGATGTCCTGCACAGGCAGCATGCTGACCACCTGGCTTTAAATG
AAGAAATTGTTAATAGAAAGAAGAATGTAGATCAAGCTATTAAAAATGGTCAGGC
TCTTCTAAAACAAACCACAGGTGAGGAGGTGTTACTTATCCAGGAAAAACTAGAT
GGTATAAAGACTCGTTACGCAGACATCACAGTTACTAGCTCCAAGGCCCTCAGAA
CTTTAGAGCAAGCCCGGCAGCTGGCCACCAAGTTCCAGTCTACTTATGAGGAACTG
ACCGGGTGGCTGAGGGAGGTGGAGGAGGAGCTGGCAACCAGTGGAGGACAGTCT
CCCACAGGGGAACAGATACCCCAGTTTCAGCAGAGACAGAAGGAATTAAAGAAG
GAGGTCATGGAGCACAGGCTGGTGTTGGACACAGTGAATGAGGTGAGCCGTGCTC
TCTTAGAGCTGGTGCCCTGGAGAGCCAGAGAAGGGCTGGATAAACTTGTGTCCGA
TGCTAACGAGCAGTACAAACTAGTCAGTGACACTATTGGACAAAGGGTGGATGAA
ATTGATGCTGCTATTCAGAGATCACAACAGTATGAGCAAGCTGCCGATGCAGAAC
TAGCTTGGGTTGCTGAAACAAAACGGAAACTGATGGCTCTGGGTCCAATTCGCCTG
GAACAGGACCAGACCACAGCTCAGCTTCAGGTACAGAAGGCTTTCTCCATTGACA
TTATTCGACACAAAGATTCAATGGATGAACTCTTCAGTCACCGTAGTGAAATCTTT
GGCACATGTGGGGAGGAGCAAAAAACTGTATTACAGGAAAAGACAGAGTCTCTAA
TACAGCAATATGAAGCCATTAGCCTACTCAATTCAGAGCGTTATGCCCGCCTAGAG
CGGGCCCAGGTCTTAGTAAACCAGTTTTGGGAAACTTATGAAGAGCTCAGCCCCTG
GATTGAGGAAACTCGGGCACTAATAGCACAGTTACCCTCTCCAGCCATTGATCATG
AGCAGCTCAGGCAGCAACAAGAGGAAATGAGGCAATTAAGGGAATCTATTGCTGA
ACACAAACCTCATATTGACAAACTACTAAAGATAGGCCCACAACTAAAGGAATTA
AACCCTGAGGAAGGGGAAATGGTGGAAGAAAAATACCAGAAAGCAGAAAACATG
TATGCCCAAATAAAGGAGGAGGTGCGCCAGCGAGCCCTGGCTCTGGATGAAGCCG
TGTCCCAGTCCACACAGATTACAGAGTTTCATGATAAAATTGAGCCTATGTTGGAG
ACACTGGAGAATCTTTCCTCTCGCCTGCGTATGCCACCACTGATCCCTGCTGAAGT
AGACAAGATCAGAGAGTGCATCAGTGACAATAAGAGTGCCACCGTGGAGCTAGAA
AAACTGCAGCCATCCTTTGAGGCCTTGAAGCGCCGTGGAGAGGAGCTTATTGGAC
GATCTCAGGGAGCAGACAAGGATCTGGCTGCAAAAGAAATCCAGGATAAATTGGA
TCAAATGGTATTCTTCTGGGAGGACATCAAAGCTCGGGCTGAAGAACGAGAAATC
AAATTTCTTGATGTCCTTGAATTAGCAGAGAAGTTCTGGTATGACATGGCAGCTCT
CCTGACCACCATCAAAGACACCCAGGATATTGTCCATGACTTGGAAAGCCCAGGC
ATTGATCCTTCCATCATCAAACAACAGGTTGAAGCTGCTGAGACTATTAAGGAAGA
GACAGATGGTCTGCATGAAGAGCTGGAGTTTATTCGGATCCTTGGAGCAGATTTGA
TTTTTGCCTGTGGAGAAACTGAGAAGCCTGAAGTGAGGAAGAGCATTGATGAGAT
GAATAATGCTTGGGAGAACTTAAACAAAACATGGAAAGAGAGGCTAGAAAAACTT
GAGGATGCTATGCAAGCTGCTGTGCAGTATCAGGACACTCTTCAGGCTATGTTTGA
CTGGCTAGATAACACTGTGATTAAACTCTGCACCATGCCCCCTGTTGGCACTGACC
TCAATACTGTTAAAGATCAGTTAAATGAAATGAAGGAGTTCAAAGTAGAAGTTTA
CCAACAGCAAATTGAGATGGAGAAGCTTAATCACCAGGGTGAACTGATGTTAAAG
AAAGCTACTGATGAGACGGACAGAGACATTATACGAGAACCACTGACAGAACTCA
AACACCTCTGGGAGAACCTGGGTGAGAAAATTGCCCACCGACAGCACAAACTAGA
AGGGGCTCTGTTGGCCCTTGGTCAGTTCCAGCATGCCTTAGAGGAACTAATGAGTT
GGCTGACTCATACCGAAGAGTTGTTAGATGCTCAGAGACCAATAAGTGGAGACCC
AAAAGTCATTGAAGTTGAGCTCGCAAAGCACCATGTCCTAAAAAATGATGTTTTGG
CTCATCAAGCCACAGTGGAAACAGTCAACAAAGCTGGCAATGAGCTTCTTGAATC
CAGTGCTGGAGATGATGCCAGCAGCTTAAGGAGCCGTTTGGAAGCCATGAACCAA
TGCTGGGAGTCAGTGTTACAGAAAACAGAGGAGAGGGAGCAGCAGCTTCAGTCAA
CTCTGCAGCAGGCCCAGGGCTTCCACAGTGAAATTGAAGATTTCCTCTTGGAACTT
ACTAGAATGGAGAGCCAGCTTTCTGCATCTAAGCCCACAGGAGGACTTCCTGAAA
CTGCTAGGGAACAGCTTGATACACATATGGAACTCTATTCCCAGCTGAAAGCCAA
GGAAGAGACTTATAATCAACTACTTGACAAGGGCAGACTCATGCTTCTAAGCCGT
GACGACTCTGGGTCTGGCTCCAAGACAGAACAGAGTGTAGCACTTTTGGAGCAGA
AGTGGCATGTGGTCAGCAGTAAGATGGAAGAAAGAAAGTCAAAGCTGGAAGAGG
CCCTCAACTTGGCAACAGAATTCCAGAATTCCCTACAAGAATTTATCAACTGGCTC
ACTCTAGCAGAGCAGAGTTTAAACATCGCTTCTCCACCAAGCCTGATTCTAAATAC
TGTCCTTTCCCAGATAGAAGAGCACAAGGTTTTTGCTAATGAAGTAAATGCTCATC
GAGACCAGATCATTGAGCTGGATCAAACTGGGAATCAATTAAAGTTCCTTAGCCA
AAAGCAGGATGTTGTTCTGATCAAGAATTTGTTGGTGAGCGTGCAGTCTCGATGGG
AGAAGGTTGTCCAGCGATCTATTGAAAGAGGGCGATCACTAGATGATGCCAGGAA
GCGGGCAAAACAATTCCATGAAGCTTGGAAAAAACTGATTGACTGGCTAGAAGAT
GCAGAGAGTCACCTGGACTCAGAACTAGAGATATCCAATGACCCAGACAAAATTA
AACTTCAGCTTTCTAAGCATAAGGAGTTTCAGAAGACTCTTGGTGGCAAGCAGCCT
GTGTATGATACCACAATTAGAACTGGCAGAGCACTGAAAGAAAAGACTTTGCTTC
CCGAAGATAGTCAGAAACTTGACAATTTCCTAGGAGAAGTCAGAGACAAATGGGA
TACTGTTTGTGGCAAGTCTGTGGAGCGGCAGCACAAGTTGGAGGAAGCCCTGCTCT
TTTCGGGTCAGTTCATGGATGCTTTGCAGGCATTGGTTGACTGGTTATACAAGGTG
GAGCCACAGCTGGCTGAGGACCAGCCCGTGCACGGGGACCTTGACCTCGTCATGA
ACCTCATGGATGCACACAAGGTTTTCCAGAAGGAACTGGGAAAGCGAACAGGAAC
CGTTCAGGTCCTGAAGCGGTCAGGCCGAGAGCTGATTGAGAATAGTCGAGATGAC
ACCACTTGGGTAAAAGGACAGCTCCAGGAACTGAGCACTCGCTGGGACACTGTCT
GTAAACTCTCTGTTTCCAAACAAAGCCGGCTTGAGCAGGCCTTAAAACAAGCGGA
AGTGTTTCGAGACACAGTCCACATGCTGTTGGAGTGGCTTTCTGAAGCAGAGCAAA
CGCTTCGCTTTCGGGGAGCACTTCCTGATGACACAGAGGCCCTGCAGTCTCTCATT
GACACCCATAAGGAATTCATGAAGAAAGTAGAAGAAAAGCGAGTGGACGTTAACT
CAGCAGTAGCCATGGGAGAAGTCATCCTGGCTGTCTGCCACCCCGATTGCATCACA
ACCATCAAACACTGGATCACCATCATCCGAGCTCGCTTCGAGGAGGTCCTGACATG
GGCTAAGCAGCACCAGCAGCGTCTTGAAACGGCCTTGTCAGAACTGGTGGCTAAT
GCTGAGCTCCTGGAAGAACTTCTGGCATGGATCCAGTGGGCTGAGACCACCCTCAT
TCAGCGGGATCAGGAGCCAATCCCGCAGAACATTGACCGAGTTAAAGCCCTTATC
GCTGAGCATCAGACATTTATGGAGGAGATGACTCGCAAACAGCCTGACGTGGACC
GGGTCACCAAGACATACAAAAGGAAAAACATAGAGCCTACTCACGCGCCTTTCAT
AGAGAAATCCCGCAGCGGAGGCAGGAAATCCCTAAGTCAGCCAACCCCTCCTCCC
ATGCCAATCCTTTCACAGTCTGAAGCAAAAAACCCACGGATCAACCAGCTTTCTGC
CCGCTGGCAGCAGGTGTGGCTGTTAGCACTGGAGCGGCAAAGGAAACTGAATGAT
GCCTTGGATCGGCTGGAGGAGTTGAAAGAATTTGCCAACTTTGACTTTGATGTCTG
GAGGAAAAAGTATATGCGTTGGATGAATCACAAAAAGTCTCGAGTGATGGATTTC
TTCCGGCGCATTGATAAGGACCAGGATGGGAAGATAACACGTCAGGAGTTTATCG
ATGGCATTTTAGCATCCAAGTTCCCCACCACCAAGTTAGAGATGACTGCTGTGGCT
GACATTTTCGACCGAGATGGGGATGGTTACATTGATTATTATGAATTTGTGGCTGC
TCTTCATCCCAACAAGGATGCGTATCGACCAACAACCGATGCAGATAAAATCGAA
GATGAGGTTACAAGACAAGTGGCTCAGTGCAAATGTGCAAAAAGGTTTCAGGTGG
AGCAGATCGGAGAGAATAAATACCGGTTTGGGGATTCTCAGCAGTTGCGGCTGGT
CCGTATTCTGCGCAGCACCGTGATGGTTCGCGTTGGTGGAGGATGGATGGCCTTGG
ATGAATTTTTAGTGAAAAATGATCCCTGCCGAGCACGAGGTAGAACTAACATTGA
ACTTAGAGAGAAATTCATCCTACCAGAGGGAGCATCCCAGGGAATGACCCCCTTC
CGCTCACGGGGTCGAAGGTCCAAACCATCTTCCCGGGCAGCTTCCCCTACTCGTTC
CAGCTCCAGTGCTAGTCAGAGTAACCACAGCTGTACATCCATGCCATCTTCTCCAG
CCACCCCAGCCAGTGGAACCAAGGTTATCCCATCATCAGGTAGCAAGTTGAAACG
ACCAACACCAACTTTTCATTCTAGTCGGACATCCCTTGCTGGTGATACCAGCAATA
GTTCTTCCCCGGCCTCCACAGGTGCCAAAACTAATCGGGCAGACCCTAAAAAGTCT
GCCAGTCGCCCTGGGAGTCGGGCTGGGAGTCGAGCCGGGAGTCGAGCCAGCAGCC
GGCGAGGAAGTGACGCTTCTGACTTTGACCTCTTAGAGACGCAGTCTGCTTGTTCC
GACACTTCAGAAAGCAGCGCTGCAGGGGGCCAAGGCAACTCCAGGAGAGGGCTA
AACAAACCTTCCAAAATCCCAACCATGTCTAAGAAGACCACCACTGCCTCCCCCAG
GACTCCAGGTCCCAAGCGATAACACTGTCTAAGCACCCCCAAGCCACTATCCACTT
TGAATCCTGCTCCATACATTGGGTGTATATTTATTCTGAACGGGAGAAGTTATATT
GTTAAAAGTGTAAAAGAATAATTGTGTTATGAAGCTGCCTTATTTTTTTTCTTTTTG
TAAGTTACTATTTTCATGTGAATATTTATGTAGATAAAATTTGCCTCCTGGTAACCC
TGTAATGGATGGGGCCCAGAAATGAAATATTTGAGAAAAACAAGTGAAAAGGTCA
AGATACAAATGTGTATTAAAAAAAAAAAAGCCTATTAATAGGGTTTCTGCGCGGT
GCAGGGTTGTAAACCTGCTTTATCTTTTAGGATTATTCCTAAATGCATCTTCTTTAT
AAACTTGACTTGCTATCTCAGCAAGATAAATTATATTAAAAAAATAAGAATCCTGC
AGTGTTTAAGGAACTCTTTTTTTGTAAATCACGGACACCTCAATTAGCAAGAACTG
AGGGGAGGGCTTTTTCCATTGTTTAATGTTTTGTGATTTTTAGCTAAAGAGAGGGA
ACCTCATCTAAGTAACATTTGCACATGATACAGCAAAAGGAGTTCATTGCAATACT
GTCTTTGGATATTGTTTCAGTACTGGGTGTTTAAAGGACAAATAGCTGCTAGAATT
CAGGGGTAAATGTAAGTGTTCAGAAAACGTCAGAACATTTGGGGTTTTAAACTGAT
TTGTTGCTCCCTATCCAGCCTAGACACCAGTAACTCTTGTGTTCACCAGGACCCAG
ACCCTTGGCAAGGGATAGGCTCGTTGGTGACATTGTGAATTTCAGATTTGTTTTATC
CACTTTTTTTGCTATTTATTTAAATGGTCGATCAACTTCCCACAAACTGAGGAATGA
ATTCCACGAGCCTGTTCTGAAAATGTGGACGTAAGACAAACACGTGCTCGTCCTTT
AATGGAGTTCACCAGCACACTTGTTAACCAGTCCTGTTTGCTTTCGTCTTTTTTTGT
GCGTAATAAAGTCAACTGACCAAGTGACCATGAAAAGGGGCTGTCTGGGGCTCCT
GTTTTTTAGCTGCTGTTCTTCAGCTCCGACCATGTTGCTGTGTGATTATCTCAATTG
GTTTTAATTGAGGCAGAAACTGAAGCTCTACCAATGAACTGTTTAGAAACAAGAC
ACACTTTTGTATTAAAATTGCTTGCAGTAACAAATATTTTGTATTTCCTGATTTTCTT
TTCAACTATTACCTTATCTATAAATGTTACCCTGGGGTATAATCATGTTGTAGGTAC
TTAAATGCATTCCGCAAATCAAAATATCTTGATGGATAAATTATAGAGCTTAATAG
ATCTTGTTTTATTTCAAAAAAAAAAAAA
MACF1 Protein
(NP_036222.3; SEQ ID NO: 2)
MSSSDEETLSERSCRSERSCRSERSYRSERSGSLSPCPPGDTLPWNLPLHEQKKRKSQDS
VLDPAERAVVRVADERDRVQKKTFTKWVNKHLMKVRKHINDLYEDLRDGHNLISLL
EVLSGIKLPREKGRMRFHRLQNVQIALDFLKQRQVKLVNIRNDDITDGNPKLTLGLIW
TIILHFQISDIYISGESGDMSAKEKLLLWTQKVTAGYTGIKCTNFSSCWSDGKMFNALIH
RYRPDLVDMERVQIQSNRENLEQAFEVAERLGVTRLLDAEDVDVPSPDEKSVITYVSSI
YDAFPKVPEGGEGISATEVDSRWQEYQSRVDSLIPWIKQHTILMSDKTFPQNPVELKAL
YNQYIHFKETEILAKEREKGRIEELYKLLEVWIEFGRIKLPQGYHPNDVEEEWGKLIIEM
LEREKSLRPAVERLELLLQIANKIQNGALNCEEKLTLAKNTLQADAAHLESGQPVQCE
SDVIMYIQECEGLIRQLQVDLQILRDENYYQLEELAFRVMRLQDELVTLRLECTNLYR
KGHFTSLELVPPSTLTTTHLKAEPLTKATHSSSTSWFRKPMTRAELVAISSSEDEGNLRF
VYELLSWVEEMQMKLERAEWGNDLPSVELQLETQQHIHTSVEELGSSVKEARLYEGK
MSQNFHTSYAETLGKLETQYCKLKETSSFRMRHLQSLHKFVSRATAELIWLNEKEEEE
LAYDWSDNNSNISAKRNYFSELTMELEEKQDVFRSLQDTAELLSLENHPAKQTVEAYS
AAVQSQLQWMKQLCLCVEQHVKENTAYFQFFSDARELESFLRNLQDSIKRKYSCDHN
TSLSRLEDLLQDSMDEKEQLIQSKSSVASLVGRSKTIVQLKPRSPDHVLKNTISVKAVC
DYRQIEITICKNDECVLEDNSQRTKWKVISPTGNEAMVPSVCFLIPPPNKDAIEMASRV
EQSYQKVMALWHQLHVNTKSLISWNYLRKDLDLVQTWNLEKLRSSAPGECHQIMKN
LQAHYEDFLQDSRDSVLFSVADRLRLEEEVEACKARFQHLMKSMENEDKEETVAKM
YISELKNIRLRLEEYEQRVVKRIQSLASSRTDRDAWQDNALRIAEQEHTQEDLQQLRSD
LDAVSMKCDSFLHQSPSSSSVPTLRSELNLLVEKMDHVYGLSTVYLNKLKTVDVIVRSI
QDAELLVKGYEIKLSQEEVVLADLSALEAHWSTLRHWLSDVKDKNSVFSVLDEEIAK
AKVVAEQMSRLTPERNLDLERYQEKGSQLQERWHRVIAQLEIRQSELESIQEVLGDYR
ACHGTLIKWIEETTAQQEMMKPGQAEDSRVLSEQLSQQTALFAEIERNQTKLDQCQKF
SQQYSTIVKDYELQLMTYKAFVESQQKSPGKRRRMLSSSDAITQEFMDLRTRYTALVT
LTTQHVKYISDALRRLEEEEKVVEEEKQEHVEKVKELLGWVSTLARNTQGKATSSETK
ESTDIEKAILEQQVLSEELTTKKEQVSEAIKTSQIFLAKHGHKLSEKEKKQISEQLNALN
KAYHDLCDGSANQLQQLQSQLAHQTEQKTLQKQQNTCHQQLEDLCSWVGQAERAL
AGHQGRTTQQDLSALQKNQSDLKDLQDDIQNRATSFATVVKDIEGFMEENQTKLSPR
ELTALREKLHQAKEQYEALQEETRVAQKELEEAVTSALQQETEKSKAAKELAENKKK
IDALLDWVTSVGSSGGQLLTNLPGMEQLSGASLEKGALDTTDGYMGVNQAPEKLDK
QCEMMKARHQELLSQQQNFILATQSAQAFLDQHGHNLTPEEQQMLQQKLGELKEQY
STSLAQSEAELKQVQTLQDELQKFLQDHKEFESWLERSEKELENMHKGGSSPETLPSL
LKRQGSFSEDVISHKGDLRFVTISGQKVLDMENSFKEGKEPSEIGNLVKDKLKDATER
YTALHSKCTRLGSHLNMLLGQYHQFQNSADSLQAWMQACEANVEKLLSDTVASDPG
VLQEQLATTKQLQEELAEHQVPVEKLQKVARDIMEIEGEPAPDHRHVQETTDSILSHF
QSLSYSLAERSSLLQKAIAQSQSVQESLESLLQSIGEVEQNLEGKQVSSLSSGVIQEALA
TNMKLKQDIARQKSSLEATREMVTRFMETADSTTAAVLQGKLAEVSQRFEQLCLQQQ
EKESSLKKLLPQAEMFEHLSGKLQQFMENKSRMLASGNQPDQDITHFFQQIQELNLEM
EDQQENLDTLEHLVTELSSCGFALDLCQHQDRVQNLRKDFTELQKTVKEREKDASSC
QEQLDEFRKLVRTFQKWLKETEGSIPPTETSMSAKELEKQIEHLKSLLDDWASKGTLV
EEINCKGTSLENLIMEITAPDSQGKTGSILPSVGSSVGSVNGYHTCKDLTEIQCDMSDV
NLKYEKLGGVLHERQESLQAILNRMEEVHKEANSVLQWLESKEEVLKSMDAMSSPTK
TETVKAQAESNKAFLAELEQNSPKIQKVKEALAGLLVTYPNSQEAENWKKIQEELNSR
WERATEVTVARQRQLEESASHLACFQAAESQLRPWLMEKELMMGVLGPLSIDPNML
NAQKQQVQFMLKEFEARRQQHEQLNEAAQGILTGPGDVSLSTSQVQKELQSINQKWV
ELTDKLNSRSSQIDQAIVKSTQYQELLQDLSEKVRAVGQRLSVQSAISTQPEAVKQQLE
ETSEIRSDLEQLDHEVKEAQTLCDELSVLIGEQYLKDELKKRLETVALPLQGLEDLAAD
RINRLQAALASTQQFQQMFDELRTWLDDKQSQQAKNCPISAKLERLQSQLQENEEFQ
KSLNQHSGSYEVIVAEGESLLLSVPPGEEKRTLQNQLVELKNHWEELSKKTADRQSRL
KDCMQKAQKYQWHVEDLVPWIEDCKAKMSELRVTLDPVQLESSLLRSKAMLNEVEK
RRSLLEILNSAADILINSSEADEDGIRDEKAGINQNMDAVTEELQAKTGSLEEMTQRLR
EFQESFKNIEKKVEGAKHQLEIFDALGSQACSNKNLEKLRAQQEVLQALEPQVDYLRN
FTQGLVEDAPDGSDASQLLHQAEVAQQEFLEVKQRVNSGCVMMENKLEGIGQFHCR
VREMFSQLADLDDELDGMGAIGRDTDSLQSQIEDVRLFLNKIHVLKLDIEASEAECRH
MLEEEGTLDLLGLKRELEALNKQCGKLTERGKARQEQLELTLGRVEDFYRKLKGLND
ATTAAEEAEALQWVVGTEVEIINQQLADFKMFQKEQVDPLQMKLQQVNGLGQGLIQS
AGKDCDVQGLEHDMEEINARWNTLNKKVAQRIAQLQEALLHCGKFQDALEPLLSWL
ADTEELIANQKPPSAEYKVVKAQIQEQKLLQRLLDDRKATVDMLQAEGGRIAQSAEL
ADREKITGQLESLESRWTELLSKAAARQKQLEDILVLAKQFHETAEPISDFLSVTEKKL
ANSEPVGTQTAKIQQQIIRHKALEEDIENHATDVHQAVKIGQSLSSLTSPAEQGVLSEKI
DSLQARYSEIQDRCCRKAALLDQALSNARLFGEDEVEVLNWLAEVEDKLSSVFVKDF
KQDVLHRQHADHLALNEEIVNRKKNVDQAIKNGQALLKQTTGEEVLLIQEKLDGIKT
RYADITVTSSKALRTLEQARQLATKFQSTYEELTGWLREVEEELATSGGQSPTGEQIPQ
FQQRQKELKKEVMEHRLVLDTVNEVSRALLELVPWRAREGLDKLVSDANEQYKLVS
DTIGQRVDEIDAAIQRSQQYEQAADAELAWVAETKRKLMALGPIRLEQDQTTAQLQV
QKAFSIDIIRHKDSMDELFSHRSEIFGTCGEEQKTVLQEKTESLIQQYEAISLLNSERYAR
LERAQVLVNQFWETYEELSPWIEETRALIAQLPSPAIDHEQLRQQQEEMRQLRESIAEH
KPHIDKLLKIGPQLKELNPEEGEMVEEKYQKAENMYAQIKEEVRQRALALDEAVSQST
QITEFHDKIEPMLETLENLSSRLRMPPLIPAEVDKIRECISDNKSATVELEKLQPSFEALK
RRGEELIGRSQGADKDLAAKEIQDKLDQMVFFWEDIKARAEEREIKFLDVLELAEKFW
YDMAALLTTIKDTQDIVHDLESPGIDPSIIKQQVEAAETIKEETDGLHEELEFIRILGADLI
FACGETEKPEVRKSIDEMNNAWENLNKTWKERLEKLEDAMQAAVQYQDTLQAMFD
WLDNTVIKLCTMPPVGTDLNTVKDQLNEMKEFKVEVYQQQIEMEKLNHQGELMLKK
ATDETDRDIIREPLTELKHLWENLGEKIAHRQHKLEGALLALGQFQHALEELMSWLTH
TEELLDAQRPISGDPKVIEVELAKHHVLKNDVLAHQATVETVNKAGNELLESSAGDD
ASSLRSRLEAMNQCWESVLQKTEEREQQLQSTLQQAQGFHSEIEDFLLELTRMESQLS
ASKPTGGLPETAREQLDTHMELYSQLKAKEETYNQLLDKGRLMLLSRDDSGSGSKTE
QSVALLEQKWHVVSSKMEERKSKLEEALNLATEFQNSLQEFINWLTLAEQSLNIASPPS
LILNTVLSQIEEHKVFANEVNAHRDQIIELDQTGNQLKFLSQKQDVVLIKNLLVSVQSR
WEKVVQRSIERGRSLDDARKRAKQFHEAWKKLIDWLEDAESHLDSELEISNDPDKIKL
QLSKHKEFQKTLGGKQPVYDTTIRTGRALKEKTLLPEDSQKLDNFLGEVRDKWDTVC
GKSVERQHKLEEALLFSGQFMDALQALVDWLYKVEPQLAEDQPVHGDLDLVMNLM
DAHKVFQKELGKRTGTVQVLKRSGRELIENSRDDTTWVKGQLQELSTRWDTVCKLSV
SKQSRLEQALKQAEVFRDTVHMLLEWLSEAEQTLRFRGALPDDTEALQSLIDTHKEFM
KKVEEKRVDVNSAVAMGEVILAVCHPDCITTIKHWITIIRARFEEVLTWAKQHQQRLE
TALSELVANAELLEELLAWIQWAETTLIQRDQEPIPQNIDRVKALIAEHQTFMEEMTRK
QPDVDRVTKTYKRKNIEPTHAPFIEKSRSGGRKSLSQPTPPPMPILSQSEAKNPRINQLS
ARWQQVWLLALERQRKLNDALDRLEELKEFANFDFDVWRKKYMRWMNHKKSRVM
DFFRRIDKDQDGKITRQEFIDGILASKFPTTKLEMTAVADIFDRDGDGYIDYYEFVAAL
HPNKDAYRPTTDADKIEDEVTRQVAQCKCAKRFQVEQIGENKYRFGDSQQLRLVRILR
STVMVRVGGGWMALDEFLVKNDPCRARGRTNIELREKFILPEGASQGMTPFRSRGRR
SKPSSRAASPTRSSSSASQSNHSCTSMPSSPATPASGTKVIPSSGSKLKRPTPTFHSSRTSL
AGDTSNSSSPASTGAKTNRADPKKSASRPGSRAGSRAGSRASSRRGSDASDFDLLETQS
ACSDTSESSAAGGQGNSRRGLNKPSKIPTMSKKTTTASPRTPGPKR
HMG20A cDNA;
(NM_018200.3; SEQ ID NO: 3)
AAACCTCCACGAAAATAAGGCTCACCTTGCGTAACCACGTAGTCCTTCGCCGCATT
GGGGCAAAATAATCCCTTCATTTTTGTGAAGGTACCGTGGAAAATATTTCATTTTTC
TTCTCACCGGAGCAATTGTAAATGCTATGCGGTAAGAGGAGTTACCTGTGGAAAG
GTGGTTAAGAGATTAGGTAAAGAAAAGGAAAGGACACCAAAATAAAGTGCTGCG
GAAGAATTTTTGTCCAGCTGTGAGACGACGAGTGCGTGAAGTGAAGGCGATTGAG
AGGGGCTGAGGGAATTGTCCTCTGTGGAAGGGACTTTCTTTTGGCCCTAGGCCCCT
TCCTGCCCCTGTCGTCAGCAGAGTCTCTACAAGGAAGATAACGGACTGTAAAATTC
TATAAAGCAAAGCTACACATCACTTGACACCATACACCATCTTGGTTACATAATGA
AGAGAGATGGAAAACTTGATGACTAGCTCCACCCTACCGCCCCTTTTTGCAGATGA
AGACGGTTCCAAGGAGAGTAATGATCTGGCTACCACTGGGTTAAATCACCCAGAG
GTTCCATACAGTAGTGGCGCCACATCATCCACCAACAATCCAGAATTTGTGGAGGA
TCTCTCTCAAGGTCAGTTGCTTCAGAGTGAGTCTTCAAATGCAGCAGAAGGCAATG
AACAGAGGCATGAAGATGAGCAACGAAGTAAACGAGGAGGTTGGTCCAAAGGAA
GAAAGAGGAAGAAACCTCTTCGAGACAGCAATGCACCCAAATCCCCCCTTACAGG
ATATGTTCGGTTCATGAATGAGCGTCGAGAACAACTTCGAGCAAAGAGACCAGAA
GTCCCATTTCCAGAAATCACAAGGATGTTAGGCAATGAATGGAGTAAACTGCCTCC
TGAGGAAAAACAGCGCTACCTTGATGAAGCAGACAGAGATAAGGAGCGTTACATG
AAGGAACTGGAACAGTATCAGAAAACAGAGGCCTACAAGGTCTTCAGTAGGAAA
ACCCAGGACCGTCAGAAAGGCAAATCTCATAGGCAAGATGCAGCCCGGCAGGCCA
CTCATGATCATGAGAAAGAAACAGAGGTAAAGGAACGGTCTGTTTTTGACATCCC
TATATTTACAGAGGAATTCTTGAACCATAGCAAAGCTCGGGAAGCAGAGCTCCGC
CAGCTTCGCAAATCCAACATGGAGTTTGAGGAGAGGAATGCAGCCCTGCAAAAGC
ACGTGGAGAGCATGCGCACAGCAGTGGAGAAGCTGGAGGTGGATGTGATCCAGG
AGCGGAGCCGCAACACAGTCTTACAGCAGCACCTGGAGACCCTGCGGCAGGTGCT
GACCAGCAGCTTTGCCAGCATGCCCTTGCCTGGAAGTGGAGAGACACCTACAGTG
GACACCATTGACTCATATATGAACAGACTGCACAGTATTATTTTAGCTAATCCCCA
AGACAATGAAAACTTCATAGCTACAGTTCGAGAAGTTGTGAACAGACTCGATCGT
TAGGGAATGGTCTTAGAACTCCAAGATGTTCCATAAGTGTTTTTACTTGTGAGGAA
TGAGAAGCCATCCATGGAAATTTGAACTGAGTGGGGGCAGAGAAAGAGTGCAGAT
CCCTTTGCTTGTGAAAGAATTATCAGTGAGTGAAAGGCCATCACCCCAGGAAGCC
AAATGAGGGAGCAGCAACATGTATATGAGCTTCCTATGGAATTGTCCTTATGTGAA
GCTTTGAAGGTGTACAGCCACTCTCCCGGGTCTTCAGGTTCCTACCATTTCCATTTC
TGTTAAAGTGGATCTGCATATCTTCAGCTTACTAGGTGACCCGGATGCTGACATCT
GCTGCTGCAGAAAGGAAGACTTTTCATTGTAATTTCGCTTAGACCCTTTTATCAGT
GGAGCTCCAGTTTTCTTACCTAGCTGTCACTTTTTTAAATGCCTCTGGGGGTTATTT
TTGCTTTCCTTGGCCCCCACCAATTTATACATCTCCATTTTCTGACCTCTGGACTAA
CTGGTTGCTCAGCAAGGTTCTGAAGGAGAGTTTCTTGCATTGGACAGGCCCAGTCT
TCTCCCATCATTGCCCTGCTGTGACTCCAAAGAAAGGAGCTTCTTGCTGACAGTGC
CCTGTGGAGCAAGGCTGTGTTTCCTACCCCACACGGTGCTCAGTGGGTGCCAGCCC
TCAGTGTGGCTTTGTGATTGCTGCCCTAAAGGAGAATGCTCTTTCCTTCCTCACTGG
TACTGCCTGCTGTTTTCTAAGCATTGCTCCTGCACAGACATGGAGTCCCAGCCCCA
GCAAGGCTCTTCTGTTCCCATCTGTTGACAATGTCTTGTGGAGCATTTTTGCTGAGG
AAAAGGTCACTTGTAAACAGAGGAGAAAGGGAAAGAGTACAAAGCCCTAAGTTT
ATTGTAAGTGAAAACTGAGGGAATTCCTGTCTTCTTTAGGAGTAATGATTCATAGA
TCTAGATAGGTGGAAATATCATTCAAAATAGTCACTTGAGCTCACAAAAAAAGCA
AGGAAGAATTCTCATGTCCTTTGTCTTCCTTCTGTAGCCATTAACTGCTGAATCCAT
GTGAGGAAGACAGGCTTCCCTTCCTTCCCCCTCCTTAGTGATTTTTTCTTTAACAGC
ATAAGTAAAGAGGACTTTCTGGTTCATTTTTGTTTGTTTTGTTTTGTTTTGTTTTGTT
TACAGATGAGGTCTTCCTGTGTTGCCCAGGCTGGAGTGCGGTGGCTATTCACAGAT
GCTATCATAGCACACTACAGCCTACAACTCTTGGGCTCAAGCATCACGCCTAGCAG
TTTCTGGTTCCTTTAACAGCAAAAGGAAAGAGAGGTTCTGATTCTTACCTCAGGGT
TTTTTGGTTGTTCATTGTTTTTGTTTTTGTTTTTGTTTTGACACTGCAGAGCACAAGG
CTAAAGGTTACAGCTGAGATCTTTGGAACCAAAGGCAGAGCAAGCAGAGCCCGTT
GTCTGGGCCCCACACCACTGCAGGCAGGTGGATAGAAGTGCGGCCCCTCTCATAG
TATGCCCATAAGTCAGGGCATAGGGCAGAACTACCTGTCATGTTGCTACACCATCC
TGTCTTCTCAGCATCTCCTTGCCTGTTTTCTTTATTAGTCCAAAGGAAAACAACAGC
AACAAAATCTGTTTTTAAAATGTCTTATATGAACATATATCAAATATCCATGCGCT
GAAACCCACATACCATCACTTGGCAATTTTTTAGAATAAGACCCCATTATTATCTA
TTGCTATAAACCTAGCCAGTTCTCTTGCTCTTCTGTATTTTCCTATTTCCCTGCCATC
ATCTGCTATTTCTGCCACTTCTCTTAGACTCCTTGTCTGCAAAGCCCAAGCTAGAAC
TCACTGTCTATGGCAGAAGGACATCCAGAGCCCATTCTGGAGTTTTGTTTTTTCCTT
CTGCCAGATGCTTTGTGTCCTGTCTTCCTTCCTCCTCATATTTCTGTTTCTCATTTGT
GTTCAGTTTTGTGCAGCATTGCTAGCACTGCTTTTGTGACCAGAAAAGGCCATAAC
ATGGTCCAGGATCATCATTCTTCTGACTCTAGATGGGACACTTGACAGTGACTTGA
AACATTTGCATATTCAGGAATGCATGAGATTTCAAGAGAGCCTACAGTATGAAATC
ATTTTCACAAAATAAGCAGCTTGCTTCTGAAATGCTGTCTTTCCCAGTAGCTACTCA
CCTGCCTCTGGTGGCTGGGATTCAGATGCCACAAAACTGTCAGTATCTATAGACCA
GGTCTGTGCCACCTCCTCTCTCCTCTGTGCTCAGTGAGGAGGCAGTAAATGAAGTT
ACAGGCTAGCACAATACCTAACTCATGTTTCCCAGTACACCTGTAGATATTACTGT
ACTTTTATGTTCTCAAGAAATAAGTTGTTGCCTATTCAGTGTTACAGATTTCTTTGT
TTCTTTTTAATTAAAATACAAGAAGCAGCTGAGGAAAGGGAGACAAGGTATTTTAT
TTCTGACTGATTTTAGAAAAAACTTGTGTACATGTGTTTGGAACTGTTGAAATGCC
AAGTTTTCTGTATAAGTGTTTTTGTAATTAAACTTTCAGATTTTCTTTGTTTTTTAAG
AAGTTGATGTGCTTGTTTGACATTTGTCTCATTAAAACTTTTCTACGTTGAAAAAAA
AAAAAAAAAAAAA
HMG20A Protein;
(NP_060670.1; SEQ ID NO: 4)
MENLMTSSTLPPLFADEDGSKESNDLATTGLNHPEVPYSSGATSSTNNPEFVEDLSQGQ
LLQSESSNAAEGNEQRHEDEQRSKRGGWSKGRKRKKPLRDSNAPKSPLTGYVRFMNE
RREQLRAKRPEVPFPEITRMLGNEWSKLPPEEKQRYLDEADRDKERYMKELEQYQKT
EAYKVFSRKTQDRQKGKSHRQDAARQATHDHEKETEVKERSVFDIPIFTEEFLNHSKA
REAELRQLRKSNMEFEERNAALQKHVESMRTAVEKLEVDVIQERSRNTVLQQHLETL
RQVLTSSFASMPLPGSGETPTVDTIDSYMNRLHSIILANPQDNENFIATVREVVNRLDR
QPCTL cDNA;
(NM 017659.4; SEQ ID NO: 5)
AATCCGTGGTCTGGTACAGGTTTCAGGGCAAAGCGGCCATGCGTTCCGGGGGCCG
CGGGCGACCCCGCCTGCGGCTGGGGGAACGTGGCCTCATGGAGCCACTCTTGCCG
CCGAAGCGCCGCCTGCTACCGCGGGTTCGGCTCTTGCCTCTGTTGCTGGCGCTGGC
CGTGGGCTCGGCGTTCTACACCATTTGGAGCGGCTGGCACCGCAGGACTGAGGAG
CTGCCGCTGGGCCGGGAGCTGCGGGTCCCATTGATCGGAAGCCTCCCCGAAGCCC
GGCTGCGGAGGGTGGTGGGACAACTGGATCCACAGCGTCTCTGGAGCACTTATCT
GCGCCCCCTGCTGGTTGTGCGAACCCCGGGCAGCCCGGGAAATCTCCAAGTCAGA
AAGTTCCTGGAGGCCACGCTGCGGTCCCTGACAGCAGGTTGGCACGTGGAGCTGG
ATCCCTTCACAGCCTCAACACCCCTGGGGCCAGTGGACTTTGGCAATGTGGTGGCC
ACACTGGACCCAAGGGCTGCCCGTCACCTCACCCTTGCCTGCCATTATGACTCGAA
GCTCTTCCCACCCGGATCGACCCCCTTTGTAGGGGCCACGGATTCGGCTGTGCCCT
GTGCCCTGCTGCTGGAGCTGGCCCAAGCACTTGACCTGGAGCTGAGCAGGGCCAA
AAAACAGGCAGCCCCGGTGACCCTGCAACTGCTCTTCTTGGATGGTGAAGAGGCG
CTGAAGGAGTGGGGACCCAAGGACTCCCTTTACGGTTCCCGGCACCTGGCCCAGCT
CATGGAGTCTATACCTCACAGCCCCGGCCCCACCAGGATCCAGGCTATTGAGCTCT
TTATGCTTCTTGATCTCCTGGGAGCCCCCAATCCCACCTTCTACAGCCACTTCCCTC
GCACGGTCCGCTGGTTCCATCGGCTGAGGAGCATTGAGAAGCGTCTGCACCGTTTG
AACCTGCTGCAGTCTCATCCCCAGGAAGTGATGTACTTCCAACCCGGGGAGCCCTT
TGGCTCTGTGGAAGACGACCACATCCCCTTCCTCCGCAGAGGGGTACCCGTGCTCC
ATCTCATCTCCACGCCCTTCCCTGCTGTCTGGCACACCCCTGCGGACACCGAGGTC
AATCTCCACCCACCCACGGTACACAACTTGTGCCGCATTCTCGCTGTGTTCCTGGCT
GAATACCTGGGGCTCTAGCGTGCTTGGCCAATGACTGTGGAGAGGACTGTGAGAG
AGAAGGTCCCAGCGGGGGCCAGTGAAGCTCAGGCAGGATCTGCCTAGGGTGTGCT
GGTTTGTCCTTTTCATACCTTTGTCTCCTAATTGTGCTACAATTGGAAGACCTTCTTT
CTTTTGATTGTCTCAAGCTGCCACCCTTCAAGGACAGGGAAGAGACCACTGTGGGA
TGACAGCCAGAGGAATAAGAACTTGCTCCCTCCCCAGAGGTAAACACTTGGTCCA
AAGGTTTGCAGGGACCAAATACTGTTCTTTTTTTTTTTGAGACGGAGTCTCACTGTG
TTGCCCAGGCTGGAGTGCAGTGGTGCGATCTCGGCTCACTGCAAACTCCGCCTCCT
GGGTTCACGCCATTCTCCTGCCTCAGCCTCCCAAGTAGCTGGGACTACAGGTGCCC
GCCACCACGCTGGCTAATTTTTTGTATTTTTAGTAGAGACGGGGTTTCACCGTGTTA
GCCAGGATGGTCTCGATCTCCTGACCTTGTAATCCGCCAGCCTCGGCCTCCCAAAG
TGCTGGGATTACAGGTGTGAGCCACCGCACCTGGCAAAATGCTGTTCTTTAAGTCA
GCGAACTGAAAAAGTAAAAGACTGCTGGGTGTGGTGGCTCACACCTGTAATCCCA
ACACTTTGAGAGGCTGAGGGGGAAGGATCACCCGAGGTCAGGAGTTTGAGACCAG
CCTGGTCAACATGGCGAAACCCTGTCTCTACTAAAAATACAATAATTAGCTGGGCA
TGGTGGTGGGCGCCTGTAATCTCAGCTGCTTGGGAGGCTGAGGCAGGAGAATCAC
TTGTACGCAGGAGGCGGAGGTTGCAGTGAGCCAAGATCACACCACTGCACTCCAG
CCTGGGCAACAGAGCGAGACTCCATCTCAATAAATAAATAAATAAATAAATATTT
TTTAAAAA
QPCTL Protein;
(NP_060129.2; SEQ ID NO: 6)
MRSGGRGRPRLRLGERGLMEPLLPPKRRLLPRVRLLPLLLALAVGSAFYTIWSGWHRR
TEELPLGRELRVPLIGSLPEARLRRVVGQLDPQRLWSTYLRPLLVVRTPGSPGNLQVRK
FLEATLRSLTAGWHVELDPFTASTPLGPVDFGNVVATLDPRAARHLTLACHYDSKLFP
PGSTPFVGATDSAVPCALLLELAQALDLELSRAKKQAAPVTLQLLFLDGEEALKEWGP
KDSLYGSRHLAQLMESIPHSPGPTRIQAIELFMLLDLLGAPNPTFYSHFPRTVRWFHRL
RSIEKRLHRLNLLQSHPQEVMYFQPGEPFGSVEDDHIPFLRRGVPVLHLISTPFPAVWH
TPADTEVNLHPPTVHNLCRILAVFLAEYLGL
SLC30A8 cDNA;
(NM_173851.3; SEQ ID NO: 7)
AAGCTCTTGAGCTCCTCTACCTCTTAGAAAGCACAATTGAATCAGATATCATATGA
AAGACATACACACTTCATGTAATGCTACCTGCAAGTCTCCCTAGAAAAGCAGTTTT
TGTAGGTGAAAACAATGAAGCCAGGTAATATTGCAAGGAGGCTGTAATTTTAGCA
GACCTACCAACAACACTGATGTAGGAAGCTCATTATTTTAATTTCTGGAGCCTTTT
AATTTTTTCTTTAGAAAGTGTATAAATAATTGCAGTGCTGCTTTGCTTCCAAAACTG
GGCAGTGAGTTCAACAACAACGACAACAACAGCCGCAGCTCATCCTGGCCGTCAT
GGAGTTTCTTGAAAGAACGTATCTTGTGAATGATAAAGCTGCCAAGATGTATGCTT
TCACACTAGAAAGTGTGGAACTCCAACAGAAACCGGTGAATAAAGATCAGTGTCC
CAGAGAGAGACCAGAGGAGCTGGAGTCAGGAGGCATGTACCACTGCCACAGTGG
CTCCAAGCCCACAGAAAAGGGGGCGAATGAGTACGCCTATGCCAAGTGGAAACTC
TGTTCTGCTTCAGCAATATGCTTCATTTTCATGATTGCAGAGGTCGTGGGTGGGCA
CATTGCTGGGAGTCTTGCTGTTGTCACAGATGCTGCCCACCTCTTAATTGACCTGAC
CAGTTTCCTGCTCAGTCTCTTCTCCCTGTGGTTGTCATCGAAGCCTCCCTCTAAGCG
GCTGACATTTGGATGGCACCGAGCAGAGATCCTTGGTGCCCTGCTCTCCATCCTGT
GCATCTGGGTGGTGACTGGCGTGCTAGTGTACCTGGCATGTGAGCGCCTGCTGTAT
CCTGATTACCAGATCCAGGCGACTGTGATGATCATCGTTTCCAGCTGCGCAGTGGC
GGCCAACATTGTACTAACTGTGGTTTTGCACCAGAGATGCCTTGGCCACAATCACA
AGGAAGTACAAGCCAATGCCAGCGTCAGAGCTGCTTTTGTGCATGCCCTTGGAGAT
CTATTTCAGAGTATCAGTGTGCTAATTAGTGCACTTATTATCTACTTTAAGCCAGAG
TATAAAATAGCCGACCCAATCTGCACATTCATCTTTTCCATCCTGGTCTTGGCCAGC
ACCATCACTATCTTAAAGGACTTCTCCATCTTACTCATGGAAGGTGTGCCAAAGAG
CCTGAATTACAGTGGTGTGAAAGAGCTTATTTTAGCAGTCGACGGGGTGCTGTCTG
TGCACAGCCTGCACATCTGGTCTCTAACAATGAATCAAGTAATTCTCTCAGCTCAT
GTTGCTACAGCAGCCAGCCGGGACAGCCAAGTGGTTCGGAGAGAAATTGCTAAAG
CCCTTAGCAAAAGCTTTACGATGCACTCACTCACCATTCAGATGGAATCTCCAGTT
GACCAGGACCCCGACTGCCTTTTCTGTGAAGACCCCTGTGACTAGCTCAGTCACAC
CGTCAGTTTCCCAAATTTGACAGGCCACCTTCAAACATGCTGCTATGCAGTTTCTG
CATCATAGAAAATAAGGAACCAAAGGAAGAAATTCATGTCATGGTGCAATGCACA
TTTTATCTATTTATTTAGTTCCATTCACCATGAAGGAAGAGGCACTGAGATCCATC
AATCAATTGGATTATATACTGATCAGTAGCTGTGTTCAATTGCAGGAATGTGTATA
TAGATTATTCCTGAGTGGAGCCGAAGTAACAGCTGTTTGTAACTATCGGCAATACC
AAATTCATCTCCCTTCCAATAATGCATCTTGAGAACACATAGGTAAATTTGAACTC
AGGAAAGTCTTACTAGAAATCAGTGGAAGGGACAAATAGTCACAAAATTTTACCA
AAACATTAGAAACAAAAAATAAGGAGAGCCAAGTCAGGAATAAAAGTGACTCTG
TATGCTAACGCCACATTAGAACTTGGTTCTCTCACCAAGCTGTAATGTGATTTTTTT
TTCTACTCTGAATTGGAAATATGTATGAATATACAGAGAAGTGCTTACAACTAATT
TTTATTTACTTGTCACATTTTGGCAATAAATCCCTCTTATTTCTAAATTCTAACTTGT
TTATTTCAAAACTTTATATAATCACTGTTCAAAAGGAAATATTTTCACCTACCAGA
GTGCTTAAACACTGGCACCAGCCAAAGAATGTGGTTGTAGAGACCCAGAAGTCTT
CAAGAACAGCCGACAAAAACATTCGAGTTGACCCCACCAAGTTGTTGCCACAGAT
AATTTAGATATTTACCTGCAAGAAGGAATAAAGCAGATGCAACCAATTCATTCAGT
CCACGAGCATGATGTGAGCACTGCTTTGTGCTAGACATTGGGCTTAGCATTGAAAC
TATAAAGAGGAATCAGACGCAGCAAGTGCTTCTGTGTTCTGGTAGCAACTCAACA
CTATCTGTGGAGAGTAAACTGAAGATGTGCAGGCCAACATTCTGGAAATCCTATGT
CAATGGGTTTGGTTTGGAACCTGGACTTCTGCATTTTTAAAAGTTACCCAGAGATG
CTTCTAAAGATGAGCCATAGTCTAGAAGATTGTCAACCACAGGAGTTCATTGAGTG
GGACAGCTAGACACATACATTGGCAGCTACAATAGTATCATGAATTGCAATGATG
TAGTGGGGTATAAAAGGAAAGCGATGGATATTGCCGGATGGGCATGGCCAGTGAT
GTTTCACGTCATTGAGGTGACAGCTCTGCTGGACTTTGAATTACATATGGAGGCTC
TCCAGGAAGACGAAGAAGAGAAGGACATTCTAGGCAAAAAGAAGACTAGGCACA
AGGCACACTTATGTTTGTCTGTTAGCTTTTAGTTGAAAAAGCAAAATACATGATGC
AAAGAAACCTCTCCACGCTGTGATTTTTAAAACTACATACTTTTTGCAACTTTATGG
TTATGAGTATTGTAGAGAACAGGAGATAGGTCTTAGATGATTTTTATGTTGTTGTC
AGACTCTAGCAAGGTACTAGAAACCTAGCAGGCATTAATAATTGTTGAGGCAATG
ACTCTGAGGCTATATCTGGGCCTTGTCATTATTTATCATTTATATTTGTATTTTTTTC
TGAAATTTGAGGGCCAAGAAAACATTGACTTTGACTGAGGAGGTCACATCTGTGC
CATCTCTGCAAATCAATCAGCACCACTGAAATAACTACTTAGCATTCTGCTGAGCT
TTCCCTGCTCAGTAGAGACAAATATACTCATCCCCCACCTCAGTGAGCTTGTTTAG
GCAACCAGGATTAGAGCTGCTCAGGTTCCCAACGTCTCCTGCCACATCGGGTTCTC
AAAATGGAAAGAATGGTTTATGCCAAATCACTTTTCCTGTCTGAAGGACCACTGAA
TGGTTTTGTTTTTCCATATTTTGCATAGGACGCCCTAAAGACTAGGTGACTTGGCAA
ACACACAAGTGTTAGTATAATTCTTTGCTTCTGCTTCTTTTTGAAAATCATGTTTAG
ATTTGATTTTAAGTCAGAAATTCACTGAATGTCAGGTAATCATTATGGAGGGAGAT
TTGTGTGTCAACCAAAGTAATTGTCCCATGGCCCCAGGGTATTTCTGTTGTTTCCCT
GAAATTCTGCTTTTTTAGTCAGCTAGATTGAAAACTCTGAACAGTAGATGTTTATAT
GGCAAAATGCAAGACAATCTACAAGGGAGATTTTAAGGATTTTGAGATGAAAAAA
CAGATGCTACTCAGGGGCTTTATGAACCATCCATCAATTCTGAAGTTCTGACTCTC
CCATTACCCTTTCCCTGGTGTGGTCAGAACTCCAGGTCACTGGAAGTTAGTGGAAT
CATGTAGTTGAATTCTTTACTTCAAGACATTGTATTCTCTCCAGCTATCAAAACATT
AATGATCTTTTATGTCTTTTTTTTGTTATTGTTATACTTTAAGTTCTGGGGTACATGT
GCGGAACATGTAGGTTTGTTACATAGGTATACATGTGCCATGGTGGTTTGCTGCAC
TCATCAACCTGTCATCTACATTCTTTTATGTCTGTCTTTCAAAGCAACACTCTGTTC
TTCTGAGTAGTGAAATCAGGTCAACTTTACCACCAGCCTCCATTTTTAATATGCTTC
ACCATCATCCAGCACCTACTTAAGATTTATCTAGGGCTCTGTGGTGATGTTAGGAC
CCATAAAAGAAATTTATGCCTTCCATATGTTTGGTTACAGATGGGAAATGGGAATG
TTGAAGGACATGAAAGAAAGGATGTTTACACATTAAGCATCAGTTCTGAAGCTAG
ATTGTCTGAGTTTGAATCTTAGCTCTTCCCTTTATTAGCTCTGTGACCTCGAGCTAG
TTACTTAAATGCTCTGATCCTCTATTTCCTGATCAGTGAAACCTCCCTATTCAAATG
TGTGAGAGTTTAATAAATTAGGACACTTAAAAATGTTGGAGCAGTGCATAGCATGT
AGTGTTCAGTACATGTTAAATGTTGTTTTTTATTATGTACAAACATGAGTGGGCAC
AGAATTTTAAATCATCTCAACTTTTGAGAAATTTTGAGTTATCAACACCGTTCCCAC
AAGACAGTGGCAAAATTATTGGTGAGAATTAAACAGCTGTTTCTCAGAGGAAGCA
ATGGAGGCTTGCTGGGATAAAGGCATTTACTGAGAGGCTGTTACCTAGTGAGAGT
GATGAATTAATTAAAATAGTCGAATCCCTTTCTGACTGTCTCTGAAAGCTTCCGCTT
TTATCTTTGAAGAGCAGAATTGTCACTCCAAGGACATTTATTAATAAAAAGAACAA
CTGTCCAGTGCAATGAAGGCAAAGTCATAGGTCTCCCAAGTCTTACCCCATTCCTG
TGAAATATCAAGTTCTTGGCTTTTCTCTGTCATGTAGCCTCAACTTTCTCTGACCGG
GTGCATTTCTTTCTCTGGTTTCTAAATTGCCAGTGGCAAATTTGGATCACTTACTTA
ATATCTGTTAAATTTTGTGACCCAACAAAGTCTTTTAGCACTGTGGTGTCAAAAAG
AAAAACACCTCCCAGGCATATACATTTTATAGATTCCTGGAGAATGTTGCTCTCCA
GCTCCATCCCCACCCAATGAAATATGATCCAGAGAGTCTTGCAAAGAGACAAGCC
TCATTTTCCACAATTAGCTCTAAAGTGCCTCCAGGAAATGATTTTCTCAGCTCATCT
CTCTGTATTCCCTGTTTTGGATCACAGGGCAATCTGTTTAAATGACTAATTACAGA
AATCATTAAAGGCACCAAGCAAATGTCATCTCTGAATACACACATCCCAAGCTTTA
CAAATCCTGCCTGGCTTGACAGTGATGAGGCCACTTAACAGTCCAGCGCAGGCGG
ATGTTAAAAAAAATAAAAAGGTGACCATCTGCGGTTTAGTTTTTTAACTTTCTGAT
TTCACACTTAACGTCTGTCATTCTGTTACTGGGCACCTGTTTAAATTCTATTTTAAA
ATGTTAATGTGTGTTGTTTAAAATAAAATCAAGAAAGAGAGA
SLC30A8 Protein;
(NP_776250.2; SEQ ID NO: 8)
MEFLERTYLVNDKAAKMYAFTLESVELQQKPVNKDQCPRERPEELESGGMYHCHSGS
KPTEKGANEYAYAKWKLCSASAICFIFMIAEVVGGHIAGSLAVVTDAAHLLIDLTSFLL
SLFSLWLSSKPPSKRLTFGWHRAEILGALLSILCIWVVTGVLVYLACERLLYPDYQIQA
TVMIIVSSCAVAANIVLTVVLHQRCLGHNHKEVQANASVRAAFVHALGDLFQSISVLIS
ALIIYFKPEYKIADPICTFIFSILVLASTITILKDFSILLMEGVPKSLNYSGVKELILAVDGV
LSVHSLHIWSLTMNQVILSAHVATAASRDSQVVRREIAKALSKSFTMHSLTIQMESPVD
QDPDCLFCEDPCD
NUCB2 cDNA;
(NM_005013.4; SEQ ID NO: 9)
AGAGCGGAGCGGTGGGCCGGGGGCTGGAGGACAGGTTTGTGCGCTGGACGCAAG
CACCAGGCGCAGCCTCGCTCGCCGAGACCCGGCCAGAACGTGTTACGAGTCAGTT
TTTAGTGAAAAAACATTGAGCTAGGAGCCAAGACCCATCTCTTCACTATTTTGGTA
TTGTGCAAGTCATCTTACCTCTCTGGATCTCAGTTGTCTCATCTGTAAAAAGGAGAT
AAAAATTATTTACCTGCCTGAACATGAGGTGGAGGACCATCCTGCTACAGTATTGC
TTTCTCTTGATTACATGTTTACTTACTGCTCTTGAAGCTGTGCCTATTGACATAGAC
AAGACAAAAGTACAAAATATTCACCCTGTGGAAAGTGCGAAGATAGAACCACCAG
ATACTGGACTTTATTATGATGAATATCTCAAGCAAGTGATTGATGTGCTGGAAACA
GATAAACACTTCAGAGAAAAGCTCCAGAAAGCAGACATAGAGGAAATAAAGAGT
GGGAGGCTAAGCAAAGAACTGGATTTAGTAAGTCACCATGTGAGGACAAAACTTG
ATGAACTGAAAAGGCAAGAAGTAGGAAGGTTAAGAATGTTAATTAAAGCTAAGTT
GGATTCCCTTCAAGATATAGGCATGGACCACCAAGCTCTTCTAAAACAATTTGATC
ACCTAAACCACCTGAATCCTGACAAGTTTGAATCCACAGATTTAGATATGCTAATC
AAAGCGGCAACAAGTGATCTGGAACACTATGACAAGACTCGTCATGAAGAATTTA
AAAAATATGAAATGATGAAGGAACATGAAAGGAGAGAATATTTAAAAACATTGA
ATGAAGAAAAGAGAAAAGAAGAAGAGTCTAAATTTGAAGAAATGAAGAAAAAGC
ATGAAAATCACCCTAAAGTTAATCACCCAGGAAGCAAAGATCAACTAAAAGAGGT
ATGGGAAGAGACTGATGGATTGGATCCTAATGACTTTGACCCCAAGACATTTTTCA
AATTACATGATGTCAATAGTGATGGATTCCTGGATGAACAAGAATTAGAAGCCCT
ATTTACTAAAGAGTTGGAGAAAGTATATGACCCTAAAAATGAAGAGGATGATATG
GTAGAAATGGAAGAAGAAAGGCTTAGAATGAGGGAACATGTAATGAATGAGGTT
GATACTAACAAAGACAGATTGGTGACTCTGGAGGAGTTTTTGAAAGCCACAGAAA
AAAAAGAATTCTTGGAGCCAGATAGCTGGGAGACATTAGATCAGCAACAGTTCTT
CACAGAGGAAGAACTAAAAGAATATGAAAATATTATTGCTTTACAAGAAAATGAA
CTTAAGAAGAAGGCAGATGAGCTTCAGAAACAAAAAGAAGAGCTACAACGTCAG
CATGATCAACTGGAGGCTCAGAAGCTGGAATATCATCAGGTCATACAGCAGATGG
AACAAAAAAAATTACAACAAGGAATTCCTCCATCAGGGCCAGCTGGAGAATTGAA
GTTTGAGCCACACATTTAAAGTCTGAAGTCCACCAGAACTTGGAAGAAAGCTGTTA
ACTCAACATCTATTTCATCTTTTTAGCTCCCTTCCTTTTTCTCTGCTCAATAAATATT
TTAAAAGCATATTTGAAATAAAGGGAGATACTTTTTAAATGAAAACACTTTTTTTG
GGACACAGATATTAAAGGATTGAAGTTTATCAGAACCAGGAAGAAAACAAACTCA
CTGTCTGCTCTCTGCTCTCACATTCACACGGCTCTTTTATTTATTTTTTTGTTCTCCT
TTAATGATTTAATTAAGTGGCTTTATGCCATAATTTAGTGAAACTATTAGGAACTAT
TTAAGTGAGAAAACTCTGCCTCTTGCTTTTAAATTAGATTGCTCTCACTTACTCGTA
AACATAGGTATTCTTTTATGGGTGCTTATCATTCCTTCTTTCAATAAATGTCTGTTT
GATATTAACAATTCTGGAAAGGCCACAGTATTTCCCTGTGTTTCCTGGTAACGTTTT
TCTAGTTTTGGCAACCTCAACTGCTAGAAATTCTTCACCTGAATCACTTTTGCTACC
ACTTCAGGTCATTTTTCATTCTTTTTTATTTTGCTCTATACTTTATCATTTAAGATTA
GGTTATGTTACATATAACAGAAAAAACAAAGATAACAGTGGTTTAAACTAAATAG
GAGTTTTTCTTCTTACATAAGTCTAGAAGTAGGTGGTGTCCAGGTTCCTATCTTTCT
GCTCTGCTATCCTCAGTGCATGATTTTTATCCTCAGATTACCTCACTCTCACTGTTC
AAGTTTGCTCCTGGAG
NUCB2 Protein;
(NP_005004.1; SEQ ID NO: 10)
MRWRTILLQYCFLLITCLLTALEAVPIDIDKTKVQNIHPVESAKIEPPDTGLYYDEYLKQ
VIDVLETDKHFREKLQKADIEEIKSGRLSKELDLVSHHVRTKLDELKRQEVGRLRMLIK
AKLDSLQDIGMDHQALLKQFDHLNHLNPDKFESTDLDMLIKAATSDLEHYDKTRHEE
FKKYEMMKEHERREYLKTLNEEKRKEEESKFEEMKKKHENHPKVNHPGSKDQLKEV
WEETDGLDPNDFDPKTFFKLHDVNSDGFLDEQELEALFTKELEKVYDPKNEEDDMVE
MEEERLRMREHVMNEVDTNKDRLVTLEEFLKATEKKEFLEPDSWETLDQQQFFTEEE
LKEYENIIALQENELKKKADELQKQKEELQRQHDQLEAQKLEYHQVIQQMEQKKLQQ
GIPPSGPAGELKFEPHI
SSR1 cDNA;
(NM_003144.5; SEQ ID NO: 11)
GGATGAAGAGTAACGCCATTACCGCCGGAGCCGCCGAGAGCCTTAGCCGACGGAA
ACTGGACACTGGACCGGCAGCGCCATGAGACTCCTCCCCCGCTTGCTGCTGCTTCT
CTTACTCGTGTTCCCTGCCACTGTCTTGTTCCGAGGCGGCCCCAGAGGCTTGTTAGC
AGTGGCACAAGATCTTACAGAGGATGAAGAAACAGTAGAAGATTCCATAATTGAG
GATGAAGATGATGAAGCCGAGGTAGAAGAAGATGAACCCACAGATTTGGTAGAA
GATAAAGAGGAAGAAGATGTGTCTGGTGAACCTGAAGCTTCACCGAGTGCAGATA
CAACTATACTGTTTGTAAAAGGAGAAGATTTTCCAGCAAATAACATTGTGAAGTTC
CTGGTAGGCTTTACCAACAAGGGTACAGAAGATTTTATTGTTGAATCCTTAGATGC
CTCATTCCGTTATCCTCAGGACTACCAGTTTTATATCCAGAATTTCACAGCTCTTCC
TCTGAACACTGTAGTGCCACCCCAGAGACAGGCAACTTTTGAGTACTCTTTCATTC
CTGCAGAGCCCATGGGCGGACGACCATTTGGTTTGGTCATCAATCTGAACTACAAA
GATTTGAACGGCAATGTATTCCAAGATGCAGTCTTCAATCAAACAGTTACAGTTAT
TGAAAGAGAGGATGGGTTAGATGGAGAAACAATCTTTATGTATATGTTCCTTGCTG
GTCTTGGGCTTCTGGTTATTGTTGGCCTTCATCAACTCCTAGAATCTAGAAAGCGTA
AGAGACCCATACAGAAAGTAGAAATGGGTACATCAAGTCAGAATGATGTTGACAT
GAGTTGGATTCCTCAGGAAACATTGAATCAAATCAATAAAGCTTCACCAAGAAGG
TTGCCCAGGAAACGGGCACAGAAGAGATCAGTGGGATCTGATGAGTAAATGTTCC
TTTGTGCAACAATTCGGTCTTTACTTAACCTGCCCTAATATTTTTCGGCCTGATGGG
AATTAGTGCAGAGAAGCCATGTCACCATAGAAGGCAACTCCTACTTGTGTGTGGA
CTGAGCAATCAGAGTCTGTGGCGATAATATTGCTGAAAATGCACTGCATTCATTTT
TCTAAAGTAACAAATTTGGTTTTTTTTTAAACCATTAAAATCTATGTGTGTGCGTGT
GTATGTATGTGAGCAGTTGGTCTTACCAGAATCATTGTTGAACTACCTGAAACAAG
TCTTTAGAATACTAAATATAATGCTGTTGTCTCTTCCTTTTTGACATTTTCTGATTTT
TTCCCCCAAAACTCAGTTAATATTTACCCACTATGATTATTGATGTCCTGCCTTGAA
CAGTTTTAAAGAAAACAATTTTTGGAATAGCTCAAATTTCAATTGATGGCACAAAT
CAGCATTTTGTTGTTGTTACTGTATTACAATTAGTATTCTAAAGGCAGAAGCAGAA
GTAGCTGCTTTTTAGCAATAGAATTGTTTCAGTATTTTGCTGCTGTTTAATGCGCAT
CTTCAGAAAACTTCCCAGTGGCTTCAAGGAATTTGGGGATCTCTCTGGCAACAAAT
TGTGAAACATGAAATTTCTGCTGACTTTAATATATGAAACCTAATCCTACCCCCTTT
TTTAACAAAAAGAAACTAGTACATTTGTGAAAATTGTGTTGTGTTGTCCATTGTTG
CTCTAGTTCTGACCCAGAGGTAGCTCTGGAGTGATTTTAGACCTACTCACTCAGTT
GTGTGTAGGTTTTTTTGTTTTGTTTTGAGAGAGAATTTTTCTCTCCTTAATAGAAGC
ATCCTTTTTAAAGAGAAGTTGCCTTGGTCCACACACTAAGCAGAAAACCAAGTTAT
CAGGACAGAGATATTTCCCAGTTACTCCTAATCAATGAAGAAAGTGAGTTGGATAT
TTTTAAAGCAGTTAACTAATTTTTTCTTACCTAATCTTTTGGGAGTTTTGCTTGTTGA
TATAACCTTTTTAGTTAACCTGAAAGATTCCAAAAATTGTTCTTAAGTGCTTGAGAC
TGGAACCAAAATTAAATTGTACTTCATAAAATCCTCTTATAGAGTTACTCTTGCCCT
AGATTGTAAATTAAGTTTGGCATTATTGTCAGACTGGATGGAGGGTGAAGTAAAAT
AGTATGAACAATTAAGAGGCTCTCCCCCTCTTGTCTTTAAGCCATATTCTCCTACAT
GTATTTTATAAGAAAATGTTAAGTCAAATTTTAGTGGCTCTTTAATTCCTGACCTCT
TCATTCTCCTTTTCAGTATAACCTCCCCTATGCTCATGCCCACACAGACAAAAAAA
CAAAACGAAATACACACAGAAAAAAGTCTTTCCAAACTGTTTAAGTATTTAAACA
TCTGAGCCAAAGCAGATAGAAGTTATTGTATAATTGTTAATCACTTTGCAAATAGG
GGCTATCAAATTACCTATATTGGCATTGCTGGATTATAAACTCTATATCTGTAATAT
AAAGTGTTTGAGTTTTTAATTGGGCTGTTATGATCAGTAGTTGATTTTGAGAAAGCT
CTATGAGCTCTAAGTAACTGCATGGTTTTTTGTTTAATGTAATATAGGAGACCCTTC
ACATTCCCAAGGAATATATTCCAAAACATTTTTGTGAATATCTAAGTTTGTGAAAC
TACTAGGGCATGATACAGTAAGGTGTAATTACAGAATTTACGAAATGTAAATGGC
CTCTACAGAGTTTTATGGAATACCTGGTACTAACGTAGGCAGCTGCAAAACCACAC
TGAGTTACAGCTGTCAGCCCTCCTCATTCCTAAATAACTTGCCTTACATATCAGCCC
TCCCACTTCTGAAGTTCAAATTAGTGCCTCGGAAATGTAGAATTTATTATTTGTCAT
TTTTTTTTTTTTAGCATAGATTGAGAACAGTTGAACTCTTAAATCCTCAGATGCCAG
GGGTCTGCTCTAGCATCAGTAAGTATTTAGCAGAAACTAACTCCGTAATGAATGGA
ATTCAATTCCACACATGGTTTGTTCAAGCACACTTAATAAGTAGCCTATTTTTTAAA
TGTCTTTTTAAAATGTAAATATTTGGATGAAGTTTTTCTTTGTTTTGATATATTCATT
TGCTACACCAACTATGTTTTCAGAATTCATCTTTTGAACAACTTGGTTTCAGAATAT
GTAAAATGACTTTAAGGATCTTGTGTATCAAACCTATCCCCGGATGTGTGAGAATA
ATGTGTTCATAAAGCATGGATCTCGCTTTGGTTGTATAGCTTCCTCATTTACTTCAT
GGTCTTACATAGCTGGTGACTTTCAGGGCTAATCTGCCCTCTAAAGCATTGTCCCA
GGAGAGGAAAAGGAAATGGGACCTCAGAAGTAGAAGCCTCAGGGAAGGAGTAAA
GTAGAAATCAGAAGAAAAGAAGCTTCACTTGATAGTAATAAGGTTTTTAACTTCAA
GTACCTTCAGAAAATGTGATTTTGATAAGAGGAAAGGGCAAATTTAGACCTTAAA
AAATATGGAAGAACTACTGCCTTAAAAGTGCATTTGTGGCACATCAGCCTAGAACT
GTATCATGGCTGTGCTGGGGAGAAGTAAATGGTGGTAATGTAACATTGCCACCTTT
ACTTAATGATGTGTTATTTTCGAGGTACAGTAGATCAATATAGTAATAGGCGAGCC
TCATATATAGCATTCATCTTGTACAATGATATCCATACCCTTGATATGAAGGAAAA
TTGACTTGGTTTGTGCATTTGAATACTGAAATAATTTTTTAAAACTCAGTGACACAT
ACCATCTCTTGCCAAGACTAGACCCTGTATTTTAGTTCCTAAATTAGATGTTTAAAT
TTAAAAACATTTTCAGATGTACTTAAGTACTTCCATAGTACTTTTTTTTTTTTTTTTT
TTTTTGCCCCCTGAGACGGAGTCTCTGTGTCACCCAGGCTGGAGTGCAGTGGTGCG
ATCTCGGCTCACTGCAACTTCTGCCTCTTGGGTTCAAGCAGTTCTCCCTGTCTCAGC
CTCCTGAGTAGCTTGGATTACAGGCGCCCGTCACCGCACCTGCCTAATTTTTGCATT
TTTAGTAGAGACGGGGTTTCGTCATTTGGCCAGGCTGGTCTTGAACTCCTGACCAC
AGGTGATACGCCTGCCTTGGCCTCCCAAAGTGTGCTGGGATTACAGGCGTGAGCCA
CTGTGCCCGGCCCACTGTTCACTTTTTGAATGGCATCATTTTATAGCTGTAGAACTA
AAATCAATGTTTGCCCCAATTTTCTTAAGTAAAACTCTACTTTGAGCTCTTACCTCC
AACTTAGTAAAAAGCAGCTTCACACACAAACAAGATTCTTACTGGTGGAATGTTA
GGTTTCGTTGTTAAGTTAATCTGTCTATAAGCTCATCCTTAGAGGATATTTGAGGAG
GAAGAACACCTTGCAGCTGACTTGCAAACATCTAAATAATTTATTTCGGGTGCTTA
TGAATGTTACTAATGGATTTTGTATGAATTTTTATCCCTTTTCATTTATACAAAAAC
CTGGGCTTTTATGTTAATTATATCATCTGAGGTTCTAAGGTTTTTTTTTTAGATTTTG
AAATTTAGGGATAATAGCTCTTAGGTTTGGGTACCACTTTGCTGCAGTTTAAGAAA
GGGGGAAGGGAACTCATTTATTAAACATCAATCACGTGCTGTGTTCTGTTTGTTTTC
TAGTCATCATATCACACACCTTTACGACAGCTCACTGAAGGAAGGTGATACTGTTC
CCATTTTGTAGATGGAATAGACAAAACCTGAATTTAAGTAGCTTGCTCAAGGTTCC
ATATTGAATATGGAAAGTTCAAATCATCTCAGTAATGAATATACCATATATACTTG
CTGTATTGTATCTATGATAATTCAGTTACCCACAATACCCTTTTAAATTTCTGTTAA
TGACATACCTTTAAATGTCTCCTTGATGAACAGAATCATGGTCTTTAAAAACATTTT
CATGGGTTGATTGCATTTTCAAGCTCTAAAGGATTGAAAGATAAATCTTCACGTTA
AAGGTAAGAGTGAAGTATCTGCTCTTGGGTTACAGAACCAGATAGTACTAGAACT
AAGATTACAGGGTAAAGCTGCTTTTATCTTTTTTCTTTTTCTTTTTCTTTTTTTTTTTG
ACATGGGGTCTCACTGTATTGCCCAGGCTGGAATGCAGTGGCATGATCTCAGCTCA
CGGCAGCCTCTGCCTCTTGGGCTCAAGCGATTCTCCTGCTTCAGCTTTCCAAGTATT
TGGGACCACAGGCGCACACCACAGGCCTGGCTAATGTTTTTGTTTTGTTTTTGGTA
GAGACGGGGTTTCACCATGTTGCCAGGCTGGTCTCGAACTCCTGAGCTCAAGTGAT
TCACCCACCTTGGCCTCACAAAGTGTCAGGCTTACAGGCGTGAGCCACTGCGCCCG
GCTCACAGGGTAAGGCTTCTGTCTGGTGTGTTGTATTACGGATTTTGCTTAATAGGC
ACAGTGAGGCATTAAAAAGAAAATTCAGTATGCCTGTAGAAAGGATAATCCTTGT
TTAAAGTCTCCAAATTGCAGTCAAAGATGTTTTGACTGTGCCTTTTTTTGTTCCCCT
GCTGTCCCTTATGTAGACTTCTGTCAGTACCCATGGCAGCCTGTCATCTTGTTGACA
TCTCCTTCTGGACTGTGAGCTCTGTATCTGGCTTGTTTTTCATCCCCAGCTTCTAGTT
CACAATTAGGTAGAACCCTATTACTCTTTGAAGAAGGAACAAGAAAATGTGGGCC
AGTTTTCATTTGCCATTCTTCCATGTGAGTTAGTATGGTTCGTAAGTATTCCTGGTG
ATACGCTAGTATTGGCAATTCTGTGAGGTTGAACAAAGGGGTGGTATGGTGTGCTA
GCGTGGGAATTAGGAGACCTCTGGGTCTTGACAGTGCCCTGGCCACTAAGCAAAG
GCAGTTCATCCTTGGAGTCTCAATGTGCTTTTTTGTTAATTGAGATATGCTTGAAGT
ATCAGCCCTAAATAGTCTGATTCTGTGACCTACAAACCCTTACTTAATTCAGTGTTA
CTATAAATGATTCTTCCCTTAAACCTACTTTTTACTTAGCAAAAGAGAAAAAAAAA
AAAAGGAAACGCTCATGTCAGGCTGCCTGGGTTTGAACCCCAGATCCCCTCTTAGT
TGGAGGCAAGTTGTATGATGCTTCAGCTTTTGGTTCCTGCCTCCTAAGGTTGTTAGG
AGTTACTGTGTGTAGCTGCTTAGAACATTGCCTGGGTCTCAGTAAGCCGCTTAAGT
GACTGCTCTCATTTTCGCTGTAAAGCACCATACTGTAATAACATCCCATGAAGCAT
GGGGCGGGGAAGAGTATATGGTACCTTATGGACTTTGATGTGGTGGGGTAGTAGG
TAAATTCTGAATATGTAAGCTACATAGTATTCTTTTTGTAACTAAAGGATAAAAGT
TTTAAGATGGGCATGTAATATGGCTAGCACTGGATTTTAATGATGAGCCAGAGTAA
TAAGGCTGGCAAGGGGAGTTTTTGTTTTGTAATAAATCTCTTACACCTGCACATCC
AGTTCTTTTTAAAAACACGTTTGAAGAGGCTCCATATTTCTCAGTGAAGCTGTTGG
GGTTCATGTTATTTTTGAAAATCATCTTGCAATTTATTTCAGCATCAGACTGAACCA
CCCAAAGAAAATTAGTCTATTTCTGAACAGTTTTTTCAGAAACCATATTGGTCTGA
TCACCCAACATTTATAGAAATAGGCGCTTAAGGCCTAGAGGCATTTCAGAAAGGA
GAATGAGAAATACTGTGGTCAAGAGTAGTGTTCAGATGGAGAACATCAAGCATCT
GCTCTGCCCTTCCACACACATCCTCATTTCATCTTCACAACTGCCCTAGGTAACCTT
GTTATCTGTAATGAAACAGCCTCAAGGAGCACAGAGGCACTCACTCCTCACGTTTG
GTCTGTTGCCATTTCCGGCTTGGTTGGAATAGGGTAGGAGCCTTTTGGCAGGGAGC
ACATTCTCAGTAATGCAGAGTGCACTCACCTGGGTTCTAGCTTCAACACTTAGGAT
TTGCTTGATATTTTGTATTCTCTGAGGCACTGCCTGTATTTGTTTCTGCTATCACAGT
CCAGAGTCAAGCTTCATTTATAAAGACTGGGCAGGGCATGGTGGCTCACTCCTGTA
ATTCCAGCACTTTGGGAGGCTGAGGTGGGTGGATCACTTTAGGTCAGGAGTTCAAG
ACCAGCCTGGCCAACATGGTGAAACCCCATCTCTACTAAAAAATACAAAAAGGCC
GGGCACGGTGGCTCACACCTGTAATCCCAACACTTTGGGAGGCCAAGGTGGGCAG
AACACCTGAGGTCAGGAGTTCAAGACCATCCTGGCGAACATAGTGAAACCTCGCC
TCTACTAAAAATACAAAAATTAGCCAGGTGTGGTGGTGCATGCCTGTAATCCCAGC
TACTTGGGAGGCTGAGGCAGGAGAATTGCTTAAACCTGGGAGGTGGAGATTGTGG
TGAGCTGAGATCGTGCCACTGCACTCCAGCCTGGGAGATAGAACGAGACTCCATC
TCAAAAAAGAACAAAAACAATTAGCCGAGCGAGGTGGTGCACGCCTGTAATCCCA
GCTACTCATGAGGCTGAGGCAGGAGAATCACTTGAACCCAGGAGGAGGAGGTTGT
AGTGAGTCAAGGTTGCACCACTGCACTCCAGCCTGGGTGACAGAGTGAGACTGTC
TCAAAAAATAAGTACATAAATAATAAGTAAAAGCTACTAACAATTAAAAAATAAA
TAAATAAAGACAAGACTGTCTGGAAAATGGCTCTCCTAAAAGGACCAGTTGCCAT
CATCCACAGTGGAAGATTCAAAGCAGTTGGTCCTTGGTACGTATGAGAAGCGGAT
TTCATTCCCTTGAATTCTACAGAGCAGTTTATTAGAGTGAATGCATTTTAAGGCCTT
GCATTTGATATGTCATCCAGTTCATAATCAAGTTGCCTTTTTCTGGCTAAAACATAA
TGATTATGTATTTTTCTCATTTGGTCCTACAAGCTGCTGGCCCTTTGTCCCTCCACT
GTGGGAATCAGATCTAGAGGAGGCTGAGCCTGCAGACACAGCAGTGGCCAAAAG
GTCACTCTAAGTGTTTTGTCTTGACTCCTTACTTGAAGTCCACCCAGCTAGCACACA
TCTGGTTTATACTGAAGCCCCCTGCCTAGAAATACTCATTTCAGGAACCACCAGTA
AGCATCTGTGACCACACAGGCTTTTTGACTGATGGCTTCCCGGATCTGGTTTCAAG
GGATAACCCCGTCTGTGTGCATCTATGGTCTTCTCTCTACAGCGAGGACTTTGCAG
TGCTGCTTGTGGTCCACACAAGGGGCTCAGAGCTGAGTCTGAACTGCTTCATGGTC
ACCAGCTCCTGTCCCTTCCAGTCTTGAGAGGCTTTTTTCTCCAGATGGAACCTTTCC
TTCCCGCCGTTTTCTCGGTCTCTGGCTGTTTTTCTCTTGTGCCCGTCTAATTGGACAC
CTCCTGGCTTCCATCTCTGTGGTTCTCCTGCCTCACTTCCTGTTCTGTTGTTTTTCCG
TTTTGTCAAAATATCTCCTATGTTCTTGGCTTCCTTTTCGTCGCCAGGTTTTCAGCTT
TCCTTTAGCTCTTCTTCTAATATGGCTTCTGCCCACAAAAGCCTGCTCTGTCAGGAT
CTCATGGTTCTCCACTTGCCAGAACCTTCTTCAGCCTCAGTTCCTCGGCCTCAACTT
GTACGTTTAACCCATTGACCACCACCCCCCAAATTCACCTTCATTTCTTTGACCCTG
CTCCTCACTCCTTTTCTGTTGAGGAATCTGTTGACTAACTCCAGGCTCACTCAGGCT
CACCGTCCTGCTCTCTGCACCAGCCTTTCCAGAGCGTGCCAGTTCTCATGGCTTCAT
CTGTTAACTGTTGATCACTTCAGTCCTGATTTTTAGACCTAAATGGTTTCCTTAACG
CCATTCTAACTGCCTGTGACTCATTTTCACTTACAGTGTTTATTGTAACGCCAAACC
AACAAATCACAGGTGCTTGCTTCTCTCCATAAATCTCCCCAGTCTAACTTTTTGTCA
TTCAACATGACTCGTTTATCCAACCTGAAATCGCATATAGCCCCAAGTATGGTGTT
TTGTACACAGGTATTTAATAAGTGACTTCCAGTTTTGGCTCTGCTATGAATAAAAA
GAGATTTCAGTTCTCTTCACTTTGAAATCTAACAACTCAGAGAACATTGAAGAAAT
TGGAATTTAGTTGGGATGAAATACTTGTGGTTTAAAATATTTCTGTTCATATTTTCT
AATTTGTTGCCGGAGGTCTTGGGTTTTCTATTTGAGTGCTTGCAAACTCAATGTGAT
TTCTGTCAGCATATCTTAGGTTTGTTTGTTATGAAACTTATGCAGTGTGAGGTTCTA
TCTGAAAATGTTATTTAGCTATCTTCTGGGACTATTTAATGAAAGTGGGGTCATGA
ATCCTTAAAATTCTTGTGCAGCTTTGAGAAACATTTCTGTTATTTGGGTATCAGTTT
GTAAGTGTGGTAAAGCCAAGATGGAAACGAGCACTTTGCTTTCTTGGTTGTTGTTA
CTGGTCTAACCTCCTGCTTGAACTAGTCTGCTGTCCTGTCAAATGCATCTTTTTATT
TACATGTCCCTTAAATTAAAGCTGATCATGAAAGTA
SSR1 Protein;
(NP_003135.2; SEQ ID NO: 12)
MRLLPRLLLLLLLVFPATVLFRGGPRGLLAVAQDLTEDEETVEDSIHIEDEDDEAEVEED
EPTDLVEDKEEEDVSGEPEASPSADTTILFVKGEDFPANNIVKFLVGFTNKGTEDFIVES
LDASFRYPQDYQFYIQNFTALPLNTVVPPQRQATFEYSFIPAEPMGGRPFGLVINLNYK
DLNGNVFQDAVFNQTVTVIEREDGLDGETIFMYMFLAGLGLLVIVGLHQLLESRKRKR
PIQKVEMGTSSQNDVDMSWIPQETLNQINKASPRRLPRKRAQKRSVGSDE
ATG16L2 cDNA;
(NM_033388.2; SEQ ID NO: 13)
GGGAGGAACGCGCCGCTAGGCGGGAGAGCGCGGCCATGGCGGGGCCGGGCGTCC
CCGGTGCCCCCGCAGCGCGCTGGAAACGCCACATCGTGCGGCAGCTGCGGCTTCG
GGACCGTACGCAAAAGGCGCTTTTCCTGGAGCTGGTGCCGGCCTATAACCATCTCT
TAGAGAAGGCTGAGCTGCTGGACAAGTTCTCAAAGAAGCTGCAGCCGGAGCCAAA
CAGTGTCACTCCCACCACCCACCAGGGCCCCTGGGAGGAGTCAGAGCTTGACTCA
GACCAAGTCCCATCACTGGTCGCACTGAGGGTGAAGTGGCAGGAGGAGGAGGAG
GGGCTCCGGCTGGTCTGTGGTGAGATGGCCTACCAGGTGGTGGAGAAGGGCGCGG
CCCTGGGCACGCTGGAGTCGGAGCTGCAGCAGAGGCAAAGCAGGCTGGCAGCCCT
GGAGGCCCGCGTGGCGCAGCTGCGAGAGGCGCGGGCGCAGCAGGCCCAGCAGGT
GGAGGAGTGGCGGGCGCAGAATGCGGTGCAGCGGGCAGCCTACGAGGCGCTGCG
CGCGCACGTCGGGCTCCGGGAGGCGGCACTGCGCAGGCTCCAGGAAGAGGCGCGC
GACCTGCTGGAGAGGCTCGTGCAGCGCAAGGCGCGCGCCGCGGCCGAGCGCAACC
TGCGCAACGAGCGCCGGGAGCGGGCCAAGCAGGCGCGGGTGTCCCAGGAGCTGA
AGAAGGCTGCCAAGCGGACCGTGAGCATCAGCGAGGGCCCGGACACCCTAGGCG
ATGGGATGAGGGAGAGAAGGGAGACTCTGGCTCTGGCCCCTGAGCCAGAGCCCCT
GGAGAAGGAAGCTTGTGAGAAGTGGAAGAGGCCCTTCAGGTCTGCCTCAGCCACC
TCCCTGACGCTGTCCCACTGTGTGGATGTGGTGAAGGGGCTTCTGGATTTTAAGAA
GAGGAGAGGTCACTCAATTGGGGGAGCCCCTGAGCAGCGATACCAGATCATCCCT
GTGTGTGTGGCTGCCCGACTTCCTACCCGGGCTCAGGATGTGCTGGATGCCCACCT
CTCTGAGGTCAATGCTGTTCGTTTTGGCCCCAACAGCAGCCTCCTGGCCACTGGAG
GGGCTGACCGCCTGATCCACCTCTGGAATGTTGTGGGAAGTCGCCTGGAGGCCAA
CCAGACCCTGGAGGGAGCTGGTGGCAGCATCACCAGTGTGGACTTTGACCCCTCG
GGCTACCAGGTTTTAGCAGCAACTTACAACCAGGCTGCCCAGCTCTGGAAGGTGG
GGGAGGCACAGTCCAAGGAGACACTGTCTGGACACAAGGATAAGGTGACAGCTGC
CAAATTCAAGCTAACGAGGCACCAGGCAGTGACTGGGAGCCGCGACCGGACAGTG
AAGGAGTGGGACCTCGGCCGTGCCTATTGCTCCAGGACCATCAATGTCCTTTCCTA
CTGTAATGACGTGGTGTGTGGGGACCATATCATCATTAGTGGCCACAATGACCAGA
AGATCCGGTTCTGGGACAGCAGGGGGCCCCACTGCACCCAGGTCATCCCTGTGCA
GGGCCGGGTCACCTCCCTGAGCCTCAGCCACGACCAACTGCACCTGCTCAGCTGTT
CCCGAGACAACACACTCAAGGTCATCGACCTGCGTGTCAGCAACATCCGCCAGGT
GTTCAGGGCCGATGGCTTCAAGTGTGGTTCTGACTGGACCAAAGCTGTGTTCAGCC
CGGACAGAAGCTATGCACTGGCAGGCTCCTGTGATGGGGCCCTTTACATCTGGGAT
GTGGACACCGGGAAACTGGAGAGCAGACTACAGGGACCCCATTGCGCTGCCGTCA
ACGCCGTGGCCTGGTGCTACTCCGGGAGCCACATGGTGAGCGTGGACCAGGGCAG
GAAGGTTGTGCTCTGGCAGTAGGGCCACGACCTGCCTGCCTGGGCTGGAGCTCTTG
CCCGAAGCCTGAAGCTTCCTTCGGCGCCATGCAGGGGTTGGGGTTGGGACTGGAG
CTGGCCTTGGGATTTAATGGGGAAGAAGGCCTGGCAGGACCTGGCCTGTTTGTTTA
AAAATGAAGTATGGGTTGGGGGATTACGCTAGTTTTTCTTTGTATTTTTATCTCTAT
CTCCTCACTTTTTCTCCCAAAGTAGAAAAAAATGATATCTGAA
ATG16L2 Protein;
(NP_203746.1; SEQ ID NO: 14)
MAGPGVPGAPAARWKRHIVRQLRLRDRTQKALFLELVPAYNHLLEKAELLDKFSKKL
QPEPNSVTPTTHQGPWEESELDSDQVPSLVALRVKWQEEEEGLRLVCGEMAYQVVEK
GAALGTLESELQQRQSRLAALEARVAQLREARAQQAQQVEEWRAQNAVQRAAYEAL
RAHVGLREAALRRLQEEARDLLERLVQRKARAAAERNLRNERRERAKQARVSQELK
KAAKRTVSISEGPDTLGDGMRERRETLALAPEPEPLEKEACEKWKRPFRSASATSLTLS
HCVDVVKGLLDFKKRRGHSIGGAPEQRYQIIPVCVAARLPTRAQDVLDAHLSEVNAV
RFGPNSSLLATGGADRLIHLWNVVGSRLEANQTLEGAGGSITSVDFDPSGYQVLAATY
NQAAQLWKVGEAQSKETLSGHKDKVTAAKFKLTRHQAVTGSRDRTVKEWDLGRAY
CSRTINVLSYCNDVVCGDHIIISGHNDQKIRFWDSRGPHCTQVIPVQGRVTSLSLSHDQ
LHLLSCSRDNTLKVIDLRVSNIRQVFRADGFKCGSDWTKAVFSPDRSYALAGSCDGAL
YIWDVDTGKLESRLQGPHCAAVNAVAWCYSGSHMVSVDQGRKVVLWQ
ADCK5 cDNA;
(NM_174922.5; SEQ ID NO: 15)
GAGACGCTAAGCGGCGCCGGGCGGGAGAAGAGCGGAGCAGTGGTCGGAGATGTG
GCGACCGGTGCAGCTCTGTCATTTCCACTCTGCTCTGCTGCACAGCAGGCAGAAGC
CCTGGCCGTCCCCTGCTGTGTTCTTCAGGAGAAACGTCAGGGGCCTTCCTCCAAGG
TTCTCCAGCCCCACACCCCTGTGGAGGAAGGTGCTCTCCACCGCGGTAGTGGGGGC
GCCCCTGCTCCTCGGAGCCCGCTATGTCATGGCAGAGGCACGGGAGAAGAGGAGG
ATGCGGCTCGTGGTGGATGGCATGGGGCGCTTTGGCAGGTCTCTGAAGGTCGGCCT
GCAGATCTCCCTGGACTACTGGTGGTGCACCAATGTTGTCCTTCGAGGGGTGGAAG
AGAACAGCCCAGGCTACTTGGAGGTGATGTCTGCGTGTCACCAGCGGGCGGCTGA
TGCCCTGGTGGCAGGGGCCATCAGCAACGGGGGCCTCTACGTGAAGCTGGGCCAG
GGGCTGTGCTCCTTCAACCACCTGCTTCCCCCCGAGTATACCCGGACCCTGCGCGT
GCTAGAGGACAGGGCCCTCAAGCGGGGCTTCCAGGAGGTGGATGAGTTGTTCCTT
GAGGACTTCCAGGCCCTCCCCCACGAGCTCTTCCAGGAGTTTGACTACCAGCCAAT
TGCTGCCGCCAGCCTGGCACAGGTGCACAGAGCCAAGCTGCACGATGGCACCAGC
GTGGCTGTGAAGGTGCAGTACATCGACCTGCGGGACCGCTTTGATGGGGACATCC
ACACCCTGGAGCTCCTGCTGCGGCTCGTTGAGGTCATGCACCCCAGCTTTGGCTTC
AGCTGGGTCCTCCAGGACCTGAAGGGGACCCTGGCCCAGGAGCTGGACTTCGAGA
ATGAGGGCCGCAACGCAGAGCGCTGTGCGCGGGAGCTGGCGCACTTCCCCTACGT
CGTGGTGCCCCGCGTGCACTGGGACAAGTCCAGCAAGCGCGTGCTCACTGCCGAC
TTCTGCGCCGGCTGCAAGGTCAACGATGTGGAGGCCATCAGGAGCCAGGGGCTGG
CAGTGCATGACATAGCAGAAAAGCTCATCAAGGCCTTTGCTGAGCAGATATTTTAC
ACCGGCTTCATCCACTCGGACCCACATCCTGGCAACGTTCTGGTGCGGAAAGGCCC
GGACGGGAAAGCGGAGCTGGTGCTGCTGGACCACGGGCTCTACCAGTTCCTGGAG
GAGAAGGACCGCGCAGCCCTCTGCCAGCTGTGGCGGGCCATCATCCTGCGGGACG
ACGCCGCCATGAGGGCGCACGCAGCCGCACTGGGGGTGCAAGACTACCTCCTGTT
CGCCGAGATGCTCATGCAGCGCCCCGTGCGCCTGGGGCAGCTGTGGGGCTCGCAC
CTACTGAGCCGCGAAGAGGCGGCCTACATGGTGGACATGGCCCGCGAGCGCTTCG
AGGCCGTCATGGCGGTGCTCAGGGAGCTGCCGCGGCCCATGCTGCTGGTGCTGCG
CAACATCAACACCGTGCGCGCTATCAACGTGGCCCTCGGCGCCCCCGTGGACCGCT
ACTTCCTTATGGCTAAAAGGGCTGTCCGGGGCTGGAGCCGCCTGGCGGGCGCCAC
GTATCGGGGTGTCTACGGCACCAGCCTCCTGCGCCACGCCAAGGTCGTCTGGGAG
ATGCTCAAGTTTGAAGTGGCGCTCAGGCTGGAGACCTTGGCCATGCGGCTGACCGC
CCTCCTGGCTCGTGCTCTGGTCCACCTGAGCCTCGTGCCCCCAGCGGAGGAGCTCT
ACCAGTACCTGGAGACCTAGGGTGCAGCCGCCCAGGGCCGGCGGGGCCCTTTTCA
CCTTGGGCTGACGGAGGTGGCGGGGCTAGAGGTGTAGACACCCCGAGCCCCGTGG
GCACTCGCACTGGGGGGCTGTGACAGCAGCTGGGCCAGGAGGCCGTGTAATGACC
ACACACTCCTCTCAAGCAAAAAA
ADCK5 Protein;
(NP_777582.4; SEQ ID NO: 16)
MWRPVQLCHFHSALLHSRQKPWPSPAVFFRRNVRGLPPRFSSPTPLWRKVLSTAVVG
APLLLGARYVMAEAREKRRMRLVVDGMGRFGRSLKVGLQISLDYWWCTNVVLRGV
EENSPGYLEVMSACHQRAADALVAGAISNGGLYVKLGQGLCSFNHLLPPEYTRTLRV
LEDRALKRGFQEVDELFLEDFQALPHELFQEFDYQPIAAASLAQVHRAKLHDGTSVAV
KVQYIDLRDRFDGDIHTLELLLRLVEVMHPSFGFSWVLQDLKGTLAQELDFENEGRNA
ERCARELAHFPYVVVPRVHWDKSSKRVLTADFCAGCKVNDVEAIRSQGLAVHDIAEK
LIKAFAEQIFYTGFIHSDPHPGNVLVRKGPDGKAELVLLDHGLYQFLEEKDRAALCQL
WRAIILRDDAAMRAHAAALGVQDYLLFAEMLMQRPVRLGQLWGSHLLSREEAAYM
VDMARERFEAVMAVLRELPRPMLLVLRNINTVRAINVALGAPVDRYFLMAKRAVRG
WSRLAGATYRGVYGTSLLRHAKVVWEMLKFEVALRLETLAMRLTALLARALVHLSL
VPPAEELYQYLET
ADCY5 cDNA;
(NM_183357.2; SEQ ID NO: 17)
ATGTCCGGCTCCAAAAGCGTGAGCCCCCCGGGCTACGCGGCGCAGAAGACTGCGG
CGCCGGCGCCCCGGGGAGGCCCCGAACACCGCTCTGCGTGGGGCGAGGCCGATTC
CCGCGCGAATGGCTACCCCCATGCCCCCGGGGGCTCTGCCCGCGGCTCCACCAAG
AAACCCGGGGGGGCGGTGACCCCGCAGCAGCAGCAGCGCCTGGCCAGCCGCTGGC
GCAGCGACGACGACGACGATCCTCCGCTGAGCGGTGACGACCCCCTGGCCGGGGG
CTTCGGCTTCAGCTTCCGCTCCAAGTCCGCCTGGCAGGAGCGCGGCGGCGACGACT
GCGGTCGCGGCAGCCGCCGGCAGCGGCGGGGCGCGGCCAGCGGGGGCAGCACCC
GGGCGCCCCCTGCGGGCGGCGGCGGCGGCTCGGCGGCGGCGGCTGCCTCGGCGGG
CGGGACGGAGGTGCGCCCTCGCTCGGTGGAGGTGGGTCTGGAGGAGCGGCGGGGC
AAGGGGCGCGCGGCCGACGAGCTGGAGGCCGGCGCCGTCGAGGGCGGCGAGGGG
TCCGGGGATGGCGGCAGCTCGGCGGACTCGGGCTCGGGCGCGGGGCCCGGCGCGG
TGCTGTCCCTGGGCGCCTGCTGCCTGGCGTTGCTGCAGATATTCCGCTCCAAGAAG
TTCCCGTCGGACAAACTGGAGCGGCTGTACCAGCGCTACTTCTTCCGCCTGAACCA
GAGCAGCCTCACCATGCTCATGGCCGTGCTGGTGCTCGTGTGCCTGGTCATGTTGG
CCTTCCACGCGGCGCGGCCCCCGCTCCAGCTGCCCTACCTGGCCGTGCTGGCGGCC
GCCGTCGGCGTGATCCTCATCATGGCTGTGCTTTGCAACCGCGCCGCCTTCCACCA
GGACCACATGGGCCTGGCCTGCTATGCGCTCATCGCCGTGGTGCTGGCCGTCCAGG
TGGTGGGCCTGCTGCTGCCGCAGCCACGCAGCGCCTCTGAGGGCATCTGGTGGACC
GTGTTCTTCATCTACACCATCTACACGCTGCTGCCCGTGCGCATGCGGGCCGCAGT
GCTCAGCGGGGTGCTCCTGTCCGCCCTCCACCTGGCCATCGCCCTGCGCACCAACG
CCCAGGACCAGTTCCTGCTGAAGCAGCTTGTCTCCAATGTTCTCATTTTCTCCTGCA
CCAACATCGTGGGTGTCTGCACCCACTATCCGGCTGAGGTCTCCCAGAGACAGGCT
TTCCAGGAGACCCGAGAGTGCATCCAGGCGCGGCTCCACTCGCAGCGGGAGAACC
AGCAGCAGGAACGGCTCCTGCTGTCTGTCCTTCCCCGTCATGTTGCCATGGAGATG
AAAGCAGACATCAACGCCAAGCAGGAGGATATGATGTTCCATAAGATTTACATCC
AGAAACATGACAACGTGAGCATCCTGTTTGCTGACATCGAGGGCTTCACCAGCCTG
GCGTCCCAGTGCACTGCACAGGAACTGGTCATGACCCTCAACGAGCTCTTCGCCCG
CTTTGACAAGCTGGCCGCAGAGAATCACTGTTTACGTATTAAGATCCTTGGGGATT
GTTATTACTGCGTCTCGGGGCTGCCTGAAGCAAGGGCTGACCACGCCCACTGCTGT
GTGGAGATGGGCATGGACATGATCGAGGCCATCTCGTTGGTCCGGGAGGTGACAG
GGGTGAACGTGAACATGCGTGTGGGAATTCACAGCGGGCGAGTACACTGCGGTGT
CCTTGGTCTCAGGAAGTGGCAGTTCGACGTCTGGTCTAACGATGTCACGCTAGCCA
ACCACATGGAGGCTGGCGGCAAGGCAGGACGCATCCACATCACCAAGGCTACACT
CAACTACCTGAATGGGGACTACGAGGTGGAGCCAGGCTGTGGGGGCGAGCGCAAC
GCCTACCTCAAGGAGCACAGTATCGAGACCTTCCTCATCCTGCGCTGCACCCAGAA
GCGGAAAGAAGAGAAGGCCATGATCGCCAAGATGAACCGCCAGAGAACCAACTC
CATCGGGCACAACCCACCACACTGGGGGGCTGAGCGCCCCTTCTACAACCACCTG
GGTGGCAACCAGGTGTCCAAGGAGATGAAGCGGATGGGCTTTGAAGACCCCAAGG
ACAAGAACGCCCAGGAGAGTGCGAACCCTGAGGATGAAGTGGATGAGTTTCTGGG
CCGTGCCATTGACGCCAGGAGCATTGATAGGCTTCGGTCTGAGCACGTCCGCAAGT
TCCTCCTGACCTTCAGGGAGCCTGACTTAGAGAAGAAGTACTCCAAGCAGGTAGA
CGACCGATTTGGTGCCTATGTGGCGTGTGCCTCGCTCGTCTTCCTCTTCATCTGCTT
TGTCCAGATCACCATCGTGCCCCACTCCATATTCATGCTCAGCTTCTACCTGACCTG
TTCCCTGCTGCTGACCTTGGTGGTGTTTGTGTCTGTGATCTACTCCTGCGTAAAGCT
CTTCCCCTCCCCACTGCAGACCCTCTCCAGGAAGATCGTGCGGTCCAAGATGAACA
GCACCCTGGTTGGGGTGTTCACCATCACCCTGGTGTTCCTGGCGGCTTTTGTCAAC
ATGTTCACGTGCAACTCCAGGGACCTGCTGGGCTGCTTGGCACAGGAGCACAACA
TCAGCGCGAGCCAGGTCAACGCGTGTCACGTGGCGGAGTCGGCCGTCAACTACAG
CCTGGGCGATGAGCAGGGCTTCTGTGGCAGCCCCTGGCCCAACTGCAACTTCCCCG
AGTACTTCACCTACAGCGTGCTGCTCAGCCTGCTGGCCTGCTCCGTGTTCCTGCAG
ATCAGCTGCATCGGGAAGCTGGTGCTCATGCTGGCCATCGAGCTCATCTACGTGCT
CATCGTGGAGGTGCCAGGTGTCACGCTCTTCGACAACGCCGACCTGCTGGTCACCG
CCAACGCCATAGACTTCTTCAACAACGGGACCTCCCAGTGCCCTGAGCATGCAACC
AAGGTGGCATTGAAGGTGGTGACGCCCATCATCATCTCAGTCTTTGTGCTGGCCCT
GTACCTGCACGCCCAGCAGGTGGAGTCCACTGCCCGCCTCGACTTCCTCTGGAAAC
TGCAGGCCACAGAGGAGAAAGAGGAGATGGAGGAGCTGCAGGCCTACAACCGGC
GGCTGCTGCACAACATCCTGCCCAAGGACGTGGCCGCTCACTTCCTGGCCCGCGAG
CGGCGCAATGATGAGCTCTACTATCAGTCCTGTGAGTGTGTGGCGGTCATGTTCGC
CTCCATCGCCAACTTCTCCGAGTTCTACGTTGAGCTGGAGGCCAACAACGAGGGTG
TCGAGTGCCTGCGGCTACTCAATGAGATCATCGCTGACTTTGATGAGATCATCAGC
GAGGATCGGTTCCGGCAGCTGGAGAAGATCAAGACCATCGGCAGCACCTACATGG
CTGCCTCCGGCCTCAACGACTCTACCTACGACAAGGTGGGCAAGACCCACATCAA
GGCACTGGCCGACTTTGCCATGAAGCTGATGGACCAGATGAAGTACATCAATGAG
CACTCCTTCAACAACTTCCAGATGAAGATCGGGCTCAACATCGGCCCCGTGGTGGC
CGGGGTGATAGGGGCACGAAAGCCTCAGTACGACATCTGGGGCAATACCGTGAAC
GTGGCCAGCCGCATGGACAGCACCGGTGTACCCGACCGCATCCAGGTCACCACAG
ACATGTACCAGGTGCTGGCTGCCAACACGTACCAGCTGGAGTGCCGGGGCGTGGT
CAAGGTCAAGGGCAAAGGCGAGATGATGACCTACTTCCTCAATGGAGGGCCCCCG
CTCAGTTAGCAGCTGTTGGCCAATGGTGCCAGGCAGCCTGGCCTCCAGAGGCATG
GAAGCAGCTTCTCTGTGTGCCGGGGGTGGCGGGGAAGCCATGCTCCAGCCCGCAG
GGCTGCGCTGCTGAGATTTTCCACTTGGACTCCAGAGCAGCTTCTGCCTTTGCTGGT
GGGCAGCGGCCTCTGTCCCAGGCCCCGGGGTGCCAGCGTCCTGCGAGCACCCAGC
TGACCAAAGATGTTTCCCTCTGTAGAAGACTCTGCTAGACTGGGTCTGAAGCTTGA
GTTTTCTAACAGGTGCTGCTGCACAGGTGGAAAGGAGCCGTGGGAATGTGTGTGT
GGCACGGCCCAGACAAGGGCAGGGCTGAGGGGCCTCCGACTCAGCTGGGGGTAG
ACGGGCTCGAATGTGGCCTGGGAGAGCCTAGGGGGCCCCAGGGGTCTGCTTTTCT
ATGTGAGCCTTTAAACTTCAGACAGGCCACCACCCTGCACCTGCAGGGGCTTTGGC
ACAGGAGTGCTGGCTTTGGAGGGACTGTGGCCTTCATCGTGGTCCTCTGCCCACAC
CTCCACGCACACAGACAGTGCCCTAGGAGGGAAACAGAACTAATTACGAGGGGGA
GGCAAGAGGACGCCAAGCAAGGAGTGGTGATTCTGAGAAAAATATTTATTAAATA
AAACAAAACAAGTTCTCCGTGCCCTTCTTTAGACTATGCTAGTTGTATGCGTGTAA
GAGACACACAAGCAAACGAGGACGCCACTCGGGGGGAGGGCGGGGATCCCCACT
TGTCTTTTTTGTATTTTTTATTTTGTATTATTGAAAGCCTTGGAGATCTCACAGATAG
ATATGCCAAATTCTATATTTTGTAAATTCTCTATATTAGAAAACAGCTGTGCACAG
CAGGGCGGGTGTGCTCATTTGTACTGTGTGTATGTCGGTGTATGTACTGGTGTATAT
GTGTGTGTGTTCATGCTGTGGAACTGGTCTCACACAGGATGTGTTTCCCTCATTTCA
GATTTGGCAGTTTTGGGTTTTCCAAGGTACCACCAGAGCAGTGGGTGTGTGCTTTT
GGGGTACCATATGCTCAGATTAAGTAGGAGGATGCATGGACACACTGCCCCATCTT
TTCTGACACACGCACACGTATGTACACACATGCACACACCCTCCTTCCCCTAAGCA
AAACGCAGATGGAATAAGAAAACAAAAAGCTGCTTTCCCATCCCAGGCCGAGCTG
GAACCAAGGGAAGCAATCTCATCCTGCGACAGGCAGTGTGGTGCCCTCCACACCC
TGAGATTTCAGACGTTTGCGGCTTACAGAGGCAGCGCCCACAGATTCCAGAGTGCT
TACAGAAGGCCAGGTGCTTTGCAGGCTGGGACGAGGAAGCCAAGCCTCCCTGGCC
TACTCAGTTGGCCAAGGTGCAGGTGGCTCTTCCTGGAGATGTTCACTCAGACTGGG
GGATGCAATGTGCAGCCTTCAGGTTTGCGGAAAGGGAGTGGCCTTGACCTCCACC
GGCAAACCAGGCAGAGGAATGGGTAGAGCCCAGCTTTAGAGTCCACAGGGAAAG
CTAGCAGGAATTTTGTTTTAGTGGGAGGGGGCAGTTAAACATACCAAGAAAAAAA
TACTATTTTTATAACCTATGAGGAAGACATTTGGAAAATGATACTCTAGCACAGAA
TTCAGTGGAATCCTTAGGGCCCATGCCCAAATCTTTCCATTGCTTCTCAGGTTAGA
ATGATCTTCACCTCCAACATGAGCTTGGAGGTGATGAGGCAGTGGCTCTGTGCCAG
CTGCCACAATGTGACTTTGATGTCCACCTGTACCACCTCTCACTGGGCTCTAGCAC
CACCCTCCCCTCCCCGCACACCAACTGAACACAGCTCTGAGAAGCAAAGTGTGTG
GACCCAAAACTGCCAAGCCTGAGTCTGTCCCGTGCTTCTGCTGCTCCATCCTTTGA
GTTCTGCATTGCCATCCTGACGTCGGCCACAGGAGGCCTGCTTTCTTCCAGCTGTTG
TTCTCAAGTTCCCTGCCCCTCCACATCGCCCGCCAGTGGTGTTGGGTTTTCGTTCTG
CTTCCAACCTGAGTAAAGTGTGTGTGCTGAGTTCATCCCATGTTCTCCCATGGTCAT
GGCTTCCCGGCCCCATGGGGACCCCTCTCCCATCCCAGCAGTGACTGGTGACAGTG
TGCAGGTGCAGTGCTAGCTCTTCGTTCCCTCTAAAGGGTGTGCACTCTTTTTATTCC
TACTCTTGCAAAAACAGATACGATTATGATTTCCCATGGAAATTGAAAAGTCTATT
TAAATAATTTAACTATTAAACACTTTCACTGGTAA
ADCY5 Protein;
(NP_899200.1; SEQ ID NO: 18)
MSGSKSVSPPGYAAQKTAAPAPRGGPEHRSAWGEADSRANGYPHAPGGSARGSTKKP
GGAVTPQQQQRLASRWRSDDDDDPPLSGDDPLAGGFGFSFRSKSAWQERGGDDCGR
GSRRQRRGAASGGSTRAPPAGGGGGSAAAAASAGGTEVRPRSVEVGLEERRGKGRA
ADELEAGAVEGGEGSGDGGSSADSGSGAGPGAVLSLGACCLALLQIFRSKKFPSDKLE
RLYQRYFFRLNQSSLTMLMAVLVLVCLVMLAFHAARPPLQLPYLAVLAAAVGVILIM
AVLCNRAAFHQDHMGLACYALIAVVLAVQVVGLLLPQPRSASEGIWWTVFFIYTIYTL
LPVRMRAAVLSGVLLSALHLAIALRTNAQDQFLLKQLVSNVLIFSCTNIVGVCTHYPA
EVSQRQAFQETRECIQARLHSQRENQQQERLLLSVLPRHVAMEMKADINAKQEDMMF
HKIYIQKHDNVSILFADIEGFTSLASQCTAQELVMTLNELFARFDKLAAENHCLRIKILG
DCYYCVSGLPEARADHAHCCVEMGMDMIEAISLVREVTGVNVNMRVGIHSGRVHCG
VLGLRKWQFDVWSNDVTLANHMEAGGKAGRIHITKATLNYLNGDYEVEPGCGGERN
AYLKEHSIETFLILRCTQKRKEEKAMIAKMNRQRTNSIGHNPPHWGAERPFYNHLGGN
QVSKEMKRMGFEDPKDKNAQESANPEDEVDEFLGRAIDARSIDRLRSEHVRKFLLTFR
EPDLEKKYSKQVDDRFGAYVACASLVFLFICFVQITIVPHSIFMLSFYLTCSLLLTLVVF
VSVIYSCVKLFPSPLQTLSRKIVRSKMNSTLVGVFTITLVFLAAFVNMFTCNSRDLLGCL
AQEHNISASQVNACHVAESAVNYSLGDEQGFCGSPWPNCNFPEYFTYSVLLSLLACSV
FLQISCIGKLVLMLAIELIYVLIVEVPGVTLFDNADLLVTANAIDFFNNGTSQCPEHATK
VALKVVTPIIISVFVLALYLHAQQVESTARLDFLWKLQATEEKEEMEELQAYNRRLLH
NILPKDVAAHFLARERRNDELYYQSCECVAVMFASIANFSEFYVELEANNEGVECLRL
LNEIIADFDEIISEDRFRQLEKIKTIGSTYMAASGLNDSTYDKVGKTHIKALADFAMKL
MDQMKYINEHSFNNFQMKIGLNIGPVVAGVIGARKPQYDIWGNTVNVASRMDSTGVP
DRIQVTTDMYQVLAANTYQLECRGVVKVKGKGEMMTYFLNGGPPLS
CPSF1 cDNA;
(NM_013291.3; SEQ ID NO: 19)
GAGTTCGCTGCTGTCCCGGTTCCTCTCGAGTCGGCTCCAACTGCCAGCCCGGGTTG
GCGCCATGTACGCCGTGTACAAACAGGCGCATCCGCCCACCGGTCTGGAGTTCTCC
ATGTACTGCAACTTCTTCAACAACAGCGAGCGCAACCTGGTAGTGGCCGGGACCTC
GCAGCTCTACGTGTACCGCCTCAACCGCGACGCCGAGGCTCTGACCAAGAATGAC
AGGAGCACAGAGGGGAAGGCCCACCGGGAGAAGCTCGAGCTTGCTGCCTCCTTCT
CCTTCTTTGGCAACGTCATGTCCATGGCCAGCGTGCAGCTGGCAGGAGCCAAGCGG
GATGCCCTGCTCCTAAGCTTCAAGGATGCCAAGCTGTCTGTGGTGGAGTACGACCC
GGGCACCCATGACCTGAAGACCCTGTCACTGCACTACTTTGAGGAGCCTGAGCTTC
GGGACGGGTTTGTGCAGAATGTACACACGCCGCGAGTGCGGGTGGACCCCGACGG
GCGCTGTGCAGCCATGCTTGTCTACGGCACGCGGCTGGTGGTCCTGCCCTTCCGCA
GGGAGAGCCTGGCTGAGGAGCACGAGGGGCTCGTGGGTGAGGGGCAGAGGTCCA
GCTTCCTGCCCAGCTACATCATCGACGTGCGGGCCCTAGACGAGAAGCTGCTCAAC
ATCATCGACCTGCAGTTCCTGCATGGCTACTACGAGCCTACCCTCCTCATCCTGTTT
GAGCCCAACCAGACCTGGCCTGGGCGCGTGGCCGTGCGGCAGGACACGTGCTCCA
TTGTGGCCATCTCACTGAACATCACGCAGAAGGTGCACCCCGTCATCTGGTCCCTC
ACCAGCCTGCCCTTTGACTGCACCCAGGCTCTGGCTGTGCCCAAGCCCATAGGTGG
GGTGGTGGTGTTTGCCGTCAACTCGCTGTTGTACCTGAACCAGAGCGTCCCCCCGT
ATGGCGTGGCTCTCAACAGCCTCACCACAGGAACCACGGCTTTCCCGCTTCGCACC
CAGGAGGGTGTGCGGATCACCCTGGACTGCGCCCAGGCCACCTTCATCTCCTACGA
CAAGATGGTCATCTCCCTCAAGGGCGGCGAGATCTACGTGCTGACCCTCATCACCG
ACGGCATGCGCAGTGTCCGAGCGTTCCACTTTGACAAGGCGGCCGCCAGCGTCCTC
ACCACCAGCATGGTCACCATGGAGCCCGGGTACCTGTTCCTGGGTTCTCGCCTGGG
CAATTCCCTCCTCCTCAAGTACACGGAGAAGCTGCAGGAGCCCCCGGCCAGTGCTG
TCCGTGAGGCTGCCGACAAGGAAGAGCCTCCCTCAAAGAAGAAGCGAGTGGATGC
GACGGCCGGCTGGTCAGCTGCGGGTAAGTCGGTGCCGCAGGATGAGGTGGACGAG
ATTGAAGTGTACGGCAGCGAGGCCCAGTCGGGAACACAGCTGGCCACCTACTCCT
TTGAGGTGTGTGACAGCATCCTGAACATTGGACCCTGTGCCAATGCCGCCGTGGGC
GAGCCTGCCTTCCTCTCTGAAGAGTTTCAGAACAGCCCCGAGCCGGACCTGGAGAT
TGTGGTTTGCTCCGGCCACGGGAAGAACGGGGCTTTGTCGGTGCTGCAGAAGAGC
ATCCGGCCCCAGGTGGTGACAACCTTTGAGCTTCCCGGCTGCTATGACATGTGGAC
AGTCATCGCCCCGGTGCGTAAGGAGGAGGAGGACAATCCCAAGGGGGAGGGCAC
AGAGCAGGAACCCAGCACCACCCCTGAAGCAGACGACGACGGCCGCAGACACGG
ATTCCTGATTCTGAGCCGGGAAGACTCCACCATGATCCTGCAGACGGGGCAGGAG
ATCATGGAGCTGGACACCAGTGGCTTCGCCACTCAGGGCCCCACGGTCTTTGCTGG
GAACATCGGGGACAACCGCTACATTGTCCAAGTGTCACCACTGGGCATCCGCCTGC
TGGAAGGAGTGAATCAGCTGCACTTCATCCCCGTGGACCTGGGCGCCCCCATCGTG
CAGTGCGCCGTGGCCGACCCCTATGTGGTCATCATGAGTGCCGAGGGCCACGTCAC
CATGTTCCTGCTGAAGAGTGACTCCTACGGTGGCCGCCACCACCGCCTGGCGCTGC
ACAAGCCCCCGCTGCACCATCAGTCCAAGGTGATTACGCTGTGCCTGTACCGAGAC
CTCAGCGGCATGTTCACCACTGAGAGCCGCCTGGGTGGGGCCCGTGACGAGCTCG
GGGGCCGCAGTGGCCCGGAGGCCGAGGGCCTGGGCTCAGAGACTAGCCCCACAGT
GGATGACGAGGAGGAGATGCTGTATGGGGATTCGGGCTCCCTCTTCAGCCCCAGC
AAGGAGGAGGCCCGAAGAAGCAGCCAGCCCCCTGCTGACCGGGACCCTGCACCCT
TCCGGGCAGAGCCTACCCACTGGTGCCTGCTGGTGCGGGAGAATGGCACCATGGA
GATCTACCAGCTTCCCGACTGGCGGCTGGTGTTCCTGGTGAAGAACTTCCCTGTGG
GGCAGCGGGTCCTTGTGGACAGCTCCTTTGGACAGCCCACTACACAGGGCGAGGC
CCGCAGGGAGGAGGCCACGCGCCAGGGGGAGCTGCCCCTCGTCAAGGAGGTGCTG
CTGGTGGCGCTGGGCAGCCGCCAGAGCAGGCCCTACCTGCTGGTGCATGTGGACC
AAGAGCTGCTTATCTACGAGGCCTTCCCCCACGACTCTCAGCTCGGCCAGGGCAAT
CTCAAAGTCCGCTTTAAGAAGGTCCCTCACAACATCAACTTCCGTGAGAAGAAGCC
AAAGCCATCCAAGAAGAAAGCAGAAGGTGGCGGCGCAGAGGAGGGGGCTGGGGC
CCGGGGCCGCGTGGCGCGTTTCCGCTACTTCGAGGATATTTATGGCTACTCAGGGG
TCTTCATCTGCGGCCCCTCCCCTCACTGGCTCTTGGTGACCGGCCGAGGGGCTCTG
CGGCTACACCCCATGGCCATCGACGGCCCGGTCGACTCTTTCGCTCCATTCCACAA
TGTCAACTGTCCCCGCGGCTTCCTGTACTTCAACAGACAGGGCGAGCTGAGGATCA
GTGTCCTGCCTGCCTACCTGTCCTATGATGCCCCATGGCCTGTCAGGAAGATCCCG
CTGCGCTGCACGGCCCACTATGTGGCTTACCACGTGGAGTCTAAGGTGTATGCTGT
GGCCACCAGCACCAACACGCCGTGTGCCCGCATCCCACGCATGACTGGCGAGGAG
AAGGAGTTTGAGACCATCGAGAGAGATGAGCGGTACATCCACCCCCAGCAGGAGG
CCTTCTCCATCCAGCTCATCTCCCCGGTCAGCTGGGAGGCTATTCCCAATGCCAGG
ATCGAGCTGCAGGAGTGGGAGCATGTGACCTGCATGAAGACAGTGTCTCTGCGCA
GTGAGGAGACCGTGTCGGGCCTCAAAGGCTACGTGGCCGCCGGGACCTGCCTCAT
GCAGGGGGAGGAGGTCACGTGCCGAGGGCGGATCTTGATCATGGATGTGATTGAG
GTGGTGCCCGAGCCTGGCCAGCCCTTGACCAAGAACAAGTTCAAAGTCCTTTACGA
GAAGGAGCAGAAGGGGCCCGTGACCGCCCTGTGCCACTGCAATGGCCACCTGGTG
TCGGCCATCGGCCAGAAGATTTTCCTGTGGAGCCTGCGGGCCAGCGAGCTGACGG
GCATGGCCTTCATCGACACGCAGCTCTACATACACCAGATGATCAGCGTCAAGAA
CTTCATCCTGGCAGCCGACGTCATGAAGAGCATTTCGCTGCTGCGCTACCAGGAGG
AAAGCAAGACGCTGAGCCTGGTGTCGCGGGATGCCAAGCCCCTGGAGGTGTACAG
CGTGGACTTCATGGTGGACAATGCCCAGCTGGGTTTTCTGGTGTCTGACCGCGACC
GCAACCTCATGGTGTACATGTACCTGCCCGAAGCCAAGGAGAGTTTCGGGGGCAT
GCGCCTGCTGCGTCGGGCAGACTTCCACGTGGGTGCCCACGTGAACACGTTCTGGA
GGACCCCGTGCCGGGGGGCCACTGAAGGGCTCAGCAAAAAGTCGGTCGTGTGGGA
GAATAAGCACATCACGTGGTTTGCCACCCTGGACGGCGGCATCGGGCTGCTGCTGC
CCATGCAGGAGAAGACCTACCGGCGGCTGCTGATGCTGCAGAACGCGCTGACCAC
CATGCTGCCACACCACGCCGGCCTCAACCCCCGCGCCTTCCGGATGCTGCACGTGG
ACCGCCGCACCCTCCAGAATGCCGTGCGCAACGTGCTGGATGGGGAGCTGCTCAA
CCGCTACCTGTACCTGAGCACCATGGAGCGCAGCGAGCTAGCCAAGAAGATCGGC
ACCACACCAGACATAATCCTGGACGACTTGCTGGAGACGGACCGCGTCACCGCCC
ACTTCTAGCCCCGTGGATGCCGTCACCACCAGCACACGGAACTACCTCCCACCCCC
TTTTTGTACAAAACACAAGGAAAAACATTTTTTGCTTGA
CPSF1 Protein;
(NP_037423.2; SEQ ID NO: 20)
MYAVYKQAHPPTGLEFSMYCNFFNNSERNLVVAGTSQLYVYRLNRDAEALTKNDRS
TEGKAHREKLELAASFSFFGNVMSMASVQLAGAKRDALLLSFKDAKLSVVEYDPGTH
DLKTLSLHYFEEPELRDGFVQNVHTPRVRVDPDGRCAAMLVYGTRLVVLPFRRESLA
EEHEGLVGEGQRSSFLPSYIIDVRALDEKLLNIIDLQFLHGYYEPTLLILFEPNQTWPGR
VAVRQDTCSIVAISLNITQKVHPVIWSLTSLPFDCTQALAVPKPIGGVVVFAVNSLLYL
NQSVPPYGVALNSLTTGTTAFPLRTQEGVRITLDCAQATFISYDKMVISLKGGEIYVLT
LITDGMRSVRAFHFDKAAASVLTTSMVTMEPGYLFLGSRLGNSLLLKYTEKLQEPPAS
AVREAADKEEPPSKKKRVDATAGWSAAGKSVPQDEVDEIEVYGSEAQSGTQLATYSF
EVCDSILNIGPCANAAVGEPAFLSEEFQNSPEPDLEIVVCSGHGKNGALSVLQKSIRPQV
VTTFELPGCYDMWTVIAPVRKEEEDNPKGEGTEQEPSTTPEADDDGRRHGFLILSREDS
TMILQTGQEIMELDTSGFATQGPTVFAGNIGDNRYIVQVSPLGIRLLEGVNQLHFIPVDL
GAPIVQCAVADPYVVIMSAEGHVTMFLLKSDSYGGRHHRLALHKPPLHHQSKVITLCL
YRDLSGMFTTESRLGGARDELGGRSGPEAEGLGSETSPTVDDEEEMLYGDSGSLFSPS
KEEARRSSQPPADRDPAPFRAEPTHWCLLVRENGTMEIYQLPDWRLVFLVKNFPVGQ
RVLVDSSFGQPTTQGEARREEATRQGELPLVKEVLLVALGSRQSRPYLLVHVDQELLI
YEAFPHDSQLGQGNLKVRFKKVPHNINFREKKPKPSKKKAEGGGAEEGAGARGRVAR
FRYFEDIYGYSGVFICGPSPHWLLVTGRGALRLHPMAIDGPVDSFAPFHNVNCPRGFLY
FNRQGELRISVLPAYLSYDAPWPVRKIPLRCTAHYVAYHVESKVYAVATSTNTPCARI
PRMTGEEKEFETIERDERYIHPQQEAFSIQLISPVSWEAIPNARIELQEWEHVTCMKTVS
LRSEETVSGLKGYVAAGTCLMQGEEVTCRGRILIMDVIEVVPEPGQPLTKNKFKVLYE
KEQKGPVTALCHCNGHLVSAIGQKIFLWSLRASELTGMAFIDTQLYIHQMISVKNFILA
ADVMKSISLLRYQEESKTLSLVSRDAKPLEVYSVDFMVDNAQLGFLVSDRDRNLMVY
MYLPEAKESFGGMRLLRRADFHVGAHVNTFWRTPCRGATEGLSKKSVVWENKHITW
FATLDGGIGLLLPMQEKTYRRLLMLQNALTTMLPHHAGLNPRAFRMLHVDRRTLQNA
VRNVLDGELLNRYLYLSTMERSELAKKIGTTPDIILDDLLETDRVTAHF
PMPCA cDNA;
(NM_015160.3; SEQ ID NO: 21)
GGAGACGCAAGATGGCGGCTGTGGTGCTGGCGGCGACGCGGTTGCTGCGGGGCTC
GGGTTCTTGGGGCTGTTCGCGGCTGAGGTTTGGACCTCCTGCGTACAGACGGTTTA
GTAGTGGTGGTGCCTATCCCAACATCCCCCTCTCTTCTCCCTTACCTGGAGTACCCA
AGCCTGTTTTTGCTACAGTTGATGGACAGGAAAAGTTTGAAACCAAAGTAACCAC
ATTGGATAATGGGCTTCGCGTGGCATCTCAGAATAAGTTTGGACAGTTTTGTACAG
TAGGAATTCTTATCAATTCAGGATCGAGATATGAAGCGAAATACCTTAGTGGAATT
GCTCACTTTTTGGAAAAATTGGCATTTTCGTCTACTGCTCGATTTGACAGCAAAGA
TGAAATTCTGCTTACGTTGGAAAAGCATGGGGGTATCTGTGACTGCCAGACATCAA
GAGACACCACCATGTATGCTGTGTCTGCTGATAGCAAAGGCTTGGACACGGTGGTT
GCCTTACTGGCTGATGTGGTTCTGCAGCCCCGGCTAACAGATGAAGAAGTCGAGAT
GACGCGGATGGCGGTCCAGTTTGAGCTGGAGGACCTGAACCTGCGGCCTGACCCA
GAGCCACTTCTCACCGAGATGATTCATGAAGCGGCTTACAGGGAGAACACAGTTG
GCCTCCACCGTTTCTGCCCCACAGAAAACGTAGCAAAGATCAACCGAGAGGTGCT
GCATTCCTACCTGAGGAACTACTACACTCCCGACCGCATGGTGCTGGCCGGCGTGG
GCGTGGAGCACGAGCATCTGGTGGACTGTGCCCGGAAGTACCTCCTGGGGGTCCA
GCCGGCCTGGGGGAGCGCAGAGGCCGTGGATATTGACAGATCTGTGGCCCAGTAC
ACTGGGGGGATTGCCAAGCTAGAAAGAGACATGTCCAATGTCAGCCTGGGCCCGA
CCCCCATCCCCGAGCTCACGCACATCATGGTTGGACTGGAGAGCTGCTCCTTCCTG
GAGGAGGACTTCATCCCCTTTGCAGTGTTGAACATGATGATGGGCGGAGGTGGCTC
CTTCTCGGCTGGTGGGCCCGGCAAGGGCATGTTCTCCAGGCTCTACCTCAACGTGC
TCAACAGGCACCACTGGATGTATAACGCGACCTCCTACCACCACAGCTACGAGGA
CACTGGCCTCCTTTGCATCCATGCCAGCGCCGACCCAAGACAGGTTCGAGAAATGG
TAGAAATCATCACAAAGGAGTTTATTTTAATGGGCGGAACCGTGGACACGGTGGA
GCTGGAACGAGCCAAGACGCAGCTGACATCAATGCTCATGATGAACCTGGAATCC
AGGCCTGTGATCTTCGAGGATGTGGGGAGGCAGGTGCTGGCCACTCGCTCCAGAA
AGCTGCCGCACGAGCTGTGCACGCTCATCCGCAACGTGAAGCCGGAAGATGTGAA
GAGAGTCGCTTCTAAGATGCTCCGAGGGAAGCCGGCAGTGGCCGCCCTGGGTGAC
CTGACTGACCTGCCCACGTATGAGCACATCCAGACCGCCCTGTCGAGTAAGGACG
GGCGCCTGCCCAGGACGTACCGGCTCTTCCGGTAGAACCGCTCCCCGGCCTGACAG
ACCCAGGGAGCTGCAGCTGGAGCCCGTTCCCGTGCGTGTTAGTTTGGACACGAATT
TAGTCTAAAAAGCTGTCTGGTTGTATAAACGGTGCAAACAATGTCGCCACAGCACC
CACGCGGTTTGCATTCTTTTGGAACTCAATGTGCCGATCAGTGGAGTCAGTATCGA
GCCTGACCACCGCAAGCCAGGAAGCAGGTGAAGTGCCCAGCGCTGGAGTGCAGCG
TGCCACGAGGAGGGCGGTCGGTGCTTCCCTCCTCGGGCTGTGGGCACATGGGGCC
CCGCAGGTTCCTTGGAGGAGCCCTGAGCTGGGAGGCAGCAAAGGCTGACCTATCA
AAGCCTCCCGGAGGCCACCGTGCTGGGTACCAGGACTCACCTCTGACAAGCAGGA
GAAGGTAAGGGCCCGGTCAGCTCCAAGGAGCGCGCTCCACGCGCGTGCACACAGC
TTCCCTGGTAATAAAGAGCTGGCATCTTTCTTA
PMPCA Protein;
(NP_055975.1; SEQ ID NO: 22)
MAAVVLAATRLLRGSGSWGCSRLRFGPPAYRRFSSGGAYPNIPLSSPLPGVPKPVFAT
VDGQEKFETKVTTLDNGLRVASQNKFGQFCTVGILINSGSRYEAKYLSGIAHFLEKLAF
SSTARFDSKDEILLTLEKHGGICDCQTSRDTTMYAVSADSKGLDTVVALLADVVLQPR
LTDEEVEMTRMAVQFELEDLNLRPDPEPLLTEMIHEAAYRENTVGLHRFCPTENVAKI
NREVLHSYLRNYYTPDRMVLAGVGVEHEHLVDCARKYLLGVQPAWGSAEAVDIDRS
VAQYTGGIAKLERDMSNVSLGPTPIPELTHIMVGLESCSFLEEDFIPFAVLNMMMGGG
GSFSAGGPGKGMFSRLYLNVLNRHHWMYNATSYHHSYEDTGLLCIHASADPRQVRE
MVEIITKEFILMGGTVDTVELERAKTQLTSMLMMNLESRPVIFEDVGRQVLATRSRKL
PHELCTLIRNVKPEDVKRVASKMLRGKPAVAALGDLTDLPTYEHIQTALSSKDGRLPR
TYRLFR
PAM cDNA;
(NM_000919.3; SEQ ID NO: 23)
GTTCTGAATGATGACTGACGCGGGTTTGGGTGATACCCCTCACAGCCCCTGTCATT
CCGGAGTCATAAGGCACCCGCGCGTCTAGCCCCAGCGCCAGGGCACGCGAGCGGC
GCTGGAGGGAGGAAAGCTTCCGCCTGCGGGCCGGACAAAAGTCCCGCCTGCCCAC
GGCTTTTTGCCCGCCGCTCGTGACCGAGACGCCTCGCCGCGGCCAGCTCGCTGCTC
TCGCTGGCGGATGGTGTGTGGCCGCCGCAGGACGCCCGCCGTGCCCGGGCCATGA
AGTAGCGGCTGCTGGCGGCGCCGCTGCCCAACCGCCAGCCCCAGCCCCGCGCTGC
GCTGCCCGGTCCTCTCCCGGCGGGGTCGTATCGGCGTGGACATGGCTGGCCGCGTC
CCTAGCCTGCTAGTTCTCCTTGTTTTTCCAAGCAGCTGTTTGGCTTTCCGAAGCCCA
CTTTCTGTCTTTAAGAGGTTTAAAGAAACTACCAGACCATTTTCCAATGAATGTCTT
GGTACCACCAGACCCGTAGTTCCTATTGATTCATCAGATTTTGCATTGGATATTCGC
ATGCCTGGGGTTACACCTAAACAGTCCGATACATACTTCTGCATGTCTATGCGAAT
ACCAGTGGATGAGGAAGCCTTCGTGATTGACTTCAAGCCTCGAGCCAGCATGGAT
ACTGTCCATCACATGTTACTTTTTGGATGCAATATGCCTTCATCCACTGGAAGTTAC
TGGTTTTGTGATGAAGGAACCTGTACAGATAAAGCCAATATTCTGTATGCCTGGGC
GAGAAATGCTCCCCCTACCCGGCTCCCCAAAGGTGTTGGATTCAGAGTTGGAGGA
GAGACTGGAAGTAAATACTTTGTACTACAGGTACACTATGGGGATATTAGTGCTTT
TAGAGATAATAACAAGGACTGTTCTGGTGTGTCCTTACACCTCACACGTCTGCCAC
AGCCTTTAATTGCTGGCATGTACCTTATGATGTCTGTTGACACTGTTATCCCAGCAG
GAGAAAAAGTGGTGAATTCTGACATTTCATGCCATTATAAAAATTATCCAATGCAT
GTCTTTGCCTATAGAGTTCACACTCACCATTTAGGTAAGGTAGTAAGTGGATACAG
AGTAAGAAATGGACAGTGGACACTGATTGGACGGCAGAGCCCTCAGCTGCCACAG
GCTTTCTACCCTGTGGGGCATCCAGTTGATGTAAGTTTTGGTGACCTACTGGCTGC
AAGATGTGTATTCACTGGTGAAGGAAGGACAGAAGCCACACACATTGGTGGCACG
TCTAGTGATGAAATGTGCAACTTATACATTATGTATTACATGGAAGCCAAGCATGC
AGTTTCTTTCATGACCTGTACCCAGAATGTAGCTCCAGATATGTTCAGAACCATAC
CACCAGAGGCCAACATTCCAATTCCCGTGAAGTCTGATATGGTTATGATGCATGAA
CATCATAAAGAAACAGAATATAAAGATAAGATTCCTTTACTACAGCAGCCAAAAC
GAGAAGAAGAAGAAGTGTTAGACCAGGGTGATTTCTATTCACTACTTTCCAAGCTG
CTAGGAGAAAGGGAAGATGTTGTTCATGTGCACAAATATAATCCTACAGAAAAGG
CAGAATCAGAGTCAGACCTGGTAGCTGAGATTGCAAATGTAGTCCAAAAAAAGGA
TCTTGGTCGATCTGATGCCAGAGAGGGTGCAGAACATGAGAGGGGTAATGCTATT
CTTGTCAGAGACAGAATTCACAAATTCCACAGACTAGTATCTACCTTGAGGCCACC
AGAGAGCAGAGTTTTCTCATTACAGCAGCCCCCACCTGGTGAAGGCACCTGGGAA
CCAGAACACACAGGAGATTTCCACATGGAAGAGGCACTGGATTGGCCTGGAGTAT
ACTTGTTACCAGGCCAGGTTTCTGGGGTGGCTCTAGACCCTAAGAATAACCTGGTG
ATTTTCCACAGAGGTGACCATGTCTGGGATGGAAACTCGTTTGACAGCAAGTTTGT
TTACCAGCAAATAGGACTCGGACCAATTGAAGAAGACACTATTCTTGTCATAGATC
CAAATAATGCTGCAGTACTCCAGTCCAGTGGAAAAAATCTGTTTTACTTGCCACAT
GGCTTGAGTATAGATAAAGATGGGAATTATTGGGTCACAGACGTGGCTCTCCATCA
GGTGTTCAAACTGGATCCAAACAATAAAGAAGGCCCTGTATTAATCCTGGGAAGG
AGCATGCAACCAGGCAGTGACCAGAATCACTTCTGTCAACCCACTGATGTGGCTGT
GGATCCAGGCACTGGAGCCATTTATGTATCAGATGGTTACTGCAACAGCAGGATTG
TGCAGTTTTCACCAAGTGGAAAGTTCATCACACAGTGGGGAGAAGAGTCTTCAGG
GAGCAGTCCTCTGCCAGGCCAGTTCACTGTTCCTCACAGCTTGGCTCTTGTGCCTCT
TTTGGGCCAATTATGTGTGGCAGACCGGGAAAATGGTCGGATCCAGTGTTTTAAAA
CTGACACCAAAGAATTTGTGAGAGAGATTAAGCATTCATCATTTGGAAGAAATGT
ATTTGCAATTTCATATATACCAGGCTTGCTCTTTGCAGTGAATGGGAAGCCTCATTT
TGGGGACCAAGAACCTGTACAAGGATTTGTGATGAACTTTTCCAATGGGGAAATT
ATAGACATCTTCAAGCCAGTGCGCAAGCACTTTGATATGCCTCATGATATTGTTGC
ATCTGAAGATGGGACTGTGTACATTGGAGATGCTCATACCAACACCGTGTGGAAG
TTCACCTTGACTGAGAAATTGGAACATCGATCAGTTAAAAAGGCTGGCATTGAGGT
CCAGGAAATCAAAGAAGCCGAGGCAGTTGTTGAAACCAAAATGGAGAACAAACC
CACCTCCTCAGAATTGCAGAAGATGCAAGAGAAACAGAAACTGATCAAAGAGCCA
GGCTCGGGAGTGCCTGTTGTTCTCATTACAACCCTTCTGGTTATTCCGGTGGTTGTC
CTGCTGGCCATTGCCATATTTATTCGGTGGAAAAAATCAAGGGCCTTTGGAGCAGA
TTCTGAACACAAACTCGAGACGAGTTCAGGAAGAGTACTGGGAAGATTTAGAGGA
AAGGGAAGTGGAGGCTTAAACCTTGGTAATTTCTTTGCAAGCCGTAAGGGCTACA
GTCGAAAAGGGTTTGACCGGCTTAGCACTGAGGGCAGTGACCAAGAGAAAGAGG
ATGATGGAAGTGAATCAGAAGAGGAGTATTCAGCACCTCTGCCTGCGCTCGCACC
TTCCTCCTCCTGAAAACCAAGCTTTGATTTAGATTGAGTAAGATTTACCCAGAATG
TCAGATTCCTTTCCCTTTAGCACGTTTAAAGTTCTGTGTATTTAATTGTAAACTGTA
CTAGTCTGTGTGGGACTGTACACACTTTATTTACTTCGTTTTGGTTAAGTTGGCTTC
TGTTTCTAGTTGAGGAGTTTCCTAAAAGTTCATAACAGTGCCATTGTCTTTATATGA
ACATAGACTAGAGAAACCGTCCTCTTTTTCCATCATAATTCTAATCTAACAATGGA
AGATTTGCCCATTTACACTTTTGAGACTTTTTGGTGGATGTAAATAACCCCATTCTT
TGCTTGAACACAGTATTTTCCCAATAGCACTTTCATTGCCAGTGTCTTTCTTTGGTG
CCTTTCCTGTTCAGCATTCTTAGCCTGTGGCAGTAAAGAGAAACTTTGTGCTACAT
GACGACAAAGCTGCTAAATCTCCTATTTTTTTAAAATCACTAACATTATATTGCAA
TGAAGGAAATAAAAAAGTCTCTATTTAAATTCTTTTTTAAATTTTCTTCAGTTGGTG
TGTTTTTGGGATGTCTTATTTTTAGATGGTTACACTGTTAGAACACTATTTTCAGAA
TCTGAATGTAATTTGTGTAATAAAGTGTTTTCAGAGCATTAGCTGTCAGTGTATTTT
CCAGTTTTTGCGTATTTGCAGATTTTACATACAACTTTTATAATAATTACACAAACC
CACAAATATTAGTGAAACTTACTCGATGTCTTCAACTAAAAGAAATGTGTGTATTG
TACAAAATTTAGAAGATACTTTAGCCAATATAAATTAAAAACCAGCCTGAGTTTAC
ATAAATTTGTAAAGTCAGGCTCTTCTAAAATCCAAAGAGGGTTTTTGCCTATATAT
CAATCAGAGGATAAATACTTTAATAAAAGGTAATCACAGCTAAGTGGATACCTGT
GTCTCAAATTACATATGCAAATGATCCATCAGTAGGGATCACTAATAATAGTTTTC
CTTTTAAAAAATAATTTCAGGGCAGGTACAGTGGCTCAAGCCTATACTTCCAGGAC
TTTGGGAGGCCAAGCAGAAGGATTGTGGGAGGCCAAGCAGAAGGATTGTTTGAGC
CCAGAAATTCAAGGCTGCAGTGAGCTATGATCAATCCACTGTACTCCAGCCTAGGC
TACAGAGTGAGATCCTTTCTCTAAAATAAAATACAATTTCCATGTATCCATAGGAA
TATATTCATCTTTTACAGTGATTGCAGATTAGTTTTAAAACGTCTTTTTCGTAATTC
GTCAATGCAAGTTAGTAAGAACCAGATAATTTTCCATTTTAAAATGATAGGAATCT
AAAACTCTTTTTCAAAAATGCCAACCTGTTTTTCCGGCATCATAGTAGTTGGAATA
ATACAGATATAATTGACTAATCATAAAGTACATGAGAGTACAGAAGGGAAATTTG
GAAACGGTAAGTCTGCTAGGGCATTACAATCCTGTCCTAGCACTCCACCTTTATTC
TGCCAACCTGGGTTAATTAAAGATGGTAGCCTGGAAAGTAATGAATGACATTGAC
TTCAGGCAATCTTTCCACTGATTATTCTTGGCTGAACTTCATTTATCGAGTCAGAGA
ATGCACTGCCTGAGAAATGTCCCAGAGGAGTGAATCCTAGGCCTGGCCACAGTAG
AATCGCCTGGAGAATTTAAGAAAAAAATTGTTGGGCACTCACTCTTTCCAGTGATC
TTTATTTAGTTGGCTTACAATGGAATACAGGTATGGTTCATGTTTTTAAATTTGTCC
AGGTGTTTCTATTGTGCAGTCACAGTTGAAAACCATTGTCTTCGAGAATAGCTATT
CTATCTTGCCAGTTACAATAAGTGGATAGCTATATTTTCACATAAATTATAGTTTAC
AGATGTTTGGAGGGGGAAGACAGGATCTGAGGTGTTTTGATATGACTGTTAGCACC
AAAATCTGAATGCCTTAATTGTTGAATGTGTTAAATTGGATAATTAAAATGGGCAT
AAATGACTTATTAAAAAAGCAAAGGGAAAAAAAAAAAAAAAAAAAAAAAA
PAM Protein;
(NP_000910.2; SEQ ID NO: 24)
MAGRVPSLLVLLVFPSSCLAFRSPLSVFKRFKETTRPFSNECLGTTRPVVPIDSSDFALDI
RMPGVTPKQSDTYFCMSMRIPVDEEAFVIDFKPRASMDTVHHMLLFGCNMPSSTGSY
WFCDEGTCTDKANILYAWARNAPPTRLPKGVGFRVGGETGSKYFVLQVHYGDISAFR
DNNKDCSGVSLHLTRLPQPLIAGMYLMMSVDTVIPAGEKVVNSDISCHYKNYPMHVF
AYRVHTHHLGKVVSGYRVRNGQWTLIGRQSPQLPQAFYPVGHPVDVSFGDLLAARC
VFTGEGRTEATHIGGTSSDEMCNLYIMYYMEAKHAVSFMTCTQNVAPDMFRTIPPEA
NIPIPVKSDMVMMHEHHKETEYKDKIPLLQQPKREEEEVLDQGDFYSLLSKLLGERED
VVHVHKYNPTEKAESESDLVAEIANVVQKKDLGRSDAREGAEHERGNAILVRDRIHK
FHRLVSTLRPPESRVFSLQQPPPGEGTWEPEHTGDFHMEEALDWPGVYLLPGQVSGVA
LDPKNNLVIFHRGDHVWDGNSFDSKFVYQQIGLGPIEEDTILVIDPNNAAVLQSSGKNL
FYLPHGLSIDKDGNYWVTDVALHQVFKLDPNNKEGPVLILGRSMQPGSDQNHFCQPT
DVAVDPGTGAIYVSDGYCNSRIVQFSPSGKFITQWGEESSGSSPLPGQFTVPHSLALVPL
LGQLCVADRENGRIQCFKTDTKEFVREIKHSSFGRNVFAISYIPGLLFAVNGKPHFGDQ
EPVQGFVMNFSNGEIIDIFKPVRKHFDMPHDIVASEDGTVYIGDAHTNTVWKFTLTEKL
EHRSVKKAGIEVQEIKEAEAVVETKMENKPTSSELQKMQEKQKLIKEPGSGVPVVLITT
LLVIPVVVLLAIAIFIRWKKSRAFGADSEHKLETSSGRVLGRFRGKGSGGLNLGNFFAS
RKGYSRKGFDRLSTEGSDQEKEDDGSESEEEYSAPLPALAPSSS
ALDOA cDNA;
(NM_001127617.2; SEQ ID NO: 25)
GAAGCACCGGTGAGTGGGCAGGGGCTCCCTCCCCATCAATAGGGCCGACCCAAGT
CTTCCTCCCCCTTCCCCCATGCCGGGCCCCACGATAGTGTGAATGTCAGGGGCTTC
AGGTTTCCCTAAATATAGGTCCCTGCCAGAGGATCCGTGGCGGGAAAAGGGCAGG
GGTCATTAGAGAAGATCGGGGACACATGTGGGGCGGGCAGGAGCTGCCTTATAAC
CAGCCCGGGAACCCCTAGCTCACTCGCTGCTGACCAGGCTCTGCCGGCTCCTTCGG
CCTCGCCGCAGGAACTTGCTACTACCAGCACCATGCCCTACCAATATCCAGCACTG
ACCCCGGAGCAGAAGAAGGAGCTGTCTGACATCGCTCACCGCATCGTGGCACCTG
GCAAGGGCATCCTGGCTGCAGATGAGTCCACTGGGAGCATTGCCAAGCGGCTGCA
GTCCATTGGCACCGAGAACACCGAGGAGAACCGGCGCTTCTACCGCCAGCTGCTG
CTGACAGCTGACGACCGCGTGAACCCCTGCATTGGGGGTGTCATCCTCTTCCATGA
GACACTCTACCAGAAGGCGGATGATGGGCGTCCCTTCCCCCAAGTTATCAAATCCA
AGGGCGGTGTTGTGGGCATCAAGGTAGACAAGGGCGTGGTCCCCCTGGCAGGGAC
AAATGGCGAGACTACCACCCAAGGGTTGGATGGGCTGTCTGAGCGCTGTGCCCAG
TACAAGAAGGACGGAGCTGACTTCGCCAAGTGGCGTTGTGTGCTGAAGATTGGGG
AACACACCCCCTCAGCCCTCGCCATCATGGAAAATGCCAATGTTCTGGCCCGTTAT
GCCAGTATCTGCCAGCAGAATGGCATTGTGCCCATCGTGGAGCCTGAGATCCTCCC
TGATGGGGACCATGACTTGAAGCGCTGCCAGTATGTGACCGAGAAGGTGCTGGCT
GCTGTCTACAAGGCTCTGAGTGACCACCACATCTACCTGGAAGGCACCTTGCTGAA
GCCCAACATGGTCACCCCAGGCCATGCTTGCACTCAGAAGTTTTCTCATGAGGAGA
TTGCCATGGCGACCGTCACAGCGCTGCGCCGCACAGTGCCCCCCGCTGTCACTGGG
ATCACCTTCCTGTCTGGAGGCCAGAGTGAGGAGGAGGCGTCCATCAACCTCAATG
CCATTAACAAGTGCCCCCTGCTGAAGCCCTGGGCCCTGACCTTCTCCTACGGCCGA
GCCCTGCAGGCCTCTGCCCTGAAGGCCTGGGGCGGGAAGAAGGAGAACCTGAAGG
CTGCGCAGGAGGAGTATGTCAAGCGAGCCCTGGCCAACAGCCTTGCCTGTCAAGG
AAAGTACACTCCGAGCGGTCAGGCTGGGGCTGCTGCCAGCGAGTCCCTCTTCGTCT
CTAACCACGCCTATTAAGCGGAGGTGTTCCCAGGCTGCCCCCAACACTCCAGGCCC
TGCCCCCTCCCACTCTTGAAGAGGAGGCCGCCTCCTCGGGGCTCCAGGCTGGCTTG
CCCGCGCTCTTTCTTCCCTCGTGACAGTGGTGTGTGGTGTCGTCTGTGAATGCTAAG
TCCATCACCCTTTCCGGCACACTGCCAAATAAACAGCTATTTAAGGGGGAGTCGGC
AAAAAAAAAAAAAAAAAA
ALDOA Protein;
(NP_001121089.1; SEQ ID NO: 26)
MPYQYPALTPEQKKELSDIAHRIVAPGKGILAADESTGSIAKRLQSIGTENTEENRRFYR
QLLLTADDRVNPCIGGVILFHETLYQKADDGRPFPQVIKSKGGVVGIKVDKGVVPLAG
TNGETTTQGLDGLSERCAQYKKDGADFAKWRCVLKIGEHTPSALAIMENANVLARYA
SICQQNGIVPIVEPEILPDGDHDLKRCQYVTEKVLAAVYKALSDHHIYLEGTLLKPNMV
TPGHACTQKFSHEEIAMATVTALRRTVPPAVTGITFLSGGQSEEEASINLNAINKCPLLK
PWALTFSYGRALQASALKAWGGKKENLKAAQEEYVKRALANSLACQGKYTPSGQA
GAAASESLFVSNHAY
FANCC cDNA;
(NM_000136.3; SEQ ID NO: 27)
AGAATGCACTGCTGACACGTGTGCGCGCGCGCGGCTCCACTGCCGGGCGACCGCG
GGAAAATTCCAAAAAAACTCAAAAAGCCAATACGAGGCAAAGCCAAATTTTCAAG
CCACAGATCCCGGGCGGTGGCTTCCTTTCCGCCACTGCCCAAACTGCTGAAGCAGC
TCCCGCGAGGACCACCCGATTTAATGTGTGCCGACCATTTCCTTCAGTGCTGGACA
GGCTGCTGTGAAGGGACATCACCTTTTCGCTTTTTCCAAGATGGCTCAAGATTCAG
TAGATCTTTCTTGTGATTATCAGTTTTGGATGCAGAAGCTTTCTGTATGGGATCAGG
CTTCCACTTTGGAAACCCAGCAAGACACCTGTCTTCACGTGGCTCAGTTCCAGGAG
TTCCTAAGGAAGATGTATGAAGCCTTGAAAGAGATGGATTCTAATACAGTCATTGA
AAGATTCCCCACAATTGGTCAACTGTTGGCAAAAGCTTGTTGGAATCCTTTTATTTT
AGCATATGATGAAAGCCAAAAAATTCTAATATGGTGCTTATGTTGTCTAATTAACA
AAGAACCACAGAATTCTGGACAATCAAAACTTAACTCCTGGATACAGGGTGTATT
ATCTCATATACTTTCAGCACTCAGATTTGATAAAGAAGTTGCTCTTTTCACTCAAGG
TCTTGGGTATGCACCTATAGATTACTATCCTGGTTTGCTTAAAAATATGGTTTTATC
ATTAGCGTCTGAACTCAGAGAGAATCATCTTAATGGATTTAACACTCAAAGGCGA
ATGGCTCCCGAGCGAGTGGCGTCCCTGTCACGAGTTTGTGTCCCACTTATTACCCT
GACAGATGTTGACCCCCTGGTGGAGGCTCTCCTCATCTGTCATGGACGTGAACCTC
AGGAAATCCTCCAGCCAGAGTTCTTTGAGGCTGTAAACGAGGCCATTTTGCTGAAG
AAGATTTCTCTCCCCATGTCAGCTGTAGTCTGCCTCTGGCTTCGGCACCTTCCCAGC
CTTGAAAAAGCAATGCTGCATCTTTTTGAAAAGCTAATCTCCAGTGAGAGAAATTG
TCTGAGAAGGATCGAATGCTTTATAAAAGATTCATCGCTGCCTCAAGCAGCCTGCC
ACCCTGCCATATTCCGGGTTGTTGATGAGATGTTCAGGTGTGCACTCCTGGAAACC
GATGGGGCCCTGGAAATCATAGCCACTATTCAGGTGTTTACGCAGTGCTTTGTAGA
AGCTCTGGAGAAAGCAAGCAAGCAGCTGCGGTTTGCACTCAAGACCTACTTTCCTT
ACACTTCTCCATCTCTTGCCATGGTGCTGCTGCAAGACCCTCAAGATATCCCTCGG
GGACACTGGCTCCAGACACTGAAGCATATTTCTGAACTGCTCAGAGAAGCAGTTG
AAGACCAGACTCATGGGTCCTGCGGAGGTCCCTTTGAGAGCTGGTTCCTGTTCATT
CACTTCGGAGGATGGGCTGAGATGGTGGCAGAGCAATTACTGATGTCGGCAGCCG
AACCCCCCACGGCCCTGCTGTGGCTCTTGGCCTTCTACTACGGCCCCCGTGATGGG
AGGCAGCAGAGAGCACAGACTATGGTCCAGGTGAAGGCCGTGCTGGGCCACCTCC
TGGCAATGTCCAGAAGCAGCAGCCTCTCAGCCCAGGACCTGCAGACGGTAGCAGG
ACAGGGCACAGACACAGACCTCAGAGCTCCTGCACAACAGCTGATCAGGCACCTT
CTCCTCAACTTCCTGCTCTGGGCTCCTGGAGGCCACACGATCGCCTGGGATGTCAT
CACCCTGATGGCTCACACTGCTGAGATAACTCACGAGATCATTGGCTTTCTTGACC
AGACCTTGTACAGATGGAATCGTCTTGGCATTGAAAGCCCTAGATCAGAAAAACT
GGCCCGAGAGCTCCTTAAAGAGCTGCGAACTCAAGTCTAGAAGGCACGCAGGCCG
TGTGGGTGCCCGGCGTGAGGGATCAGGCTCGCCAGGGCCACAGGACAGGTGATGA
CCTGTGGCCACGCATTTGTGGAGTAAGTGCCCTCGCTGGGCTGTGAGAATGAGCTG
TACACATCTTGGGACAATCTGCTAGTATCTATTTTACAAAATGCAGAGCCAGGTCC
CTCAGCCCAGACTCAGTCAGACATGTTCACTAATGACTCAAGTGAGCCTTCGGTAC
TCCTGGTGCCCGCCCGGCCAGACCGTCAGCTTGATAATTACTAAAGCAAAGGCCTG
GGTGGGAGAACAGGTTTCTAGTTTTTACCCAAGTCAAGCTGCACATCTATTATTTA
AAAATTCAAAGTCTTAGAACCAAGAATTTGGTCATGAACCATTAAAGAATTTAGA
GAGAACTTAGCTCTTTTTAGACTCTTTTTAGGAGTCAGGGATCTGGGATAAAGCCA
CACTGTCTTGCTGTATGGAGAAATTCTTCAAGGGGAGTCAGGGTCCCTCAGGCTTC
CCTTGTGTCTCCCTGGACCTGCCTGACAGGCCACAGGAGCAGACAGCACACCCAA
GCCCGGGCCTCCGGCACACTCTTTCCACTCTGTATTTGCTAAATGATGCTAACTGCT
ACCAAAAGGCCCTTGGGACATCAGAGGAGCCGGCAGGCGAAGGTAGAGGATGTG
TTCCAGAAACATTAGAAGGCAGGATTAATTCAGTTAGTTAGTTCTCTTGTTAAATG
GAAATGGGAATTGGAAATTCCTGATAAAGAATTGGCCTGGCTGGGTGCAGTGGCT
CACACCTGTGATCCCAGCACTTTGGGAGGCCAAGGCAGGGGGATTACTTCAGCCC
AGGAGTTCCAGACTGCCTGGCTAACATGGCAATACCCTATCTCTACTAAAAATACA
AAAATTATCGGGGTGCAATGGCATGCATCTGTAATCCCAGCTATTCAAGAGGCTGA
GGCATGAGGATCTCTTGAACCCGGGAGGTGGGAGTTGTAGTGAGCCGAGATCATG
ACACTGCACTCCAGCCTGGGCAACAGAGCGAGACCATCTCTTAAAAAAAGGCATT
GTTAGTGTAATCTCAAGGTTAACATTTATTTCATGTCAGTACAGGGTGCTTTTTCCT
TTCAGGGACATTCTGGAATTGTATTGGTTGTACATTCTTTTGTGTCTATTCTGTTTGT
CAAGTGAGTCAAGACTTGCTTTTGTCCATTTTGATTTGTGTGTATTAGTCTGAGTCT
TGGCTCCGTTTTGAGGTATGAGCAAAGTTTTGCTGGATTAGAAGTTAACCTTTAGG
GAAATTCCTTATTTTGGTATGTGGCAATGCTAATAGATCCACTGAAGATCTGGAAA
ATTCCAGGAACTTTTCACCTGAGCCTTTCTTCTGAGAAATGCTGCAGTCAGAAGGG
TGTGCTGGTAAAGTATTTTGGTGGCAGCTGCCATCATGGTCATTGCCTTCATATAA
CATGCTTCGTGCTCATGGTCATTGCCTTCATATAACATGCTTCGTGCCATCATGATC
CTTGCCTTCATATAACAAACATGCTTCGTCAGAGGTGTTGGGGTTGAAAAAGGAGC
TGCATGCTTCACTGGAGTTGAGGGCCTCTCTCCTGTTCTGACTTTAAGCCAGAACTT
GTGGCTGGGCCATGGAAGCTGTGACTCCTCTGTGGACATGGTGGCAGCAGGGAAC
CCCTAGAGAGAGGGGCCACTGGGACCAGGCCTCCTGTTGTGGAGGGACTCCTGGG
ACAGTCCTCCACCCTGTCCTGTGGTCCTGTGTACAGGGTTGGCCTCTTCCTCCTCCC
CTGCCAGGCCTCTGCCCATGCCCCTTCCTTCCTTCTCCTGGGACTGGTGAAGCTAGG
CATCTGGAAGACTTCTTCCTAGCCTGGAAGCCCTGACCTCGGCCCATCTGCAGAAT
CTCCCAGTTCCTTCACAGCTGCCGAGTCCTCTCACGGGTGCGGTGGAGGCGGCCTT
GCCGGTGGTGCTTTCTGGGCAGCCAGGGGTTCCTGGGTGGGAGGACTGTCCCTCTG
GGGACGTGGCACTGAAGTGCCTGCTGGCTTCATGTGGCCCTTTGCCCTTTCCCAGC
CTGAGAGATGCTCAAAGGTGGGGAGCTGGGGGAGCCACCCCTCGGCCATTCCCTC
CACCTCCAAGACAGGTGGCGGCCGGGCAGGCACTCTTAAGCCCACCTCCCCCTCTT
GTTGCCTTCGATTTCGGCAAAGCCTGGGCAGGTGCCACCGGGAAGGAATGGCATC
CGAGATGCTGGGCGGGGACGCGGCGTGGCCGAGGGGGCCTTGACGGCGTTGGCGG
GGCCTGGGCACAGGGGCAGCCGCAGGGAGGCAGGGATGGCAAGGCGTGAAGCCA
CCCTGGAAGGAACTGGACCAAGGTCTTCAGAGGTGCGACAGGGTCTGGAATCTGA
CCTTACTCTAGCAGGAGTTTTTGTAGACTCTCCCTGATAGTTTAGTTTTTGATAAAG
CATGCTGGTAAAACCACTACCCTCAGAGAGAGCCAAAAATACAGAAGAGGCGGA
GAGCGCCCCTCCAACCAGGCTGTTATTCCCCTGGACTCCGTGACATCTGTGGAATT
TTTTAGCTCTTTAAAATCTGTAATTTGTTGTCTATTTTTTCATTCTAAATAAAACTTC
AGTTTGCACCTAA
FANCC Protein;
(NP_000127.2; SEQ ID NO: 28)
MAQDSVDLSCDYQFWMQKLSVWDQASTLETQQDTCLHVAQFQEFLRKMYEALKEM
DSNTVIERFPTIGQLLAKACWNPFILAYDESQKILIWCLCCLINKEPQNSGQSKLNSWIQ
GVLSHILSALRFDKEVALFTQGLGYAPIDYYPGLLKNMVLSLASELRENHLNGFNTQR
RMAPERVASLSRVCVPLITLTDVDPLVEALLICHGREPQEILQPEFFEAVNEAILLKKISL
PMSAVVCLWLRHLPSLEKAMLHLFEKLISSERNCLRRIECFIKDSSLPQAACHPAIFRVV
DEMFRCALLETDGALEIIATIQVFTQCFVEALEKASKQLRFALKTYFPYTSPSLAMVLL
QDPQDIPRGHWLQTLKHISELLREAVEDQTHGSCGGPFESWFLFIHFGGWAEMVAEQL
LMSAAEPPTALLWLLAFYYGPRDGRQQRAQTMVQVKAVLGHLLAMSRSSSLSAQDL
QTVAGQGTDTDLRAPAQQLIRHLLLNFLLWAPGGHTIAWDVITLMAHTAEITHEIIGFL
DQTLYRWNRLGIESPRSEKLARELLKELRTQV
PRC1 cDNA;
(NM_003981.4; SEQ ID NO: 29)
AACGGCTCGCGGAGCGGCTACGCGGAGTGACATCGCCGGTGTTTGCGGGTGGTTG
TTGCTCTCGGGGCCGTGTGGAGTAGGTCTGGACCTGGACTCACGGCTGCTTGGAGC
GTCCGCCATGAGGAGAAGTGAGGTGCTGGCGGAGGAGTCCATAGTATGTCTGCAG
AAAGCCCTAAATCACCTTCGGGAAATATGGGAGCTAATTGGGATTCCAGAGGACC
AGCGGTTACAAAGAACTGAGGTGGTAAAGAAGCATATCAAGGAACTCCTGGATAT
GATGATTGCTGAAGAGGAAAGCCTGAAGGAAAGACTCATCAAAAGCATATCCGTC
TGTCAGAAAGAGCTGAACACTCTGTGCAGCGAGTTACATGTTGAGCCATTTCAGGA
AGAAGGAGAGACGACCATCTTGCAACTAGAAAAAGATTTGCGCACCCAAGTGGAA
TTGATGCGAAAACAGAAAAAGGAGAGAAAACAGGAACTGAAGCTACTTCAAGAG
CAAGATCAAGAACTGTGCGAAATTCTTTGTATGCCCCACTATGATATTGACAGTGC
CTCAGTGCCCAGCTTAGAAGAGCTGAACCAGTTCAGGCAACATGTGACAACTTTG
AGGGAAACAAAGGCTTCTAGGCGTGAGGAGTTTGTCAGTATAAAGAGACAGATCA
TACTGTGTATGGAAGCATTAGACCACACCCCAGACACAAGCTTTGAAAGAGATGT
GGTGTGTGAAGACGAAGATGCCTTTTGTTTGTCTTTGGAGAATATTGCAACACTAC
AAAAGTTGCTACGGCAGCTGGAAATGCAGAAATCACAAAATGAAGCAGTGTGTGA
GGGGCTGCGTACTCAAATCCGAGAGCTCTGGGACAGGTTGCAAATACCTGAAGAA
GAAAGAGAAGCTGTGGCCACCATTATGTCTGGGTCAAAGGCCAAGGTCCGGAAAG
CGCTGCAATTAGAAGTGGATCGGTTGGAAGAACTGAAAATGCAAAACATGAAGAA
AGTGATTGAGGCAATTCGAGTGGAGCTGGTTCAGTACTGGGACCAGTGCTTTTATA
GCCAGGAGCAGAGACAAGCTTTTGCCCCTTTCTGTGCTGAGGACTACACAGAAAG
TCTGCTCCAGCTCCACGATGCTGAGATTGTGCGGTTAAAAAACTACTATGAAGTTC
ACAAGGAACTCTTTGAAGGTGTCCAGAAGTGGGAAGAAACCTGGAGGCTTTTCTT
AGAGTTTGAGAGAAAAGCTTCAGATCCAAATCGATTTACAAACCGAGGAGGAAAT
CTTCTAAAAGAAGAAAAACAACGAGCCAAGCTCCAGAAAATGCTGCCCAAGCTGG
AAGAAGAGTTGAAGGCACGAATTGAATTGTGGGAACAGGAACATTCAAAGGCATT
TATGGTGAATGGGCAGAAATTCATGGAGTATGTGGCAGAACAATGGGAGATGCAT
CGATTGGAGAAAGAGAGAGCCAAGCAGGAAAGACAACTGAAGAACAAAAAACAG
ACAGAGACAGAGATGCTGTATGGCAGCGCTCCTCGAACACCTAGCAAGCGGCGAG
GACTGGCTCCCAATACACCGGGCAAAGCACGTAAGCTGAACACTACCACCATGTC
CAATGCTACGGCCAATAGTAGCATTCGGCCTATCTTTGGAGGGACAGTCTACCACT
CCCCCGTGTCTCGACTTCCTCCTTCTGGCAGCAAGCCAGTCGCTGCTTCCACCTGTT
CAGGGAAGAAAACACCCCGTACTGGCAGGCATGGAGCCAACAAGGAGAACCTGG
AGCTCAACGGCAGCATCCTGAGTGGTGGGTACCCTGGCTCGGCCCCCCTCCAGCGC
AACTTCAGCATTAATTCTGTTGCCAGCACCTATTCTGAGTTTGCGAAGGATCCGTC
CCTCTCTGACAGTTCCACTGTTGGGCTTCAGCGAGAACTTTCAAAGGCTTCCAAAT
CTGATGCTACTTCTGGAATCCTCAATTCAACCAACATCCAGTCCTGAGAAGCCCTG
ATCAGTCAACCAGCTGTGGCTTCCTGTGCCTAGACTGGACCTAATTATATGGGGGT
GACTTTAGTTTTTCTTCAGCTTAGGCGTGCTTGAAACCTTGGCCAGGTTCCATGACC
ATGGGCCTAACTTAAAGATGTGAATGAGTGTTACAGTTGAAAGCCCATCATAGGTT
TAGTGGTCCTAGGAGACTTGGTTTTGACTTATATACATGAAAAGTTTATGGCAAGA
AGTGCAAATTTTAGCATATGGGGCCTGACTTCTCTACCACATAATTCTACTTGCTG
AAGCATGATCAAAGCTTGTTTTATTTCACCACTGTAGGAAAATGATTGACTATGCC
CATCCCTGGGGGTAATTTTGGCATGTATACCTGTAACTAGTAATTAACATCTTTTTT
GTTTAGGCATGTTCAATTAATGCTGTAGCTATCATAGCTTTGCTCTTACCTGAAGCC
TTGTCCCCACCACACAGGACAGCCTTCCTCCTGAAGAGAATGTCTTTGTGTGTCCG
AAGTTGAGATGGCCTGCCCTACTGCCAAAGAGGTGACAGGAAGGCTGGGAGCAGC
TTTGTTAAATTGTGTTCAGTTCTGTTACACAGTGCATTGCCCTTTGTTGGGGGTATG
CATGTATGAACACACATGCTTGTCGGAACGCTTTCTCGGCGTTTGTCCCTTGGCTCT
CATCTCCCCCATTCCTGTGCCTACTTTGCCTGAGTTCTTCTACCCCCGCAGTTGCCA
GCCACATTGGGAGTCTGTTTGTTCCAATGGGTTGAGCTGTCTTTGTCGTGGAGATCT
GGAACTTTGCACATGTCACTACTGGGGAGGTGTTCCTGCTCTAGCTTCCACGATGA
GGCGCCCTCTTTACCTATCCTCTCAATCACTACTCTTCTTGAAGCACTATTATTTAT
TCTTCCGCTGTCTGCCTGCAGCAGTACTACTGTCAACATAGTGTAAATGGTTCTCA
AAAGCTTACCAGTGTGGACTTGGTGTTAGCCACGCTGTTTACTCATACAGTACGTG
TCCTGTTTTTAAAATATACAATTATTCTTAAAAATAAATTAAAATCTGTATACTTAC
ATTTCAAAAA
PRC1 Protein;
(NP_003972.2; SEQ ID NO: 30)
MRRSEVLAEESIVCLQKALNHLREIWELIGIPEDQRLQRTEVVKKHIKELLDMMIAEEE
SLKERLIKSISVCQKELNTLCSELHVEPFQEEGETTILQLEKDLRTQVELMRKQKKERK
QELKLLQEQDQELCEILCMPHYDIDSASVPSLEELNQFRQHVTTLRETKASRREEFVSIK
RQIILCMEALDHTPDTSFERDVVCEDEDAFCLSLENIATLQKLLRQLEMQKSQNEAVCE
GLRTQIRELWDRLQIPEEEREAVATIMSGSKAKVRKALQLEVDRLEELKMQNMKKVIE
AIRVELVQYWDQCFYSQEQRQAFAPFCAEDYTESLLQLHDAEIVRLKNYYEVHKELFE
GVQKWEETWRLFLEFERKASDPNRFTNRGGNLLKEEKQRAKLQKMLPKLEEELKARI
ELWEQEHSKAFMVNGQKFMEYVAEQWEMHRLEKERAKQERQLKNKKQTETEMLYG
SAPRTPSKRRGLAPNTPGKARKLNTTTMSNATANSSIRPIFGGTVYHSPVSRLPPSGSKP
VAASTCSGKKTPRTGRHGANKENLELNGSILSGGYPGSAPLQRNFSINSVASTYSEFAK
DPSLSDSSTVGLQRELSKASKSDATSGILNSTNIQS
SPRED2 cDNA;
(NM_181784.3; SEQ ID NO: 31)
GCACTCCACCACCTCTGGCCGCTGGGAAGACGTCATCGCTTCCGCCCTACTTCCTC
CTCCTGTTGGCGGCGGTGAGAATCCAATATGGAGTAAATCGGTGGAGTCGAGAGA
TGGGGCTCGGACGGGGCGCCCTGCACCGCCTCCCAGGCGCACCTCCCCCGGCCGC
CGACCGCTCCCCGGAACCGTGATTGGCCCGGCCCCCCGGGGGCGACCCCGGACTG
AGCCCCCCCACCCGCGGCCGGCGCTCTCCTCCTCCTCGCAGCAGTCCCTGCCGCTC
CGAGGCGCGCTTGTTTTTCTCCTGCTCTCCCCCACCGCCGTCCCCTTCCCAAATCTC
GCCCCCTCTGCCCGCTCCCGAGCCCCCGGAGCCTCCGCCCGCCTCTCCCCACGGCG
CTGCGGCGTTCACTCCCCGCGCAGCCTGCCTTTGCACCCCTTCCCCCAAACCCTATC
CCGCGCCCTGCTTCCCCTTCTGCTGCGGCGCCCTCTTCATCTCTAGCCGCCCCCCCT
CCCCAAATCAGGCGATCTCCGGAGATGTGAAGAAGGGGGGCGAGCGGACAGGAA
GATGAAGGGAGCAAAGCTGCCCGCCGCGGGACAGGCGTCTAGGTGAACAAGAAA
ATGACCGAAGAAACACACCCAGACGATGACAGCTATATTGTGCGTGTCAAGGCTG
TGGTTATGACCAGAGATGACTCCAGCGGGGGATGGTTCCCACAGGAAGGAGGCGG
GATCAGTCGCGTCGGGGTCTGTAAGGTCATGCACCCCGAAGGCAATGGACGAAGC
GGCTTTCTCATCCATGGTGAACGACAGAAAGACAAACTGGTGGTATTGGAATGCT
ATGTAAGAAAGGACTTGGTCTACACCAAAGCCAATCCAACGTTTCATCACTGGAA
GGTCGATAATAGGAAGTTTGGACTTACTTTCCAAAGCCCTGCTGATGCCCGAGCCT
TTGACAGGGGAGTAAGGAAAGCAATCGAAGACCTTATAGAAGGTTCAACAACGTC
ATCTTCCACCATCCATAATGAAGCTGAGCTTGGCGATGATGACGTTTTTACAACAG
CTACAGACAGTTCTTCTAATTCCTCTCAGAAGAGAGAGCAACCTACTCGGACAATC
TCCTCTCCCACATCCTGTGAGCACCGGAGGATTTATACCCTGGGCCACCTCCACGA
CTCATACCCCACAGACCACTATCACCTCGATCAGCCGATGCCAAGGCCCTACCGCC
AGGTGAGCTTCCCGGACGACGACGAGGAGATCGTGCGCATCAACCCCCGGGAGAA
GATCTGGATGACGGGGTACGAGGATTACCGGCACGCACCCGTCAGGGGCAAGTAC
CCGGACCCCTCGGAGGACGCGGACTCCTCCTACGTGCGCTTCGCCAAGGGCGAGG
TCCCCAAGCATGACTACAACTACCCCTACGTGGACTCCTCAGACTTTGGCCTAGGC
GAGGACCCCAAAGGCCGCGGGGGCAGCGTGATCAAGACGCAGCCCTCCCGGGGC
AAGTCGCGGCGGCGGAAGGAGGACGGAGAGCGCTCGCGGTGCGTGTACTGCAGG
GACATGTTCAACCACGAGGAGAACCGCCGGGGCCACTGCCAGGACGCGCCCGACT
CCGTGAGAACTTGCATCCGCCGGGTGAGCTGCATGTGGTGCGCGGACAGCATGCT
CTATCACTGTATGTCGGACCCCGAGGGAGACTATACAGACCCTTGCTCGTGCGATA
CTAGCGACGAGAAGTTTTGCCTCCGGTGGATGGCTCTTATTGCCTTGTCTTTCCTGG
CCCCCTGTATGTGCTGTTACCTGCCCCTTCGGGCCTGCTACCACTGCGGAGTGATGT
GCAGGTGCTGTGGCGGGAAGCACAAAGCGGCCGCGTGACTCAGTTTCCCTCCCTTC
TCCCTCCATCCGCAGCCACAGGGGAACTCGTCTCTTACATACTCTCATCTTCTCCCC
CGCTCCCTTCCACTCCAAGGAGCGAGGAGGGCAAGCGGCCTCCCAGCTCCCTGGT
ACCTCGAGGCACCATTCCAGCCAGGGACGCTGCCGGGTAGACTCTCCACTCCCCCT
GCCGCCCACACTGCAGCAGCCACATCCATACACACACGCTCGCACAGTGTTCTGAG
GAAGGAACCTTCGCCACAGACTCCTGTACTATTAACAATCTGTAACCAAGCTAACT
GTCTCATCCATGTGTTGATTTCCTGTTTCCTCCTCCCCCGCCTCTTCCAGTTCAAAG
GAGTCTGCAATTGGAACTGCTGATTTTCGGTGGGTTTTGTAGTTGATTTTTCCAAGA
GCGTCGAAGACTCTCTTTCTCTTGGTTCACCTTGCCTGTCGCTAGCAAGCATCTGGT
TCAGCGGAAATGGGATGTGAGAATGATGAAACCCGACAGAAGTATCTCAGCCTGC
AGTCAGTTATTATGTATAGGAGGTGAGCTAGTTAACAAACTTGTACCACAAAACA
ATATCGCTTTAACTTTTCTAAAGCCAAATTTCCCATGTAAGCTGCAGTTTCTATCTT
TAGCCCATCATCATTTTCTGCCCCCCCAAAATCTGTTGAAATGATTCACTGATGCA
AAACATTCACCCGTAATAGACTGAGAATTGAGCCTTAACTTCAGATTAACTTGTGA
GAATCAGGAAAATTCCTTCAACTGCATTGCATCCTCTTGGACCAGGGCTAGAATGG
GGATTTCAGGTTTCTATGAGCCTCCCCATTACCCCTAAAGTAGAACATTTTTTAAAT
TGTGTTGCCACCACTTCTAAAAATTTAGCAATATTAGAATAGGATACTAGTGAAGT
AAGAAATTTTGCTTGTTGTTTTGCAAACCAAATAGTTTCCTCAAACACAAATCAGT
GTTCCCACGAACAAGTTCCAGTTGAGAAACACTAAGGTTATGGTGAATAAAACCA
TAGGGAGCTTCTTCCCCCCAACCCCTGGCTATTTTATATTAATGGGGAGAGGGGAT
TTTTAAATGTCATAAATTTGAAGAGTGGTGGGTTGCATTTTCTTCATGGGTTTATGT
TTTCGTTTCATTTTGGACTCAATTTCACATCACCAAATTCCTCATTTATACTTGGGG
AAAAAACAAGGCCATATGTAAAAACCCTTCCAATGCCTAAGTGTCTTTCTCCTGCA
ACTCCAAACCCAGACTCGCCACTTTGGGTGCACAGGTGGTTAGGTCAGCCAACTGG
TTCTGCCTGTCGCCTTGCCACGGAGGAGGCTTCTAATTAGCTGGAAAAGAGTATTT
TTCTAATACGTTGCAAGGATTAGCCAAATCTTCTTATTGAAGAAAGAAGAAAAGTG
AAGAGTGGTTACCTATACCTAGCATAGTACAATCAGAACCTCGTGGAGACCACCG
GGGACAGGCTTGCGGACGCCGGCTGTTCTTCCCGCCACGATCTTTCTGTGGTAGCG
GCCAGCAGAAGACATGGCCTGCTCCCCACTCCCTTTCCCCACTCCTTCTTTATTGCA
CACAGGACACCAGTCTTCAAGGAAAGGGACTTTTTTCCAGTCTGCCAATCATATTG
GGAAAGTGCTAGCTGTGCTCACCTTCATGGGGCTGTTTCCAGCTCGTCCACAAGCT
CATCGATTTTCTTTAGTAGATTACCGGTGTAAATACCCAGTGTGCTTATGAGTCAGT
TAGTAGACGTCTTCATTCATTGGAGTAACTGGTTTAGGCTTTCCAGTTTGGAAAAG
GAGCAGAGAGCTGTCCATCTGGATTGATGGAAGAAAAGAGAACCTCATCCATGCC
TGGAGAACATCTAGAAAACCTTCAGCCAGCCTCCAGTGCTGTCGAGAGACCACCTT
CCCCCGACCCGGAGGCACTTCCTTGGGGTCTTTCTCTAGGGTCTCTTCTCTACAAAG
CACAACACTAATGTTCGTTTCCTTAGACCTCAGTTCAAGTGCCCCTATTTATTTCAA
TAAGAACGCACATATCCCAGCTGTTTTTTGTTTGTCACCTCTATTTAGTTGTTACCT
GTTTCTCTCTTCTTTCACCCCTTGTCCTTTTCCACCCTTTTAAGAGTTACGCTAGCAG
ATCTTACTCCACGTATACTTTTTGGTTTGTGAAGGCATCGGTTAAGGGCACAAAGA
CAGCCATGGGGACATTTATGTAAATACGTCTCTAATTGCCACACTGCAGCTGAACA
GTGTGTAGTATTTTCCCAGTCAGCTTTGCCATACTGACGTCAATCATTTGAGAGAA
ATTATTCAGATTTTATTTTTGTATCTGTGGTAACAAAACATTAACCAAAAGATTTTC
TGTCCAGAAGCCTCCCCGACCCCCCAAGCTATTTGCTCACATTAACAAATTAAAGT
GCCTGAAGCATAATTCATTCTTTACCTGTATACTAAAAACCCTGTTGTATTGATTTT
TTTATAATAAGCCTTTTTACCTCTGTGTAAAAAATATATATACAAGTGTATGATGTA
CATTTTAGTTCTTAACTTTTTTTTATGGTTTCTAATATGTATGACCAATGTAGCCATT
GCTTTAAAATGTACCGTGTAAATATAAACACATCCTATCAGA
SPRED2 Protein;
(NP_861449.2; SEQ ID NO: 32)
MTEETHPDDDSYIVRVKAVVMTRDDSSGGWFPQEGGGISRVGVCKVMHPEGNGRSG
FLIHGERQKDKLVVLECYVRKDLVYTKANPTFHHWKVDNRKFGLTFQSPADARAFDR
GVRKAIEDLIEGSTTSSSTIHNEAELGDDDVFTTATDSSSNSSQKREQPTRTISSPTSCEH
RRIYTLGHLHDSYPTDHYHLDQPMPRPYRQVSFPDDDEEIVRINPREKIWMTGYEDYR
HAPVRGKYPDPSEDADSSYVRFAKGEVPKHDYNYPYVDSSDFGLGEDPKGRGGSVIK
TQPSRGKSRRRKEDGERSRCVYCRDMFNHEENRRGHCQDAPDSVRTCIRRVSCMWC
ADSMLYHCMSDPEGDYTDPCSCDTSDEKFCLRWMALIALSFLAPCMCCYLPLRACYH
CGVMCRCCGGKHKAAA
GLP1R cDNA;
(NM_002062.5; SEQ ID NO: 33)
ATCAGTCTCCGCACGCGGTTCCGCAGGTGGCAGCGATGGCCCAGTCCTGAACTCCC
CGCCATGGCCGGCGCCCCCGGCCCGCTGCGCCTTGCGCTGCTGCTGCTCGGGATGG
TGGGCAGGGCCGGCCCCCGCCCCCAGGGTGCCACTGTGTCCCTCTGGGAGACGGT
GCAGAAATGGCGAGAATACCGACGCCAGTGCCAGCGCTCCCTGACTGAGGATCCA
CCTCCTGCCACAGACTTGTTCTGCAACCGGACCTTCGATGAATACGCCTGCTGGCC
AGATGGGGAGCCAGGCTCGTTCGTGAATGTCAGCTGCCCCTGGTACCTGCCCTGGG
CCAGCAGTGTGCCGCAGGGCCACGTGTACCGGTTCTGCACAGCTGAAGGCCTCTG
GCTGCAGAAGGACAACTCCAGCCTGCCCTGGAGGGACTTGTCGGAGTGCGAGGAG
TCCAAGCGAGGGGAAAGAAGCTCCCCGGAGGAGCAGCTCCTGTTCCTCTACATCA
TCTACACGGTGGGCTACGCACTCTCCTTCTCTGCTCTGGTTATCGCCTCTGCGATCC
TCCTCGGCTTCAGACACCTGCACTGCACCAGGAACTACATCCACCTGAACCTGTTT
GCATCCTTCATCCTGCGAGCATTGTCCGTCTTCATCAAGGACGCAGCCCTGAAGTG
GATGTATAGCACAGCCGCCCAGCAGCACCAGTGGGATGGGCTCCTCTCCTACCAG
GACTCTCTGAGCTGCCGCCTGGTGTTTCTGCTCATGCAGTACTGTGTGGCGGCCAA
TTACTACTGGCTCTTGGTGGAGGGCGTGTACCTGTACACACTGCTGGCCTTCTCGG
TCTTATCTGAGCAATGGATCTTCAGGCTCTACGTGAGCATAGGCTGGGGTGTTCCC
CTGCTGTTTGTTGTCCCCTGGGGCATTGTCAAGTACCTCTATGAGGACGAGGGCTG
CTGGACCAGGAACTCCAACATGAACTACTGGCTCATTATCCGGCTGCCCATTCTCT
TTGCCATTGGGGTGAACTTCCTCATCTTTGTTCGGGTCATCTGCATCGTGGTATCCA
AACTGAAGGCCAATCTCATGTGCAAGACAGACATCAAATGCAGACTTGCCAAGTC
CACGCTGACACTCATCCCCCTGCTGGGGACTCATGAGGTCATCTTTGCCTTTGTGAT
GGACGAGCACGCCCGGGGGACCCTGCGCTTCATCAAGCTGTTTACAGAGCTCTCCT
TCACCTCCTTCCAGGGGCTGATGGTGGCCATATTATACTGCTTTGTCAACAATGAG
GTCCAGCTGGAATTTCGGAAGAGCTGGGAGCGCTGGCGGCTTGAGCACTTGCACA
TCCAGAGGGACAGCAGCATGAAGCCCCTCAAGTGTCCCACCAGCAGCCTGAGCAG
TGGAGCCACGGCGGGCAGCAGCATGTACACAGCCACTTGCCAGGCCTCCTGCAGC
TGAGACTCCAGCGCCTGCCCTCCCTGGGGTCCTTGCTGCAGGCCGGGTGGCCAATC
CAGGTGGGAGAGACACTCCCAGGGACAAGGGAAGGAAGGGACACACACACACAC
ACACACACACACACACACACACACATACATCCTGCTTTCCCTCCCCAAACCCATCA
GACAGGTAAATGGGCAGTGCCTCCTGGGACCATGGACACATTTTCTCCTAGGAGA
AGCAGCCTCCTAATTTGATCACAGTGGCGAGAGGAGAGGAAAAACGATCGCTGTG
AAAATGAGGAGGATTGCTTCTTGTGAAACCACAGGCCCTTGGGGTTCCCCCAGAC
AGAGCCGCAAATCAACCCCAGACTCAAACTCAAGGTCAACGGCTTATTAGTGAAA
CTGGGGCTTGCAAGAGGAGGTGGTTCTGAAAGTGGCTCTTCTAACCTCAGCCAAAC
ACAGAGCGGGAGTGACGGGAGCCTCCTCTGCTTGCATCACTTGGGGTCACCACCCT
CCCCTGTCTTCTCTCAAAGGGAAGCTGTTTGTGTGTCTGGGTTGCTTATTTCCCTCA
TCTTGCCCCCTCATCTCACTGCCCAGTTTCTTTTTGAGGGGCTTTGTTTGGGCCACT
GCCAGCAGCTGTTTCTGGAAATGGCTGTAGGTGGTGTTGAGAAAGAATGAGCATT
GAGACGGTGCTCGCTTCTCCTCCAGGTATTTGAGTTGTTTTGGTGCCTGCCTCTGCC
ATGCCCAGAGAATCAGGGCAGGCTTGCCACCGGGGAACCCAGCCCTGGGGTATGA
GCTGCCAAGTCTATTTTAAAGACGCTCAAGAATCCTCTGGGGTTCATCTAGGGACA
CGTTAGGAATGTCCAGACTGTGGGTGTAGATTACCTGCCACTTCCAGGAGCCCAGA
GGGCCAAGAGAGACATTGCCTCCACCTCTCCTTGGAAATACTTTATCTGTGACCAC
ACGCTGTCTCTTGAGAATTTGGATACACTCTCTAGCTTTAGGGGACCATGAAGAGA
CTCTCTTAGGGAAACCAATAGTCCCCATCAGCACCATGGAGGCAGGCTCCCCCTGC
CTTTGAAATTCCCCCACTTGGGAGCTTGTATATACTTCACTCACTTTTCTTTATTGCT
GTGAATAGTCTGTGTGCACAATGGGCAATTCTGACTTCTCCCATCTAGTGGAAATG
AGCGAAATCATGGTTGTAGTGATGTTGTTTGGGAGAGTGCAGTAGTAATTGATTTG
ACCCACTCACACTTGGAGCTAATTAAGGTTTGCCCTGCCTGCAGCCTCCCCCACAA
ATAATGAACAGCAGAAAGACTGGACGGGGAAACCTATCAATCCTGCCCCCAGCCA
TGGTGAGGAAGCCCCAAGCCATGGTGACACACAGCAGCACTGCAGATAGCCAGAC
ACATGGCTATCCTAGAGAGGCTGGCAAGGAGTTCGTGGCTGCAAAAGAAGTTTCT
GGAGCAAGAGAGAGCTCGCTCTTGGGAGTCAGGACCTCCGGGGAGAGCAGAGGG
TTCCGACGGATTCCTTTATGAGTCAGTCTCTCTCTCCCTTTTAAATGGTGGGAACCC
TCCCCAAAACCTTTCCCCAGACACATTCTCCTGTGCCCCTCAGAGAGGCATGTGAT
GTGCAAGGAAAATAATAGGATATAAAACACATCAAGTAGAAAATTTCTTATACTT
CAGCTTCAGTGAAGTGTTGTCTATGTTAATAGGCAAGTTGAACCTCGGGCTAAAGA
AAGGAATTGTGTGGATGGCTGCCTTGACTGAGACAATATGGGCAGGGGGGCGTTC
CTGCTTCCCCAGAACAAGGGCAGGCTTCCCAAAGGCACCCCTTATTTGCTGTCTCT
TCGTAAGCGTGGGCACCAGTGGGTTGATGTGGGAAACAGTGCTGGTGCAAGTGTT
TAAGTTTTGGATAAACTGAGGATTTTGAGAATCATTATTACATTACATAGCAACAA
AGAAACAGATTGATATAATCTGTGTGAAATAAATTGCTGAGGAGAGGGATTGTAG
GGAAGAAGTTGCTTAGTTAGGCACCTGGGAAGCTCAAATCATTTAGTTTAAACTGT
AAGTAGAGTTGTCTTCCCAACGAGAACTGGTGATTCCTGATTTCAGAAGGCTCATC
AGACCCCTACACCCAAGTCTTTTAACACGGAGGAAGTTTTTCCTTCATGAATACAT
ACAAGATACCGAACTGAAGAAATCTTTTCTTTGCTCCAACCCCCGCTCCCCCGGCC
CCCCGAAGCTATTAATAGTCATTCAAATGGCACTGCACTCTTTCCAAGCTTTCCTA
AGCTAAATATGGCTCCAACAGGTCTGGGAACTATTGAATGCAATGTACTCTGAAAT
TCCAGCAGCTGTTTACATGCCTGATGCCTAATGCCATACGCCTGGAACTTGCTGAG
AACTCTCGGCTGCCTCTGTCCTGGGGTACTATTGCTGACCAGCAGGGTGTAGGCGC
TGCTTTTGTTTGAGCTTTGTAGCCACAAGGTCTGATGTCTGTTGTGAACAGCAGCTG
ACTTCAGCACTCTGGCCAGCTTCCTCAAACTTGTTAGTGGCTTGCATTTTGTCAATT
TTTCTTTGCCATTTCAGTCTTTCAAGCAGCTGAATCGCAGAACTGAAACAAAACAC
TTATATTGCAAAGTTCCTTCCCTTTAAAATGAGCTTCTGGATTGCAGAGATGATTTT
TTATGGTCTTAGCGTGCCTCATGCTGTAGACCACATCGTTGGAGCTCTGGGAGAGC
TCTGAGCAGAGAAAGACCTTGGTGATCCCCCCTGGCCCGATCTATAGGATTGAGG
AATGAGGCTTGTAGAGGTGGCTTGCCCAAGGTCACAACACTAGAGGGTGGTTGAG
CTGGGCCAGGACCCAGACAAGCCCTTTTCTCTCAGTCTAGTGAGGCCTCTGGGAAG
GATAGGTGAAAACTATAAATAGCTGTTCAAGATACTCTGAGTAAAAGTCAGTGGT
GTTATCTGGAATTTCTACTGAAGAGGAAATACAGCAGCAAAGGTAGACTCATCAG
ATGGTGAAAGTGTCATCTCCTTTGTAATTTATACTGGTGAGAGGAGGGGAAGGATG
TGCTTTCCTATTTTTACACTGGTACTTCATTCATCCTTGTCTTAAACTTAAGAAATG
AAATGCATCAGGGCACACATATACATAGAAACAGCACATAAGCCATTTTGGATGG
CAATTCCAGCACTCTTTTAATGGATTGGCTTAATTTTTTTTTCTGATGCCTCTCTATG
TGTTTGAAAAGTTGTATTGATAGTCACACAGAGTGTACTAAAATAACCTAAAAGGT
CTTGAACCTCCCCTTTACCTCTTTAGAACCTCTGAATTTGGAGACAGCTAGCAAAC
GGCAACTGTAGCCTAGCAAAGATGGCACAGCACCACAAAGGGCAAATGGAGTCA
ACTCCCTTCCATGCTCTGAAGGTCTTGGCCACTCTCATCAGGGTTGGCTGTGTACTA
TCCAGCAGCTCACAGTCAGGAGACCTCCCCTCCTCAGTCCCTCCCATTTCTTTCTTC
CGCAAGGAAAGAACATTGCATGTGGGCGTGTGTGTGTGTATGTGTGTGCATGTGTG
TGTGCATGTTTCTTCTGTGCATAGCAGCATAAAGTTAGGAGATGATGACTCCAGAA
TCATCACACCCGCTGTGCAGGTCATAGGACCGCACACCCGAGGCTGGGAGTCTAA
GTGGTAGATGCTTCCTCTCCATGCGCAGACAGGGCTCCCATTATTTGCTAATGCCT
GCGACGTGCTCAGGGAAGGCAAAATCTATTCCAAAGGTTTATTTTCCCTCTGTTGA
ATTGATTCTAATCTACAGTGCTTACCCTGCTTTTGGCTCCTACGAGGCAAACTATAA
AAATTCTCAAATAAAATACAGAGGCTTACTCTGCAAAGACACCTGTGTGAATGAC
GTGCTAAACATTCAGCAGAAACCAAATTAGGATCAACTCTTAACATTTTTTCCCTC
TTTTGTGGCTGGAATGAGTTCCCCTGGGAAAGAAAATCGCCCAAGTGAGAAACTT
ATGCCAACCACATTCCCATGCCCCTACAGTTACCTGCTGGCCCAGATCAGCTCTCT
GGGTCTGCAGAGGGCCGAGACAGGAAGCATTATTAAGCAGTATAATTTCAAACAC
TTTACTTAGTGCCACAGTGTGTCTGCAGTATCTATAAGTACTTCCGCTCTGTCAAGG
AATACAGACTGGCAGGAAGCAGATAAGCATAATTGTTTTCCAATAACTCCCCAAG
TCTCTTGGAATATATATGTATCTGAATCTCTCAGAGGAGAGAGTTTTCTCTCTTCTT
TGGCCATTTTAGCCTATTTTCCAGGACCTTGTCTAGCAGTGGCTCCATTTTTCCCCG
ATGCATCCAACTTCCCTCCCTGCACTCCTCCTCCCCGACAGCTGCCTCAGGAATAG
GTGACCCTGCTGGCCATACTGTGAATGCTGTTCTTTCAGCTGTGACCAATTTGGGG
GTGCAGCTGAGAGTGAGGTGCTGTCAGGGCTGAAAAGGACGCACCTCATTCTACA
TTTGGATGTGATGAGGAGCAGGGCAGCCACCAGCTTGGATCCTAACTTACATTGGG
TGGTGCAACCTTTCTGACGGGGCCTGCCAAACTATCAGTAATTAGCCAGCCTAGAC
AATGTAGCATGACTAATTATTGCTGTGTCAAAGCAATTCAGGACAAGAAAAACAT
ATCTAAATGAGAATGTTTAGATGTGGTGAGAAAAGAGCTCTCACCACAATTTGAA
ACATATAAAAAGTTGAGTTCTTTGCAGGGGAGCCAACGGGGGCATTGAGGGAAGG
GCAAAATTCACCCCAGACAGCCAGTGCTTGATCTTGCCAAACAGTTTACACTTTAC
CTCCAAACCTGCAGTTCTAAGCTACTCCACTAAAATAATCATCCCATCCTCAAAGC
AGACCAACTCCAGCATCCCATCGGTTCTGGAGGGATTTGATGCAAACCTTTAAATG
CACCAACAGAGAGGTAAGCCAAGCTCTCAGGAGGGACAAAGATAGATTTCTATCT
TCAGTGCTCTTGGGGGATGTTTGCTCCTCATGAGTGTTTAAACAAGATCATTTCCA
GGTGATTTCTTAACATATAAATTCCTGCAAAATAGGACCAGCTTCTATAGGTAGCG
AATGTGTAGGTCATATAATTCCTTGTACAGATGTGAATGGATGCAGACTCTTTCCT
AATTTGCCTTGTAAGCCAAATAAAAGTCTGTCTCTACTGTC
GLP1R Protein;
(NP_002053.3; SEQ ID NO: 34)
MAGAPGPLRLALLLLGMVGRAGPRPQGATVSLWETVQKWREYRRQCQRSLTEDPPP
ATDLFCNRTFDEYACWPDGEPGSFVNVSCPWYLPWASSVPQGHVYRFCTAEGLWLQ
KDNSSLPWRDLSECEESKRGERSSPEEQLLFLYIIYTVGYALSFSALVIASAILLGFRHLH
CTRNYIHLNLFASFILRALSVFIKDAALKWMYSTAAQQHQWDGLLSYQDSLSCRLVFL
LMQYCVAANYYWLLVEGVYLYTLLAFSVLSEQWIFRLYVSIGWGVPLLFVVPWGIVK
YLYEDEGCWTRNSNMNYWLIIRLPILFAIGVNFLIFVRVICIVVSKLKANLMCKTDIKC
RLAKSTLTLIPLLGTHEVIFAFVMDEHARGTLRFIKLFTELSFTSFQGLMVAILYCFVNN
EVQLEFRKSWERWRLEHLHIQRDSSMKPLKCPTSSLSSGATAGSSMYTATCQASCS
ACVR1C cDNA;
(NM_145259.3; SEQ ID NO: 35)
AGTGGCAGGAGCGCCGCGCACCGCCAGCCGCAGGGGGCGTGGGATGGGGGCGGC
CGGGGAGGGGGGCGCCCACACTGACTAGAGCCAACCGCGCACTTCAAAAGGGTGT
CGGTGCCGCGCTCCCCTCCCGCGGCCCGGGAACTTCAAAGCGGGCCGTGCTGCCCC
GGCTGCCTCGCTCTGCTCTGGGGCCTCGCAGCCCCGGCGCGGCCGCCTGGTGGCGA
TGACCCGGGCGCTCTGCTCAGCGCTCCGCCAGGCTCTCCTGCTGCTCGCAGCGGCC
GCCGAGCTCTCGCCAGGACTGAAGTGTGTATGTCTTTTGTGTGATTCTTCAAACTTC
ACCTGCCAAACAGAAGGAGCATGTTGGGCATCAGTCATGCTAACCAATGGAAAAG
AGCAGGTGATCAAATCCTGTGTCTCCCTTCCAGAACTGAATGCTCAAGTCTTCTGT
CATAGTTCCAACAATGTTACCAAAACCGAATGCTGCTTCACAGATTTTTGCAACAA
CATAACACTGCACCTTCCAACAGCATCACCAAATGCCCCAAAACTTGGACCCATGG
AGCTGGCCATCATTATTACTGTGCCTGTTTGCCTCCTGTCCATAGCTGCGATGCTGA
CAGTATGGGCATGCCAGGGTCGACAGTGCTCCTACAGGAAGAAAAAGAGACCAAA
TGTGGAGGAACCACTCTCTGAGTGCAATCTGGTAAATGCTGGAAAAACTCTGAAA
GATCTGATTTATGATGTGACCGCCTCTGGATCTGGCTCTGGTCTACCTCTGTTGGTT
CAAAGGACAATTGCAAGGACGATTGTGCTTCAGGAAATAGTAGGAAAAGGTAGAT
TTGGTGAGGTGTGGCATGGAAGATGGTGTGGGGAAGATGTGGCTGTGAAAATATT
CTCCTCCAGAGATGAAAGATCTTGGTTTCGTGAGGCAGAAATTTACCAGACGGTCA
TGCTGCGACATGAAAACATCCTTGGTTTCATTGCTGCTGACAACAAAGATAATGGA
ACTTGGACTCAACTTTGGCTGGTATCTGAATATCATGAACAGGGCTCCTTATATGA
CTATTTGAATAGAAATATAGTGACCGTGGCTGGAATGATCAAGCTGGCGCTCTCAA
TTGCTAGTGGTCTGGCACACCTTCATATGGAGATTGTTGGTACACAAGGTAAACCT
GCTATTGCTCATCGAGACATAAAATCAAAGAATATCTTAGTGAAAAAGTGTGAAA
CTTGTGCCATAGCGGACTTAGGGTTGGCTGTGAAGCATGATTCAATACTGAACACT
ATCGACATACCTCAGAATCCTAAAGTGGGAACCAAGAGGTATATGGCTCCTGAAA
TGCTTGATGATACAATGAATGTGAATATCTTTGAGTCCTTCAAACGAGCTGACATC
TATTCTGTTGGTCTGGTTTACTGGGAAATAGCCCGGAGGTGTTCAGTCGGAGGAAT
TGTTGAGGAGTACCAATTGCCTTATTATGACATGGTGCCTTCAGATCCCTCGATAG
AGGAAATGAGAAAGGTTGTTTGTGACCAGAAGTTTCGACCAAGTATCCCAAACCA
GTGGCAAAGTTGTGAAGCACTCCGAGTCATGGGGAGAATAATGCGTGAGTGTTGG
TATGCCAACGGAGCGGCCCGCCTAACTGCTCTTCGTATTAAGAAGACTATATCTCA
ACTTTGTGTCAAAGAAGACTGCAAAGCCTAATGATGATAATTATGTTAAAAAGAA
ATCTCTCATAGCTTTCTTTTCCATTTTCCCCTTTATGTGAATGTTTTTGCCATTTTTTT
TTTGTTCTACCTCAAAGATAAGACAGTACAGTATTTAAGTGCCCATAAGGCAGCAT
GAAAAGATAACTCTAAAGTTAAGCATGGGCAGGAGTTGACTTCATCCAATCTCTAT
GTTATGTTTAATTTTATTTTGAAAGCAACACCTCAACTCATCTTTTTATTTAATAAG
GAAGAAATATATTACAAAAGTATAAAATAAGCTCTATAAAAATGTTATAGTCATTA
AGTTTTTATTTTACTTGAACCAAGAGCACATGAATGAACAGGAAAAGATGTAAAA
ACATTTTTTTCTGAGATGAAAACATATTAATTAAACATGCAAATTAGAGCATGCTA
TCTTTAGGTGATGCAATCTATGTTTCCCCCTTTTTAAGTTAGCAGGACTTTTTAAAA
ATAAATATTGCTCTAAACTTTAATATATCGAACGTGAGAGTGGAGCTGCTTAGTGG
AAGATGTAAGTGAGGTGGGTGTCCCATGTGCTTGGTCTCCCCTTCTGCTGTTCTCCT
GTTCTTCATAATCCACTACTGCAGCAGTCCCTGAACCACTAAACTTGTTCCTTTCAT
TTACAAAAGAGATACCTGACATCCTGAGACACTGAGAAATGTCCTGAAGTCACAC
AGCTAATGGCAGAACTGGCACTAGGTCCAAATCTTGTGATAATGAACACCGTAAG
GTTAGCTAGCTTCCTACTTTCCCTTGAATAGTGCTTTTCTCCCTATGTAATATCTTTT
ATTATGATATTTGTGGTTTAGAAGGCATATTGAGTTATTTTGCAGAATCATAATGG
ACCCGCACAAAATCTCAGAACCATATCTGTTGACATTTTTTCTCATAGAAATATCA
TGGTTACCCCATTTGTTAATGAGCATTAATGTTTTCTGAACACTTCCAAAGATTAAT
CAAACATAAATATTCATTGTCTGAAAATGTCTTTAAGATACAATTCAGAGGTCCCT
ATTTCCTTTGTACATACACACTTAGAAAGAAAAGACAGAAAAGGAAGAGGAAGGA
AGGAAATATTTTGAGAATATATTGAGAAGAATTAAGAAAACTCTTCAATGAAGTG
TTAACAACCAAACCCTACAGACGGTATCAGAAACAGCAAATAGATATTCCTCTAC
CCTTTCACAGTGAGTGAGTGAGTACAGAAGAATGCTCATGATAGTTTTGCCTTCAT
TCTACTTTCTGTGGACACAGAGTAATGAATATTTAATGGGACATTAAATATGCCCT
TCAAATCTATAATTTTACTTTGGTAAACGAGATTTAACATGATGTCTTTTATGCTCC
TAAAACATCTTTTTTCAAACTCCATTCCTTAGAACATTCTTCTACTGAGATGATCCA
AGACCAAAAGTGTTCTTTGGTACTTGCTTATAAAGTGATAGTACATGTTAGCATAT
AATGTATTTTGAAGAGTGAAGTAAATGCTATTGATAACAGTAAAAAAAAAAAAAA
AAACTAATAACAGTAAAGAAATGCTACTTGATTTTTTTTTAAACTGGATTGCTCAG
ATTACCTGATCGTGGTGGAACCCTTTTATTAAGAGGAGGGGAAACTTTTTACTATC
CCATATTTAACTGTTCTATAAAGCAAAGCACAGCTTGGGTAATAGCGTTCTGAAGG
ATATACTTCTGTATTTTCTCATAGAGTACAATTTAGTGATTATGCTTCATTTCACTA
TGGAAATATGTTACTGAATCTATCTTCATTTTACTGAGTTGAAATAAGGAAGGCAA
AAAACTGACAGCTATGGAGTTTGCGTGTACTTCCATACTCGTTAATGCTCTCATCC
ACTTATTAAATAATCATAGAGCACCCATATCTTGCTTGCCACAATATCGGGTACAA
GAGGGAATACAAAGATAGATAGGTCCTGCCCTCAGGGATTTTAAAGTCTAATTTG
GGAATGGGAATAGGGATGTGAGTGTGTGGGGGAAGAGATTAAATTGACAGATAAA
ATACGAAGTGGGATGTCTTGAGTTCTGCATGACAGTGGGTTTCTAGGATAGGTCTG
AAAATTGCTTTCATTTGCAACACATTTAGAAAGTAGCTTTATTTGGATATTACAGA
CAATCTAAATATATCATCAGTTTTTAAAAGTGCCTATGTGAAGTGATTTTTAAAAA
GAGCCTATGTGAAGGGGTAATCTTGCTTGTTCTTGTTACTAATTTCTCATAGATTGT
TTTTCTGCATATATAAGAACGAAATTATTTATTTACTATGGTTGTACGTGCCTCAAA
TAAACAAGAATGATATTTCCTGTTTTATTTACTTATGTTGGGTAAATATGCTTATTG
AATTTTTAAGAGAGGATTTTTTACCATCTCCATTTTTCTTGTCATTATGTTTTGTAGC
TTATTTGAGGGTGTCTAAATATAATTTCATATTTTATTGGTTCAACTTTCACTCTGA
AGAAATCCGTATGTTAGTACATTTTGAGGTATTTTTCTTGTTCTTGTGTTGTTTAACT
ATGACTCCTAACTGAGTAGTCTTATATTTCAATTACAAAATACATTTTTTAAGAAA
GGGAATAGAGCAGCAAAAATGATAAGGAAAATGTTAAAAGTTGTAATATTTCCTT
TACTCTTAACAGGATTATATATAGAACATGCTCACTTACAAAAATAGGATGATGAA
GTTTAGAGCATAAGGCAGGCTTCTTGTATATACTTATGCTGTCAAATGTTATATTG1
TTTTAATGGAGTCCCATTGTGTAATATTTATTTCTTTTACATTTTGTTATAAGCAAA
AAAAAAAAAAATTCTCCTTAGGTTATGTTCAGAGTATCAGTGTTCTTTACTCCTTAC
AGATATTTTGGCTTTGGGGTATAATACAAGACTTGGGAAAACACTATTATGAATTT
TCAGTACTGTATAAAGTGGTGATGGGATTTAAATGCAGCATCACTTTCTGAAAATA
AGAGAAACATTATTTGTTGTCAGTATTTCAGCATGAACTTGTTGCCTTGTAAATTTT
GCCTTTAAGTTTGTAATTGGTACAGATTCTGTTGTATGCTTTCTTCTATGTCTAAAA
TATTTGGCATGTCACATCTAGAATTCTTAATTTATGTTCTGACTTGAGAGTTAAGTG
AAACATGACTGTCGTGCACTATTTTAGGCATAGCACTTGCTTTTCATCTTTATACTT
AGTATCTGAAAAAATTAAAATTAGCTTTTATTTAAAAATGAAAATCTACAATAGAT
TCATTAGGTTAGAAGTTCCAGAATAATTTATTATTTTATTACACCTACTATTGTAGA
ATTACTAATAAAACAAAACAAAACTACTCCTATTTTCCTGGAATTTTGCCACCATG
TGACTTATTGGGGCAGAGAAAACTCAGGGTTGTCTTTGAGTCTGCACAAAAGCACC
AGGGAACCTGCTTAGCAAATCGTCTGAAAACAGGGAGCTGATGTTTGCCATTATAC
AAAGTTTGAGTAAACAACTTAAAATTGCTTGTTAGGGCATAGTCTTTGATTGAAAT
AAGTATGAGAATGTATTTGGCTAAATAAATGTATTTAAAATATACAAATTTTATTT
CCCACTGGAAAATTAAGAAAGTAGCAGTACCAAATGAATAAAAGCTGGCAGTTGA
TGTCTTCAATAATCATTCCTTTAAAATAAATTCACAAACACATCATTACAAGCTAC
TTAGAAATGTTTAGTATTCGTATCTTAAAATGGCACCATTGTGGTTTTCAAAGATAT
TTTAAACATCTTTTGTGAAACATACTATTTCTGTTTGACAATGCTTTGTTCTAGCTCT
GTGTGCTGATACCTACATGGAGTTTTTCTGCTATTTTAGTGAAAACAGTAATTCTTT
TATCTTGAGAGCTGCTTCTAAATAACAAAAAAGTTAATTGGAATGTAAGTTTTTAA
AAAATGTTAATATTAAATAGAATTTTTATAATATGGGCATTTTTCAAAACATTTTAA
TGAAAATATATAGATATTTGACTTTCTTTTTTTCATCTAGCCTTGCTTTATATTTCAT
TATTTTTCTCACTTTTTTTCTTAATATTCCCTCACTATCTTTCAACATTATCATTAGTT
CCTAATTTTAAAAATAACTCTTATTTAACTTAACCGTCTGGAATTTAGCCTTCTAAT
GAAAGAGAATCCCTTAGTACTCTCAGAGATTATATAAAGTTATTCCAATTTTGTGT
AGATGACGAAACCAAGGATCAGAGATTAAATGACTACTAGTTACTAGCAGAACTC
TTATGAAAGCCTGGTGGTGACACCCAGCACTGTTCCCTGCCATATCCCCAACTTTT
ACTGATTAAAAATTAATTCGCATGCATCTAAACCTACCTTGAATGCACACTAACGT
AATGTGCCATTCAATGATGATGATAAAATCCCACTTTTCTTTGGGTTTCTACTAAAA
TAATTTACTCACTCAGAGTGAGGTCAATGAGAAAAACTAAACTAGGCTAAGAAAG
AGTTGTAGAATGGTTGTTGAGCTAAGTAGGCAACCACTGGGGTGCTGATAAAATTA
ATGGATAAAATTTAGGAACTGTGAGAGTATAGAATTTCTTAGTGCAAGTAATGATT
AGAGAAGCTTCCTGACATCCCCCACCCTTTCTGTAAGGACTGTTCTTTCTCTTTGTA
CCTTGGAATGGGGGTGAAAGGTGATTTGATAGCTGAATTTGGGACTATGTCCAGTG
GGATATTATCTAACTTTTCTCTCTTTCTCTTTTTTTTCCCCTCAGTTTCTCATTAGTTT
GTCTTTGGCATTCATCTTCTTTTAGTGCATTAAAAAATGTTTGGCAGATCTCATCAA
TCCCAAGTCACTCTATAATTCCTGTATTTCTTTAGTTGTCGTTTAACCTGTCCAAAC
TTCTACACAATGAACTTCTTAACAAGATTTTAAGTTCTCTGTATAAGAGGTTTCACC
TCATAACTTCTCCAGTAATCCTTCATTTGGCACTATAGAGTATTTGTTAATGGCAGA
GATGATTTTTCTTTTAAAACCTAAAAAGACTAGCTGTTATTTGTATTCTAGCTTTTA
GCTAACATATAAAGAATGTCTACTTTTGCTTTAATGCTAAATTCCGCTTGAGAAAT
AGTAACTGGGAAAGACAATTTGAAATATATGCCCCATAATGTGATTGTTAAATTTA
TTTCTGCTGTTCCATACCATTGCTTTGTTTTGCTTTTGACAAACTAAGCCATTATGCT
ATTAGTTTGGAATATAATACTACAGCAAAATTAGGTAACAATCCCTATTTTAAAAT
TCCCCTAACAATAATAGAACTGCCAGCATACTTTTCTCTTTCAGTTGTAGATGAAT
ACATTCGAGAGAATATGAGCTGTATTTCATCCTAGATTTTAATATTTTCAGATGTGA
CTGTATTTCCTGATCATTGGTCCAAGTTGTCCTAAAAGAAATTTTTCTCTCCAGACC
TAACAGTTTTAACTGCAAGAGTTTACTGTGGGTTATGTTAATCTGAATTTTAATAGG
GCCACTAAGAATCTGAGTGCCTTAGGAGATTACCCTTATACCCACTGCCATCACAT
CCAGTCAGGCCTGTTGTGCTCTATATAAATCTTCCCAGCTGAGGGGCAGGTGCGGG
CTAAAATCCAACTGCAATTGGCTCCCAGACATAATTTTATATTTTACAGAGAAGCA
TCTTATTGGCTTATATGTGTTTAAAGAATGGTCTGGCTTATACATCTTCAGAAAATG
AGAATTAAAAAGTCAAAATAATTCTTGACATCTACAGATTGAACAAAGAACTTAG
AAGAAATAATACTTTATCTTTTCATCCTGGCATTCCTGAGAGAAGAGAAATTGATT
GTTTATCATGTTGGTTTAATTTTTCAACCCAGACAATCTGCAGCAAGGCACATGGA
CCCCAATTTTGATATCGTCCATACAGTTTTCATTCTATGCATGGAGCTAATTACTGA
CTTTGCCTGTAAAGAGAGGATTGTGTGCCTAAATTTTGTCTAACAAATGCAAGCGT
AGAATGACATTTACTAATATTTCTATTTCTTCCATAGGCTAAATAATAGTAACTAA
GTATTTTTAAGGACACAGCCCTTTTTTTCTCTTTATACAAAATGAGAGTATCTGAGC
CAAAATATTAAATTCTAGTTCTTTTCCGCAATGACTAGTGTCAAGCTCATGTACTCT
TCTGATTCTAGACTGGAGAAGATTATTCAAACTTGATCTGTGTTTCAGGTTTTTAAA
TGTCCTAAAAACAGAAAATTAGATTCAGATCTCAAAAAAGGAATTTTGGATTGACT
TTCAAAGTACTAATACTAATTATACTTTTCTTTTGGTAGCGTGACTCTTCTTATACC
TAAGAACATATTACAAATGTCAAAACCATTGCATTTTGACATTGCAAAACATGCCT
TGAACTCTTGAACTACTGTGAAAAGAATCACCGTTGTAAAGACTTTTTGTAAGCTA
GCTGATACTCTTAAGTATGTAAAAAGATTGTCTTTCAGCCGACAGGCCCAAAGGAA
TGTATATAAGGAAGGAATATGAAAAAATAAATTAGGTTTTAAAATAGGAATTGGG
CAATAAACTGTATCAAAAATATGTAGATGGATTTTAGTAGTTGTAATTTAAATGTG
GAAGGTGAAGAGAATTTCAAACTCCAAAGAGAAATGAATGATATTCAGATGTTTC
ATTAATTTCTAGTCTGTGAAAATATGCATTTTATAGTAATATGTATAGACTTATTTT
ATTTAGAAATAATAGTGTTTTAGAATTTATTAAAAACTCAGTGATAGCCTTTATAC
CAAAATGTTTAACTTTACCAACAGCAAGTCATAAAAGTATTTATTTTAAAGCTTTTT
AATATTATCGTGTAACTTTCATCTGTCTTCAGATGTAAATAATTATCTGCCTAAATG
TTATATTTTTATGTATGCATTTTCTGAAAATGTATTGTTTTGTAAAGTGGGAAAGAT
AATAAATCAAGCACTTCTTGCACTTGTTTCTGTGAAGCATATAGAACTCTATTTTAA
ATAAAGGAAGATGTGTCGTA
ACVR1C Protein;
(NP_660302.2; SEQ ID NO: 36)
MTRALCSALRQALLLLAAAAELSPGLKCVCLLCDSSNFTCQTEGACWASVMLTNGKE
QVIKSCVSLPELNAQVFCHSSNNVTKTECCFTDFCNNITLHLPTASPNAPKLGPMELAIII
TVPVCLLSIAAMLTVWACQGRQCSYRKKKRPNVEEPLSECNLVNAGKTLKDLIYDVT
ASGSGSGLPLLVQRTIARTIVLQEIVGKGRFGEVWHGRWCGEDVAVKIFSSRDERSWF
REAEIYQTVMLRHENILGFIAADNKDNGTWTQLWLVSEYHEQGSLYDYLNRNIVTVA
GMIKLALSIASGLAHLHMEIVGTQGKPAIAHRDIKSKNILVKKCETCAIADLGLAVKHD
SILNTIDIPQNPKVGTKRYMAPEMLDDTMNVNIFESFKRADIYSVGLVYWEIARRCSVG
GIVEEYQLPYYDMVPSDPSIEEMRKVVCDQKFRPSIPNQWQSCEALRVMGRIMRECW
YANGAARLTALRIKKTISQLCVKEDCKA
CMIP cDNA;
(NM_198390.2; SEQ ID NO: 37)
GGGGGCCCCGCCGCCCCAGCAGCCCAGGACAGCCCCCTCTCCCCGCCCCCAGCCC
CCTCCCCCGGCGCGGCCATGGATGTGACCAGCAGCTCGGGCGGCGGCGGCGACCC
CCGGCAGATCGAGGAGACCAAGCCGCTGCTGGGGGGCGACGTGTCGGCCCCCGAA
GGCACGAAGATGGGCGCCGTGCCCTGCCGCCGGGCTCTTCTGCTTTGCAACGGGAT
GAGGTACAAACTGCTGCAGGAGGGCGACATTCAGGTCTGTGTCATCCGGCACCCG
CGGACCTTTCTCAGCAAGATCCTCACCTCGAAATTCCTGAGGCGCTGGGAGCCGCA
CCACCTAACGCTGGCCGACAACAGCCTGGCGTCCGCCACGCCAACTGGGTACATG
GAAAACTCAGTCTCCTACAGCGCAATTGAAGACGTTCAGCTGCTGTCCTGGGAGA
ATGCCCCGAAGTACTGTTTACAGCTCACGATTCCTGGGGGAACTGTCTTACTGCAG
GCTGCCAATAGCTACCTGCGAGACCAGTGGTTCCATTCTCTGCAATGGAAGAAAA
AGATTTACAAATATAAGAAAGTGCTGAGTAACCCAAGCCGCTGGGAAGTTGTCTT
GAAAGAGATCCGGACCCTGGTGGACATGGCCCTGACATCCCCCCTGCAGGATGAC
TCCATCAACCAGGCCCCACTGGAAATCGTCTCGAAACTGCTCTCAGAGAACACAA
ACTTGACCACCCAGGAGCATGAAAACATCATTGTGGCAATCGCTCCTTTGCTGGAA
AACAACCACCCACCACCAGATCTCTGTGAATTCTTTTGCAAGCACTGCAGAGAGCG
GCCCCGGTCCATGGTGGTCATCGAGGTGTTCACCCCCGTGGTGCAGCGAATCCTCA
AGCATAACATGGACTTTGGGAAGTGCCCGCGACTGAGGCTGTTTACTCAGGAGTA
CATCCTTGCCTTGAACGAGCTCAACGCGGGGATGGAAGTGGTGAAGAAGTTCATT
CAGAGCATGCACGGCCCCACAGGGCACTGCCCCCACCCCCGGGTCCTGCCCAACC
TGGTGGCCGTGTGCCTGGCTGCCATCTACTCCTGCTATGAAGAGTTCATCAACAGC
CGCGACAATTCCCCAAGCCTGAAGGAAATCCGGAACGGCTGCCAGCAGCCGTGCG
ACCGGAAGCCCACTTTACCTCTGCGCCTTCTGCACCCCAGCCCGGACCTGGTGTCT
CAGGAAGCCACGCTGTCTGAGGCCCGGCTCAAGTCGGTGGTCGTGGCCTCCAGTG
AGATCCACGTGGAGGTGGAACGCACCAGCACTGCCAAGCCGGCGCTGACGGCCAG
CGCAGGCAACGACAGCGAGCCCAACCTCATCGACTGCCTCATGGTCAGCCCCGCC
TGCAGCACCATGAGCATCGAGCTGGGCCCCCAGGCCGACCGCACGCTCGGCTGCT
ACGTGGAAATCCTCAAGCTGCTGTCAGACTATGATGACTGGAGACCGTCTCTGGCC
AGTTTGCTTCAACCCATTCCATTCCCCAAAGAAGCTCTCGCACATGAGAAGTTCAC
CAAGGAACTGAAGTACGTGATTCAGAGGTTCGCCGAAGACCCCAGGCAAGAGGTC
CACTCATGCCTGCTGAGCGTGCGGGCCGGCAAAGATGGCTGGTTCCAGCTCTACAG
CCCCGGAGGGGTGGCCTGCGACGATGACGGGGAGCTGTTCGCCAGCATGGTGCAC
ATCCTCATGGGCTCCTGTTACAAGACCAAAAAATTCCTGCTCTCCCTGGCAGAAAA
CAAGCTGGGTCCCTGCATGCTCCTGGCACTGAGGGGGAACCAGACCATGGTGGAG
ATCCTGTGCTTGATGCTGGAATACAACATCATCGACAACAACGACACCCAACTGCA
GATCATCTCAACCCTGGAGAGCACAGACGTGGGGAAGCGCATGTACGAGCAGCTG
TGTGACCGGCAGCGGGAGCTGAAGGAGCTGCAAAGGAAAGGCGGGCCCACCAGG
CTAACACTGCCCTCCAAGTCCACAGACGCTGACTTGGCTCGTTTGCTGAGCTCCGG
CTCCTTCGGAAACCTGGAGAACCTCAGTTTGGCCTTCACCAATGTAACCAGTGCCT
GCGCCGAGCACCTCATCAAACTGCCTTCGCTCAAGCAGCTGAACCTGTGGTCCACT
CAGTTTGGAGACGCTGGCCTTCGGCTCCTGTCGGAACACCTCACCATGCTCCAGGT
GCTGAACCTGTGCGAGACCCCGGTCACAGACGCTGGCCTGCTGGCCCTGAGCTCCA
TGAAGAGTCTCTGCAGTTTAAACATGAACAGCACCAAGCTCTCAGCTGACACCTAC
GAAGATCTGAAGGCCAAGCTTCCCAATTTGAAGGAAGTGGACGTCCGCTACACCG
AAGCCTGGTGAAGCTCCCAGCTCAAGGCAGGAAGACGTTTGCAACCGCGACAAAA
TAACTCTTGACTAACAGCCGCAGAGCAGCCGGTCCTGGGGTCCCACCCTGGTGCCC
TGGCTGTGAGATAGATGGGGAGTCTTTCTGGGGGCGGAGGGGGGAGGGGGTGGGG
AGGGGGCCCACAAGCACGCCCAGCCCCCGCCGAATTCTTTTAGCTTCGTAATTGGA
ACCTTTGACCTGATCTAAAGTGGACTTTGTAGCAACAAGAGGAGCATCAGCGGGT
CGGGGAGGGGTTTGGGGGTGGGCTGGGGGGTGGGGGACCCTTTGTGGATTTTCTTT
GCCTTTGTGTTTGATGCCGTCGTGTGGGAAAAGTCAACTCCGATGCCACCATTGCG
GGCCGGACGAAGGATGCTTTCTTCCTAGAGGCTCCGAGCTGAGCTGCGAACTCGCC
CCCCGCCCTTGGGACAAGAAGACCCAGTCACATCACTGCACCCGTCCTGTGTCCTC
ACCATTGCTATGCAAAGTGATTCTTGTTGTACATAAGATTTAAATAATGCACCTATT
TAAGACATGTTGACAAATTGCGGGTCTGGGACCCGCCTCTTATTTATGAAGTCTTT
GACCGTCCCCCCCGCCCGACCCCACCGCCCTCCCGCCCCCACCTGGCGTGTAGTAC
TGTATAAACCAGTCAGCTGTCGGGTTAGTGGTAGTATTATTGTTATTTTTTTAAAGG
AAACAAACAGACAACAAAAAGAAGAAAAAAAAAAAGAACCTCCTTGGAAAAATT
AATTGCTTTTTCGTAATGGATTCTCTATGCTAATGCTCTCTCGTCTGTCTGTCTGTCT
GCCCACTCCCCCACCCACCACTGTGCGTTTCTGATTTCCAAATGTCTCCAACTCCCT
CACGAGGTGGGGCTCAGGCTGGAGGAGGAGGGATTAAGATCCCCTTGCTCCACTA
AGGCCCAAGCTCTTTCTCTCGGCACCTTTTAGACTTGAATGGGAGGCTGCTAACCC
GCCCTCTCCAGTCCACCCCGGTAAAAGAGCTGTTCCCCACCCCCAGGGAGCTCCTG
TCCCTGTCAGCCTTTGCTGTCCCCTGTCCCCAACGGAGACTCTGTCACCCCTGGGCT
CCCCCTGCCATCGTGTGCTTCACGTGGCCCCATGCATGCCCGCCTCTCTGCATGGTC
TCTTGGGAAAAGAGAGATGTGTCGCCTCCGCCAGTCCGACTGCCCTCCCCACCCCA
CCCCCGCCACCCCCCACATGTGACCACTGCAACGAAGACACTCCTTCTGTCCCCAC
CTGCTCCGAAGACAAACCAACCTCCGTTTCTTTTATAAACAGTCGGCTTTTTCTTAA
TAAGCCCTCACTGTACAGAACAGCCCGTTGATGGTTTATTTGGGGTCCCCCTCTCC
CCCCAGCCCTTTTTTCTGTTGGTTTAGCACAAATACTTCCCTCCTCCGGCACCTCCA
AACCTACCCCACAGTCAGTGTACTTGTTTTATATATATTTAATCTTATTCAATGGAA
ACCATGCTTTTGTCGTTTTATACTTTGCTAGGTAGACTTTATTACCCCCCCACTATG
CCCTCATTTTTTTAAAAAAGGAAAAAAAAAAGAAACTGGGTTCCAGTCTTAATTCA
TTTTCCGTGCCAGGTTTTATTTCGTGTGTGTGTGAGTGTGTTCTGTTTTGTGTTTTGT
TTTTTGTTGTTGTTTTTAGTTGTTTGGTTTTCTTTTCTTTCCCCCCTCCGGTCCCATAC
TTCACAGCACTCTGGTGCGGGAAGAAGCAGAAGCAAAAAAAATAAAAATAAAAA
AATAAATAAAAATAAAAAAAATAAAAAAGGAAAAAAAAAAAAGAAGAAACAAG
ACATGCCACCTTTCCCCTCGCACTGTTGCTTTTCCTGATGGTTAATACTACTGTCAC
GTAGCTGTGTACAAAGAGATGTGAAATACTTTCAGGCAAAAATAAACTGTAAGTG
ACTCATCAAAA
CMIP Protein;
(NP_938204.2; SEQ ID NO: 38)
MDVTSSSGGGGDPRQIEETKPLLGGDVSAPEGTKMGAVPCRRALLLCNGMRYKLLQE
GDIQVCVIRHPRTFLSKILTSKFLRRWEPHHLTLADNSLASATPTGYMENSVSYSAIED
VQLLSWENAPKYCLQLTIPGGTVLLQAANSYLRDQWFHSLQWKKKIYKYKKVLSNPS
RWEVVLKEIRTLVDMALTSPLQDDSINQAPLEIVSKLLSENTNLTTQEHENIIVAIAPLL
ENNHPPPDLCEFFCKHCRERPRSMVVIEVFTPVVQRILKHNMDFGKCPRLRLFTQEYIL
ALNELNAGMEVVKKFIQSMHGPTGHCPHPRVLPNLVAVCLAAIYSCYEEFINSRDNSP
SLKEIRNGCQQPCDRKPTLPLRLLHPSPDLVSQEATLSEARLKSVVVASSEIHVEVERTS
TAKPALTASAGNDSEPNLIDCLMVSPACSTMSIELGPQADRTLGCYVEILKLLSDYDD
WRPSLASLLQPIPFPKEALAHEKFTKELKYVIQRFAEDPRQEVHSCLLSVRAGKDGWF
QLYSPGGVACDDDGELFASMVHILMGSCYKTKKFLLSLAENKLGPCMLLALRGNQT
MVEILCLMLEYNIIDNNDTQLQIISTLESTDVGKRMYEQLCDRQRELKELQRKGGPTRL
TLPSKSTDADLARLLSSGSFGNLENLSLAFTNVTSACAEHLIKLPSLKQLNLWSTQFGD
AGLRLLSEHLTMLQVLNLCETPVTDAGLLALSSMKSLCSLNMNSTKLSADTYEDLKA
KLPNLKEVDVRYTEAW
DCAF7 cDNA;
(NM_005828.5; SEQ ID NO: 39)
GTCGTCCGTTCCCAAGCTGGTTTGAAACTAGGGGTCGGGCTCGGCCGTCGTCGTTG
TTTGTCGCCGCATCCCCGCTTCCGGGTTAGGCCGTTCCTGCCCGCCCCCTCCTCTCC
TCCCTTCGGACCCATAGATCTCAGGCTCGGCTCCCCGCCCGCCGCAGCCCACTGTT
GACCCGGCCCGTACTGCGGCCCCGTGGCCACCATGTCCCTGCACGGCAAACGGAA
GGAGATCTACAAGTATGAAGCGCCCTGGACAGTCTACGCGATGAACTGGAGTGTG
CGGCCCGATAAGCGCTTTCGCTTGGCGCTGGGCAGCTTCGTGGAGGAGTACAACA
ACAAGGTTCAGCTTGTTGGTTTAGATGAGGAGAGTTCAGAGTTTATTTGCAGAAAC
ACCTTTGACCACCCATACCCCACCACAAAGCTCATGTGGATCCCTGACACAAAAGG
CGTCTATCCAGACCTACTGGCAACAAGCGGTGACTATCTCCGTGTGTGGAGGGTTG
GTGAAACAGAGACCAGGCTGGAGTGTTTGCTAAACAATAATAAGAACTCTGATTT
CTGTGCTCCCCTGACCTCCTTTGACTGGAATGAGGTGGATCCTTATCTTTTAGGTAC
CTCAAGCATTGATACGACATGCACCATCTGGGGGCTGGAGACAGGGCAGGTGTTA
GGGCGAGTGAATCTCGTGTCTGGCCACGTGAAGACCCAGCTGATCGCCCATGACA
AAGAGGTCTATGATATTGCATTTAGCCGGGCCGGGGGTGGCAGGGACATGTTTGC
CTCTGTGGGTGCTGATGGCTCGGTGCGGATGTTTGACCTCCGCCATCTAGAACACA
GCACCATCATTTACGAAGACCCACAGCATCACCCACTGCTTCGCCTCTGCTGGAAC
AAGCAGGACCCTAACTACCTGGCCACCATGGCCATGGATGGAATGGAGGTGGTGA
TTCTAGATGTCCGGGTTCCCTGCACACCTGTCGCCAGGTTAAACAACCATCGAGCA
TGTGTCAATGGCATTGCTTGGGCCCCACATTCATCCTGCCACATCTGCACTGCAGC
GGATGACCACCAGGCTCTCATCTGGGACATCCAGCAAATGCCCCGAGCCATTGAG
GACCCTATCCTGGCCTACACAGCTGAAGGAGAGATCAACAATGTGCAGTGGGCAT
CAACTCAGCCCGACTGGATCGCCATCTGCTACAACAACTGCCTGGAGATACTCAGA
GTGTAGTGTTGGTGGCGCTGTGCCCACGAGGCAGGGGCTTTTGTATTTCCTGCCTC
TGCCCCACCCCCAAAGTAAGAAGAAACATGTTTCCAGTGGCCAGTATGTCTTTCAT
TGCTTTGCACCCACTGTTACCAGAAGCTGCTCTAGGAGTTCCTGGCCAGTCACCCC
ATCGCCCTCTGTGGCAGACTCAGTGCTGTGTGGCGCCTCCTCAGCCCAGGGCTGAG
TTTTAAGATTTTCTCTCCTTTCCTCTTCTCCTTTGGTTCCTCAATTAAAAAATGTGTG
TATATTTGTTTGTCAGGCGTTGTGTTGAGGAGCAGTTCACGCACTGGCTGTGTCTAT
TCCTCTGCCCAGGTGTCTCTGTTTGCTGCCCAAGGCAGCAGTTCATGTCTCGTCCAT
GTCCATGTTCGTGTTAGCACTTACGTGGGAACAAATACCAATTTGTCTTTTCTCCTA
GTATCAGTGTGTTTAACAAATTTTAACTTTGTATATTTGTTATCTATCAGGCTAATT
TTTTTATGAAAAGAATTTTACTCTCCTGCTTCATTTCTTTGTCTTATAGTCCTCCCTC
TTTGCACCTTCTTCTCTTCCCTCAGTGCCTGGAGCTGGTACTGGGCCCCTGGCCCCA
TGAGCAGTTTGCCTTCTTGAGTCACTGCCTGTGTAGTACATACCTGACCGGGAGTC
CAAACCACCTTGGTGCTCTGAAGTCCACTGACTCATCACACCTTTCTTAGCCTGGCT
CCTCTCAAGGGCATTCTGGGCTTGTAAACAGACATAGGAAGCCTCTGTTTACCCTG
AAGCACCACTGTCCAGCCCATTGGTTCCCACTGGCAGCATGGTAGAGCTGAGAGA
AACAGGCTCTCAGGGTACCTGACTTGAGGGGAATCGTTTCATGAAGCTGAACTTCA
AGCATATTTCCAGTACATTCTTTCAGAGTCTGTTTTTCCATCCAAATATAAGCCCCA
GGCCATTCCACTTAGTGTCTTTTCAATGATAGGCAAGAATGATATCTGAGTTGAAC
TTCGGTGCTTCTGTTGTTTGAGTTTACTGTGCCTGGTGGTATATTGGGCATTCTTTG
GATTGAGTGTTCTGAGGTGAGAGAGTCTTCCCGAGGCATCCTGTCTGTGCTTCCAA
CCCTGAACAAGACCTTACATGAGAGATGGACTGATGGACTGCGGCAATCCTGGGC
TGTCAAGTGGATAGATAGTTAAAAAGCATTATACTGTGGGTAATGAAAAGGGAGG
AAAAAAAAAGAAGGAAAAGGAATTATAGACCCCCAGGGTCAGCCAGTTAAGAGC
TCTACCCACACCTGTCAACCCCTCTCTCCCCCAGTTTAGGTTCTGAGCAGTATTGGA
CTTGTAGCCTGCAGTTGTCTTTTGACTTGCAGGCCGCAGGTGTCTTTCTGTTATGTG
AATGAGTTCCATGGAGGGGCATATGTGTGATTCCACCGTTAGATGAGCCCTTGGGG
CAGGCAGTTTGGGATGTGCTCTTGGGGGAAAGTTGGCTGTTTCCTTGCGCTCTGCT
CCTACCCGAAGGTTTTTAAGTCCCTCTGAATTGCTCATCTGAGATTAGTAGAGTAG
CAGGCCTGAAGGATGATGGTTTTGTCCTCTTTGGTTCTCACCTGCTTGAGAAGTAA
AACAGTAACTTTGTTCTTCTGGGCCCTTAAGCTTTTTTGGTTAAGTCTTCCTTTTCAG
AAGTAGATGTCATTATATGCCAAAAGTCTAGCTCTTTGCTTTACCATACAGGGACC
TGTCCCAAAGAAAAAGGCTCTTTTTTTAGCCAGCATATTTCCCCTTCTACCCTTTTA
CTTTGTTGTTCTGATTTTAGGACTCTGGCTGGCCATGTGCTTGTGGTTGCCTCTCCT
GCATTTGCCACTGGATTTGCACTGCATCGTTTGGAGATACAAAGCGAGCAGTTCTT
GGTCAGAACCCTCCTCTGCTTTTCATTGTGTTTGATAATGGTTACTGGGTCCTTCTC
TCAAGGGTAGCAAGGCCAAGCTGATGGCTGCTTGTTTAGGAGGCCATCAGTTCCTT
CCTGTGGAGAAGGGTCTGAAATGGAAGTCAGTGGTAGAAGGGGCTGGTCTGCTGG
GCAGGGCTTACATCCACTGAGTTCTAAGATTCCTTTCCTGATCTGCACCTACGCCTG
GTCTGTATGGTGGAATTTGTCAGCTGGAACTCAGAAACAACAACTTGAAAAAAAA
ATAATAATTAGAACATATTTGCATAAGATAGCTATTTACTCTGGAAACCAACAACT
TTTGAGATTTCCCTTGCCCTGTGGACGCCCAGCTCCTGTCATCCTTCCTTAGGTCCT
GCAGTACAGTCTTCCCCTGAATGCCACCGGGGACCCAGGGGGACTCCACCCCCCTA
AGCAAGCACACACATACTCACAGTTGATGAGTTGCTGGTCTTTGAGTCCCAGCTCT
CTTACCCTCCCTTTACTCCACCAGCCCGACGACCCATGACTGAGGAGGGGATTTCT
ACAGTCTCAGGATTTAGAAAGTCTGTAAGCCATCCATGCTCCAGAAAGCACCGATC
TGTTGTAGTTGCAAAAACAACTCTGTAATTTGTTGAGGTTCTCAAACTGACAGCCA
GCGAGACTGGGTGGGAGGCCCTGGATCTGTTCTCCCTGACTGCGGGAGGAGCAGC
CACTAGGACTTTAGCAGGAAGCCCACATGGAGGCTCCGCCAGGCTGTGGCCCAGC
TGGTGATGGCCCTTTTGCTCCTGGCAGCCTGAGGCACAGCTGCCTGTATTGTCCTC
ATCTGTTCTGACTGAAGGATGGAGGTGCTGAATAAATTAGGCCTCAGGCCTCTACC
ACCAGAGAGCTGGAGAATGGGTCCACGTCATTCAAGGACCTGAATTTTTTATGCTC
AGGAGCATTGGAATCCTCTTCTTCCAGGGAGGAATTAGCCTGCAAGGTTAGGACTT
GAAGAGGGAAGGTATTTAATAACTGGGCGAGGATGGGTGTGGTGGCTCACACCTG
TAATCCCAGCATTTTGGGAGGCTGAGGTGGCCAGATCCCAAGGTCAGAAGATCGA
GACCATCCTGGCTAACATGGTGAAACCCCATCTCTACTAAAAATACAAAAAAAAA
TTAGCCGGGGGTGGTGGCGGGTACCTGTAGTCCTAGCTACTTGGGAGGCTGAGGC
AGGAGAATGGCGTGAACCTGGGAGGTGGAGCTTGCAGTGAGCCAAGATCGTGCCA
CTGCACTCCAGCCTGGGCGACAGAGCAAGACTCCGTCTCAAAAAATAAAAAAAAA
AAAAAAATAGGTGAAAATTCCTTATAAATCCAGGATTGGCTCTGAGAGAACTGGC
TAAGATTCAGGAAGAAACAAAAAATTCAGAATCCTACAAGGTTTTGATGACAATT
AGGGCCAAAATTTTAGGAGGAGATGTAGGATGCAGGAGAAAATTAAAGTGTTTTC
TTTATATCAGAGGAGGAAATAGTAGAGGTCAGTGAAGGTCTGGGGTAGGGAAACA
TTCAGACTGTCCATTGCATGGCTGTGGAGTGAGACTGCCCTTAGCCTGGGTCAGCC
TTCCTGGGCCATAAATTGGGCATCCGTGATGCTAGGTAACTGTGGGAACAAAATG
ACAGCTTAGAGCAGCCATGGGTGATGTTTGGTGGTAAAAAACCTACAGGCGTTTG
GGGTCCCATGATTGTTCCAGACCATGACTCTTCCTGGTTGTGGGTTTGTTACAGAG
CAGGAGAAGCAGAGGTTATGACAGTTATGCAGACTTTCCCCCTCCTTTTTCTCTTTT
CTCTTCCCCTTGCTTTTCCACTGTTTCTTCCTGCTGCCACCTGGGCCTTGAATTCCTG
GGCTGTGAAGACATGTAGCAGCTGCAGGGTTTACCACACGTGGGAGGGCAGCCCA
GTACTGTCCCTCTGCCTTCCCCACTTTGAGAATATGGCAGCCCCTTTCATTCCTGGC
TTGGGGTAGGGGAGACCATTGAAGTAGAAGCCTCAAAGCAGACTTTTCCCTTTACT
GTGTGTACTCCAGGACGAAGAAGGAAGATCATGCTTGATACTTAGATTGGTTTTCC
CAGGGAAGAGGGCGGAGCAGAGCAAAGTCACTGTGAACCCTGGGCCAGGCCCTG
GCTGGGCCAGCTCCTGAGAGCGTCTCGTGTTGCAGACCCTTGCCCACTTCACCCAC
CTGCACCTTCTCCCCCTCTCACAGTGTCACTGCTGCTAATGGTCAAAGTCAAATGT
GTGGCCACATGGGATGGGCCAGGTCCTCTCAGGCTACTTTCTGGATGTCATTTTTA
AAATATGGAAACATGCAGGTGCCTTCCCAAAGAGGCTTGGACTGGTATATCCAAC
GAGAAACAAATAAGCTAAAGAAAGTTTAAACTCAAGAAGAAAGATGTTGACAGTC
TATGTAACAGCTGGAAAGTTTATAGGCACCCACCTTTGGGACAACCCAGTGATTAT
GAACATGTGATATCTACTATTTAAAAGAAATGTTCTCACCTTGGGTTGATTGTGGT
ATACCATGTGTTATGAAAATTGTTGAGCTGAAGCTTTGAATCGATTTAGTTGAGTC
TGACTCACTTGCTTTGGTTCCTGTGTATTTTACTACCCCTCTTGTCAGTGACCTTCCT
TCCCCACCCCACCCAGAGTGAATTTGTAGCATGATTGTATAAACCTCTATGTAGAA
AATGGAGATTTCTTGCTCTGAAATGTTAAGCTCTAACTGATCCATTTCTGTGTCCTT
TAGCCTAGTATGTCTGAACTTCCATTCTTGTTATATATTTAAACTTTCCCTCTATATT
ATAGGTTTTGTGGCATCCACGGTCAGGTGTAGAGGAAGCTGCCCCTTGCAGAACTG
TACTGTAATATTTTTCTTTTATAAATATTTTCACAGGACTGATTGTACACAGGGCTT
GTAATAAAATTTTAACACTGTGCTGTGAAACAACTATGGGGAATCTCCATTGAAGG
CTACTTCATGGGCACCTGAAAGTGGAGTGTTATAGCTATGACTTTCTATTTCTTGTT
TCCTAAGTAAATTAAACCTAATTTTCACCCTTTCATTCTGTTTCAGCCTCCTGTATA
AGAAGTACCGTATTTTCTGCCCATCATACTTTGTAATAAAACTTGAACATGTA
DCAF7 Protein;
(NP_005819.3; SEQ ID NO: 40)
MSLHGKRKEIYKYEAPWTVYAMNWSVRPDKRFRLALGSFVEEYNNKVQLVGLDEES
SEFICRNTFDHPYPTTKLMWIPDTKGVYPDLLATSGDYLRVWRVGETETRLECLLNNN
KNSDFCAPLTSFDWNEVDPYLLGTSSIDTTCTIWGLETGQVLGRVNLVSGHVKTQLIA
HDKEVYDIAFSRAGGGRDMFASVGADGSVRMFDLRHLEHSTIIYEDPQHHPLLRLCW
NKQDPNYLATMAMDGMEVVILDVRVPCTPVARLNNHRACVNGIAWAPHSSCHICTA
ADDHQALIWDIQQMPRAIEDPILAYTAEGEINNVQWASTQPDWIAICYNNCLEILRV
MAPK3 cDNA;
(NM_002746.3; SEQ ID NO: 41)
GAGGAGTGGAGATGGCGGCGGCGGCGGCTCAGGGGGGCGGGGGCGGGGAGCCCC
GTAGAACCGAGGGGGTCGGCCCGGGGGTCCCGGGGGAGGTGGAGATGGTGAAGG
GGCAGCCGTTCGACGTGGGCCCGCGCTACACGCAGTTGCAGTACATCGGCGAGGG
CGCGTACGGCATGGTCAGCTCGGCCTATGACCACGTGCGCAAGACTCGCGTGGCC
ATCAAGAAGATCAGCCCCTTCGAACATCAGACCTACTGCCAGCGCACGCTCCGGG
AGATCCAGATCCTGCTGCGCTTCCGCCATGAGAATGTCATCGGCATCCGAGACATT
CTGCGGGCGTCCACCCTGGAAGCCATGAGAGATGTCTACATTGTGCAGGACCTGAT
GGAGACTGACCTGTACAAGTTGCTGAAAAGCCAGCAGCTGAGCAATGACCATATC
TGCTACTTCCTCTACCAGATCCTGCGGGGCCTCAAGTACATCCACTCCGCCAACGT
GCTCCACCGAGATCTAAAGCCCTCCAACCTGCTCATCAACACCACCTGCGACCTTA
AGATTTGTGATTTCGGCCTGGCCCGGATTGCCGATCCTGAGCATGACCACACCGGC
TTCCTGACGGAGTATGTGGCTACGCGCTGGTACCGGGCCCCAGAGATCATGCTGAA
CTCCAAGGGCTATACCAAGTCCATCGACATCTGGTCTGTGGGCTGCATTCTGGCTG
AGATGCTCTCTAACCGGCCCATCTTCCCTGGCAAGCACTACCTGGATCAGCTCAAC
CACATTCTGGGCATCCTGGGCTCCCCATCCCAGGAGGACCTGAATTGTATCATCAA
CATGAAGGCCCGAAACTACCTACAGTCTCTGCCCTCCAAGACCAAGGTGGCTTGG
GCCAAGCTTTTCCCCAAGTCAGACTCCAAAGCCCTTGACCTGCTGGACCGGATGTT
AACCTTTAACCCCAATAAACGGATCACAGTGGAGGAAGCGCTGGCTCACCCCTAC
CTGGAGCAGTACTATGACCCGACGGATGAGCCAGTGGCCGAGGAGCCCTTCACCT
TCGCCATGGAGCTGGATGACCTACCTAAGGAGCGGCTGAAGGAGCTCATCTTCCA
GGAGACAGCACGCTTCCAGCCCGGAGTGCTGGAGGCCCCCTAGCCCAGACAGACA
TCTCTGCACCCTGGGGCCTGGACCTGCCTCCTGCCTGCCCCTCTCCCGCCAGACTGT
TAGAAAATGGACACTGTGCCCAGCCCGGACCTTGGCAGCCCAGGCCGGGGTGGAG
CATGGGCCTGGCCACCTCTCTCCTTTGCTGAGGCCTCCAGCTTCAGGCAGGCCAAG
GCCTTCTCCTCCCCACCCGCCCTCCCCACGGGGCCTCGGGACCTCAGGTGGCCCCA
GTTCAATCTCCCGCTGCTGCTGCTGCGCCCTTACCTTCCCCAGCGTCCCAGTCTCTG
GCAGTTCTGGAATGGAAGGGTTCTGGCTGCCCCAACCTGCTGAAGGGCAGAGGTG
GAGGGTGGGGGGCGCTGAGTAGGGACTCAGGGCCATGCCTGCCCCCCTCATCTCA
TTCAAACCCCACCCTAGTTTCCCTGAAGGAACATTCCTTAGTCTCAAGGGCTAGCA
TCCCTGAGGAGCCAGGCCGGGCCGAATCCCCTCCCTGTCAAAGCTGTCACTTCGCG
TGCCCTCGCTGCTTCTGTGTGTGGTGAGCAGAAGTGGAGCTGGGGGGCGTGGAGA
GCCCGGCGCCCCTGCCACCTCCCTGACCCGTCTAATATATAAATATAGAGATGTGT
CTATGGCTGA
MAPK3 Protein;
(NP_002737.2; SEQ ID NO: 42)
MAAAAAQGGGGGEPRRTEGVGPGVPGEVEMVKGQPFDVGPRYTQLQYIGEGAYGM
VSSAYDHVRKTRVAIKKISPFEHQTYCQRTLREIQILLRFRHENVIGIRDILRASTLEAM
RDVYIVQDLMETDLYKLLKSQQLSNDHICYFLYQILRGLKYIHSANVLHRDLKPSNLLI
NTTCDLKICDFGLARIADPEHDHTGFLTEYVATRWYRAPEIMLNSKGYTKSIDIWSVG
CILAEMLSNRPIFPGKHYLDQLNHILGILGSPSQEDLNCIINMKARNYLQSLPSKTKVA
WAKLFPKSDSKALDLLDRMLTFNPNKRITVEEALAHPYLEQYYDPTDEPVAEEPFTFA
MELDDLPKERLKELIFQETARFQPGVLEAP
NFIX cDNA;
(NM_001271043.2; SEQ ID NO: 43)
AGACGGACACTGTGCCGGGGCGAGCTGACAGGAGTTCACGGCTGCGATAGAACAT
GGAGATGTCATGGGCGCGACAGAGCCTGGCGGGGATACCAGCAGCGTGTGATGAG
TTCCACCCGTTCATCGAGGCACTGCTGCCTCACGTCCGCGCTTTCTCCTACACCTGG
TTCAACCTGCAGGCGCGGAAGCGCAAGTACTTCAAGAAGCATGAAAAGCGGATGT
CGAAGGACGAGGAGCGGGCGGTGAAGGACGAGCTGCTGGGCGAGAAGCCCGAGA
TCAAGCAGAAGTGGGCATCCCGGCTGCTGGCCAAGCTGCGCAAGGACATCCGGCC
CGAGTTCCGCGAGGACTTCGTGCTGACCATCACGGGCAAGAAGCCCCCCTGCTGC
GTGCTCTCCAACCCCGACCAGAAGGGCAAGATCCGGCGGATTGACTGCCTGCGCC
AGGCTGACAAGGTGTGGCGGCTGGACCTGGTCATGGTGATTTTGTTTAAGGGGATC
CCCCTGGAAAGTACTGATGGGGAGCGGCTCTACAAGTCGCCTCAGTGCTCGAACC
CCGGCCTGTGCGTCCAGCCACATCACATTGGAGTCACAATCAAAGAACTGGATCTT
TATCTGGCTTACTTTGTCCACACTCCGGAATCCGGACAATCAGATAGTTCAAACCA
GCAAGGAGATGCGGACATCAAACCACTGCCCAACGGGCACTTAAGTTTCCAGGAC
TGTTTTGTGACTTCCGGGGTCTGGAATGTGACGGAGCTGGTGAGAGTATCACAGAC
TCCTGTTGCAACAGCATCAGGGCCCAACTTCTCCCTGGCGGACCTGGAGAGTCCCA
GCTACTACAACATCAACCAGGTGACCCTGGGGCGGCGGTCCATCACCTCCCCTCCT
TCCACCAGCACCACCAAGCGCCCCAAGTCCATCGATGACAGTGAGATGGAGAGCC
CTGTTGATGACGTGTTCTATCCCGGGACAGGCCGTTCCCCAGCAGCTGGCAGCAGC
CAGTCCAGCGGGTGGCCCAACGATGTGGATGCAGGCCCGGCTTCTCTAAAGAAGT
CAGGAAAGCTGGACTTCTGCAGTGCCCTCTCCTCTCAGGGCAGCTCCCCGCGCATG
GCTTTCACCCACCACCCGCTGCCTGTGCTTGCTGGAGTCAGACCAGGGAGCCCCCG
GGCCACAGCATCAGCCCTGCACTTCCCCTCCACGTCCATCATCCAGCAGTCGAGCC
CGTATTTCACGCACCCGACCATCCGCTACCACCACCACCACGGGCAGGACTCACTG
AAGGAGTTTGTGCAGTTTGTGTGCTCGGATGGCTCGGGCCAGGCCACCGGACAGC
CCAACGGTAGCGGCCAGGGCAAAGTCCCGGGGTCATTTTTGCTACCGCCGCCGCCT
CCAGTGGCCAGACCTGTGCCCCTTCCTATGCCTGATTCCAAATCCACCAGCACTGC
CCCAGACGGCGCCGCCTTGACTCCTCCATCACCTTCATTCGCAACGACAGGCGCCT
CCTCTGCCAACCGGTTTGTCAGCATCGGACCCCGGGACGGCAACTTTCTGAACATC
CCACAGCAGTCTCAGTCCTGGTTCCTCTGATAAGATCGACAAAAGAAACAACAAA
ATGAGAAGAAGAGGTTCCTCGAAAGGGGGGAGAAGAAATTTTGAGAATGGAAAA
ATCCCCCAGCCCAGCCCAGCCCCACCGAAAAGCAAAAATTACACGTCGTCAGCCA
CTCAGCCCTTCTCTCCTCCAGCCCGGGGACCCCCGCGGGCCCCAGAAGCAGCCCAG
TTCTCAGAGAGCCCTTGGAAGGGGTCTCGGTGGAGCTGTGCACCAGCAGCCAAGC
AGAAAGAAACACGCGACATGGACTCTGTCAAGTAGAGGACAGAAAGCAAGAAAG
GATGCAGAACTGCCTTCCTCCCCCTGACCCCGCCCCGGCCTTCTGGGGAAGGAACA
AAGTCCCCAAACAAAGCAACCAGCACAATTCTGAAGGGGCCTGGCCTCCACCCTC
ACCCCTTCCTAGGGGAACCCCACCCTCCACACAGCCGGAGCTGCCCTAGGGAGCCT
GGAGGGCCAGCTTGTAAAGATGATGGGGTTTAGATCCCTCAGGCTCTCCCCTCCAG
ACTCCGCCCTTCCCTCCCTCCCTCCCTCCCTCCCTCTCTGCCAAGGCTCCAGCTTCTT
CCCCCAGCTGCTCCCGACCAGGAGGGGGAGAGCAGCCTCCACTTACCCCACCCCA
CCCTTGGGCTAAAAGCCCCCAGGCGGGCAGGGGGTGACCCCTGGAGCTAGTTGCG
TGTCCCAGAATGGAGGGTGTTCTGACACCCCACCCTGAGCCGCAAGAGCAGTCCT
GGGGCCCTGGACCCCTCTGTACAGTCCGTAGGAAAAAGTCGGAATGCTCTCGACG
GCCTCGTCCCAGCCTGGGACAGGCCCCCTTTCCCCTCTCTCTGCAGGCCAGGAGGG
CCTCCTTCCTGCCACGAGGGAGGGGAGTCGGGCCCCAGGTCGCCCCCGCCCCCAG
CCCTGCATGCAGGTGCCCTCGCTCCGCCCCATCAGTTCCTGCCCCTGCCCCTCATGC
AGACTGCCCTGCTGGGGCCGGGCCGGAGGGTGGAGCAGAAAGGGGACCCCGGAG
CCGAGCGAGGAGGACCAGGCAGCCGCCGCTGCCGCGCTAAGCCACCACCTGCGCT
TAGGTAGGCGTCCTGCTCGCCGACTTTCAGTTCCTTGGGAGGGTGTTGGGTGTCGT
CCTTTTCAAAAGTGTTTTGGAGCTTTCTGTGCCCCCCGACTTTCCCCCGCCTCCCCG
CCCCCCACGTGGCCACTTTTCTCTGGATTTTAGCTGTAATGTCTTTACTCTTTATTTA
GGGGTGGGGCATTCATTGTTTGGGTCTTTTGCTGTTGGAATGGGAACTCCTCCTCC
ATTTGAGCAACTTGGGAACAATTTGGTAACACACCACAGGAAGTAGCTCTCCCCCC
CAGCCCCCTCCTCCCTCAAGGGAGGGTTGGGGGGCCTGTCCAGAGGGTCTTCAGA
AGCCCCCCTGGGAGGGAGGGGAGGATGAGCACGCCCAGCTCCCCTCCAGGGTGTG
ACTTGGCCCCTCTGGCTTGTCTTTCTGTGCCTTACTCCTCCTCCTGCGTCTCCCGTTC
CTGGCCCCTTCTTGAGTCCTTGTGCCTCTCTCTTTCTCTCTCTTTCTTAATTGTATGA
AAACACAAAGCACAGGTCAGGATCCTCTGAGAGAAAATCAACATTGCACCACGTA
GGGGTGGGCTATGGGCTGTATTTATTGTGAATCTAGTTTGTGAGGCTGTGGCCCCG
AGCTGGCGGAGGGAGGGAAGAGGAGGGAGTGACGGGAGGGGAGGAGGTCAGCG
ACCTGGGGCCGTAGCGGCAGGCGAACGGTGCCTGCTACCCAGCTGGAAGCCACAA
GGTGGCTGGCTCCAGGGGCGGCTTTTGTTGGAAGTTGAGTGAAGCCCTCCCCCTGT
CCTCAGCGTGCAGCCCTAGAGGACCCCAGGGCTGAGGGGCAGTGGATCCTGCGGG
AGTCTCCCGGGGCGTGGGGAGTAAGGCCCCGGGGGTGGGGGGCCGGGTGGGCCG
GGCGTGACGCGCGGTCAAAGTGCAATGATTTTTCAGTTCGGTTGGCTAAACAGGGT
CAGAGCTGAGAGCGAAGCAGAAGGGGCTCCCTGTCCGGCCCACGTGCCCTTTCCC
TCGACGACAGTCGAGGGCTCGGGCTCTGTGGGACTGTGGGAGCTAGGGTCTGCGG
GGCGCCTGCCCGGGCGAGGTCGGAAGCTGCAGGCCAGCTGGGCCCGGGCCGGAGC
GTGCCCGGCGGGGCTGCCCGGGCGGGCAGGGGGTGGGGGCTGCTCCTTTCCCAAG
TGGTGTTGTGAGGGGCAATGAGGGCAACAGGAGATGTGGGGACGTGTTAGGAGAG
AAAAAAAAAAAAACAAAAATATATATGGGGGAAATTAACTTTTTTTTTTCATTGAA
CCAAGTGCAATGCATCAGAGAGTTTTCCTATCTTTGTATGTTAAGAGATTAAGAAA
AAAAAATTCTATTTTTGTTGTAATGTCCTCGCGGCTCTGGGGACGCTAAAAGAACC
GGGCCTGCCCCGCCCTGCGCGGGGATAACGAAAGCTGAGTGTTTTTCCCTTTTTTTT
GTTCGTTTTTAGTTTTTTTTTTTTTAAGTCGTTTTCCTGCGTTGACGAGGATGATCTG
GGGTTTTTATTTGTTTCGTCGTTCGTTCTGTTTCGGTGGGAGGGCTGAAGGAAACGT
TCACATTTTAGAGTTTAAAAAAAACACCTCGACATTTAAAAAATCAACCAACACA
AGATCAAAAAGGAAAAGGACGAGAGAAAAATTATTTTTAAGATAATTAAACATAA
AACCCTGGTGCTTCTTACATTATAAAGTACGTTTTAAAGAACCCACAAACTATTAT
ACATAAGTTTATGAATCAATTAAATATCCTGCACTTGTTAGGAATACGCATATCCC
TTCTTTGTTGAGTTTAACGGAACGGGACAGCGGCGTGCCCCCGGCGGCTGGACTGC
TCCGGCCGCGGGTCTCCCCGGGCGCCCCTCCCTGGGGCCCAGCACCCCTCCTCGCC
CCATCCCCGTCCGGGTACGGGGGCGCGGCAGGGGTCCCCGGCCCCTCCCCCGCAG
AGGTCAATGCCAACGAACAAACGTCCCCTCCCTCCCTCCCTCTCCGCCCCGAGCGC
CCTTCTTTGAGCCAGACGCCAACTTGACCCTCACCAGCATTATCAGGAGCGCGCTC
AGCAAGTTGGTAGTTTCCTCCCCCCTTTCCCGGCGCCCCTCCCGCCCCCATTCAACA
TCTCTCATCCTATCCCCGACCCCCTCCGGGGAACACCGGGAAGGCTCGACGCTCCA
GGACAGGACCAGCCACGCTGACAGGTCGATTTGCCCAGGCCCGCGCCCGCACGCA
CGCACGCACACGGCCCCGCACACAGCCCCGCCCCACCCCGCAACCAGCCCTGTCG
ACTGCCTTATACACCCGCCCCCGCGCTGGCCGGCCGACCTAGTGCCTTGTTCTCAC
CCCCGTGCTGGCGGAGCGGACGCCGCGCTCTGGGTCCCAGAGGGGCCGGGTGGCT
CAGACGACCCACCACTCCCCCACCCTGACCGTGCTGAACAGACCCCCCCACACGA
GAGAAAATAAAGGAGCAATAAAGTCACGAGAACTTTCGTCCCCCAATCGAGAGCC
CGAGGGGCACCCCAGCCCCGCCTCTGCTCCCCCCCACCCCACCCACCCTCGGGGCG
CCCCCCTCCCCCCGCAAGCCAGCCTGGGCCAGCCCCGCTTCGGCCCCTCCCGGGAG
ATCCGTGCGCCCGACCAGCACCAGCATCGCGGACCGCAAAGGCCGCCCGTCCCGT
CAAACAAGTTTCTTCTTAGGCTAAGAAACGCAGTATATACGAGTATCTCTATATAT
AGTACTAATGGATTTGGTGTGCTTCCCCCTTAGCGTCCCCCTCCCTCTGCTCCTCCT
CCTTCAGCCTGGTCTCCCCCTCTTCTCTGCCCTCCACCCCCGTCTCTGCACTGAGAT
ACATAAGAAACAAGGGTAGTTTACTGTCTGTTTTGTTTTCTGGGTTTTCAGTGTCCT
AGCGGAATGCAAGTAGGCAGCCAGCCCGTCTGTTCCCTCTCCGCCCCGCCCCGCCC
CGCCCCCGTCACTGCGCTTCTGTTATACCATCTTTGCCTGACTCTCTCCGGCTTCTC
CATTGAATGGCTAATGTGTATGTGAAATAAAGAAATAAAGAAAAACAAACGCGA
NFIX Protein;
(NP_001257972.1; SEQ ID NO: 44)
MEMSWARQSLAGIPAACDEFHPFIEALLPHVRAFSYTWFNLQARKRKYFKKHEKRMS
KDEERAVKDELLGEKPEIKQKWASRLLAKLRKDIRPEFREDFVLTITGKKPPCCVLSNP
DQKGKIRRIDCLRQADKVWRLDLVMVILFKGIPLESTDGERLYKSPQCSNPGLCVQPH
HIGVTIKELDLYLAYFVHTPESGQSDSSNQQGDADIKPLPNGHLSFQDCFVTSGVWNV
TELVRVSQTPVATASGPNFSLADLESPSYYNINQVTLGRRSITSPPSTSTTKRPKSIDDSE
MESPVDDVFYPGTGRSPAAGSSQSSGWPNDVDAGPASLKKSGKLDFCSALSSQGSSPR
MAFTHHPLPVLAGVRPGSPRATASALHFPSTSIIQQSSPYFTHPTIRYHHHHGQDSLKEF
VQFVCSDGSGQATGQPNGSGQGKVPGSFLLPPPPPVARPVPLPMPDSKSTSTAPDGAA
LTPPSPSFATTGASSANRFVSIGPRDGNFLNIPQQSQSWFL
HAPLN4 cDNA;
(NM_023002.3; SEQ ID NO: 45)
AGTCTTAACCGGGTGTGCGGGGAGCGCAGTCCGGGTGCGTAGGGGCCGCTCGGCG
GGGGCCGCGCGGGCAAGATGGTGTGCGCTCGGGCGGCCCTCGGTCCCGGCGCGCT
CTGGGCCGCGGCCTGGGGCGTCCTGCTGCTCACAGCCCCTGCGGGGGCGCAGCGT
GGCCGGAAGAAGGTCGTGCACGTGCTGGAGGGTGAGTCGGGCTCGGTAGTGGTAC
AGACAGCGCCTGGGCAGGTGGTAAGCCACCGTGGTGGCACCATCGTCTTGCCCTG
CCGCTACCACTATGAGGCAGCCGCCCACGGTCACGACGGCGTCCGGCTCAAGTGG
ACAAAGGTGGTGGACCCGCTGGCCTTCACCGACGTCTTCGTGGCACTAGGCCCCCA
GCACCGGGCATTCGGCAGCTACCGTGGGCGGGCTGAGCTGCAGGGCGACGGGCCT
GGGGATGCCTCCCTGGTCCTCCGCAACGTCACGCTGCAAGACTACGGGCGCTATGA
GTGCGAAGTCACCAATGAGCTGGAAGATGACGCTGGCATGGTCAAGCTGGACCTG
GAAGGCGTGGTCTTTCCCTACCACCCCCGTGGAGGCCGATACAAGCTGACCTTCGC
GGAGGCGCAGCGCGCGTGCGCCGAGCAGGACGGCATCCTGGCATCTGCAGAACAG
CTGCACGCGGCCTGGCGCGACGGCCTGGACTGGTGCAACGCGGGCTGGTTGCGCG
ACGGCTCAGTGCAATACCCCGTGAACCGGCCCCGGGAGCCCTGCGGCGGCCTGGG
GGGGACCGGGAGTGCAGGGGGCGGCGGTGATGCCAACGGGGGCCTGCGCAACTA
CGGGTATCGCCATAACGCCGAGGAACGCTACGACGCCTTCTGCTTCACGTCCAACC
TGCCGGGGCGCGTGTTCTTCCTGAAGCCGCTGCGACCTGTACCCTTCTCCGGAGCT
GCGCGCGCGTGTGCTGCGCGTGGCGCGGCCGTGGCCAAGGTGGGGCAGCTGTTCG
CCGCGTGGAAGCTGCAGCTGCTAGACCGCTGCACCGCGGGTTGGCTGGCCGATGG
CAGTGCGCGCTACCCCATCGTGAACCCGCGAGCGCGCTGCGGAGGCCGCAGGCCT
GGTGTGCGCAGCCTCGGCTTCCCGGACGCCACCCGACGGCTCTTCGGCGTCTACTG
CTACCGCGCTCCAGGAGCACCGGACCCGGCACCTGGCGGCTGGGGCTGGGGCTGG
GCGGGCGGCGGCGGCTGGGCAGGGGGCGCGCGCGATCCTGCTGCCTGGACCCCTC
TGCACGTCTAGGCTGGGAGTAGGCGGACAGCCAGGGCGCTTGACCACTGGTCTAG
AGCCCTGTGGTCCCCTGGAGCCTGGCCACGCCCTTGAAGCCCTGGACACTGGCCAC
ATTCCCTGTGGTCCCTTACAAACTAACTGTGCCCCTGGGGTCCCTGAAGACTGGCT
AGTCCTGGCAGAACAGTACTTTGGAGTTCCCTGGAGCCTGGCCAGCCCTCACCTCT
TCTGGATAGAGGATTCCCCCAACTCCCCAACTTTCTCCATGAGGGTCACGCCCCCT
GAGGACCTCAGGAGGCCAGCAGAACCCGCAGGCTCCTGAAGACTGGCCACGCCTC
CTGAGACCACTTGGAAACAGACCAACTGCCCCCGTGGTCGCCTGGTGGCTGGACC
CCCGGGATTGACTAGAGACCGGCCGTACACCTTCTGCATCTCACTGGAGACTGAAC
ACTAGTCCCTTGCGGTCACGTGGGACACTGGGCGCCTCCTCCTCCCCCTCCTCCTCA
CCTGGAGAGACTACAGGAACTTCAGGGTCACTCCCCGTGGTCACATGGAGGTTGT
GGGCCGAGGCGCTTATTTTCCCTTATGGTGACCTGAGTCCTGGAGACTCCCATTCT
CCCCCTCTCCCTGAGAGTCCCCTGCAGTTTCTGGGTAACAGGGCACACCCCTCTAG
TTTCATGGGCGAGCACCCCCATCTGCCACCTCAGACTGACACACAGCCAGCTGGCT
CACTTACTGGGGGCCACGTCCCACCCCTCAGATATTTCTTTGAAGGGAGAGCAAAC
CCACCCTGTCCTCTGACGTCCCTTTCCCAACTGTCACCAAACAGACCATCTTCCCAG
GCCTGGGGACCGGTAAGATCCATGTCACTAGTTATGCAGAGCAGTTGCCTTGGGTC
CCACTGTCACCAAGGCAACCAGTCCTGCTGCTACCTGTCACCTAGAGTCACACACC
CCTTCCCTCATCAGGCACACCCATGAAGACAGTGCCTCCCTCCTCCAGCTGTAACC
ATGGATACCACACATTTCTCATCTCATTGGCCCCCACCCCAGAGACCTCCACCTCA
ACTTCTGGCTGTCCCTACCCTGACTCACCGCCATGGAGATCACCCTCCCCGAAGCT
GTCGCCAGGGTGACCCAACATCCAGTTCTCCGGCTCTCACCATGGAAACAAACTGT
CCCTGTCCCCAGGCCCACTCCAGTTCCAGACCACCCTCCATGCTCCACCCCCAGGC
GGTTTGGACCCCACCACTGTTGCCATGGTGACCAAACTCTGGAGTCCGAGGTAACA
GAACACCTGTCCCCCTAGGCTTTTCCTTGTGGACAACGGGGCCCTGTTCACCAAGC
TGTTGCCATAGAGACTGTCAACGTTGTCCTCATGACAACCAGACTTCCAGTTCTCA
GGAACTTCTCATTGTGGGCCAGAAGTCCTGGGTGCCTCCTACTAGGGCTACCCTAC
TGCACCCCATCAGGGGCCTGATGGCTGCCCCTTCCCCAGACAGGGCTGGACTTCTG
GAGCTGCTAAGCCACCCTCCGTTTGCACGTTAACTCTATGCCGGATAGCAGCTGTG
CACGAGACAATCTTGCAACACCCGGGCATGTTTGTCGTCGTCCTACAAATGAGGAA
ACCGAGCCTATGGCGTGCCCTGGTCTGTTGAGATATGCAAGCACTGAGCTCCTCTT
TTGTCCTCTGAGACCCCATCTCCATTCTCACCCAGTTCCTCTCTCCTTCCCTGACCC
CCACCCACATTTCCCTCCTTAGAGATCCAGGAGGGATGGAATGTTCTTTAAAATTC
AACACCCACCAGGCTCTAAGCGGCGATCTGTGCTAAGAGGTCAGGACCCAGCCGA
AGTCCTCGGCGTTGACAGGCAGCTGGGGGGACATGATCCATGGACAAGGCCATCC
CGGCCGTGGGAGACCCCAGTCCCGAAGTCTTGCCTGCAGGAGTACTGGGGTCCCC
CTGGGGCCCTCTTTACTGTCACGTCATCTCTAGGAAACCTATCTCTGAGTTTTGGGA
CCAGGTCGGTTTGGGTTTGAATTCTGCCTCTTCTTGCTCACTGTGTGACCAAGTGAC
AAACTCCTTCTGAACCTGTGTTCTCCCACTGTACCAGGGCTGTTCTGTGGTCCCCGT
GAGTGCCAAGCATACAGTAGGGGCTCAATAAATCCTTGTTTCTTTTGATGAATGAG
AAAATGAGGCAGCCAGTGGGTAATTCCTGTATAAATGCACTTTGGTAGATAAGAT
GTTACAAGCTTGGGGGGCTTGGGGTTTTTTTTGTTTTGTTTTTTTGAGATGGAGTCC
TGCCCTGTCGCCCAGGCTGGAGTGCAGTCGTGCAATCTTGGCTCACTGCAACCTCT
GCCTCCCGGGTTCAAGTGATTCTCCTGCCTCAGCCTCCCGAGTAGCTGGGATTACA
GGTGCCTGCCACCACACCCGGCTAATTTTTGTATTTTTAGTAGAGACAGGGTTTCA
CCATGTTGGCCAGGCTGGTCTTGAACTCCTGACCTCAGGTGATCTACCCACCTCGG
CCTCCCAAAGTGTTGGGGTTACAGGTGTGAGCCACTGCGCCTGGCCGGGCTTGATT
TTTATTCTCTTTGGAAGTGGGGTCCTTCATTCCTCCCCCACCTCCCCAATCTCTTGCT
CCTCTTTCTTCCCCCATCCTGTGCCCACTGTCTCCCTTTAGCCACCCACCATGGGTC
TGCTCCTTGTCAACTCTGTCCTGACTGGGTCATTGACAAGATCCAGGGCATGAAAT
TCGGGAAACTGGAAGGGGGGTTCCTGTACCAGGAAGGGAGAGACATTACAAACTT
TCCCTGACATGAATATGTGGGCTGAGGGCAGGGGCTGAGGAAGCACTGGAAGTTT
CTAGGATACAACAAAGCATGAAGAGAAAAAATAGTGTAAGGTCCTGACCGTGCAC
AGTGGATCATGCCTATAATCCCAGCACTTTAAGAGACCAAGGTGGGAGGATTGCTT
GAGCCAGAAGGTCGAGGCTGCAGTGAGCCATGATTGTACCACTGCACTCCAGCTT
GGGCGACGGAGTGAGACCCTGTCTCAAAAAATAAAAATAAGTAAATCTCC
HAPLN4 Protein;
(NP_075378.1; SEQ ID NO: 46)
MVCARAALGPGALWAAAWGVLLLTAPAGAQRGRKKVVHVLEGESGSVVVQTAPGQ
VVSHRGGTIVLPCRYHYEAAAHGHDGVRLKWTKVVDPLAFTDVFVALGPQHRAFGS
YRGRAELQGDGPGDASLVLRNVTLQDYGRYECEVTNELEDDAGMVKLDLEGVVFPY
HPRGGRYKLTFAEAQRACAEQDGILASAEQLHAAWRDGLDWCNAGWLRDGSVQYP
VNRPREPCGGLGGTGSAGGGGDANGGLRNYGYRHNAEERYDAFCFTSNLPGRVFFLK
PLRPVPFSGAARACAARGAAVAKVGQLFAAWKLQLLDRCTAGWLADGSARYPIVNP
RARCGGRRPGVRSLGFPDATRRLFGVYCYRAPGAPDPAPGGWGWGWAGGGGWAGG
ARDPAAWTPLHV
CYHR1 cDNA;
(NM_138496.1; SEQ ID NO: 47)
AATGGGGACCTGGAACCTGGGCTTACTAGAGTGCCGCGCGTAGGGCTCCAGGTCG
CTGGCTTCTGCGCTTCCTTCCTCTCCAAAGTTGAGTATCTCCTATCTGTGTCCTCGT
ACATACTGCCGCCTGAGGTGCCATGGCCCCCAAGCCGGGGGCCGAGTGGAGCACA
GCCCTGTCCCATCTGGTGCTGGGAGTGGTGTCTCTGCACGCAGCCGTGAGCACAGC
CGAGGCAAGTCGAGGGGCTGCTGCTGGCTTCCTGCTCCAGGTCTTGGCTGCCACCA
CCACGCTGGCCCCAGGGCTGAGCACACACGAAGACTGCCTTGCTGGAGCCTGGGT
GGCCACCGTCATCGGCCTTCCCCTTCTGGCCTTCGATTTCCACTGGTGTACTAATGG
TCACTTGATGTGCGCTGGCTGTTTTATCCACCTACTAGCAGATGCCCGGCTGAAGG
AGGAGCAGGCCACGTGCCCCAATTGTCGTTGTGAGATCAGTAAGAGCCTCTGCTGC
CGGAACCTGGCCGTGGAGAAAGCCGTGAGCGAGCTGCCTTCAGAGTGTGGCTTCT
GCCTGCGCCAGTTTCCCCGCTCCCTCCTGGAGAGGCACCAGAAAGAGGAATGCCA
GGACAGGGTAACCCAGTGCAAGTACAAACGCATCGGCTGCCCATGGCACGGCCCC
TTCCATGAGCTGACGGTGCACGAGGCTGCGTGCGCCCACCCGACCAAGACAGGCA
GTGAGCTGATGGAGATCCTGGATGGGATGGACCAGAGCCACCGCAAGGAGATGCA
GCTGTACAACAGCATCTTCAGCCTGCTCAGCTTCGAGAAGATTGGCTACACAGAGG
TCCAGTTCCGGCCGTACCGCACAGACGACTTCATCACGCGCCTGTACTATGAGACG
CCCAGGTTCACAGTGCTGAACCAGACGTGGGTCCTGAAGGCTCGAGTCAACGACT
CGGAGCGTAACCCCAACCTGTCCTGCAAGCGTACGCTCTCCTTCCAGCTCCTCCTC
AAGAGCAAGGTCACGGCACCGCTGGAGTGCTCCTTCCTGCTGCTCAAGGGCCCCTA
CGACGACGTGAGGATCAGCCCCGTCATCTACCACTTTGTCTTCACCAACGAGAGCA
ACGAGACGGACTACGTGCCACTGCCCATCATTGACTCCGTGGAGTGCAACAAGCT
GCTGGCTGCCAAGAACATCAACCTGCGGCTCTTCCTGTTCCAGATACAGAAGTAGG
GCGGGGCCTCAGGATGTCCGAGGAGCCCACGGGCGGCATCCCAGCACCGCTGCCC
TGTCCACCTGGCTGGCAGCTGCTTCACAGGACTATCTGATCACTTTAGCAAAGGAG
GAGAACAAACGAAGCCAACACAGGGCAAGTCTGCATGCGTGCGCGACGGGGCCC
CCGCCTCCGGCTCACCCCCCCGACCCCTGCCTCCCCTCCTTCCGAGGGCCGCCAGA
GGCTGGGCTGACCCGAAGAGGAGACGGTGCACCAGGCGCCCCGAGGCTCAGAGA
CGGTGGCAGCAAGGAGGCCGAGAGGCACAGCGACCCTGCCCCAGCCCTTCTGTGC
AGTCAGGCGGCGGTGCTGCTCCATCCCTGCGGGTTCCGGCGGGGCGCGGGGGCCT
TGCTGACATCAGACGGGATATCCGAATATCTGATAGCAATTAAAAGGCAGCCTTGT
TTCGT
CYHR1 Protein;
(NP_612505.1; SEQ ID NO: 48)
MAPKPGAEWSTALSHLVLGVVSLHAAVSTAEASRGAAAGFLLQVLAATTTLAPGLST
HEDCLAGAWVATVIGLPLLAFDFHWCTNGHLMCAGCFIHLLADARLKEEQATCPNCR
CEISKSLCCRNLAVEKAVSELPSECGFCLRQFPRSLLERHQKEECQDRVTQCKYKRIGC
PWHGPFHELTVHEAACAHPTKTGSELMEILDGMDQSHRKEMQLYNSIFSLLSFEKIGY
TEVQFRPYRTDDFITRLYYETPRFTVLNQTWVLKARVNDSERNPNLSCKRTLSFQLLL
KSKVTAPLECSFLLLKGPYDDVRISPVIYHFVFTNESNETDYVPLPIIDSVECNKLLAAK
NINLRLFLFQIQK
C9orf3 (AOPEP) cDNA;
(NM_001193329.1; SEQ ID NO: 49)
GAGACTGAAAGGAACCATAATTTGTGACATCAGTTGTTTTCTTTGATAAGCAGCTA
TTTATGATTCTGGAAGATTAAGGCAGATAGGAAACCCCATCTGAGATTTTAATAAA
TCCCTCAAACAATAAACCACATCATGGACATACAGCTGGACCCTGCCAGAGATGA
CCTGCCTCTCATGGCCAACACCAGCCACATACTTGTGAAGCACTATGTACTGGATT
TGGATGTGGATTTTGAAAGTCAAGTCATTGAGGGGACCATAGTGCTTTTCCTCGAG
GATGGAAACAGATTCAAGAAACAGAATAGCTCTATTGAGGAAGCCTGCCAATCAG
AATCAAACAAAGCCTGCAAATTTGGGATGCCTGAACCCTGCCATATTCCCGTGACA
AATGCAAGGACCTTCTCATCTGAAATGGAATATAATGATTTTGCAATCTGTAGTAA
AGGTGAAAAAGATACTTCTGATAAAGATGGTAACCATGACAACCAGGAACATGCT
TCTGGGATTTCTAGCTCAAAGTACTGCTGTGACACAGGGAATCATGGGAGTGAGG
ATTTTTTGCTAGTGTTGGACTGCTGTGATTTATCTGTGTTAAAAGTCGAGGAGGTGG
ATGTTGCTGCTGTGCCAGGTCTGGAAAAATTTACAAGGTCTCCTGAGCTCACGGTT
GTTTCTGAGGAGTTCAGGAATCAGATTGTACGTGAACTTGTGACTTTGCCTGCAAA
TCGTTGGAGGGAGCAGTTAGACTATTACGCTCGCTGCAGCCAGGCTCCTGGCTGTG
GGGAACTCCTCTTTGACACTGACACTTGGAGCTTGCAGATAAGGAAGACAGGGGC
TCAGACAGCTACTGACTTTCCTCATGCTATCAGGATATGGTACAAAACTAAACCTG
AAGGGCGATCGGTTACATGGACCTCAGACCAGAGTGGCAGGCCATGTGTTTATAC
TGTGGGATCTCCCATAAACAACAGGGCCCTTTTTCCATGCCAGGAGCCACCCGTTG
CCATGTCAACATGGCAGGCTACAGTTCGAGCAGCTGCATCTTTTGTTGTTTTAATG
AGTGGGGAAAATTCTGCCAAACCAACGCAGCTTTGGGAAGAGTGCTCAAGCTGGT
ATTACTATGTAACTATGCCAATGCCAGCCTCCACCTTCACAATTGCAGTGGGATGC
TGGACAGAAATGAAGATGGAGACATGGTCATCAAATGATTTGGCAACAGAGAGAC
CCTTCTCACCTTCTGAGGCCAACTTCAGGCATGTTGGTGTTTGCAGTCACATGGAA
TACCCCTGCCGCTTCCAGAATGCTTCTGCCACCACCCAGGAGATCATTCCTCATCG
GGTCTTTGCCCCTGTGTGCCTCACGGGTGCCTGCCAAGAGACCCTTCTGCGGCTGA
TCCCTCCTTGCCTCTCAGCAGCACATTCTGTTCTGGGAGCACACCCGTTCTCTCGGC
TGGATGTTCTCATCGTCCCTGCCAACTTTCCAAGTCTGGGGATGGCCAGCCCACAC
ATCATGTTCCTCTCTCAGAGCATCTTGACAGGAGGGAACCATCTCTGTGGGACCCG
CCTCTGCCATGAAATTGCCCATGCCTGGTTTGGCCTAGCCATCGGGGCCCGAGACT
GGACGGAGGAGTGGCTGAGTGAAGGCTTCGCCACTCACTTGGAGGATGTGTTTTG
GGCCACAGCACAGCAGCTGGCCCCCTATGAGGCCCGGGAGCAGCAGGAGCTGAGG
GCTTGTCTGCGCTGGCGTCGCCTCCAGGACGAGATGCAATGCTCCCCCGAGGAGAT
GCAGGTGTTAAGACCCAGTAAAGACAAAACTGGCCACACAAGTGACTCGGGAGCA
TCTGTTATCAAGCATGGACTTAATCCGGAGAAGATCTTCATGCAGGTGCATTATTT
AAAGGGCTACTTCCTTCTTCGGTTTCTTGCCAAAAGACTTGGAGATGAAACCTATT
TTTCATTTTTAAGAAAATTTGTGCACACATTTCATGGACAGCTGATTCTTTCCCAGG
ATTTCCTTCAAATGCTACTGGAGAACATTCCAGAAGAAAAAAGGCTTGAGCTGTCT
GTTGAAAACATCTACCAAGACTGGCTTGAGAGTTCCGGAATACCAAAGCCGCTGC
AGAGGGAGCGTCGCGCCGGGGCGGAGTGCGGGCTTGCGCGGCAAGTGCGCGCCG
AGGTCACGAAATGGATTGGAGTGAACCGGAGACCCCGAAAACGGAAGCGCAGGG
AGAAGGAAGAGGTGTTTGAAAAGCTTCTTCCAGACCAGCTGGTCTTGCTTCTGGAG
CATCTCTTGGAGCAGAAGACTCTGAGCCCCCGAACTCTGCAAAGCCTCCAGAGGA
CATACCACCTCCAGGATCAGGATGCAGAGGTTCGCCATCGGTGGTGTGAACTCATT
GTTAAGCACAAGTTCACGAAAGCCTACAAAAGTGTGGAGAGGTTCCTTCAGGAGG
ATCAGGCCATGGGTGTGTACCTCTACGGGGAGCTGATGGTGAGTGAGGACGCCAG
ACAGCAGCAGCTCGCCCGTAGGTGCTTCGAGCGGACCAAGGAGCAGATGGATAGG
TCCTCAGCCCAGGTGGTGGCCGAAATGTTATTTTAACGAGGAAAGACCACAGCAA
GATTCTTTCATTCGTCTCCTCCTAGCCTGGGGGACCAGGCTCGAACTGACCCTGGA
CATCAAAGGAGGGATTATGTGGCTGCTAAAGCCATCGGCCCACAGCCCTGTTCAC
ATCTTGGTGCTTCTCTTTCCCAGAGGCTGGTCCCAGCCAGGCACACACAAAAGGCA
GATTCTCGTAAACGCAGCCTCCCTCCCTGGAGGCTGCCTCCTGCCCTGGATCTGGA
GTGGAGCTGCTCTGAGATTTTGAGTTCTTCTGCAGAGATGATTAAATATATCCAAG
AGACATTGGAAAACCTGCTGAACATTTTACATTGGTCTGCTCAGCACATGGCTGGA
TGCGGATATTTCTATAATTCCAGAAAGTCACACAGCTCCTCTGTATGAGACCAGTG
GGCGCCATTTAAAAGAACAGGATGAGAATCTAAGATATATTATTAATAAATGTAA
TGGATTTTTTTTTTGTATACGTGTTTGCTTCTAAATTTCATACTGTTTAAAAATAATA
AAGGCCAGGTGCGGTGGCAAAAAAAAA
C9orf3 (AOPEP) Protein;
(NP_001180258.1; SEQ ID NO: 50)
MDIQLDPARDDLPLMANTSHILVKHYVLDLDVDFESQVIEGTIVLFLEDGNRFKKQNS
SIEEACQSESNKACKFGMPEPCHIPVTNARTFSSEMEYNDFAICSKGEKDTSDKDGNHD
NQEHASGISSSKYCCDTGNHGSEDFLLVLDCCDLSVLKVEEVDVAAVPGLEKFTRSPE
LTVVSEEFRNQIVRELVTLPANRWREQLDYYARCSQAPGCGELLFDTDTWSLQIRKTG
AQTATDFPHAIRIWYKTKPEGRSVTWTSDQSGRPCVYTVGSPINNRALFPCQEPPVAM
STWQATVRAAASFVVLMSGENSAKPTQLWEECSSWYYYVTMPMPASTFTIAVGCWT
EMKMETWSSNDLATERPFSPSEANFRHVGVCSHMEYPCRFQNASATTQEIIPHRVFAP
VCLTGACQETLLRLIPPCLSAAHSVLGAHPFSRLDVLIVPANFPSLGMASPHIMFLSQSI
LTGGNHLCGTRLCHEIAHAWFGLAIGARDWTEEWLSEGFATHLEDVFWATAQQLAPY
EAREQQELRACLRWRRLQDEMQCSPEEMQVLRPSKDKTGHTSDSGASVIKHGLNPEK
IFMQVHYLKGYFLLRFLAKRLGDETYFSFLRKFVHTFHGQLILSQDFLQMLLENIPEEK
RLELSVENIYQDWLESSGIPKPLQRERRAGAECGLARQVRAEVTKWIGVNRRPRKRKR
REKEEVFEKLLPDQLVLLLEHLLEQKTLSPRTLQSLQRTYHLQDQDAEVRHRWCELIV
KHKFTKAYKSVERFLQEDQAMGVYLYGELMVSEDARQQQLARRCFERTKEQMDRSS
AQVVAEMLF

### Modulators of Lipotoxic Disease-Related Genes

For a subset of the above-recited genes herein identified as high value lipotoxic disease-related target genes, small molecule modulators have been described in the art. Such modulators of T2D lipotoxic genes include: PQ912 (Vivoryon), which has been described as an isoQC inhibitor (QPCTL inhibitor); fatty acid glycolates (PAM inhibitors); M2698 (a PAM inhibitor); 2-pyridine-3-yl-methylene-indan-1,3-dione (PRT4165; identified to be a small molecule inhibitor of PRC1-mediated histone ubiquitylation (Ismail et al. J. Biol. Chem. 288: 26944-54)); BC1753 (DCAF7 inhibitor); PD98059 (MAPK3/MAPK1 inhibitor; Di Paola et al. Int. J. Immunopathol. Pharmacol. 22: 937-50); arphamenine A (inhibitor of aminopeptidases, such as C9orf3 aka aminopeptidase 0); and TDZD-8 (an ALDOA inhibitor).

miRNA hsa-miR-4458 has also been characterized as regulating ACVR1C (see U.S. Patent Application No. 2014/0221463). Oligonucleotide antagonists of hsa-miR-4458 (anti-miR-4458 antagomirs) are therefore also contemplated for regulation of ACVR1C.

In addition to the above agents, it is expressly contemplated that nucleic acid agents, such as siRNA (siRNA-mediated downregulation), antisense oligonucleotides, and CRISPR (e.g., CRISPR-mediated up- or down-regulation), can be employed to modulate the above-recited genes identified herein as high value T2D/lipotoxic targets. Further, antibody therapies which target the protein product of T2D/lipotoxic genes are also contemplated herein, e.g., to lower cell and/or tissue levels of T2D/lipotoxic associated proteins.

Oligonucleotide inhibitors of the above-listed genes are therefore also explicitly contemplated for use herein, including, e.g., antisense oligonucleotides, dsNA agents (including, e.g., siRNAs, hairpin oligonucleotides, etc.), and sgRNA (e.g., implementing CRISPR/Cas9 as a delivery approach for sequence-specific inhibition or upregulation of a specific gene). In certain embodiments, sequence-specific oligonucleotide inhibitors of a target gene of the instant disclosure are delivered as naked oligonucleotides, as modified oligonucleotides (e.g., as GalNAc conjugates), within lipid nanoparticles (LNPs), or as components of other art-recognized delivery modalities for oligonucleotide therapeutics.

Protein levels of the product of a target gene can be quantitated in a variety of ways well known in the art, such as immunoprecipitation, western blot analysis (immunoblotting), ELISA or fluorescence-activated cell sorting (FACS). Antibodies directed to the product of a target gene can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of a high value target gene inhibitor which achieves a half-maximal inhibition of symptoms) as determined in cell culture. Such information can be used to more accurately determine useful doses in humans. Levels in plasma may be measured, for example, by high performance liquid chromatography.

### Pharmaceutical Compositions

Agents of the present disclosure can be incorporated into a variety of formulations for therapeutic use (e.g., by administration) or in the manufacture of a medicament, by combining the agent(s) with appropriate pharmaceutically acceptable carriers or diluents, and may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols.

Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Examples of such diluents include, without limitation, distilled water, buffered water, physiological saline, PBS, Ringer's solution, dextrose solution, and Hank's solution. A pharmaceutical composition or formulation of the present disclosure can further include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

Further examples of formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249: 1527-1533 (1990).

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink.

Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J Pharmaceutical Sciences 66 (1977):1-19, incorporated herein by reference. The salts can be prepared in situ during the final isolation and purification of the compounds of the application, or separately by reacting a free base or free acid function with a suitable reagent, as described generally below. For example, a free base function can be reacted with a suitable acid. Furthermore, where the compounds to be administered of the application carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

Formulations may be optimized for retention and stabilization in a subject and/or tissue of a subject, e.g., to prevent rapid clearance of a formulation by the subject. Stabilization techniques include cross-linking, multimerizing, or linking to groups such as polyethylene glycol, polyacrylamide, neutral protein carriers, etc. in order to achieve an increase in molecular weight.

Other strategies for increasing retention include the entrapment of the agent, such as a high value T2D target gene modulator, in a biodegradable or bioerodible implant. The rate of release of the therapeutically active agent is controlled by the rate of transport through the polymeric matrix, and the biodegradation of the implant. The transport of drug through the polymer barrier will also be affected by compound solubility, polymer hydrophilicity, extent of polymer cross-linking, expansion of the polymer upon water absorption so as to make the polymer barrier more permeable to the drug, geometry of the implant, and the like. The implants are of dimensions commensurate with the size and shape of the region selected as the site of implantation. Implants may be particles, sheets, patches, plaques, fibers, microcapsules and the like and may be of any size or shape compatible with the selected site of insertion.

The implants may be monolithic, i.e. having the active agent homogenously distributed through the polymeric matrix, or encapsulated, where a reservoir of active agent is encapsulated by the polymeric matrix. The selection of the polymeric composition to be employed will vary with the site of administration, the desired period of treatment, patient tolerance, the nature of the disease to be treated and the like. Characteristics of the polymers will include biodegradability at the site of implantation, compatibility with the agent of interest, ease of encapsulation, a half-life in the physiological environment.

Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use. The polymers will be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. By employing the L-lactate or D-lactate, a slowly biodegrading polymer is achieved, while degradation is substantially enhanced with the racemate. Copolymers of glycolic and lactic acid are of particular interest, where the rate of biodegradation is controlled by the ratio of glycolic to lactic acid. The most rapidly degraded copolymer has roughly equal amounts of glycolic and lactic acid, where either homopolymer is more resistant to degradation. The ratio of glycolic acid to lactic acid will also affect the brittleness of in the implant, where a more flexible implant is desirable for larger geometries. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the individual instant disclosure. Hydrogels are typically a copolymer material, characterized by the ability to imbibe a liquid. Exemplary biodegradable hydrogels which may be employed are described in Heller in: Hydrogels in Medicine and Pharmacy, N. A. Peppes ed., Vol. III, CRC Press, Boca Raton, Fla., 1987, pp 137-149.

### Pharmaceutical Dosages

Pharmaceutical compositions of the present disclosure containing an agent described herein may be used in accord with known methods, such as oral administration, intravenous administration as a bolus or by continuous infusion over a period of time, by intramuscular, intraperitoneal, intracerobrospinal, intracranial, intraspinal, subcutaneous, intraarticular, intrasynovial, intrathecal, topical, or inhalation routes.

Dosages and desired drug concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles described in Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the agents of the present disclosure, normal dosage amounts may vary from about 10 ng/kg up to about 100 mg/kg of an individual's and/or subject's body weight or more per day, depending upon the route of administration. In some embodiments, the dose amount is about 1 mg/kg/day to 10 mg/kg/day. For repeated administrations over several days or longer, depending on the severity of the disease, disorder, or condition to be treated, the treatment is sustained until a desired suppression of symptoms is achieved.

An effective amount of an agent of the instant disclosure may vary, e.g., from about 0.001 mg/kg to about 1000 mg/kg or more in one or more dose administrations for one or several days (depending on the mode of administration). In certain embodiments, the effective amount per dose varies from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, and from about 10.0 mg/kg to about 150 mg/kg.

An exemplary dosing regimen may include administering an initial dose of an agent of the disclosure of about 200 μg/kg, followed by a weekly maintenance dose of about 100 μg/kg every other week. Other dosage regimens may be useful, depending on the pattern of pharmacokinetic decay that the physician wishes to achieve. For example, dosing an individual from one to twenty-one times a week is contemplated herein. In certain embodiments, dosing ranging from about 3 μg/kg to about 2 mg/kg (such as about 3 μg/kg, about 10 μg/kg, about 30 μg/kg, about 100 μg/kg, about 300 μg/kg, about 1 mg/kg, or about 2 mg/kg) may be used. In certain embodiments, dosing frequency is three times per day, twice per day, once per day, once every other day, once weekly, once every two weeks, once every four weeks, once every five weeks, once every six weeks, once every seven weeks, once every eight weeks, once every nine weeks, once every ten weeks, or once monthly, once every two months, once every three months, or longer. Progress of the therapy is easily monitored by conventional techniques and assays. The dosing regimen, including the agent(s) administered, can vary over time independently of the dose used.

Pharmaceutical compositions described herein can be prepared by any method known in the art of pharmacology. In general, such preparatory methods include the steps of bringing the agent or compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, Poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxy ethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macadamia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (0 absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of an agent (e.g., a high value T2D target gene modulator) described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Drugs provided herein can be formulated in dosage unit form for ease of administration and uniformity of dosage. It will be understood, however, that the total daily usage of the agents described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The agents and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the agent or pharmaceutical composition described herein is suitable for topical administration to the eye of a subject.

The exact amount of an agent required to achieve an effective amount will vary from subject to subject, depending, for example, on species, age, and general condition of a subject, severity of the side effects or disorder, identity of the particular agent, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of an agent (e.g., a modulator of a high value target gene identified herein) described herein.

As noted elsewhere herein, a drug of the instant disclosure may be administered via a number of routes of administration, including but not limited to: subcutaneous, intravenous, intrathecal, intramuscular, intranasal, oral, transepidermal, parenteral, by inhalation, or intracerebroventricular.

The term “injection” or “injectable” as used herein refers to a bolus injection (administration of a discrete amount of an agent for raising its concentration in a bodily fluid), slow bolus injection over several minutes, or prolonged infusion, or several consecutive injections/infusions that are given at spaced apart intervals.

In some embodiments of the present disclosure, a formulation as herein defined is administered to the subject by bolus administration.

A drug or other therapy of the instant disclosure is administered to the subject in an amount sufficient to achieve a desired effect at a desired site, and/or in the subject as a whole, determined by a skilled clinician to be effective. In some embodiments of the disclosure, the agent is administered at least once a year. In other embodiments of the disclosure, the agent is administered at least once a day. In other embodiments of the disclosure, the agent is administered at least once a week. In some embodiments of the disclosure, the agent is administered at least once a month.

Additional exemplary doses for administration of an agent of the disclosure to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 μg/kg/day, at least 100 μg/kg/day, at least 250 μg/kg/day, at least 500 μg/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 μg/kg/day, and less than 500 μg/kg/day.

In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of an agent (e.g., a high value T2D target gene modulator) described herein. In certain embodiments, a dose described herein includes independently between 1 mg and 3 mg, inclusive, of an agent (e.g., a high value T2D target gene modulator) described herein. In certain embodiments, a dose described herein includes independently between 3 mg and 10 mg, inclusive, of an agent (e.g., a high value T2D target gene modulator) described herein. In certain embodiments, a dose described herein includes independently between 10 mg and 30 mg, inclusive, of an agent (e.g., a high value T2D target gene modulator) described herein. In certain embodiments, a dose described herein includes independently between 30 mg and 100 mg, inclusive, of an agent (e.g., a high value T2D target gene modulator) described herein.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

It will be also appreciated that an agent (e.g., a high value T2D target gene modulator) or composition, as described herein, can be administered in combination with one or more additional pharmaceutical agents (e.g., therapeutically and/or prophylactically active agents), which are different from the agent or composition and may be useful as, e.g., combination therapies.

Combination therapies explicitly contemplated for the instant disclosure include, e.g., administration of a high value T2D target gene modulator with insulin, other T2D therapeutic agent, or with other pharmaceutical agent.

The agent or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease described herein. Each additional pharmaceutical agent may be administered at a dose and/or on a time schedule determined for that pharmaceutical agent. The additional pharmaceutical agents may also be administered together with each other and/or with the agent or composition described herein in a single dose or administered separately in different doses. The particular combination to employ in a regimen will take into account compatibility of the agent described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Dosages for a particular agent of the instant disclosure may be determined empirically in individuals who have been given one or more administrations of the agent.

Administration of an agent of the present disclosure can be continuous or intermittent, depending, for example, on the recipient's physiological condition, whether the purpose of the administration is therapeutic or prophylactic, and other factors known to skilled practitioners. The administration of an agent may be essentially continuous over a preselected period of time or may be in a series of spaced doses.

Guidance regarding particular dosages and methods of delivery is provided in the literature; see, for example, U.S. Pat. Nos. 4,657,760; 5,206,344; or 5,225,212. It is within the scope of the instant disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

### Kits

The instant disclosure also provides kits containing agents of this disclosure for use in the methods of the present disclosure. Kits of the instant disclosure may include one or more containers comprising, e.g., a FFA array for screening, and/or an agent for modulating a gene identified herein as a high value T2D target gene.

Where a therapeutic agent is included in the kit, the instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). The container may further comprise a second pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Cell Culture

MIN6 cells were purchased (Addex Bio, #C0018008) and cultured as described previously (21). In short, cells were maintained in DMEM with 4.5 g/l glucose, supplemented with 10% FBS (fetal bovine serum, Life Technologies, #26140079), 100 U/ml penicillin and 100 μg/ml streptomycin (#15140 Invitrogen) and 55 μM beta-mercaptoethanol (Sigma, #M6250). MIN6 cells were cultured and used for experiments up to passage 30 and regularly tested for mycoplasma.

### Human Pancreatic Islet Cells

Human pancreatic islets from a deceased 54 yo female donor (Islet Research Resource ID: SAMN15400953) were purchased from the Integrated Islet Distribution program, dissociated, and cultured as described previously (Walpita and Wagner. Curr. Protocols in Chem. Biol. 6: 157-168). In brief, 384 well plates (Perkin Elmer, CellCarrier Ultra, #6057300) were coated in ECM 2-3 days before the start of the experiment. HTB-9 cells (ATTC, 5637) were seeded at 10K/well into the 384 well plates and grown to confluence. All culture media was aspirated and 50 ul/well of 20 mM NH₄OH in PBS (Sigma) was added. The plates were incubated at 37 C for 6 minutes, after which the NH₄OH was pipetted up and down 4-5 times before being aspirated. The wells were then filled with 50 ul/well PBS until seeding with human islets. Human islets were pelleted and dissociated with StemPro Accutase (Life Technologies, A11105-01) at 37 C for 20-25 minutes followed by pipetting to mix 5-7 times. The dissociated islets were resuspended in CMRL 1066 medium (CellGro, cat. no. 15-110-CV) supplemented with 10% FBS, 1× L-glutamine, and 1× penicillin/streptomycin, filtered through a nylon mesh to remove large cell aggregates, and seeded at 10K/well density in the coated 384w plate. These islets were maintained in full CMRL media at 37 C, 95% humidity, and 5% CO2 with media changes every third day.

### FFA Preparation

Enzo SCREEN-WELL® Fatty Acid library (#BML-2803-0100) containing FFAs dissolved in DMSO ([FFA]_{stock}=10 μM) was stored in glass vials at −20° C. in the compound management facility of the Broad Institute. Template plates for HTS were stored up to 4 weeks at 4° C. To prepare compound plates, small volumes of DMSO dissolved FFAs were transferred into microplates containing fatty acid free BSA (Sigma #A8806) solutions in ddH₂O in a molecular ratio of 1:6.67 (BSA:FFA, [FFA]_{final}=500 μM) with an automated simultaneous pipettor (analytikjena CyBio® Well vario). Plates were incubated overnight for 24 h at 37° C. to ensure complete conjugation of FFAs to BSA. Next, DMSO and ddH₂O were completely removed with the GeneVac HT-12 evaporator for 12 h with full vacuum at 37° C. and continuous centrifugation at 400 g. Plates with dry FFA conjugated BSA crystals in the wells were resuspended in MIN6 culture medium at room temperature for 4-8 h on an orbital plate shaker. After resuspension compound plates were spun down at 5000 g for 10 min and manually transferred to 384 MultiScreenHTS HV Filter Plates (0.45 μm, Millipore, #MZHVNOW10) and spun down again for 1 min at 500 g into an empty compound plate. Resulting filtered compound plates were transferred into assay plates of the same format with the analytikjena CyBio® Well vario simultaneous pipettor. Representative FFAs were ordered from Nu-Chek Prep, Inc., manually dissolved in DMSO ([FFA]_{stock}=10 μM) and prepared in glass vials according to the same protocol described above.

Differential Scanning calorimetry

All differential scanning calorimetry (DSC) measurements were performed with a MicroCal VP-Capillary DSC Automated system provided by Malvem Panalytical. Selected FFAs were conjugated to BSA in microplates according to protocol described above and resuspended in PBS to a final concentration of [BSA]_{final}=50 μM. Sample measurements included one measurement of PBS vs. PBS to record a baseline reference curve at the scan rate of 200° C./h. The samples were heated from Tₛₜₐᵣₜ=10° C. up to T_{end}=90° C. at the same scan rate. The melting temperature Tm was determined from the resulting single-peak melting curve using FFA-free BSA as a control.

### Lipid Profiling

40,000 MIN6 cells/well were seeded in 96 well plates 24 h prior to treatment in three replicates. FFA library compound plates were transferred into assay plates and incubated for 24 h. The lipid fraction of cells was isolated with isopropanol after washing the plates 3 times with ice cold PBS. After the addition of Isopropanol plates were incubated for 1 h at 4° C. IPA extracts were then manually transferred to autosampler vials (Waters), capped, and stored at −80° C. until analysis. Lipid profiling was done as previously described (49). Briefly, non-targeted liquid chromatography mass spectrometry (LC-MS) data were acquired using a system comprised of a Nexera X2 U-HPLC system (Shimadzu Scientific Instruments; Marlborough, Mass.) coupled to a Q Exactive Focus Orbitrap mass spectrometer (Thermo Fisher Scientific; Waltham, Mass.). Cell extracts (2 μL) were injected directly onto a 100×2.1 mm, 1.7 μm ACQUITY BEH C8 column (Waters; Milford, Mass.). The column was then eluted isocratically with 80% mobile phase A (95/5/0.1; vol/vol/vol 10 mM ammonium acetate/methanol/formic acid) for 1 minute followed by a linear gradient to 80% mobile-phase B (99.9/0.1; vol/vol methanol/formic acid) over 2 minutes, a linear gradient to 100% mobile phase B over 7 minutes, then 3 minutes at 100% mobile-phase B. Mass spectrometry analyses were carried out using ESI in the positive ion mode using full scan analysis over 220-1100 m/z. Raw data were processed and visually inspected using TraceFinder 3.3 software (Thermo Fisher Scientific; Waltham, Mass.) and Progenesis QI (Nonlinear Dynamics; Newcastle upon Tyne, UK). The identity of individual metabolites and lipid families was confirmed by matching their retention time to that of authentic reference standards.

### RNASeq/SmartSeq 2

40,000 MIN6 cells/well were seeded in 96 well plates 24 h prior to treatment in three replicates. FFA library compound plates were transferred into assay plates and incubated for 24 h (n=6). Then RNA was extracted from cells using TCL buffer (#1031576, Qiagen) with 1% beta-mercaptoethanol followed by an RNA clean-up with Agencourt RNA cleanup XP (#A63987, Beckman Coulter). Bulk RNA (1 ul) was added to a 3 step cDNA synthesis reaction with a 3′RT (5′-AGCAGTGGTATCAACGCAGAGTAC(T30)VN-3′, SEQ ID NO: 51, IDT), template switching (5′-AAGCAGTGGTATCAACGCAGAGTACrGrG+G-3′, SEQ ID NO: 52, Qiagen), and ISPCR (5′-AAGCAGTGGTATCAACGCAGAGT-3′, SEQ ID NO: 53, IDT) oligos from the SMART-seq2 protocol (24). cDNA was purified using AMPure XP Agencourt (#100609, Beckman Coulter) and quantified using Qubit dsDNA High Sensitivity (#102689, Life Technologies). Samples were diluted to 0.2 ng/ul in TE and tagmented (Nextera XT DNA Library Preparation Kit (#FC-131-1096, Illumina). Indexing was performed using the Nextera XT Index Kit (#FC-131-1001, Illumina). Final libraries were QCed using the Qubit dsDNA High Sensitivity kit and Bioanalyzer High Sensitivity DNA Kit (#5067-4627, Agilent). Libraries were sequenced at a concentration of 1.8 pM on a NextSeq with a 75 cycle v2 kit (#TG-160-2002, Illumina) with a read structure of Read 1 37 bp, Read 2 37 bp, Index 1 8 bp, and Index 2 8 bp. Each sample was given about 4M reads.

### Bioinformatics and Data Analysis

Unless otherwise stated, all computational and statistical analysis in this study were performed in python. The following openly available python software packages were used: scipy, numpy, pandas, scikit learn, statsmodels, gseapy, matplotlib and seaborn.

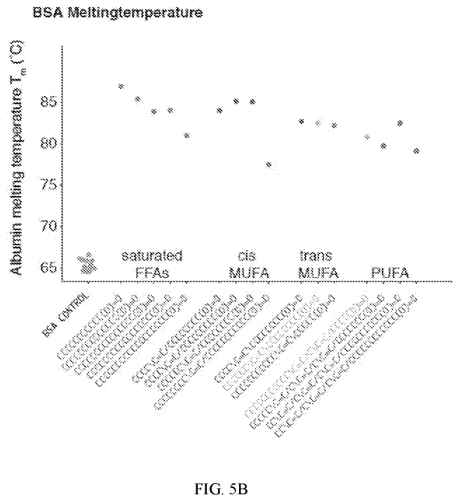

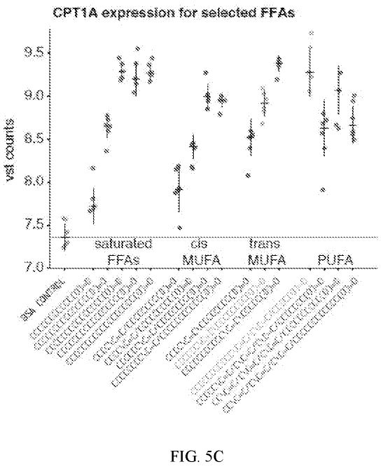

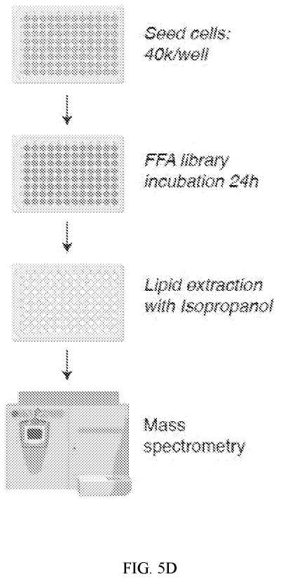

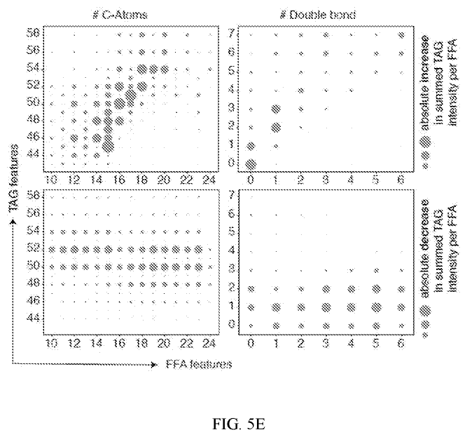

(1) Lipidomics
A blocked experimental design with one replicate of each FFA in the library, together with multiple BSA controls per 96 well plate was chosen (n=3). Raw lipidomic profiles received from the Metabolomics Platform at the Broad Institute were filtered for samples with strongly deviating sample medians (manual cutoff, 7 out of 280 or 3% of the samples were discarded). Lipid metabolites that exhibited more than 30% of missing data points were removed, otherwise missing values were substituted with 50% of the minimum value of the respective metabolite's intensity. To account for variations in total amount of captured metabolites, samples were scaled towards the global sample median. Only annotated lipid metabolites were used for further differential abundance analysis. A primary goal was to understand the relationship between structural features of externally added FFAs and changes in the triglyceride fraction of the cells (FIG. 5E). For each externally added FFA, triglyceride intensity deviations from the BSA control were summed based on the structural feature of interest (number of C-atoms, number of double bonds). Then, triglyceride profiles of externally added FFAs were summarized based on the structural feature of interest of the FFA (number of C-atoms, number of double bonds) and normalized to the number of FFAs making up each group.

(2) RNASeq Pipeline and Gene Set Enrichment Analysis
A blocked experimental design with one replicate of each FFA in the library together with multiple BSA controls per 96 well plate was chosen (n=6). Raw data from NextSeq runs were de-multiplexed and converted to sample specific fastq files. Alignment was performed with STAR (50), reads were counted with HTSeq (51) and QC metrics were generated with RNA-SeQC (52). The resulting count matrix was filtered by column for samples with more than 10³ detected genes (counts >0) and by row for coding genes (as defined by the MGI database) with a row sum across all samples>500 counts (with a total number of 500 samples). The resulting normalized and filtered count matrix was then variance stabilized using the vst method from the DESeq2 R package (53). Then, surrogate variable analysis (SVA, R package) (54) was performed on the vst count matrix to account for linear batch effects. In addition, differential expression analysis was performed using DESeq2 for each sample, including derived surrogate variables to the linear model. To cluster the samples, the top 500 most commonly significantly differentially expressed genes (p_{adj}<0.05) across the whole dataset were chosen. Samples were either transformed to z-scores or replicates were collapsed by calculating their signal to noise ratio (with respect to the BSA control) before performing hierarchical clustering based on Euclidean distance and Ward's linkage method. Clusters were extracted with the Dynamic Tree Cut function (55). After assigning each FFA to a cluster, differential expression analysis was performed based on cluster labels and BSA controls (based on the vst count matrix). For gene set enrichment analysis (GSEA), cluster centric DE gene lists were ranked based on log 2 fold change and analyzed for enrichment with the MsigDB Hallmark gene sets (26, 27).

(3) MAGMA analysis pipeline, gene functional readout correlations
The MAGMA software (v 1.07) (40) was used to perform SNP annotation, gene analysis to generate ranked lists of genes from GWAS summary statistics and gene set analysis according to the instructions. To calculate the FDR for gene set enrichment, a permutation-based approach was employed to generate an empirical Null Hypothesis. For each gene set, 1000 randomly sampled gene sets were generated of the same size from the transcriptomics gene list and calculated the FDR accordingly. To correlate expression profiles and functional readouts across all samples, data points were filtered with significant increase or decrease in the functional readout and calculated parsons correlation coefficient. P-values were corrected for multiple testing (considering all genes, Bonferroni).

### Cell Viability

For high throughput cell viability assay, cells were seeded in 384 well plates (Perkin Elmer, CellCarrier Ultra, #6057300) and treated for 24, 48 and 72 h with the FFA library. Just before readout, cell nuclei were stained with Hoechst (Thermo Fisher Scientific) for 1 h at 37° C. and imaged with the Opera Phenix High Content Screening System (#HH14000000, Perkin Elmer). Number of counted nuclei was determined with the image analysis software Harmony (PerkinElmer) and used as a proxy for cell viability. For low throughput validation experiments, cells were treated for 48 h with representative FFAs in CellCarrier-384 Ultra Microplates. Caspase 3/7 (Thermo Fisher Scientific, #C10423) activation and propidium iodide (Thermo Fisher Scientific, #P1304MP) staining were used to calculate the fraction of apoptotic cells and dead cells, respectively. Single cells were identified and counted after staining their nuclei with Hoechst. Fluorescence intensities were then measured and the threshold for Caspase 3/7 and propidium iodide positive staining was determined manually. Cell viability was calculated as the fraction of cells that were neither Caspase 3/7 nor propidium iodide positive.

For testing of free fatty acids for toxicity against human pancreatic islet cells (FIG. 8), C2, C3, and C4 free fatty acids were incubated with human pancreatic islet cells at concentrations of 250 μM, 500 μM, and 1 mM for 120 hours with one media change at 72 hours. The islets were subsequently fixed with 3% paraformaldehyde for 20 minutes, permeabilized with 0.2% TritonX-100 for another 20 minutes, and blocked for 3 hours at RT in 2% BSA in PBS (SeraCare, AP-45100-80). The islets were stained with C-peptide antibody (Developmental studies hybridoma bank at University of Iowa) at 1:100 in 2% BSA/PBS overnight rocking at 4 C, washed with PBS three times followed by once with 1% BSA/PBS, and then incubated with 568 Goat anti-rat (Life technologies, A11077) at 1:1000 and Hoechst (Thermo Fisher Scientific) at 1:1000 for one hour at RT. These islets were then imaged with the Operetta CLS High Content Screening System (#HH16000000, Perkin Elmer). The number of C-peptide positive cells in each well was quantified using Harmony software (PerkinElmer) and used as a proxy for cell viability.

### Immunoblotting

MIN6 cells were lysed (#9803, Cell Signaling Technology) in the presence of protease inhibitors (#05892791001, Roche) and phosphatase inhibitors (#04906837001, Roche). Protein concentrations were quantified with the Pierce BCA Protein Assay Kit (#23225, Thermo Fisher Scientific). NuPAGE LDS sample buffer (#NP0008, Thermo Fisher Scientific) was added to normalized protein lysates together with NuPAGE reducing agent (#NP0004, Thermo Scientific). Lysates were heated to 95° C. for 5 min prior to SDS-PAGE gel electrophoresis (NuPAGE MES SDS running buffer, Thermo Fisher Scientific, #NP0002). Proteins were transferred to a nitrocellulose membrane (#1704158, BioRad) with the Trans-Blot® Turbo™ Blotting System (#1704155, BioRad) according to the manufacturer's protocol. Membranes were blocked in 5% Nonfat Dry Milk (#9999S, Cell Signaling Technology) in PBS with 0.1% Tween® 20 (PBS-T). Primary antibodies were incubated at 4° C. overnight, secondary antibodies were incubated at room temperature for 1 h. Super Signal West Dura (#34076, Thermo Fisher Scientific) or Super Signal West Pico (#34087, Thermo Fisher Scientific) were used to visualize immunoreactive bands imaged by G:BOX Chemi XT4:BOX-CHEMI-XT4, Syngene). Primary Antibodies used in this study: CPT1A: (Abcam #ab128568), ATF4: (CST #11815), CHOP: (CST #2895).

### Immunofluorescence (IF) Staining

Cells grown on 384 well CellCarrier Ultra microplates (#6057308, PerkinElmer) were fixed 10 min in PBS containing 4% PFA (Electron Microscopy Sciences), permeabilized 15 min in 0.5% Triton X-100 (Sigma-Aldrich), blocked for 1 h in blocking reagent (100 mM Tris HCL pH8; 150 mM NaCL; 5 g/L Blocking Reagent (#11096176001, Roche)) and treated for 1.5 h with primary antibody diluted in blocking reagent (NF-κB p65/RELA, Rabbit monoclonal antibody, 1:200, (#8242, Cell Signaling Technology). Cells were washed three times in PBS and incubated for 0.5 h with fluorescent-labeled secondary antibody in blocking solution (1:500, Alexa Fluor 568 Goat anti-Rabbit IgG, (#A11036, Thermo Fisher Scientific)). Cytoplasmic actin filaments were stained with Phalloidin conjugated with Alexa 647 (1:40, #A22287, Thermo Fisher Scientific) and nuclei were counterstained with Hoechst (1:2000, #H3570, Thermo Fisher Scientific). Cells were washed three times in PBS and imaged using the Opera Phenix High Content Screening System, #HH14000000, Perkin Elmer). A minimum of nine fields was acquired per well using 20× water immersion objectives in a confocal mode. Image analysis was performed using the Harmony software (PerkinElmer). Cell nuclei were first identified using Hoechst staining and nuclear region was defined for each cell. Phalloidin staining was then used to detect and define the cytoplasmic region of the cell. RELA fluorescent intensity was measured separately in the nuclear and cytoplasmic regions and a threshold for a nuclear translocation was defined using negative (BSA) and positive (TNFα) controls. For each well the fraction of cells identified for RELA nuclear translocation was calculated.

### ER Calcium Levels

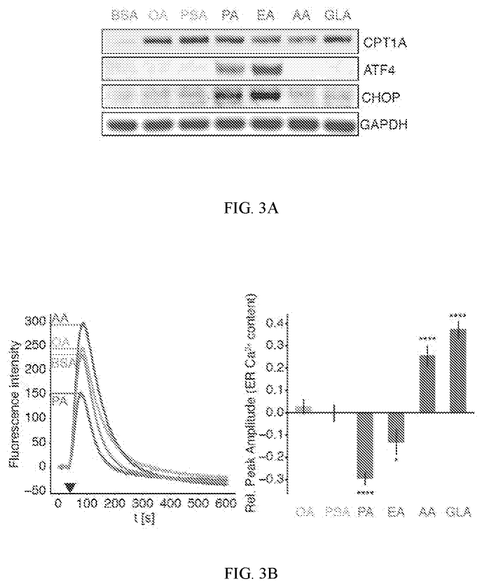

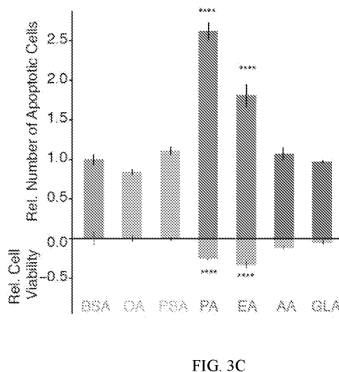

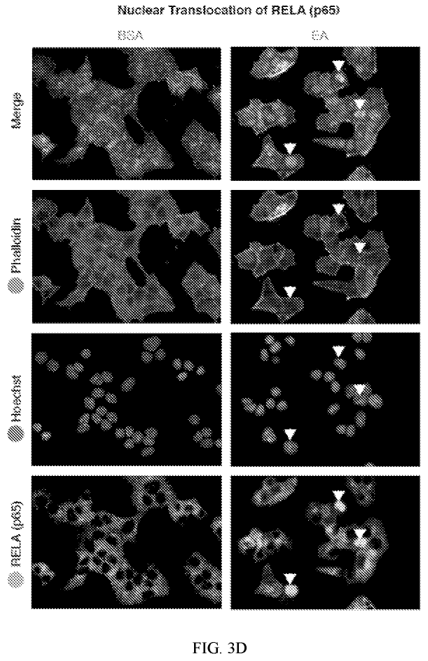

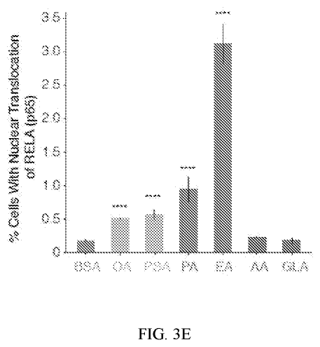

MIN6 cells were plated in 384 well plates (Aurora, Black 384 SQ Well 188 micron Film, #1022-10110) and treated with the FFA library for 24 h prior to readout. Cells were carefully washed three times with HBSS (with calcium, Thermo Fisher Scientific, #14025076) using an automated simultaneous pipettor (analytikjena CyBio® Well vario) and incubated with the fluorescent calcium indicator Fluo4 (2 μM, Life Technologies, #F14202) in DMEM without additions for 1 h at room temperature. Then, cells were washed again in HBSS (with calcium) and incubated for another 30 min at room temperature in DMEM without additions. Just before the readout, cells were washed in final calcium free assay buffer solution (140 mM NaCl, 5 mM Kcl, 10 mM HEPES, 2 mM MgCl₂, 10 mM EGTA, 10 mM Glucose) and left with 25 ul assay volume per well. Assay plates were immediately transferred to the FLIPR Tetra® High-Throughput Cellular Screening System. The plate was recorded with a frequency of 1 Hz for 10 min. Baseline was recorded for 30 s before the automated liquid transfer system of the FLIPR added the SERCA inhibitor Thapsigargin (final concentration 10 μM) in calcium free assay buffer. The resulting passive efflux of calcium from the ER induced a transient cytosolic fluorescence signal and the peak amplitudes were used to indirectly quantify ER calcium levels (See Extended Data FIG. 3d). The resulting trajectories were corrected for a pipetting artifact and baseline normalized. Log 2 Fold changes were calculated according to plate location specific negative (BSA) controls. The exclusion of one outlier/FFA/plate (n=5) based on a 3 sigma cutoff was allowed. P-values were calculated with Student's t-test (two-sided) and corrected for multiple testing (Benjamin & Hochberg).

### Glucose Stimulated Insulin Secretion

Stable MIN6 cell lines were generated with adenoviral delivery of the Proinsulin-NanoLuc in pLX304 and Blasticidin selection. The plasmid was Addgene plasmid #62057; http://n2t.net/addgene:62057; RRID:Addgene 62057. Glucose stimulated insulin secretion was measured as described previously (21). In brief, cells were plated in 384 well plates (Perkin Elmer, CellCarrier Ultra, #6057300) and treated with the FFA library for 24 h prior to readout. First, cells were preincubated in Krebs Ringer Buffer (KRB) with 2.8 mM glucose for 1 h and then stimulated with fresh KRB containing 16.7 mM glucose. The supernatant was then transferred to white 384 well assay plates (Corning #3574) preloaded with Coelenterazine substrate (NanoLight Technology, #303) solution and immediately imaged with the ViewLux® uHTS Microplate Imager.

### Example 2: Identification of Lipotoxic FFAs

An initial challenge confronted in attempting to systematically investigate the spectrum of FFAs for lipotoxicity was to design an effective system for working with FFAs at scale. In the blood stream, hydrophobic FFAs are transported either as part of complex lipids in lipoproteins or conjugated to serum albumin (18). A library of 61 structurally diverse FFAs (Table 1) were employed and a protocol was developed to prepare solvent-free BSA-conjugated FFA solutions in microplates (see FIG. 5A, and Example 1). This approach was validated in three orthogonal ways: (i) differential scanning calorimetry (DSC) (19) to assess the shift in BSA melting temperature (Tm) for a set of structurally representative FFAs, confirming that they were bound to and stabilized by BSA (FIG. 5B); (ii) Carnitine Palmitoyltransferase I (CPT1A), the rate-limiting enzyme of FFA beta-oxidation (20), was strongly induced in MIN6 pancreatic beta cells (a widely used mouse pancreatic beta cell line (21-23)), indicating that the FFAs in the library were successfully delivered and metabolized; (iii) a mass spectrometry-based lipidomic analysis of lysates from cells treated with each of the 61 FFAs in the library (FIG. 5D) and found that the structural features of the detected triglycerides (number of C atoms and double bonds) changed as a function of the structural features of externally applied FFAs (FIG. 5E), confirming successful incorporation of FFAs into cellular lipids. In summary, a novel protocol to successfully and reproducibly deliver FFAs into cells at scale was developed.

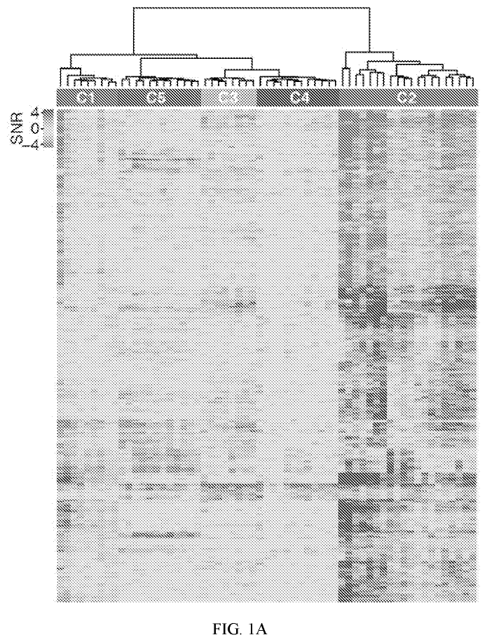

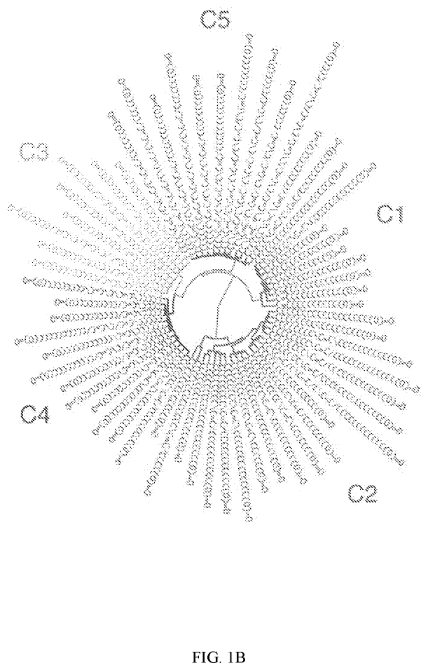

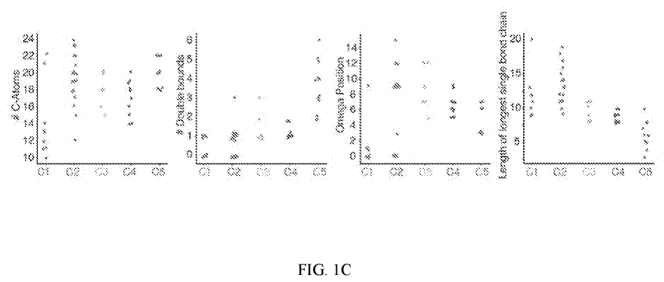

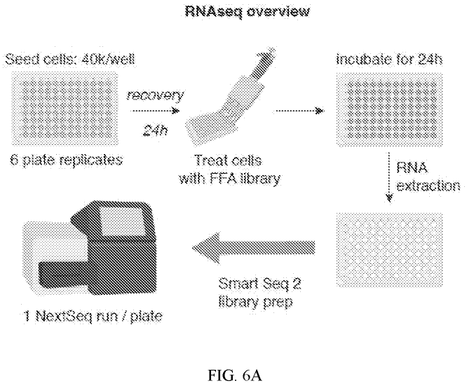

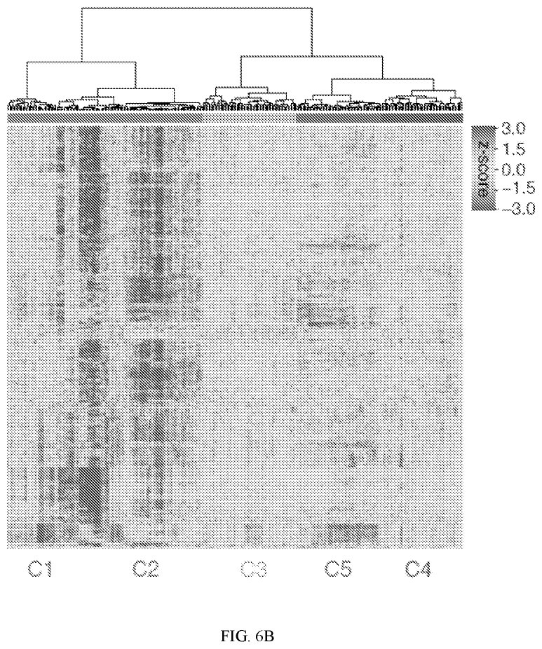

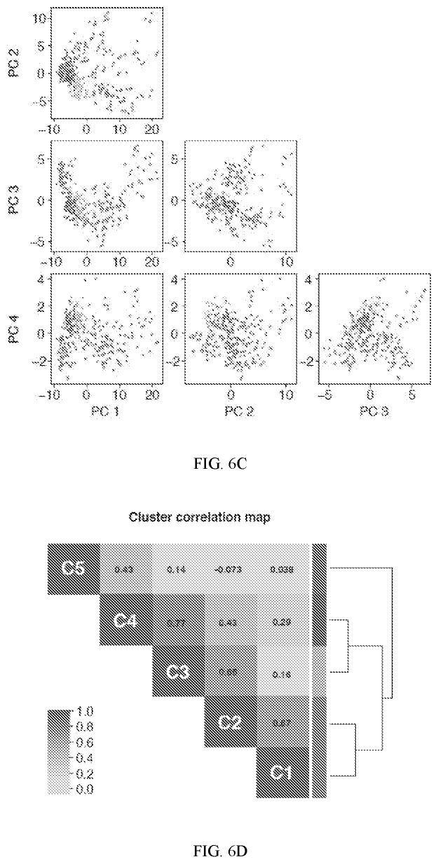

To investigate the biological effects mediated by each FFA in the library, RNAseq was used to generate transcriptomic profiles (24) (FIG. 6A) and data were visualized as a heatmap of highest variable genes across all samples (FIG. 1B, n=6 biological replicates; FIG. 6B). Of note, hierarchical clustering revealed distinct transcriptomic signatures (suggesting distinct biological effects) even among FFAs previously thought to be members of the same group of lipid molecules. For example, 3 distinct signatures for FFAs that have been historically grouped into a single monounsaturated FFA group (MUFAs; clusters 2, 3 and 4). This result was also visually captured in the principal component analysis (FIG. 6C). Next, a process to quantify cluster similarity was generated. Specifically, the first principal component of each cluster was calculated based on the respective expression profiles of member FFAs, essentially generating a “meta-sample” for each cluster (25). The correlation matrix of representative “meta-samples” (FIG. 6D) revealed how different clusters are related to each other. The clusters were then ordered based on their calculated correlation proximity. The composition of the identified FFA clusters was visualized (FIG. 1B). It was then investigated whether classical structural features of FFAs correlated with the newly-defined clusters. Characteristic structural features were then plotted of identified FFA clusters (FIG. 1C), ordered by their transcriptomic adjacency (FIG. 6D). None of these features predicted cluster membership individually, but several interesting observations were notable. Apart from an overrepresentation of shorter chain FFAs (10-14 C-atoms) in cluster 1 (C1), the number of C atoms did not separate the transcriptomically derived FFA clusters. Second, in terms of double bond content, saturated FFAs (SFAs) were divided between cluster 1 (C1) and cluster 2 (C2), and mono-unsaturated FFAs (MUFAs) were divided between three clusters (C2, 3 and 4). The exception was cluster 5 (C5) that exclusively contained poly-unsaturated FFAs (PUFAs). The omega position of double bonds (defined as the position closest to the last carbon in the chain) in unsaturated FFAs appeared to decrease from cluster 2 to cluster 5. It was noted that the length of the longest single bond chain, a structural feature rarely used to characterize FFAs, was the most distinctive feature of cluster 2. In summary, this unbiased transcriptomic analysis broke a long-held paradigm by showing that structurally different FFAs cluster together; for example, based on transcriptomic analyses, some mono-unsaturated FFAs appeared to cluster together with saturated FFAs (in cluster 2).

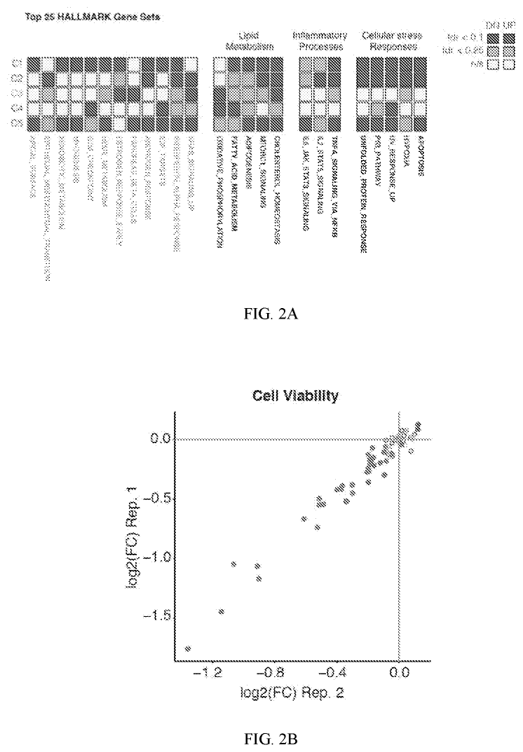

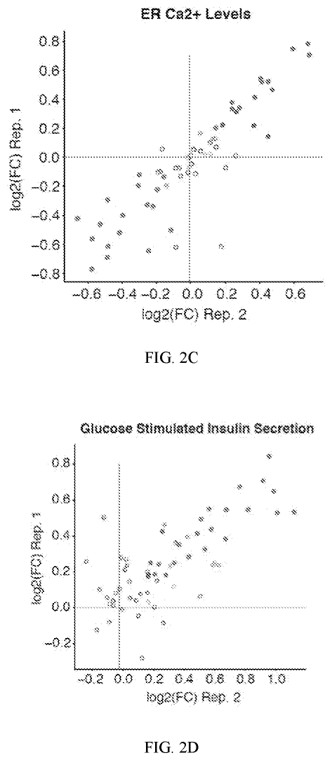

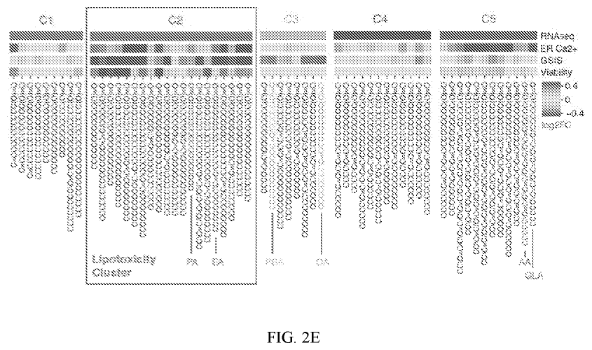

To further explore the new, transcriptome-based FFA clusters, the underlying biological signatures driving differential FFA clustering were investigated. A cluster-centric differential expression analysis was performed and the genes were ranked according to their log₂ Fold Change (LFC). Gene Set Enrichment Analysis of (GSEA) (26) of Hallmark Gene Sets from MSigDB (27) revealed differentially enriched gene sets related to cellular stress responses, inflammation and lipid metabolism (FIG. 2a). These results provided several important insights. First, across all clusters, an enrichment of genes involved in fatty acid metabolism were captured consistently, providing additional confirmation for the successful delivery of FFAs into cells. Second, signatures of inflammatory processes were identified, including activation of NFkB signaling, enriched specifically in C1 and C2. This result is consistent with previous work showing inflammatory signaling in a variety of tissues (28-30) in response to a subset of saturated FFAs (included here in clusters 1 and 2). Most importantly, cellular responses enriched in C1 and C2 were the unfolded protein response (UPR) and apoptosis, both well-established hallmarks of the lipotoxic state (31). In summary, this high level transcriptomic analysis of biological signatures revealed differential perturbation of pancreatic beta cells after exposure to different FFAs.

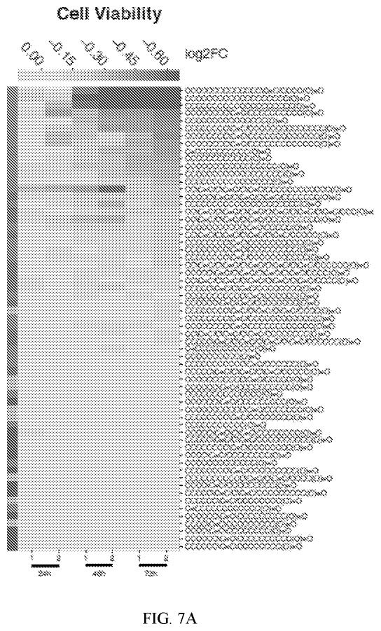

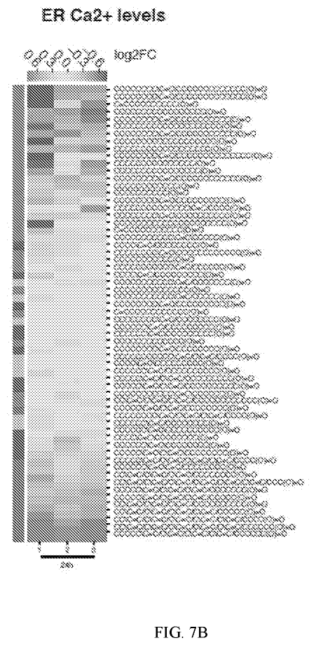

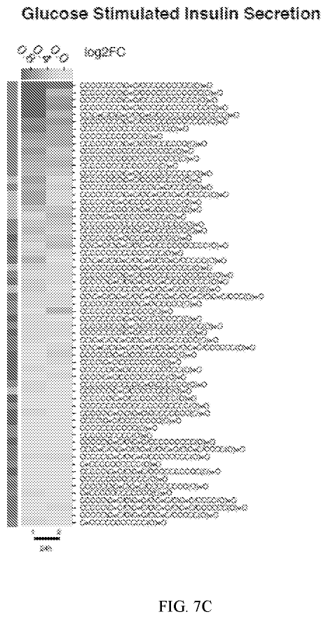

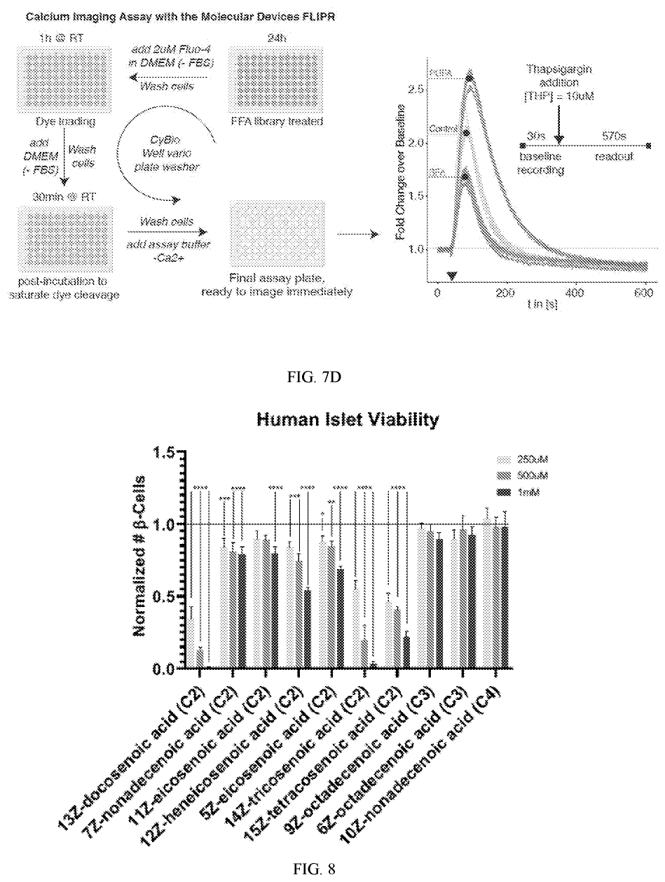

To better understand the lipotoxicity traits derived from transcriptomics, three assays were designed to functionally characterize cellular stress states. First, cell viability was measured in MIN6 cells incubated with each of 61 FFAs for 72 hours (FIG. 2B, FIG. 7A). C2 FFAs showed a consistent and significant decrease in cell viability. A modest reduction in cell viability was noted for C1 and C5. Incubation with FFAs from C3 and C4 did not affect cell viability. Second, based on the observation that FFA-induced lipotoxicity is associated with decreased levels of ER Ca²⁺ (32, 33), a fluorometric high throughput assay was developed to measure ER Ca²⁺ after treatment with each of 61 FFAs (FIG. 2C, FIG. 7B). Decreased ER Ca²⁺ levels were detected in C2 FFAs and, to a smaller extent, in C1. Of note, a consistent increase in ER Ca²⁺ levels for C5 was found, which indicated that these FFAs might have injured cells through excess intracellular Ca²⁺ accumulation. C3 and C4 did not affect ER Ca²⁺ stores, consistent with a non-harmful (or even protective) role for these FFAs; indeed oleic acid, previously reported as a protective FFA (15), was in C3. Third, it was investigated how FFAs affected insulin secretion, a fundamental physiological function of pancreatic beta cells (21). An established method (21) to measure glucose stimulated insulin secretion (GSIS) was used after exposure to each of 61 FFAs (FIG. 2D, FIG. 7C). While previous work suggested that FFAs generally increase insulin secretion (23), it was found that the effect on GSIS was most pronounced in C2. All physiological responses (cell viability, ER Ca²⁺ and GSIS) measured for each of the 61 FFAs were summarized in a heatmap (FIG. 2E). From this analysis, C2 FFAs emerged as most highly associated with lipotoxicity, both by transcriptomics and by the three orthogonal functional features measured (cell viability, ER Ca²⁺ and GSIS). It was also noted that palmitic acid (PA), the saturated FFA that had been traditionally used to study lipotoxicity in vitro and in vivo (15), was a member of this newly defined lipotoxicity cluster, serving as a positive control. In summary, 20 structurally diverse (saturated and mono-unsaturated) FFAs were identified that comprised a newly-defined lipotoxicity cluster (C2).

To validate these findings, 6 representative FFAs from the most distinctive FFA clusters (C2, C3 and C5, highlighted in FIG. 2E) were selected to perform independent cell biological assays. By western blot, induction of the UPR (34) was assayed by detecting the upregulation of ATF4 and CHOP protein abundance specifically after treatment with erucic acid (EA) and PA, two representative FFAs from the lipotoxicity cluster (C2, FIG. 3A). This result confirmed (at the protein level) the transcriptomic UPR profile that helped define this cluster (FIG. 2A). None of the other FFAs (OA and petroselenic acid (PSA) for C3; arachidonic acid (AA) or gamma linoleic acid (GLA) for C5) induced the UPR. CPT1A protein abundance was increased in all cases, serving as a control for successful intracellular delivery of the selected FFAs. In line with previous studies (32, 33), the induction of the UPR was uniquely associated with a significant reduction of ER Ca²⁺ levels in cells treated with PA or EA, in contrast to near-baseline (OA, PSA) or increased (AA, GLA) ER Ca²⁺ levels in cells treated with FFAs from other clusters (FIG. 3B). To validate the cell count-based viability assay, caspase activity was measured as a marker of apoptosis and propidium iodide-positive nuclei as a marker for cell death (FIG. 3C). The lipotoxic FFAs EA and PA were the only FFAs which consistently induced apoptosis and cell death (FIG. 3C). Finally, since lipotoxic inflammation (or metaflammation) is thought to play a central role in the development of metabolic diseases and T2D (28), and inflammatory signaling through NFkB also emerged from the transcriptomic analysis (FIG. 2A), it was necessary to evaluate it experimentally. NFkB signaling, previously implicated in PA-induced inflammatory responses (30), was assessed by detection of nuclear translocation of RELA (p65), a major component of NFkB transcription (35). RELA translocated to the nucleus after treatment with EA (FIGS. 3D and 3E) and PA (FIG. 3E), in line with the detection of transcriptomic signatures of NFkB activity (FIG. 2A). PSA and OA also triggered moderate RELA translocation to the nucleus (FIG. 3E), but this event was not associated with changes in cell viability and was thus unrelated to lipotoxicity (FIG. 3C).

C2 lipotoxicity cluster free fatty acids also greatly decreased human pancreatic islet cell viability (FIG. 8). 13Z-docosenoic acid, 7Z-nonadecenoic acid, 11Z-eicosenoic acid, 12Z-heneicosenoic acid, 5Z-eicosenoic acid, 14Z-tricosenoic acid, and 15Z-tetracosenoic acid all decreased human pancreatic cell viability relative to the C3 free fatty acids 9Z-octadecenoic acid and 6Z-octadecenoic acid, and the C4 free fatty acid 10Z-nonadecenoic acid. In particular, 13Z-docosenoic acid, 14Z-tricosenoic acid, and 15Z-tetracosenoic acid decreased human pancreatic cell viability by more than 50% relative to the C3 and C4 free fatty acids.

In summary, the deleterious cellular effects of the lipotoxicity cluster (C2) were functionally and independently validated as compared to FFAs from other clusters, bolstering the conclusion that the instant disclosure's systematic analysis identified a previously unrecognized group of 20 FFAs that drive cellular lipotoxicity.

### Example 3: Identification of Lipotoxic Genes

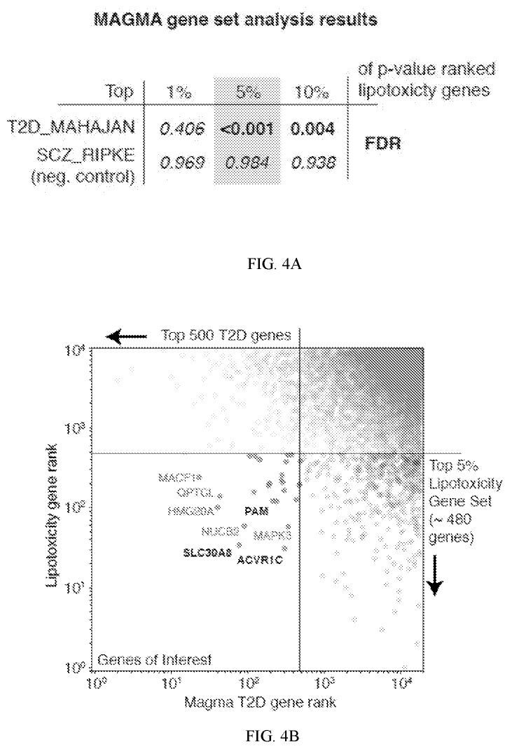

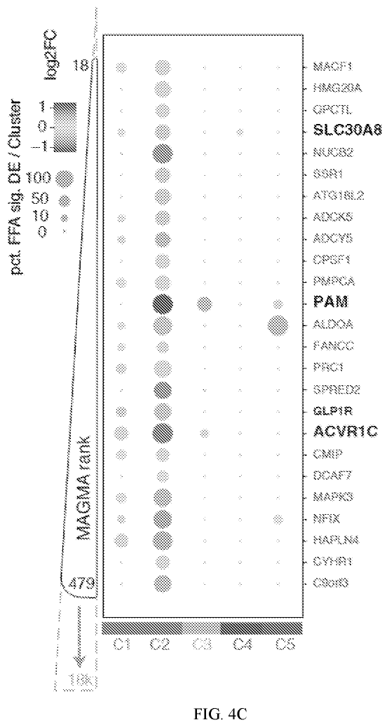

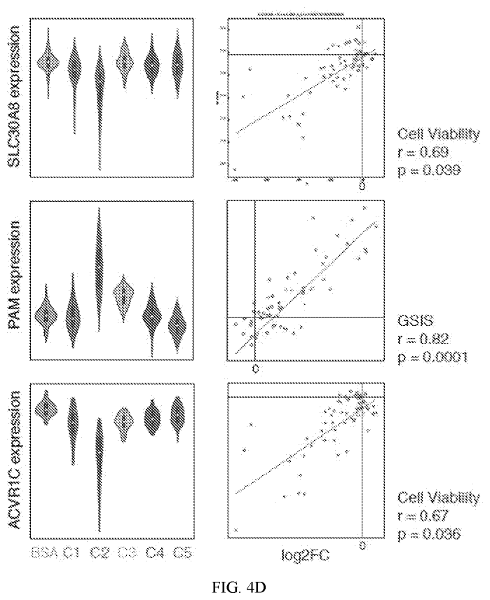

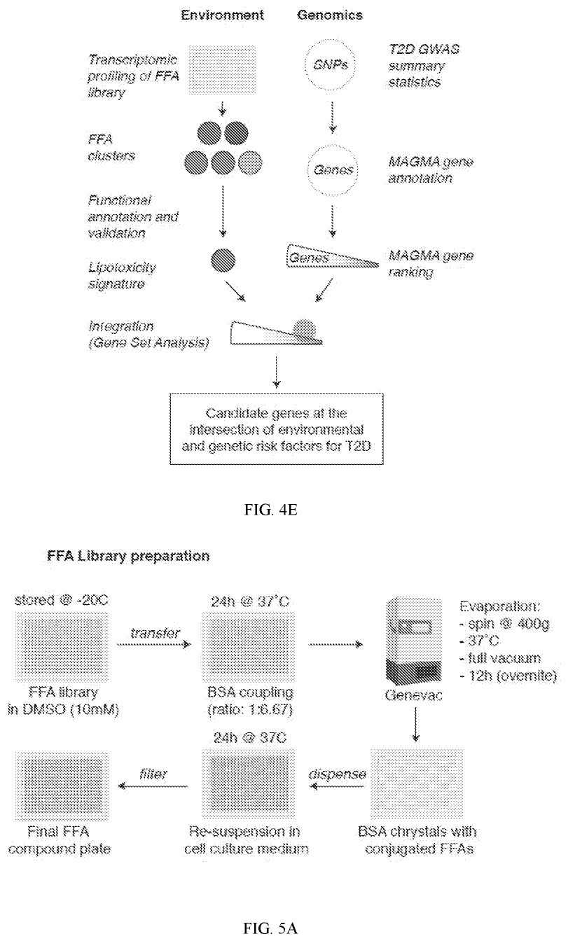

The motivation to systematically evaluate the spectrum of FFA-mediated cell biology was founded on the strong association between the lipotoxic environment and risk for T2D (8). In line with this hypothesis, it was found that the newly defined FFA lipotoxicity cluster (C2) affected insulin secretion and pancreatic beta cell survival (FIGS. 2B and 2D), two fundamental processes also strongly associated with T2D in several genomic studies (36-39). It was then investigated whether integrating the transcriptomic lipotoxicity profile with recent T2D GWAS data would identify genes at the intersection of environmental and genetic risk for T2D. An analysis pipeline was developed to test whether the lipotoxicity signature showed an enrichment among genes emerging from the largest T2D GWAS study to date (2). First, gene analysis using the MAGMA software (40) was performed to rank genes based on their proximity to identified T2D SNPs. Second, the top 1%, 5% and 10% of differentially expressed genes in the lipotoxicity cluster (based on p-value) were extracted. The resulting lipotoxicity gene sets were then tested against the ranked MAGMA gene list using gene set analysis (GSA) (40). It was found that the 5% and 10% lipotoxicity gene sets were strongly enriched in the T2D GWAS dataset (FDR <0.05, FIG. 4A). No enrichment was evident in a GWAS dataset for schizophrenia (41), which served as a negative control. This finding had major immediate implications, because (a) it is an unbiased approach that re-derived lipotoxicity as an important contributor to T2D pathogenesis; and (b) it validated the human relevance of the cell-based platform as a valuable and scalable tool to study lipotoxicity in the context of human metabolic disease.

Next, specific genes that drove the significance of the lipotoxicity gene sets in the instant analysis were examined. All genes in a scatter plot were plotted according to their MAGMA rank on the x-axis and their lipotoxicity rank on the y-axis (based on DE p-value). The 5% lipotoxicity gene set was conservatively selected as the y-axis boundary and the top 500 genes of the ranked MAGMA list was arbitrarily selected as the x-axis boundary. This approach led to a list of 25 leading genes of interest (FIG. 4B). The expression profile of these genes was plotted across all FFA clusters (FIG. 4C); confirming that C2 showed the highest levels of differential expression for these genes. Several specific genes emerged from this analysis. For example, GLP1R is the target of incretin mimetics, which are a well-known class of drugs for diabetes treatments (9, 10). Additional genes of interest were PAM and SLC30A8. Landmark studies, including a published T2D exome sequencing study (11) and a T2D coding variant fine mapping study (12), showed the significance of the association of coding variants in PAM and SLC30A8 with T2D. Two PAM variants decreased PAM activity and insulin secretion in a human pancreatic beta cell model (36), which was in agreement with the lipotoxicity (C2) dataset in MIN6 cells showing a strong correlation between PAM expression and insulin secretion (GSIS)(FIG. 4D). These findings suggested that PAM was an important mediator of increased insulin secretion in a lipotoxic environment, and thus a good candidate therapeutic target. SLC30A8 encodes for a zinc transporter predominantly found in pancreatic islets (42). Based on in vitro and in vivo studies, as well as data from human genetics, it has been suggested that changes in zinc transport through SLC30A8 increase the risk for T2D (38, 43-35). In the instant disclosure dataset, SLC30A8 downregulation was negatively correlated with beta cell viability in cluster 2 (FIG. 4D), a result that heightens interest in its value as a candidate therapeutic target. Finally, a novel gene of interest was activin receptor-like kinase 7 (ACVR1C), previously implicated in pancreatic beta cell injury and apoptosis (46, 47). In the instant disclosure dataset, ACVR1C was strongly downregulated in the lipotoxicity cluster and negatively correlated with beta cell viability (FIG. 4D). In conclusion, the analysis presented herein nominated 25 genes at the intersection of environmental and genetic risks for T2D that can be further explored to identify novel therapeutic targets.

This approach (FIG. 4E) can be generalized to address several other metabolic diseases by generating lipotoxicity signatures in relevant cell lines such as endothelial cells (to address CVD), hepatocytes (to address non-alcoholic fatty liver disease, NAFLD), macrophages (to address obesity-mediated inflammation/metaflammation), skeletal muscle cells (to address insulin resistance) and adipocytes (to address obesity). These efforts will likely elucidate additional genes and help annotate the ever-increasing list of genomic risk variants for metabolic diseases. Importantly, the generation of lipotoxicity profiles in different cell types will reveal conserved versus tissue-specific features of lipotoxicity across different metabolic diseases, which has major therapeutic implications.

### REFERENCES

1. Kahn S E, Hull R L, Utzschneider K M (2006) Mechanisms linking obesity to insulin resistance and type 2 diabetes. Nature
2. Mahajan A, Taliun D, Thurner M, et al (2018) Fine-mapping type 2 diabetes loci to single-variant resolution using high-density imputation and islet-specific epigenome maps. Nat Genet. https://doi.org/10.1038/s41588-018-0241-6
3. Evangelou E, Warren H R, Mosen-Ansorena D, et al (2018) Genetic analysis of over 1 million people identifies 535 new loci associated with blood pressure traits. Nat Genet. https://doi.org/10.1038/s41588-018-0205-x
4. Nikpay M, Goel A, Won H H, et al (2015) A comprehensive 1000 Genomes-based genome-wide association meta-analysis of coronary artery disease. Nat Genet. https://doi.org/10.1038/ng.3396
5. Yang W, Dall T M, Beronjia K, et al (2018) Economic costs of diabetes in the U.S. in 2017. Diabetes Care. https://doi.org/10.2337/dci18-0007
6. Ogurtsova K, da Rocha Fernandes J D, Huang Y, et al (2017) IDF Diabetes Atlas: Global estimates for the prevalence of diabetes for 2015 and 2040. Diabetes Res Clin Pract. https://doi.org/10.1016/j.diabres.2017.03.024
7. Unger R H, Clark G O, Scherer P E, Orci L (2010) Lipid homeostasis, lipotoxicity and the metabolic syndrome. Biochim. Biophys. Acta—Mol. Cell Biol. Lipids
8. Unger R H (2002) Lipotoxic Diseases. Annu Rev Med. https://doi.org/10.1146/annurev.med.53.082901.104057
9. Lovshin J A, Drucker D J (2009) Incretin-based therapies for type 2 diabetes mellitus. Nat. Rev. Endocrinol.
10. Drucker D J, Nauck M A (2006) The incretin system: glucagon-like peptide-1 receptor agonists and dipeptidyl peptidase-4 inhibitors in type 2 diabetes. Lancet
11. Flannick J, Mercader J M, Fuchsberger C, et al (2019) Exome sequencing of 20,791 cases of type 2 diabetes and 24,440 controls. Nature. https://doi.org/10.1038/s41586-019-1231-2
12. Mahajan A, Wessel J, Willems S M, et al (2018) Refining the accuracy of validated target identification through coding variant fine-mapping in type 2 diabetes article. Nat Genet. https://doi.org/10.1038/s41588-018-0084-1
13. Randle P J, Garland P B, Hales C N, Newsholme E A (1963) THE GLUCOSE FATTY-ACID CYCLE ITS ROLE IN INSULIN SENSITIVITY AND THE METABOLIC DISTURBANCES OF DIABETES MELLITUS. Lancet. https://doi.org/10.1016/S0140-6736(63)91500-9
14. Rhee E P, Cheng S, Larson M G, et al (2011) Lipid profiling identifies a triacylglycerol signature of insulin resistance and improves diabetes prediction in humans. J Clin Invest. https://doi.org/10.1172/JCI44442
15. Palomer X, Pizarro-Delgado J, Barroso E, Vazquez-Carrera M (2018) Palmitic and Oleic Acid: The Yin and Yang of Fatty Acids in Type 2 Diabetes Mellitus. Trends Endocrinol. Metab.
16. Lovejoy J C, Smith S R, Champagne C M, et al (2002) Effects of diets enriched in saturated (palmitic), monounsaturated (oleic), or trans (elaidic) fatty acids on insulin sensitivity and substrate oxidation in healthy adults. Diabetes Care
17. Piccolis M, Bond L M, Kampmann M, et al (2019) Probing the Global Cellular Responses to Lipotoxicity Caused by Saturated Fatty Acids. Mol Cell. https://doi.org/10.1016/j.molcel.2019.01.036
18. Spector A A (1975) Fatty acid binding to plasma albumin. J Lipid Res
19. Michnik A (2003) Thermal stability of bovine serum albumin DSC study. J Therm Anal Calorim. https://doi.org/10.1023/A:1022851809481
20. Assimacopoulos-Jeannet F, Thumelin S, Roche E, et al (1997) Fatty acids rapidly induce the carnitine palmitoyltransferase I gene in the pancreatic β-cell line INS-1. J Biol Chem. https://doi.org/10.1074/jbc.272.3.1659
21. Burns S M, Vetere A, Walpita D, et al (2015) High-throughput luminescent reporter of insulin secretion for discovering regulators of pancreatic beta-cell function. Cell Metab. https://doi.org/10.1016/j.cmet.2014.12.010
22. Eguchi K, Manabe I, Oishi-Tanaka Y, et al (2012) Saturated fatty acid and TLR signaling link 13 cell dysfunction and islet inflammation. Cell Metab. https://doi.org/10.1016/j.cmet.2012.01.023
23. Itoh Y, Kawamata Y, Harada M, et al (2003) Free fatty acids regulate insulin secretion from pancreatic 13 cells through GPR40. Nature. https://doi.org/10.1038/nature01478
24. Picelli S, Faridani O R, Bjorklund A K, et al (2014) Full-length RNA-seq from single cells using Smart-seq2. Nat Protoc. https://doi.org/10.1038/nprot.2014.006
25. Langfelder P, Horvath S (2008) WGCNA: An R package for weighted correlation network analysis. BMC Bioinformatics. https://doi.org/10.1186/1471-2105-9-559
26. Paulovich A, Mesirov J P, Tamayo P, et al (2005) Gene set enrichment analysis: A knowledge-based approach for interpreting genome-wide expression profiles. Proc Natl Acad Sci. https://doi.org/10.1073/pnas.0506580102
27. Liberzon A, Subramanian A, Pinchback R, et al (2011) Molecular signatures database (MSigDB) 3.0. Bioinformatics. https://doi.org/10.1093/bioinformatics/btr260
28. Hotamisligil G S (2017) Inflammation, metaflammation and immunometabolic disorders. Nature
29. Baker R G, Hayden M S, Ghosh S (2011) NF-κB, inflammation, and metabolic disease. Cell Metab.
30. Eguchi K, Manabe I, Oishi-Tanaka Y, et al (2012) Saturated fatty acid and TLR signaling link 13 cell dysfunction and islet inflammation. Cell Metab. https://doi.org/10.1016/j.jallcom.2018.09.383
31. Hotamisligil G S (2010) Endoplasmic Reticulum Stress and the Inflammatory Basis of Metabolic Disease. Cell
32. Fu S, Yang L, Li P, et al (2011) Aberrant lipid metabolism disrupts calcium homeostasis causing liver endoplasmic reticulum stress in obesity. Nature. https://doi.org/10.1038/nature09968
33. Marmugi A, Parnis J, Chen X, et al (2016) Sorcin links pancreatic β-cell lipotoxicity to ER Ca2+ stores. Diabetes. https://doi.org/10.2337/db15-1334
34. Han J, Back S H, Hur J, et al (2013) ER-stress-induced transcriptional regulation increases protein synthesis leading to cell death. Nat Cell Biol. https://doi.org/10.1038/ncb2738
35. Hayden M S, Ghosh S (2008) Shared Principles in NF-κB Signaling. Cell
36. Thomsen S K, Raimondo A, Hastoy B, et al (2018) Type 2 diabetes risk alleles in PAM impact insulin release from human pancreatic β-cells. Nat Genet. https://doi.org/10.1038/s41588-018-0173-1
37. Udler M S, Kim J, von Grotthuss M, et al (2018) Type 2 diabetes genetic loci informed by multi-trait associations point to disease mechanisms and subtypes: A soft clustering analysis. PLoS Med. https://doi.org/10.1371/journal.pmed.1002654
38. Flannick J, Thorleifsson G, Beer N L, et al (2014) Loss-of-function mutations in SLC30A8 protect against type 2 diabetes. Nat Genet. https://doi.org/10.1038/ng.2915
39. Pasquali L, Gaulton K J, Rodriguez-Segui S A, et al (2014) Pancreatic islet enhancer clusters enriched in type 2 diabetes risk-associated variants. Nat Genet. https://doi.org/10.1038/ng.2870
40. de Leeuw C A, Mooij J M, Heskes T, Posthuma D (2015) MAGMA: Generalized Gene-Set Analysis of GWAS Data. PLoS Comput Biol. https://doi.org/10.1371/journal.pcbi.1004219
41. Ripke S, Neale B M, Corvin A, et al (2014) Biological insights from 108 schizophrenia-associated genetic loci. Nature. https://doi.org/10.1038/nature13595
42. Chimienti F, Devergnas S, Pattou F, et al (2006) In vivo expression and functional characterization of the zinc transporter ZnT8 in glucose-induced insulin secretion. J Cell Sci. https://doi.org/10.1242/jcs.03164
43. Rutter G A (2010) Think zinc: New roles for zinc in the control of insulin secretion. Islets
44. Nicolson T J, Bellomo E A, Wijesekara N, et al (2009) Insulin storage and glucose homeostasis in mice null for the granule zinc transporter ZnT8 and studies of the type 2 diabetes-associated variants. Diabetes. https://doi.org/10.2337/db09-0551
45. Kleiner S, Gomez D, Megra B, et al (2018) Mice harboring the human SLC30A8 R138X loss-of-function mutation have increased insulin secretory capacity. Proc Natl Acad Sci. https://doi.org/10.1073/pnas.1721418115
46. Zhang N, Kumar M, Xu G, et al (2006) Activin receptor-like kinase 7 induces apoptosis of pancreatic beta cells and beta cell lines. Diabetologia. https://doi.org/10.1007/s00125-005-0095-1
47. Bertolino P, Holmberg R, Reissmann E, et al (2008) Activin B receptor ALK7 is a negative regulator of pancreatic-cell function. Proc Natl Acad Sci. https://doi.org/10.1073/pnas.0801285105
48. Khera A V., Chaffin M, Aragam K G, et al (2018) Genome-wide polygenic scores for common diseases identify individuals with risk equivalent to monogenic mutations. Nat. Genet.
49. Paynter N P, Balasubramanian R, Giulianini F, et al (2018) Metabolic predictors of incident coronary heart disease in women. Circulation. https://doi.org/10.1161/CIRCULATIONAHA.117.029468
50. Dobin A, Davis C A, Schlesinger F, et al (2013) STAR: Ultrafast universal RNA-seq aligner. Bioinformatics. https://doi.org/10.1093/bioinformatics/bts635
51. Anders S, Pyl P T, Huber W (2015) HTSeq-A Python framework to work with high-throughput sequencing data. Bioinformatics. https://doi.org/10.1093/bioinformatics/btu638
52. Deluca D S, Levin J Z, Sivachenko A, et al (2012) RNA-SeQC: RNA-seq metrics for quality control and process optimization. Bioinformatics. https://doi.org/10.1093/bioinformatics/bts196
53. Love M I, Huber W, Anders S (2014) Moderated estimation of fold change and dispersion for RNA-seq data with DESeq2. Genome Biol. https://doi.org/10.1186/s13059-014-0550-8
54. Leek J T, Johnson W E, Parker H S, et al (2012) The SVA package for removing batch effects and other unwanted variation in high-throughput experiments. Bioinformatics. https://doi.org/10.1093/bioinformatics/bts034
55. Langfelder P, Zhang B, Horvath S (2008) Defining clusters from a hierarchical cluster tree: The Dynamic Tree Cut package for R. Bioinformatics. https://doi.org/10.1093/bioinformatics/btm563

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

1. A method for producing a bovine serum albumin (BSA)-conjugated free fatty acid (FFA) crystal, the method comprising:
a) providing a FFA dissolved in a solvent;
b) transferring the FFA to a well of a plate, wherein the plate well comprises a BSA solution, thereby forming a FFA-BSA solution;
c) incubating the FFA-BSA solution for a duration of time and under conditions suitable to conjugate the FFA to the BSA; and
d) drying the FFA-BSA solution to form a FFA-BSA crystal,
thereby producing a BSA-conjugated free fatty acid (FFA) crystal.

2. The method of claim 1, wherein the solvent is selected from the group consisting of DMSO and ethanol.

3. The method of claim 1, wherein the BSA solution comprises ddH₂O.

4. The method of claim 1, wherein the FFA-BSA solution has a FFA:BSA concentration ratio of approximately 6.67:1, optionally wherein the FFA concentration in the FFA-BSA solution is approximately 500 μM.

5. The method of claim 1, wherein the FFA-BSA solution is incubated for 12-48 hours, optionally about 24 hours, optionally at about 37° C.

6. The method of claim 1, wherein drying of the FFA-BSA solution in step (d) is performed with a high-throughput evaporator.

7. The method of claim 1, wherein the FFA-BSA crystal formed in step (d) is free of the solvent.

8. The method of claim 1, wherein drying of the FFA-BSA solution is performed under vacuum, optionally for a duration of approximately 6-24 hours, optionally approximately 12 hours, optionally at about 37° C., optionally wherein the drying step further comprises centrifugation, optionally at about 400 g.

9. The method of claim 1, further comprising resuspending the FFA-BSA crystal in cell culture media, thereby creating a resuspended FFA-BSA solution, optionally wherein the cell culture media is pancreatic beta cell culture media (optionally MIN6 cell culture media), endothelial cell culture media, hepatocyte cell culture media, macrophage cell culture media, skeletal muscle cell culture media, or adipocyte cell culture media.

10. The method of claim 9, further comprising filtering the resuspended FFA-BSA solution through a filter, optionally wherein the filter has an approximately 0.45 μm pore size, optionally wherein the filter is a spin filter.

11. The method of claim 10, wherein the resuspended FFA-BSA solution is filtered into a well of an array plate, optionally a microwell of a 384 well microarray plate.

12. The method of claim 1, wherein the method is repeated to produce an array of BSA-conjugated FFA crystals (optionally wherein said array of BSA-conjugated FFA crystals is produced by drying with a high-throughput evaporator in step (d)) and/or resuspended FFA-BSA solutions.

13. The method of claim 12, wherein preparation of the array of BSA-conjugated FFA crystals and/or resuspended FFA-BSA solutions is performed in parallel, optionally wherein said array of BSA-conjugated FFA crystals is produced by drying with a high-throughput evaporator in step (d).

14. The method of claim 9, further comprising contacting the resuspended FFA-BSA solution(s) with a cell or array of cells, optionally wherein the cell or array of cells is a pancreatic beta cell or array of cells (optionally a MIN6 cell or array of cells), an endothelial cell or array of cells, a hepatocyte cell or array of cells, a macrophage cell or array of cells, a skeletal muscle cell or array of cells, or an adipocyte cell or array of cells.

15. The method of claim 14, wherein the FFA is delivered into the cell, optionally wherein the FFA is incorporated into lipids of the cell.

16. A composition comprising an array of FFA-BSA crystals or FFA-BSA solutions, wherein each element of the array comprises a single FFA and the array comprises two or more distinct FFAs.

17. The composition of claim 16, wherein:
the array comprises five or more FFAs selected from Table 1, optionally ten or more FFAs selected from Table 1, optionally twenty or more FFAs selected from Table 1, optionally 30 or more FFAs selected from Table 1, optionally 40 or more FFAs selected from Table 1, optionally 50 or more FFAs selected from Table 1, optionally all FFAs of Table 1;
the array is assembled in wells of a plate, optionally in wells of a 96 well plate or in microwells of a 384 well microplate, and/or
each element of the array further comprises cells or tissues in culture, optionally wherein the cells or tissues in culture are selected from the group consisting of pancreatic beta cells (optionally MIN6 cells) or pancreatic tissue, endothelial cells or endothelial tissue, hepatocyte cells or liver tissue, macrophage cells, skeletal muscle cells or skeletal muscle tissue, and adipocyte cells or fat tissue and/or each cell or tissue in culture comprises a distinct FFA that has been incorporated into lipids of the cell or tissue.

18-21. (canceled)

22. A composition comprising an array of FFA-BSA crystals or FFA-BSA solutions, wherein each element of the array comprises a single FFA and the array comprises two or more distinct FFAs, wherein the composition is prepared by the method of claim 11.

23. A method selected from the group consisting of:
A method for identifying a lipotoxic FFA, the method comprising:
a) providing a composition comprising an array of FFA-BSA crystals or FFA-BSA solutions, wherein each element of the array comprises a single FFA and the array comprises two or more distinct FFAs;
b) contacting the composition with cells or tissues in culture;
c) assessing levels of cell death and/or biomarkers of apoptosis and/or lipotoxicity in the cells or tissues in culture contacted with the composition, as compared to an appropriate control,
thereby identifying a lipotoxic FFA;
A method for identifying a lipotoxic FFA disease or disorder-associated gene, the method comprising:
a) providing a composition comprising an array of FFA-BSA crystals or FFA-BSA solutions, wherein each element of the array comprises a single FFA and the array comprises two or more distinct FFAs;
b) contacting the composition with cells or tissues in culture;
c) measuring the transcriptome of the cells or tissues in culture and identifying transcripts that are differentially expressed between lipotoxic FFAs and non-lipotoxic FFAs;
d) producing a rank ordered list of genes that encode for the transcripts identified as most differentially expressed between lipotoxic FFAs and non-lipotoxic FFAs;
e) comparing the rank ordered list of genes of step (d) with a rank ordered list of genes identified as most genetically associated with the lipotoxic FFA disease or disorder; and
f) identifying a gene that both (i) encodes for a transcript identified as highly differentially expressed between lipotoxic FFAs and non-lipotoxic FFAs and (ii) is highly genetically associated with the lipotoxic FFA disease,
thereby identifying a lipotoxic FFA disease or disorder-associated gene; and
A method for treating or preventing a lipotoxic FFA disease or disorder in a subject having or at risk of developing the lipotoxic FFA disease or disorder, the method comprising administering to the subject an agent capable of modulating expression of a gene selected from the group consisting of MACF1, HMG20A, QPCTL, NUCB2, SSR1, ATG16L2, ADCK5, ADCY5, CPSF1, PMPCA, ALDOA, FANCC, PRC1, SPRED2, ACVR1C, CMIP, DCAF7, MAPK3, NFIX, HAPLN4, CYHR1 and C9orf3, thereby treating or preventing the lipotoxic FFA disease or disorder in the subject.

24. (canceled)

25. The method of claim 23, wherein:
the lipotoxic FFA disease or disorder is selected from the group consisting of type 2 diabetes (T2D), obesity, cardiovascular diseases (CVD), non-alcoholic fatty liver disease (NAFLD), obesity-mediated inflammation/metaflammation and insulin resistance;
the cells or tissues in culture are selected from the group consisting of pancreatic beta cells (optionally MING cells) or pancreatic tissue, endothelial cells or endothelial tissue, hepatocyte cells or liver tissue, macrophage cells, skeletal muscle cells or skeletal muscle tissue, and adipocyte cells or fat tissue;
the lipotoxic FFAs are selected from the group consisting of:

| | |
|---|---|
| CC\C═C/C\C═C/C\C═C/CCCCCCCCCCCC(O)═O | 13(Z),16(Z),19(Z)-Docosatrienoic acid |
| CCCCCCCC\C═C/CCCCCCCCCC(O)═O | 11(Z)-Eicosenoic acid |
| CCCCCCCC\C═C/CCCCCCCCCCC(O)═O | 12(Z) Heneicosenoic acid |
| CCCCCCCC\C═C/CCCCCCCCCCCC(O)═O | 13(Z)-Docosenoic acid |
| CCCCCCCC\C═C/CCCCCCCCCCCCC(O)═O | 14(Z)-Tricosenoic acid |
| CCCCCCCC\C═C/CCCCCCCCCCCCCC(O)═O | 15(Z)-Tetracosenoic acid |
| CCCCCCCC\C═C\CCCCCCCCC(O)═O | 10(E)-Nonadecenoic acid |
| CCCCCCCC\C═C\CCCCCCCCCC(O)═O | 11(E)-Eicosenoic acid |
| CCCCCCCC\C═C\CCCCCCCCCCCCC(O)═O | 14(E)-Tricosenoic acid |
| CCCCCCCCCCC\C═C/CCCCCC(O)═O | 7(Z)-Nonadecenoic acid |
| CCCCCCCCCCC\C═C\CCCCC(O)═O | 6(E)-Octadecenoic acid |
| CCCCCCCCCCC\C═C\CCCCCC(O)═O | 7(E)-Nonadecenoic acid |
| CCCCCCCCCCCC(O)═O | Dodecanoic acid |
| CCCCCCCCCCCCCC\C═C/CCCC(O)═O | 5(Z)-Eicosenoic acid |
| CCCCCCCCCCCCCCC(O)═O | Pentadecanoic acid |
| CCCCCCCCCCCCCCCC(O)═O | Hexadecanoic acid |
| CCCCCCCCCCCCCCCCC(O)═O | Heptadecanoic acid |
| CCCCCCCCCCCCCCCCCC(O)═O | Octadecanoic acid |
| CCCCCCCCCCCCCCCCCCC(O)═O | Nonadecanoic acid |
| CCCCCCCCCCCCCCCCCCCC(O)═O | Eicosanoic acid; |

the non-lipotoxic FFAs are selected from the group consisting of:

| | |
|---|---|
| CCCCCCCC\C═C\CCCCCCCCCCCC(O)═O | 13(E)-Docosenoic acid |
| CCCCCCCCCC(O)═O | Decanoic acid |
| CCCCCCCCCCC(O)═O | Undecanoic acid |
| CCCCCCCCCCCCC(O)═O | Tridecanoic acid |
| CCCCCCCCCCCCCC(O)═O | Tetradecanoic acid |
| CCCCCCCCCCCCCCCCCCCCC(O)═O | Heneicosanoic acid |
| C═CCCCCCCCCC(O)═O | 10-Undecenoic acid |
| C═CCCCCCCCCCC(O)═O | 11-Dodecenoic acid |
| C═CCCCCCCCCCCC(O)═O | 12-Tridecenoic acid |
| CCCC\C═C\CCCCCCCCC(O)═O | 10(E)-Pentadecenoic acid |
| CCCCCC\C═C/CCCCCCCC(O)═O | 9(Z)-Hexadecenoic acid |
| CCCCCC\C═C/CCCCCCCCCC(O)═O | 11(Z)-Octadecenoic acid |
| CCCCCCCC\C═C/C\C═C/C\C═C/CCCC(O)═O | 5(Z),8(Z),11(Z)-Eicosatrienoic Acid |
| CCCCCCCC\C═C/CCCCCCCC(O)═O | 9(Z)-Octadecenoic acid |
| CCCCCCCCCCC\C═C/C\C═C/CCCC(O)═O | 5(Z),8(Z)-Eicosadienoic acid |
| CCCCCCCCCCC\C═C/CCCCC(O)═O | 6(Z)-Octadecenoic acid |
| CCCCCCCCCCC\C═C/CCCCCCC(O)═O | 8(Z)-Eicosenoic acid |
| CCCC\C═C/CCCCCCCC(O)═O | 9(Z)-Tetradecenoic acid |
| CCCC\C═C/CCCCCCCCC(O)═O | 10(Z)-Pentadecenoic acid |
| CCCC\C═C\CCCCCCCC(O)═O | 9(E)-Tetradecenoic acid |
| CCCCC\C═C/C\C═C/CCCCCCCCC(O)═O | 10(Z),13(Z)-Nonadecadienoic acid |
| CCCCC\C═C/C\C═C/CCCCCCCCCC(O)═O | 11(Z),14(Z)-Eicosadienoic acid |
| CCCCC\C═C\C\C═C\CCCCCCCC(O)═O | 9(E),12(E)-Octadecadienoic acid |
| CCCCCC\C═C/CCCCCCCCC(O)═O | 10(Z)-Heptadecenoic acid |
| CCCCCC\C═C\CCCCCCCC(O)═O | 9(E)-Hexadecenoic acid |
| CCCCCC\C═C\CCCCCCCCC(O)═O | 10(E)-Heptadecenoic acid |
| CCCCCC\C═C\CCCCCCCCCC(O)═O | 11(E)-Octadecenoic acid |
| CCCCCCCC\C═C/CCCCCCCCC(O)═O | 10(Z)-Nonadecenoic acid |
| CCCCCCCC\C═C\CCCCCCCC(O)═O | 9(E)-Octadecenoic acid |
| CC\C═C/C\C═C/C\C═C/C\C═C/C\C═C/C\C═C/CCC(O)═O | 4(Z),7(Z),10(Z),13(Z),16(Z),19(Z)- |
| | Docosahexaenoic acid |
| CC\C═C/C\C═C/C\C═C/C\C═C/C\C═C/CCCC(O)═O | 5(Z),8(Z),11(Z),14(Z),17(Z)- |
| | Eicosapentaenoic acid |
| CC\C═C/C\C═C/C\C═C/C\C═C/C\C═C/CCCCCC(O)═O | 7(Z),10(Z),13(Z),16(Z),19(Z)- |
| | Docosapentaenoic acid |
| CC\C═C/C\C═C/C\C═C/C\C═C/CCCCC(O)═O | 6(Z),9(Z),12(Z),15(Z)-Octadecatetraenoic |
| | acid |
| CC\C═C/C\C═C/C\C═C/CCCCCCCC(O)═O | 9(Z),12(Z),15(Z)-Octadecatrienoic Acid |
| CC\C═C/C\C═C/C\C═C/CCCCCCCCCC(O)═O | 11(Z),14(Z),17(Z)-Eicosatrienoic Acid |
| CCCCC\C═C/C\C═C/C\C═C/C\C═C/CCCC(O)═O | 5(Z),8(Z),11(Z),14(Z)-Eicosatetraenoic |
| | Acid |
| CCCCC\C═C/C\C═C/C\C═C/C\C═C/CCCCCC(O)═O | 7(Z),10(Z),13(Z),16(Z)-Ocosatetraenoic |
| | Acid |
| CCCCC\C═C/C\C═C/C\C═C/CCCCC(O)═O | 6(Z),9(Z),12(Z)-Octadecatrienoic Acid |
| CCCCC\C═C/C\C═C/C\C═C/CCCCCCC(O)═O | 8(Z),11(Z),14(Z)-Eicosatrienoic Acid |
| CCCCC\C═C/C\C═C/CCCCCCCC(O)═O | 9(Z),12(Z)-Octadecadienoic acid |
| CCCCCC\C═C\C═C/CCCCCCCC(O)═O | 9(Z),11(E)-octadecadienoic acid; |

comparing step (e) comprises comparing a rank ordered list of 500 genes that encode for transcripts identified as the most differentially expressed between lipotoxic FFAs and non-lipotoxic FFAs with a rank ordered list of genes identified as most genetically associated with the lipotoxic FFA disease or disorder;
comparing step (e) comprises comparing a rank ordered list of genes that encode for transcripts identified as the most differentially expressed between lipotoxic FFAs and non-lipotoxic FFAs with a rank ordered list of genes identified as in the top 5% or top 10% of genes most genetically associated with the lipotoxic FFA disease or disorder;
the agent is a nucleic acid that specifically targets the gene;
the agent is a small molecule; and/or
the agent is selected from the group consisting of PQ912, 2-pyridine-3-yl-methylene-indan-1,3-dione (PRT4165), BC1753, PD98059, arphamenine A, and TDZD-8.

26-34. (canceled)
